SEQUENCE LISTING (235 sequences)

SEQ ID NO: 1 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 2 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgacatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccccctcttctctgggcacccagacctacatctgcaatgtgaaccacccccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 4 - protein, 472 aa, synthetic construct
MDWTWRILFLVAAATGTHAQMQLVQSGPEVKKPGTSVKVSCKASGFTFMSSAVQWVRQARGQRLEWIGWIDIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPPSSLGTQTYICNVNHPPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgcccggcagcggcaataccaactacgcccagaagtttcaggagcgggtgaccatcacaagagacatgagcaccagcaccgcctatatggaactgtccagcctgaccagtgaagataccgccgtgtactactgtgccgccccatactgcagcagcatcagctgcaacgacggcttcgacatctggacccaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 6 - protein, 472 aa, synthetic construct
MDWTWRILFLVAAATGTHAQMQLVQSGPEVKKPGTSVKVSCKASGFTFMSSAVQWVRQARGQRLEWIGWIVPGSGNTNYAQKFQERVTITRDMSTSTAYMELSSLTSEDTAVYYCAAPYCSSISCNDGFDIWTQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgacatcggcagcggcaataccaactacgcccagaagtttcaggagcgggtgaccatcacaagagaccccagcaccagcaccgcctatatggaactgtccagcctgaccagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 8 - protein, 472 aa, synthetic construct
MDWTWRILFLVAAATGTHAQMQLVQSGPEVKKPGTSVKVSCKASGFTFMSSAVQWVRQARGQRLEWIGWIDIGSGNTNYAQKFQERVTITRDPSTSTAYMELSSLTSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacagccagaagtttcaggagcgggtgaccatcacaagagaccccagcaccagcaccgcctatatggaactgtccagcctgaccagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacccccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 10 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYSQKFQERVTITRDPSTSTAYMELSSLTSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHPPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgacatcggcagcggcaataccaactacagccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggacccaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccccctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 12 - protein, 472 aa, synthetic construct
MDWTWRILFLVAAATGTHAQMQLVQSGPEVKKPGTSVKVSCKASGFTFMSSAVQWVRQARGQRLEWIGWIDIGSGNTNYSQKFQERVTITRDMSTSTAYMELSSLRSEDTAVYYCAAPYCSSISCNDGFDIWTQGTMVTVSSASTKGPSVFPLAPSSKSTSGGTAALGCLVKDYFPEPVTVSWNSGALTSGVHTFPAVLQSSGLYSLSSVVTVPPSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgcccggcagcggcaataccaactacgcccagaagtttcaggagcgggtgaccatcacaagagaccccagcaccagcaccgcctatatggaactgtccagcctgagaagtgaagataccgccgtgtactactgtgccgccccatactgcagcagcatcagctgcaacgacggcttcgacatctggacccaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccccctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 14 - protein, 472 aa, synthetic construct
MDWTWRILFLVAAATGTHAQMQLVQSGPEVKKPGTSVKVSCKASGFTFMSSAVQWVRQARGQRLEWIGWIVPGSGNTNYAQKFQERVTITRDPSTSTAYMELSSLRSEDTAVYYCAAPYCSSISCNDGFDIWTQGTMVTVSSASTKGPSVFPLAPSSKSTSGGTAALGCLVKDYFPEPVTVSWNSGALTSGVHTFPAVLQSSGLYSLSSVVTVPPSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgacatcggcagcggcaataccaactacgcccagaagtttcaggagcgggtgaccatcacaagagaccccagcaccagcaccgcctatatggaactgtccagcctgaccagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccccctcttctctgggcacccagacctacatctgcaatgtgaaccacccccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 16 - protein, 472 aa, synthetic construct
MDWTWRILFLVAAATGTHAQMQLVQSGPEVKKPGTSVKVSCKASGFTFMSSAVQWVRQARGQRLEWIGWIDIGSGNTNYAQKFQERVTITRDPSTSTAYMELSSLTSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPPSSLGTQTYICNVNHPPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgcccggcagcggcaataccaactacagccagaagtttcaggagcgggtgaccatcacaagagaccccagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccccctcttctctgggcacccagacctacatctgcaatgtgaaccacccccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 18 - protein, 472 aa, synthetic construct
MDWTWRILFLVAAATGTHAQMQLVQSGPEVKKPGTSVKVSCKASGFTFMSSAVQWVRQARGQRLEWIGWIVPGSGNTNYSQKFQERVTITRDPSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPPSSLGTQTYICNVNHPPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 20 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 21 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcagccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 22 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFSQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 23 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacacccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 24 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHTPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctaacacctccggcgtgcacacattccccgctgttctgcagtcttctggactgtacagcctgtcttctgtggtgacagtccccagctcttctctgggcacccagacctacatctgcaatgtgaaccacacccctagcaacaccaaaaacgataccaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 26 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSLGTQTYICNVNHTPSNTKNDTKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgaactcttctggactgtacagcctgtcttctgtggtgacagtccccagctcttctaacggcacccagacctacatctgcaatgtgaaccacacccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 28 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLNSSGLYSLSSVVTVPSSSNGTQTYICNVNHTPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctaacacctccggcgtgcacacattccccgctgttctgcagtcttctggactgtacagcctgtcttctgtggtgacagtccccagctcttctaacggcacccagacctacatctgcaatgtgagccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 30 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSNGTQTYICNVSHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 1422 bp, synthetic construct
  source 1..1422: mol_type = other DNA
atggactggacctggagaatcctgttcctggtggctgctgctacaggcacccacgcccaaatgcagctggtccagagcggccctgaggtgaaaaagcctggcacatctgtgaaggtgtcctgcaaggccagcgggtttacattcatgagctctgccgtgcagtgggtgcggcaggccagaggccagagactggaatggatcggctggatcgtgatcggcagcggcaataccaactacgcccagaagtttcaggagcgggtgaccatcacaagagacatgagcaccagcaccgcctatatggaactgtccagcctgagaagtgaagataccgccgtgtactactgtgccgccccatactgcagcagcatcagctgcaacgacggcttcgacatctggggacaaggcaccatggtgaccgtgtcaagcgcctctaccaagggccccagcgtgttccctcttgctccttcctccaaaagcaccagcggaggcaccgccgccctgggatgtctggttaaagactacttccccgaacctgtgacagtgtcttggaacagcggtgctaacacctccggcgtgcacacattccccgctgttctgcagtcttctggactgtacagcctgtcttctgtggtgacagtccccagctcttctaacggcacccagacctacatctgcaatgtgaaccacacccctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 32 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSNGTQTYICNVNHTPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - DNA, 714 bp, synthetic construct; identical to SEQ ID NO: 19
  source 1..714: mol_type = other DNA

SEQ ID NO: 34 - protein, 236 aa, synthetic construct; identical to SEQ ID NO: 20

SEQ ID NO: 35 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcaacgtgacacaccagggcctgtcaagccccgtgaccaaaagcttcaacagaggagaatgttgatga

SEQ ID NO: 36 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACNVTHQGLSSPVTKSFNRGEC

SEQ ID NO: 37 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgaaccacagcggcctgtcaagccccgtgaccaaaagcttcaacagaggagaatgttgatga

SEQ ID NO: 38 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVNHSGLSSPVTKSFNRGEC

SEQ ID NO: 39 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcagaacagcaccgactccacctacagcctgagcaacaccctgactctgtctaaggccgactatgagaagcacaaggtctatgcctgcaacgtgacacaccagggcctgtcaagccccgtgaccaaaagcttcaacagaggagaatgttgatga

SEQ ID NO: 40 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSNTLTLSKADYEKHKVYACNVTHQGLSSPVTKSFNRGEC

SEQ ID NO: 41 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcagaacagcaccgactccacctacagcctgagcaacaccctgactctgtctaaggccgactatgagaagcacaaggtctatgcctgcgaggtgaaccacagcggcctgtcaagccccgtgaccaaaagcttcaacagaggagaatgttgatga

SEQ ID NO: 42 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSNTLTLSKADYEKHKVYACEVNHSGLSSPVTKSFNRGEC

SEQ ID NO: 43 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcagaacagcaccgactccacctacagcctgagcaacaccctgaacctgtctaaggccgactatgagaagcacaaggtctatgcctgcgaggtgaaccacagcggcctgtcaagccccgtgaccaaaagcttcaacagaggagaatgttgatga

SEQ ID NO: 44 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSNTLNLSKADYEKHKVYACEVNHSGLSSPVTKSFNRGEC

SEQ ID NO: 45 - DNA, 1446 bp, synthetic construct
  source 1..1446: mol_type = other DNA
atggactggacctggagaatcctgttcctggtggctgctgctacaggcacccacgcccagggcagactgttccagagtggcgcagaggtgaagagaccaggagcaagcgtgaggattagttgtcgggcagatgatgacccttacaccgacgatgacaccttcacaaagtactggacacactggatcaggcaggcaccaggacagcgccctgagtggctgggcgtgatctctccacacttcgccaggcccatctacagctataagtttagggatagactgaccctgacacgcgacagctccctgaccgccgtgtacctggagctgaagggcctgcagcctgatgacagcggcatctatttctgcgccagggaccccttcggcgacagggccccacactataactaccacatggatgtctggggaggaggaaccgcagtcattgtctcttctgcctctaccaagggccccagcgtgttccctcttgctccttcctccaaaagcaccagcggaggcaccgccgccctgggatgtctggttaaagactacttccccgaacctgtgacagtgtcttggaacagcggtgctctgacctccggcgtgcacacattccccgctgttctgcagtcttctggactgtacagcctgtcttctgtggtgacagtccccagctcttctctgggcacccagacctacatctgcaatgtgaaccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 46 - protein, 480 aa, synthetic construct
MDWTWRILFLVAAATGTHAQGRLFQSGAEVKRPGASVRISCRADDDPYTDDDTFTKYWTHWIRQAPGQRPEWLGVISPHFARPIYSYKFRDRLTLTRDSSLTAVYLELKGLQPDDSGIYFCARDPFGDRAPHYNYHMDVWGGGTAVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - DNA, 1446 bp, synthetic construct
  source 1..1446: mol_type = other DNA
atggactggacctggagaatcctgttcctggtggctgctgctacaggcacccacgcccagggcagactgttccagagtggcgcagaggtgaagagaccaggagcaagcgtgaggattagttgtcgggcagatgatgacccttacaccgacgatgacaccttcacaaagtactggacacactggatcaggcaggcaccaggacagcgccctgagtggctgggcgtgatctctccacacttcgccaggcccatctacagctataagtttagggatagactgaccctgacacgcgacagctccctgaccgccgtgtacctggagctgaagggcctgcagcctgatgacagcggcatctatttctgcgccagggaccccttcggcgacagggccccacactataactaccacatggatgtctggggaggaggaaccgcagtcattgtctcttctgcctctaccaagggccccagcgtgttccctcttgctccttcctccaaaagcaccagcggaggcaccgccgccctgggatgtctggttaaagactacttccccgaacctgtgacagtgtcttggaacagcggtgctaacacctccggcgtgcacacattccccgctgttctgcagtcttctggactgtacagcctgtcttctgtggtgacagtccccagctcttctaacggcacccagacctacatctgcaatgtgagccacaagcctagcaacaccaaagttgataagaaggtggagcctaagagctgcgataaaacacatacatgtcctccgtgccctgctcctgagctgctgggcggcccaagcgtgttcctttttcctcctaagcccaaggacaccctgatgatcagtagaaccccagaggtgacctgtgtggtggtggacgtgtcccacgaggaccctgaggtgaagttcaactggtacgtggatggcgtggaagttcataatgccaagacaaaacctagggaagagcagtacaacagcacctatagagtggtctccgtcctgaccgtgctgcaccaggactggctgaacggcaaggaatacaagtgcaaggtgagcaacaaggccctgcctgcccctatcgagaagaccattagcaaggctaagggccagcctagagagccccaggtgtacaccctgccccccagcagagatgagctgacaaagaaccaggtgagtctgacatgcctggtcaaaggcttctacccatccgatatcgccgtggagtgggagagcaatggccagcctgaaaacaactacaagaccacccctcctgtgctggacagcgacggcagcttcttcctgtacagcaagctgacagtggacaagtcccggtggcaacagggcaacgtgttcagctgtagcgtgatgcacgaggccctgcacaaccactacacacagaaaagcctctccctgagccctggaaagtgatga

SEQ ID NO: 48 - protein, 480 aa, synthetic construct
MDWTWRILFLVAAATGTHAQGRLFQSGAEVKRPGASVRISCRADDDPYTDDDTFTKYWTHWIRQAPGQRPEWLGVISPHFARPIYSYKFRDRLTLTRDSSLTAVYLELKGLQPDDSGIYFCARDPFGDRAPHYNYHMDVWGGGTAVIVSSASTKGPSVFPLAPSSKSTSGGTAALGCLVKDYFPEPVTVSWNSGANTSGVHTFPAVLQSSGLYSLSSVVTVPSSSNGTQTYICNVSHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 711 bp, synthetic construct
  source 1..711: mol_type = other DNA
atggtgctgcaaacacaggtgttcattagcctgctcctgtggatctccggcgcctacggcgaggtggtgctgacccagagccccgccattctgagcgtgagcccaggagaccgagtgattctgagttgtagagcaagccagggactggacagctcccacctggcctggtaccggttcaagcgcggccagatccccaccctggtgatctttggcacatctaacagggccagaggcacccctgacaggttctctggcagcggctccggagcagacttcaccctgacaatcagccgggtggagcccgaggatttcgccacatactattgtcagagatacggaggcacacccatcacctttggcggcggcacaaccctggataaaaaacggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 50 - protein, 235 aa, synthetic construct
MVLQTQVFISLLLWISGAYGEVVLTQSPAILSVSPGDRVILSCRASQGLDSSHLAWYRFKRGQIPTLVIFGTSNRARGTPDRFSGSGSGADFTLTISRVEPEDFATYYCQRYGGTPITFGGGTTLDKKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 51 - DNA, 711 bp, synthetic construct
  source 1..711: mol_type = other DNA
atggtgctgcaaacacaggtgttcattagcctgctcctgtggatctccggcgcctacggcgaggtggtgctgacccagagccccgccattctgagcgtgagcccaggagaccgagtgattctgagttgtagagcaagccagggactggacagctcccacctggcctggtaccggttcaagcgcggccagatccccaccctggtgatctttggcacatctaacagggccagaggcacccctgacaggttctctggcagcggctccggagcagacttcaccctgacaatcagccgggtggagcccgaggatttcgccacatactattgtcagagatacggaggcacacccatcacctttggcggcggcacaaccctggataaaaaacggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgaaccacagcggcctgtcaagccccgtgaccaaaagcttcaacagaggagaatgttgatga

SEQ ID NO: 52 - protein, 235 aa, synthetic construct
MVLQTQVFISLLLWISGAYGEVVLTQSPAILSVSPGDRVILSCRASQGLDSSHLAWYRFKRGQIPTLVIFGTSNRARGTPDRFSGSGSGADFTLTISRVEPEDFATYYCQRYGGTPITFGGGTTLDKKRTVAAPSVFIFPPSDEQLKSGTASVVCLLNNFYPREAKVQWKVDNALQSGNSQESVTEQDSKDSTYSLSNTLTLSKADYEKHKVYACEVNHSGLSSPVTKSFNRGEC

SEQ ID NO: 53 - DNA, 711 bp, synthetic construct
  source 1..711: mol_type = other DNA
atggtgctgcaaacacaggtgttcattagcctgctcctgtggatctccggcgcctacggcgaggtggtgctgacccagagccccgccattctgagcgtgagcccaggagaccgagtgattctgagttgtagagcaagccagggactggacagctcccacctggcctggtaccggttcaagcgcggccagatccccaccctggtgatctttggcacatctaacagggccagaggcacccctgacaggttctctggcagcggctccggagcagacttcaccctgacaatcagccgggtggagcccgaggatttcgccacatactattgtcagagatacggaggcacacccatcacctttggcggcggcacaaccctggataaaaaacggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcagaacagcaccgactccacctacagcctgagcaacaccctgaacctgtctaaggccgactatgagaagcacaaggtctatgcctgcgaggtgaaccacagcggcctgtcaagccccgtgaccaaaagcttcaacagaggagaatgttgatga

SEQ ID NO: 54 - protein, 235 aa, synthetic construct
MVLQTQVFISLLLWISGAYGEVVLTQSPAILSVSPGDRVILSCRASQGLDSSHLAWYRFKRGQIPTLVIFGTSNRARGTPDRFSGSGSGADFTLTISRVEPEDFATYYCQRYGGTPITFGGGTTLDKKRTVAAPSVFIFPPSDEQLKSGTASVVCLLNNFYPREAKVQWKVDNALQSGNSQESVTEQNSTDSTYSLSNTLNLSKADYEKHKVYACEVNHSGLSSPVTKSFNRGEC

SEQ ID NO: 55 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 56 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgagccacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 58 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVSHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaccccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 60 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHTPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaagaacgacaccagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 62 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKNDTRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgaactccagctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 64 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVNSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgcagtcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 66 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgaactcctctggcctgtacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 68 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLNSSGLYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggctacagcctgagctccgtggtgaccgtgccatctagctccctgggcacccagacatatatctgtaacgtgaatcacaagccttccaatacaaaggtggacaagagggtggagccaaagtcttgcgataagacccacacatgccctccctgtccagcacctgagctgctgaacggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 70 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGNYSLSSVVTVPSSSLGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccaacggcacccagacatatatctgtaacgtgaatcacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 72 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLQSSGLYSLSSVVTVPSSSNGTQTYICNVNHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgaactccagctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcctgtacagcctgagctccgtggtgaccgtgccatctagctccaacggcacccagacatatatctgtaacgtgagccacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 74 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVNSSSTKGPSVFPLAPSSKSTSGGTAALGCLVKDYFPEPVTVSWNSGALTSGVHTFPAVLQSSGLYSLSSVVTVPSSSNGTQTYICNVSHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgcagtcctctggcctgtacagcctgagctccgtggtgaccgtgccatctagctccctgggcacccagacatatatctgtaacgtgaatcacaccccttccaatacaaagaacgacaccagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 76 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSLGTQTYICNVNHTPSNTKNDTRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgaactcctctggcctgtacagcctgagctccgtggtgaccgtgccatctagctccaacggcacccagacatatatctgtaacgtgaatcacaccccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 78 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGALTSGVHTFPAVLNSSGLYSLSSVVTVPSSSNGTQTYICNVNHTPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgcagtcctctggcctgtacagcctgagctccgtggtgaccgtgccatctagctccaacggcacccagacatatatctgtaacgtgagccacaagccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 80 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSNGTQTYICNVSHKPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgcagtcctctggcctgtacagcctgagctccgtggtgaccgtgccatctagctccaacggcacccagacatatatctgtaacgtgaatcacaccccttccaatacaaaggtggacaagagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 82 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLQSSGLYSLSSVVTVPSSSNGTQTYICNVNHTPSNTKVDKRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgaactccagctctaccaagggcccaagcgtgtttcccctggccccttctagcaagagcacctccggcggcacagccgccctgggctgtctggtgaaggactacttccctgagccagtgacagtgagctggaactccggcgccctgacctccggagtgcacacatttcctgccgtgctgcagtcctctggcaactacagcctgagctccgtggtgaccgtgccatctagctccaacggcacccagacatatatctgtaacgtgagccacaagccttccaatacaaagaacgacaccagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 84 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVNSSSTKGPSVFPLAPSSKSTSGGTAALGCLVKDYFPEPVTVSWNSGALTSGVHTFPAVLQSSGNYSLSSVVTVPSSSNGTQTYICNVSHKPSNTKNDTRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgaactccagctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgcagtcctctggcctgtacagcctgagctccgtggtgaccgtgccatctagctccaacggcacccagacatatatctgtaacgtgaatcacaccccttccaatacaaagaacgacaccagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 86 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVNSSSTKGPSVFPLAPSSKSTSGGTAALGCLVKDYFPEPVTVSWNSGANTSGVHTFPAVLQSSGLYSLSSVVTVPSSSNGTQTYICNVNHTPSNTKNDTRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1422 bp, synthetic construct
  source 1..1422: mol_type = other DNA
atggactggacatggagaatcctgttcctggtcgccgccgcaactggcactcacgctcagatgcagctggtgcagtctggacccgaggtgaagaagcccggcaccagcgtgaaggtgtcctgtaaggcctctggcttcacctttatgagctccgccgtgcagtgggtgaggcaggccagaggccagcggctggagtggatcggatggatcgtgatcggctccggaaacaccaattacgcccagaagttccaggagcgcgtgaccatcacaagggacatgtccacctctacagcctatatggagctgtctagcctgcggtccgaggatacagccgtgtactattgcgccgccccttactgttcctctatcagctgcaacgacggcttcgatatctggggccagggcaccatggtgacagtgagctccgcctctaccaagggcccaagcgtgtttcccctggccccttctagcaagagcacctccggcggcacagccgccctgggctgtctggtgaaggactacttccctgagccagtgacagtgagctggaactccggcgccaacacctccggagtgcacacatttcctgccgtgctgaactcctctggcctgtacagcctgagctccgtggtgaccgtgccatctagctccaacggcacccagacatatatctgtaacgtgagccacaagccttccaatacaaagaacgacaccagggtggagccaaagtcttgcgataagacccacacatgccctccctgtccagcacctgagctgctgggcggcccaagcgtgttcctgtttccacccaagcccaaggacaccctgtacatcaccagggagccagaggtgacatgcgtggtggtggacgtgtcccacgaggaccccgaggtgaagtttaactggtacgtggatggcgtggaggtgcacaatgccaagaccaagcccagagaggagcagtacaattccacctatcgggtggtgtctgtgctgacagtgctgcaccaggactggctgaacggcaaggagtacaagtgtaaggtgtctaataaggccctgccagcccccatcgagaagaccatcagcaaggcaaagggacagccacgcgagccacaggtgtatacactgcctccatctagggaggagatgaccaagaaccaggtgagcctgacatgcctggtgaagggcttctaccccagcgatatcgccgtggagtgggagtccaatggccagcctgagaacaattacaagaccacaccccctgtgctggactctgatggcagcttctttctgtattctaagctgaccgtggacaagagcagatggcagcagggcaacgtgttttcttgtagcgtgatgcacgaggccctgcacaatcactacacccagaagtccctgtctctgagccctggcaagtgataa

SEQ ID NO: 88 - protein, 472 aa, synthetic construct
MDWTWRILFLVAAATGTHAQMQLVQSGPEVKKPGTSVKVSCKASGFTFMSSAVQWVRQARGQRLEWIGWIVIGSGNTNYAQKFQERVTITRDMSTSTAYMELSSLRSEDTAVYYCAAPYCSSISCNDGFDIWGQGTMVTVSSASTKGPSVFPLAPSSKSTSGGTAALGCLVKDYFPEPVTVSWNSGANTSGVHTFPAVLNSSGLYSLSSVVTVPSSSNGTQTYICNVSHKPSNTKNDTRVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagaacaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcgaggtgacccatcaggggctgtcttctccagtgaccaaatccttcaatcgcggggaatgttgataa

SEQ ID NO: 90 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQNKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctagcgagagcgtgaccgagcaggactccaaggattctacatactccctgtctagcaccctgacactgagcaaggccgattacgagaagcacaaggtgtatgcctgcgaggtgacccatcaggggctgtcttctccagtgaccaaatccttcaatcgcggggaatgttgataa

SEQ ID NO: 92 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSSESVTEQDSKDSTYSLSSTLTLSKADYEKHKVYACEVTHQGLSSPVTKSFNRGEC

SEQ ID NO: 93 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcagaactccaccgattctacatactccctgtctagcaccctgacactgagcaaggccgattacgagaagcacaaggtgtatgcctgcgaggtgacccatcaggggctgtcttctccagtgaccaaatccttcaatcgcggggaatgttgataa

SEQ ID NO: 94 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSSTLTLSKADYEKHKVYACEVTHQGLSSPVTKSFNRGEC

SEQ ID NO: 95 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgaacctgagcaaggccgattacgagaagcacaaggtgtatgcctgcgaggtgacccatcaggggctgtcttctccagtgaccaaatccttcaatcgcggggaatgttgataa

SEQ ID NO: 96 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLNLSKADYEKHKVYACEVTHQGLSSPVTKSFNRGEC

SEQ ID NO: 97 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcaacgtgacccatcaggggctgtcttctccagtgaccaaatccttcaatcgcggggaatgttgataa

SEQ ID NO: 98 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACNVTHQGLSSPVTKSFNRGEC

SEQ ID NO: 99 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcgaggtgaaccatagcgggctgtcttctccagtgaccaaatccttcaatcgcggggaatgttgataa

SEQ ID NO: 100 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVNHSGLSSPVTKSFNRGEC

SEQ ID NO: 101 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcagaactccaccgattctacatactccctgtctagcaccctgacactgagcaaggccgattacgagaagcacaaggtgtatgcctgcaacgtgacccatcaggggctgtcttctccagtgaccaaatccttcaatcgcggggaatgttgataa

SEQ ID NO: 102 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSSTLTLSKADYEKHKVYACNVTHQGLSSPVTKSFNRGEC

SEQ ID NO: 103 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcagaactccaccgattctacatactccctgtctagcaccctgacactgagcaaggccgattacgagaagcacaaggtgtatgcctgcgaggtgaaccatagcgggctgtcttctccagtgaccaaatccttcaatcgcggggaatgttgataa

SEQ ID NO: 104 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSSTLTLSKADYEKHKVYACEVNHSGLSSPVTKSFNRGEC

SEQ ID NO: 105 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagaacaagagcggcacagcctccgtggtgtgcctgctgaacaatttctatccccgcgaggccaaggtgcagtggaaggtggataacgccctgcagtccggcaattctcaggagagcgtgaccgagcagaactccaccgattctacatactccctgtctagcaccctgacactgagcaaggccgattacgagaagcacaaggtgtatgcctgcaacgtgacccatcaggggctgtcttctccagtgaccaaatccttcaatcgcggggaatgttgataa

SEQ ID NO: 106 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQNKSGTASVVCLLNNFYPREAKVQWKVDNALQSGNSQESVTEQNSTDSTYSLSSTLTLSKADYEKHKVYACNVTHQGLSSPVTKSFNRGEC

SEQ ID NO: 107 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcagaactccaccgattctacatactccctgtctagcaccctgaacctgagcaaggccgattacgagaagcacaaggtgtatgcctgcgaggtgaaccatagcgggctgtcttctccagtgaccaaatccttcaatcgcggggaatgttgataa

SEQ ID NO: 108 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQNSTDSTYSLSSTLNLSKADYEKHKVYACEVNHSGLSSPVTKSFNRGEC

SEQ ID NO: 109 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagaacaagagcggcacagcctccgtggtgtgcctgctgaacaatttctatccccgcgaggccaaggtgcagtggaaggtggataacgccctgcagtccggcaattctagcgagagcgtgaccgagcaggactccaaggattctacatactccctgtctagcaccctgacactgagcaaggccgattacgagaagcacaaggtgtatgcctgcgaggtgaaccatagcgggctgtcttctccagtgaccaaatccttcaatcgcggggaatgttgataa

SEQ ID NO: 110 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQNKSGTASVVCLLNNFYPREAKVQWKVDNALQSGNSSESVTEQDSKDSTYSLSSTLTLSKADYEKHKVYACEVNHSGLSSPVTKSFNRGEC

SEQ ID NO: 111 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagagcgtgtctagctcctacctggcctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagaacaagagcggcacagcctccgtggtgtgcctgctgaacaatttctatccccgcgaggccaaggtgcagtggaaggtggataacgccctgcagtccggcaattctagcgagagcgtgaccgagcagaactccaccgattctacatactccctgtctagcaccctgaacctgagcaaggccgattacgagaagcacaaggtgtatgcctgcaacgtgacccatcaggggctgtcttctccagtgaccaaatccttcaatcgcggggaatgttgataa

SEQ ID NO: 112 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQNKSGTASVVCLLNNFYPREAKVQWKVDNALQSGNSSESVTEQNSTDSTYSLSSTLNLSKADYEKHKVYACNVTHQGLSSPVTKSFNRGEC

SEQ ID NO: 113 - DNA, 714 bp, synthetic construct; identical to SEQ ID NO: 19
  source 1..714: mol_type = other DNA

SEQ ID NO: 114 - protein, 236 aa, synthetic construct; identical to SEQ ID NO: 20

SEQ ID NO: 115 - DNA, 711 bp, synthetic construct
  source 1..711: mol_type = other DNA
atggtgctgcagacccaggtgttcatcagcctgctgctgtggatctccggcgcctacggcgagatcgtgctgacccagtccccaggcacactgagcctgtcccctggagagagagccaccctgagctgcagggcatctcagaccatttctaataattttgttgcctggtatcagcagaagcctggccaggccccaagactgctgatctacggagccagcacccgcgccaccggcatccccgacaggttctccggctctggcagcggcacagacttcaccctgacaatctcccggctggagcctgaggacttcgccgtgtactactgtcagcagtatggatcctctccctacacctttggccagggcacaaagctggagatcaagcgtacagtggccgcccccagcgtgttcatctttccacccagcgacgagcagctgaagtccggcaccgcctctgtggtgtgcctgctgaacaatttctaccctcgggaggccaaggtgcagtggaaggtggataacgccctgcagtccggcaattctcaggagagcgtgaccgagcaggactccaaggattctacatatagcctgagctccaccctgacactgagcaaggccgactacgagaagcacaaggtgtatgcctgtgaggtcacccaccaggggctgtcaagtccagtcactaaaagtttcaataggggagaatgttgataa

SEQ ID NO: 116 - protein, 235 aa, synthetic construct
MVLQTQVFISLLLWISGAYGEIVLTQSPGTLSLSPGERATLSCRASQTISNNFVAWYQQKPGQAPRLLIYGASTRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcagtacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 118 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 119 - DNA, 711 bp, synthetic construct
  source 1..711: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccccctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 120 - protein, 235 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 121 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaccctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 122 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSRPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 123 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccggcccctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 124 - protein, 236 aa, synthetic construct
MVLQTQVFISLLLWISGAYGEIVLTQSPGTLSLSPGERATLSCRASQSVSSSYLAWYQQKPGQAPRLLIYGASSRATGIPDRFSGSGSGTDFTLTISRLEPEDFAVYYCQHYGSSGPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 125 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccatttccaataattttgttgcttggtaccagcagaaacctggccaggcccctagactgctgatctacggcgctagcaccagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 126 - protein, 236 aa, synthetic construct
MVLQTQVFISLLLWISGAYGEIVLTQSPGTLSLSPGERATLSCRASQTISNNFVAWYQQKPGQAPRLLIYGASTRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 127 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccatttccaatagctacgttgcttggtaccagcagaaacctggccaggcccctagactgctgatctacggcgctagcaccagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 128 - protein, 236 aa, synthetic construct
MVLQTQVFISLLLWISGAYGEIVLTQSPGTLSLSPGERATLSCRASQTISNSYVAWYQQKPGQAPRLLIYGASTRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 129 - DNA, 711 bp, synthetic construct
  source 1..711: mol_type = other DNA
atggtgctgcagacccaggtgttcatcagcctgctgctgtggatctccggcgcctacggcgagatcgtgctgacccagtccccaggcacactgagcctgtcccctggagagagagccaccctgagctgcagggcatctcagaccatttctaatagctacgttgcctggtatcagcagaagcctggccaggccccaagactgctgatctacggagccagcacccgcgccaccggcatccccgacaggttctccggctctggcagcggcacagacttcaccctgacaatctcccggctggagcctgaggacttcgccgtgtactactgtcagcagtatggatcctctccctacacctttggccagggcacaaagctggagatcaagcgtacagtggccgcccccagcgtgttcatctttccacccagcgacgagcagctgaagtccggcaccgcctctgtggtgtgcctgctgaacaatttctaccctcgggaggccaaggtgcagtggaaggtggataacgccctgcagtccggcaattctcaggagagcgtgaccgagcaggactccaaggattctacatatagcctgagctccaccctgacactgagcaaggccgactacgagaagcacaaggtgtatgcctgtgaggtcacccaccaggggctgtcaagtccagtcactaaaagtttcaataggggagaatgttgataa

SEQ ID NO: 130 - protein, 235 aa, synthetic construct
MVLQTQVFISLLLWISGAYGEIVLTQSPGTLSLSPGERATLSCRASQTISNSYVAWYQQKPGQAPRLLIYGASTRATGIPDRFSGSGSGTDFTLTISRLEPEDFAVYYCQQYGSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - DNA, 711 bp, synthetic construct
  source 1..711: mol_type = other DNA
atggtgctgcagacccaggtgttcatcagcctgctgctgtggatctccggcgcctacggcgagatcgtgctgacccagtccccaggcacactgagcctgtcccctggagagagagccaccctgagctgcagggcatctcagaccatttctaatagctacgttgcctggtatcagcagaagcctggccaggccccaagactgctgatctacggagccagcacccgcgccaccggcatccccgacaggttctccggctctggcagcggcacagacttcaccctgacaatctcccggctggagcctgaggacttcgccgtgtactactgtcagcagtatggatcctctccctggacctttggccagggcacaaagctggagatcaagcgtacagtggccgcccccagcgtgttcatctttccacccagcgacgagcagctgaagtccggcaccgcctctgtggtgtgcctgctgaacaatttctaccctcgggaggccaaggtgcagtggaaggtggataacgccctgcagtccggcaattctcaggagagcgtgaccgagcaggactccaaggattctacatatagcctgagctccaccctgacactgagcaaggccgactacgagaagcacaaggtgtatgcctgtgaggtcacccaccaggggctgtcaagtccagtcactaaaagtttcaataggggagaatgttgataa

SEQ ID NO: 132 - protein, 235 aa, synthetic construct
MVLQTQVFISLLLWISGAYGEIVLTQSPGTLSLSPGERATLSCRASQTISNSYVAWYQQKPGQAPRLLIYGASTRATGIPDRFSGSGSGTDFTLTISRLEPEDFAVYYCQQYGSSPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 711 bp, synthetic construct
  source 1..711: mol_type = other DNA
atggtgctgcagacccaggtgttcatcagcctgctgctgtggatctccggcgcctacggcgagatcgtgctgacccagtccccaggcacactgagcctgtcccctggagagagagccaccctgagctgcagggcatctcagagcgtgtctagctcctacctgggctggtatcagcagaagcctggccaggccccaagactgctgatctacggagcctctagccgcgccaccggcatccccgacaggttctccggctctggcagcggcacagacttcaccctgacaatctcccggctggagcctgaggacttcgccgtgtactactgtcagcagtatggctcctctccctggacctttggccagggcacaaaggtggagatcaagcgtacagtggccgcccccagcgtgttcatctttccacccagcgacgagcagctgaagtccggcaccgcctctgtggtgtgcctgctgaacaatttctaccctcgggaggccaaggtgcagtggaaggtggataacgccctgcagtccggcaattctcaggagagcgtgaccgagcaggactccaaggattctacatatagcctgagctccaccctgacactgagcaaggccgactacgagaagcacaaggtgtatgcctgtgaggtcacccaccaggggctgtcaagtccagtcactaaaagtttcaataggggagaatgttgataa

SEQ ID NO: 134 - protein, 235 aa, synthetic construct
MVLQTQVFISLLLWISGAYGEIVLTQSPGTLSLSPGERATLSCRASQSVSSSYLGWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - DNA, 1422 bp, synthetic construct; identical to SEQ ID NO: 1
  source 1..1422: mol_type = other DNA

SEQ ID NO: 136 - protein, 472 aa, synthetic construct; identical to SEQ ID NO: 2

SEQ ID NO: 137 - DNA, 21 bp, synthetic construct
  source 1..21: mol_type = other DNA
cagaccatttctaattcctac

SEQ ID NO: 138 - protein, 7 aa, synthetic construct
QTISNSY

SEQ ID NO: 139-140 - skipped (2 entries)

SEQ ID NO: 141 - DNA, 30 bp, synthetic construct
  source 1..30: mol_type = other DNA
cagcactatggctcctctcggggctggacc

SEQ ID NO: 142 - protein, 10 aa, synthetic construct
QHYGSSRGWT

SEQ ID NO: 143 - DNA, 327 bp, synthetic construct
  source 1..327: mol_type = other DNA
gagatcgtgctgacccagtccccaggcacactgagcctgtcccctggagagagagccaccctgagctgcagggcatctcagaccatttctaattcctacgttgcctggtatcagcagaagcctggccaggccccaagactgctgatctacggagcctctacccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaag

SEQ ID NO: 144 - protein, 109 aa, synthetic construct
EIVLTQSPGTLSLSPGERATLSCRASQTISNSYVAWYQQKPGQAPRLLIYGASTRATGIPDRFSGSGSGTDFTLTISRLEPEDFAVYYCQHYGSSRGWTFGQGTKVEIK

SEQ ID NO: 145 - DNA, 714 bp, synthetic construct
  source 1..714: mol_type = other DNA
atggtgctgcagacccaggtgtttatcagcctgctgctgtggatctccggagcatatggagagatcgtgctgacccagtccccaggcacactgagcctgtcccctggagagagagccaccctgagctgcagggcatctcagaccatttctaattcctacgttgcctggtatcagcagaagcctggccaggccccaagactgctgatctacggagcctctacccgcgccaccggcatccccgacaggttctccggctctggcagcggcacagacttcaccctgacaatctcccggctggagcctgaggacttcgccgtgtactactgtcagcactatggctcctctcggggctggacctttggccagggcacaaaggtggagatcaagaggaccgtggcagcaccatccgtgttcatctttccaccctctgacgagcagctgaagagcggcacagcctccgtggtgtgcctgctgaacaatttctatccccgcgaggccaaggtgcagtggaaggtggataacgccctgcagtccggcaattctcaggagagcgtgaccgagcaggactccaaggattctacatactccctgtctagcaccctgacactgagcaaggccgattacgagaagcacaaggtgtatgcctgcgaggtgacccatcaggggctgtcttctccagtgaccaaatccttcaatcgcggggaatgttgataa

SEQ ID NO: 146 - protein, 236 aa, synthetic construct
MVLQTQVFISLLLWISGAYGEIVLTQSPGTLSLSPGERATLSCRASQTISNSYVAWYQQKPGQAPRLLIYGASTRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - DNA, 21 bp, synthetic construct
  source 1..21: mol_type = other DNA
cagaccgtgtccagcagctac

SEQ ID NO: 148 - protein, 7 aa, synthetic construct
QTVSSSY

SEQ ID NO: 149 - skipped

SEQ ID NO: 150 - DNA, 30 bp, synthetic construct
  source 1..30: mol_type = other DNA
cagcactacggcagctccagaggctggacc

SEQ ID NO: 151 - DNA, 327 bp, synthetic construct
  source 1..327: mol_type = other DNA
gaaatcgtgctgacccagtctcctggaacactgagcctgtctcccggcgaacgggccacactgagctgcagagcttctcagaccgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 152 - protein, 109 aa, synthetic construct
EIVLTQSPGTLSLSPGERATLSCRASQTVSSSYLAWYQQKPGQAPRLLIYGASSRATGIPDRFSGSGSGTDFTLTISRLEPEDFAVYYCQHYGSSRGWTFGQGTKVEIK

SEQ ID NO: 153 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccgtgtccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 154 - protein, 236 aa, synthetic construct
MVLQTQVFISLLLWISGAYGEIVLTQSPGTLSLSPGERATLSCRASQTVSSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 155 - DNA, 21 bp, synthetic construct
  source 1..21: mol_type = other DNA
cagagcatttccagcagctac

SEQ ID NO: 156 - protein, 7 aa, synthetic construct
QSISSSY

SEQ ID NO: 157 - DNA, 327 bp, synthetic construct
  source 1..327: mol_type = other DNA
gaaatcgtgctgacccagtctcctggaacactgagcctgtctcccggcgaacgggccacactgagctgcagagcttctcagagcatttccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 158 - protein, 109 aa, synthetic construct
EIVLTQSPGTLSLSPGERATLSCRASQSISSSYLAWYQQKPGQAPRLLIYGASSRATGIPDRFSGSGSGTDFTLTISRLEPEDFAVYYCQHYGSSRGWTFGQGTKVEIK

SEQ ID NO: 159 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcatttccagcagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 160 - protein, 236 aa, synthetic construct
MVLQTQVFISLLLWISGAYGEIVLTQSPGTLSLSPGERATLSCRASQSISSSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 161 - DNA, 21 bp, synthetic construct
  source 1..21: mol_type = other DNA
cagagcgtgtccaatagctac

SEQ ID NO: 162 - protein, 7 aa, synthetic construct
QSVSNSY

SEQ ID NO: 163 - DNA, 327 bp, synthetic construct
  source 1..327: mol_type = other DNA
gaaatcgtgctgacccagtctcctggaacactgagcctgtctcccggcgaacgggccacactgagctgcagagcttctcagagcgtgtccaatagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 164 - protein, 109 aa, synthetic construct
EIVLTQSPGTLSLSPGERATLSCRASQSVSNSYLAWYQQKPGQAPRLLIYGASSRATGIPDRFSGSGSGTDFTLTISRLEPEDFAVYYCQHYGSSRGWTFGQGTKVEIK

SEQ ID NO: 165 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccaatagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 166 - protein, 236 aa, synthetic construct
MVLQTQVFISLLLWISGAYGEIVLTQSPGTLSLSPGERATLSCRASQSVSNSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 167 - DNA, 21 bp, synthetic construct
  source 1..21: mol_type = other DNA
cagagcgtgtccagcaattac

SEQ ID NO: 168 - protein, 7 aa, synthetic construct
QSVSSNY

SEQ ID NO: 169 - DNA, 327 bp, synthetic construct
  source 1..327: mol_type = other DNA
gaaatcgtgctgacccagtctcctggaacactgagcctgtctcccggcgaacgggccacactgagctgcagagcttctcagagcgtgtccagcaattacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 170 - protein, 109 aa, synthetic construct
EIVLTQSPGTLSLSPGERATLSCRASQSVSSNYLAWYQQKPGQAPRLLIYGASSRATGIPDRFSGSGSGTDFTLTISRLEPEDFAVYYCQHYGSSRGWTFGQGTKVEIK

SEQ ID NO: 171 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcgtgtccagcaattacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 172 - protein, 236 aa, synthetic construct
MVLQTQVFISLLLWISGAYGEIVLTQSPGTLSLSPGERATLSCRASQSVSSNYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 173 - DNA, 21 bp, synthetic construct
  source 1..21: mol_type = other DNA
cagaccatttccaatagctac

SEQ ID NO: 174 - DNA, 327 bp, synthetic construct
  source 1..327: mol_type = other DNA
gaaatcgtgctgacccagtctcctggaacactgagcctgtctcccggcgaacgggccacactgagctgcagagcttctcagaccatttccaatagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 175 - protein, 109 aa, synthetic construct
EIVLTQSPGTLSLSPGERATLSCRASQTISNSYLAWYQQKPGQAPRLLIYGASSRATGIPDRFSGSGSGTDFTLTISRLEPEDFAVYYCQHYGSSRGWTFGQGTKVEIK

SEQ ID NO: 176 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccatttccaatagctacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 177 - protein, 236 aa, synthetic construct
MVLQTQVFISLLLWISGAYGEIVLTQSPGTLSLSPGERATLSCRASQTISNSYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 178 - DNA, 327 bp, synthetic construct
  source 1..327: mol_type = other DNA
gaagtggtgctgacccagtctcctggaacactgagcctgtctcccggcgaacgggccacactgagctgcagagcttctcagaccatttccaatagctacgttgcttggtaccagcagagacctggccaggcccctagactgctgatctacggcgctagcaccagagccgccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 179 - protein, 109 aa, synthetic construct
EVVLTQSPGTLSLSPGERATLSCRASQTISNSYVAWYQQRPGQAPRLLIYGASTRAAGIPDRFSGSGSGTDFTLTISRLEPEDFAVYYCQHYGSSRGWTFGQGTKVEIK

SEQ ID NO: 180 - DNA, 714 bp, synthetic construct
  source 1..714: mol_type = other DNA
atggtgctgcaaacacaggtgttcattagcctgctcctgtggatctccggcgcctacggcgaagtggtgctgacccagtctcctggaacactgagcctgtctcccggcgaacgggccacactgagctgcagagcttctcagaccatttccaatagctacgttgcttggtaccagcagagacctggccaggcccctagactgctgatctacggcgctagcaccagagccgccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 181 - protein, 236 aa, synthetic construct
MVLQTQVFISLLLWISGAYGEVVLTQSPGTLSLSPGERATLSCRASQTISNSYVAWYQQRPGQAPRLLIYGASTRAAGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 182 - DNA, 30 bp, synthetic construct
  source 1..30: mol_type = other DNA
cagcactacggcagctccggcccctggacc

SEQ ID NO: 183 - protein, 10 aa, synthetic construct
QHYGSSGPWT

SEQ ID NO: 184 - DNA, 327 bp, synthetic construct
  source 1..327: mol_type = other DNA
gaaatcgtgctgacccagtctcctggaacactgagcctgtctcccggcgaacgggccacactgagctgcagagcttctcagaccatttccaatagctacgttgcttggtaccagcagaaacctggccaggcccctagactgctgatctacggcgctagcaccagagccaccggcatccctgatagattcagcggctctggatctggcaccgatttcaccctgaccatcagccgcctggaaccagaggacttcgccgtgtactactgtcagcactacggcagctccggcccctggaccttcggccaaggcacaaaggtcgagatcaag

SEQ ID NO: 185 - protein, 109 aa, synthetic construct
EIVLTQSPGTLSLSPGERATLSCRASQTISNSYVAWYQQKPGQAPRLLIYGASTRATGIPDRFSGSGSGTDFTLTISRLEPEDFAVYYCQHYGSSGPWTFGQGTKVEIK

SEQ ID NO: 186 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccatttccaatagctacgttgcttggtaccagcagaaacctggccaggcccctagactgctgatctacggcgctagcaccagagccaccggcatccctgatagattcagcggctctggatctggcaccgatttcaccctgaccatcagccgcctggaaccagaggacttcgccgtgtactactgtcagcactacggcagctccggcccctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 187 - protein, 236 aa, synthetic construct
MVLQTQVFISLLLWISGAYGEIVLTQSPGTLSLSPGERATLSCRASQTISNSYVAWYQQKPGQAPRLLIYGASTRATGIPDRFSGSGSGTDFTLTISRLEPEDFAVYYCQHYGSSGPWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 188 - DNA, 21 bp, synthetic construct
  source 1..21: mol_type = other DNA
cagagcgtgtccagcagctac

SEQ ID NO: 189 - protein, 7 aa, synthetic construct
QSVSSSY

SEQ ID NO: 190 - DNA, 327 bp, synthetic construct
  source 1..327: mol_type = other DNA
gaagtggtgctgacccagtctcctggaacactgagcctgtctcccggcgaacgggccacactgagctgcagagcttctcagagcgtgtccagcagctacctggcttggtaccagcagaaacctggccaggcccctagactgctgatcttcggcgctagctctagagccaccggcatccctgatagattcagcggctctggatctggcaccgatttcaccctgaccatcacccgcctggaaccagaggacagcgccgtgtactactgtcagcactacggcagctccagaggctggaccttcggccaaggcacaaaggtcgagatcaag

SEQ ID NO: 191 - protein, 109 aa, synthetic construct
EVVLTQSPGTLSLSPGERATLSCRASQSVSSSYLAWYQQKPGQAPRLLIFGASSRATGIPDRFSGSGSGTDFTLTITRLEPEDSAVYYCQHYGSSRGWTFGQGTKVEIK

SEQ ID NO: 192 - DNA, 714 bp, synthetic construct
  source 1..714: mol_type = other DNA
atggtgctgcaaacacaggtgttcattagcctgctcctgtggatctccggcgcctacggcgaagtggtgctgacccagtctcctggaacactgagcctgtctcccggcgaacgggccacactgagctgcagagcttctcagagcgtgtccagcagctacctggcttggtaccagcagaaacctggccaggcccctagactgctgatcttcggcgctagctctagagccaccggcatccctgatagattcagcggctctggatctggcaccgatttcaccctgaccatcacccgcctggaaccagaggacagcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 193 - protein, 236 aa, synthetic construct
MVLQTQVFISLLLWISGAYGEVVLTQSPGTLSLSPGERATLSCRASQSVSSSYLAWYQQKPGQAPRLLIFGASSRATGIPDRFSGSGSGTDFTLTITRLEPEDSAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 194 - DNA, 327 bp, synthetic construct
  source 1..327: mol_type = other DNA
gaaatcgtgctgacccagtctcctggaacactgagcctgtctcccggcgaacgggccacactgagctgcagagcttctcagagcatttccagcagctacctggcttggtaccagcagagacctgaccaggcccctagactgctgatctacggcgctagctctagagccaccggcatccctgatagattcagcggctctggatctggcaccgatttcaccctgaccatcagccgcctggaaccagaggacttcgccgtgtacttctgtcagcactacggcagctccagaggctggaccttcggccaaggcacaaaggtcgagatcaag

SEQ ID NO: 195 - protein, 109 aa, synthetic construct
EIVLTQSPGTLSLSPGERATLSCRASQSISSSYLAWYQQRPDQAPRLLIYGASSRATGIPDRFSGSGSGTDFTLTISRLEPEDFAVYFCQHYGSSRGWTFGQGTKVEIK

SEQ ID NO: 196 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagagcatttccagcagctacctggcttggtaccagcagagacctgaccaggcccctagactgctgatctacggcgctagctctagagccaccggcatccctgatagattcagcggctctggatctggcaccgatttcaccctgaccatcagccgcctggaaccagaggacttcgccgtgtacttctgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 197 - protein, 236 aa, synthetic construct
MVLQTQVFISLLLWISGAYGEIVLTQSPGTLSLSPGERATLSCRASQSISSSYLAWYQQRPDQAPRLLIYGASSRATGIPDRFSGSGSGTDFTLTISRLEPEDFAVYFCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 198 - DNA, 21 bp, synthetic construct
  source 1..21: mol_type = other DNA
cagaccatttccagcaattac

SEQ ID NO: 199 - protein, 7 aa, synthetic construct
QTISSNY

SEQ ID NO: 200 - DNA, 327 bp, synthetic construct
  source 1..327: mol_type = other DNA
gaaatcgtgctgacccagtctcctggaacactgagcctgtctcccggcgaacgggccacactgagctgcagagcttctcagaccatttccagcaattacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaag

SEQ ID NO: 201 - protein, 109 aa, synthetic construct
EIVLTQSPGTLSLSPGERATLSCRASQTISSNYLAWYQQKPGQAPRLLIYGASSRATGIPDRFSGSGSGTDFTLTISRLEPEDFAVYYCQHYGSSRGWTFGQGTKVEIK

SEQ ID NO: 202 - DNA, 714 bp, synthetic construct
  source 1..714: mol_type = other DNA
atggtgctgcaaacacaggtgttcattagcctgctcctgtggatctccggcgcctacggcgaaatcgtgctgacccagtctcctggaacactgagcctgtctcccggcgaacgggccacactgagctgcagagcttctcagaccatttccagcaattacctggcttggtaccagcagaaacctggccaggcccctagactgctgatctacggcgctagctctagagccaccggcatccctgatagattcagcggctctggatctggcaccgatttcaccctgaccatcagccgcctggaaccagaggacttcgccgtgtactactgtcagcactacggcagctccagaggctggaccttcggccaaggcacaaaggtcgagatcaagcggaccgtggccgcccctagcgtgtttatctttcctccatccgacgagcagctgaagagcggcaccgccagcgtggtgtgcctgctgaacaacttctacccccgggaagccaaggtgcagtggaaggtggacaacgccctgcagagcggaaatagccaggagagcgttacagagcaggatagcaaggactccacctacagcctgagcaacaccctgactctgtctaaggccgactatgagaagcacaaggtctatgcctgcgaggtgacacaccagggcctgtcaagccccgtgaccaaaagcttcaacagaggagaatgttgatga

SEQ ID NO: 203 - protein, 236 aa, synthetic construct
MVLQTQVFISLLLWISGAYGEIVLTQSPGTLSLSPGERATLSCRASQTISSNYLAWYQQKPGQAPRLLIYGASSRATGIPDRFSGSGSGTDFTLTISRLEPEDFAVYYCQHYGSSRGWTFGQGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

SEQ ID NO: 204 - DNA, 36 bp, synthetic construct
  source 1..36: mol_type = other DNA
cagaccatcttctattcctctaacagcaagaattat

SEQ ID NO: 205 - protein, 12 aa, synthetic construct
QTIFYSSNSKNY

SEQ ID NO: 206-207 - skipped (2 entries)

SEQ ID NO: 208 - DNA, 27 bp, synthetic construct
  source 1..27: mol_type = other DNA
cagcagtattacagcaccctgaccttc

SEQ ID NO: 209 - protein, 8 aa, synthetic construct
QQYYSTLT

SEQ ID NO: 210 - DNA, 336 bp, synthetic construct
  source 1..336: mol_type = other DNA
gacatcgtgatgacccagtctcctgatagcctggccgtgtctctgggagagagggcaacaatcaactgtaagtctagccagaccatcttctattcctctaacagcaagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaag

SEQ ID NO: 211 - protein, 112 aa, synthetic construct
DIVMTQSPDSLAVSLGERATINCKSSQTIFYSSNSKNYLAWYQQKPGQPPKLLMYWASTRESGVPDRFSGSGSGAEFTLTISSLQAEDVAIYYCQQYYSTLTFGGGTKVEIK

SEQ ID NO: 212 - DNA, 723 bp, synthetic construct
  source 1..723: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagaccatcttctattcctctaacagcaagaattatctggcatggtaccagcagaagccaggacagccacccaagctgctgatgtactgggcatctacccgggagagcggagtgcctgaccgcttctctggcagcggctccggagcagagtttaccctgacaatcagctccctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataa

SEQ ID NO: 213 - protein, 239 aa, synthetic construct
MVLQTQVFISLLLWISGAYGDIVMTQSPDSLAVSLGERATINCKSSQTIFYSSNSKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - DNA, 36 bp, synthetic construct
  source 1..36: mol_type = other DNA
cagagcatcctgtattcctctaacaataagaattat

SEQ ID NO: 215 - protein, 12 aa, synthetic construct
QSILYSSNNKNY

SEQ ID NO: 216 - DNA, 27 bp, synthetic construct
  source 1..27: mol_type = other DNA
cagcagtattacaccaccctgaccttc

SEQ ID NO: 217 - protein, 8 aa, synthetic construct
QQYYTTLT

SEQ ID NO: 218 - DNA, 336 bp, synthetic construct
  source 1..336: mol_type = other DNA
gacatcgtgatgacccagtctcctgatagcctggccgtgtctctgggagagagggcaacaatcaactgtaagtctagccagagcatcctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagcaacctgcaggccgaggatgtggccatctattactgccagcagtattacaccaccctgaccttcggcggcggcaccaaggtggagatcaag

SEQ ID NO: 219 - protein, 112 aa, synthetic construct
DIVMTQSPDSLAVSLGERATINCKSSQSILYSSNNKNYLAWYQQKPGQPPKLLMYWASTRESGVPDRFSGSGSGAEFTLTISNLQAEDVAIYYCQQYYTTLTFGGGTKVEIK

SEQ ID NO: 220 - DNA, 723 bp, synthetic construct
  source 1..723: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcatcctgtattcctctaacaataagaattatctggcatggtaccagcagaagccaggacagccacccaagctgctgatgtactgggcatctacccgggagagcggagtgcctgaccgcttctctggcagcggctccggagcagagtttaccctgacaatcagcaacctgcaggccgaggatgtggccatctattactgccagcagtattacaccaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataa

SEQ ID NO: 221 - protein, 239 aa, synthetic construct
MVLQTQVFISLLLWISGAYGDIVMTQSPDSLAVSLGERATINCKSSQSILYSSNNKNYLAWYQQKPGQPPKLLMYWASTRESGVPDRFSGSGSGAEFTLTISNLQAEDVAIYYCQQYYTTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 222 - DNA, 36 bp, synthetic construct
  source 1..36: mol_type = other DNA
cagagcgtgctgtattcctctaacaataagaattat

SEQ ID NO: 223 - protein, 12 aa, synthetic construct
QSVLYSSNNKNY

SEQ ID NO: 224 - DNA, 336 bp, synthetic construct
  source 1..336: mol_type = other DNA
gacatcgtgatgacccagtctcctgatagcctggccgtgtctctgggagagagggcaacaatcaactgtaggtctagccagagcgtgctgtattcctctaacaataagaattatctggcatggtaccagcagaagccaggacagccacccaaggtgctgatgtactgggcatctacccgggagagcggagtgcctgaccgcttctctggcagcggctccggagcagagtttaccctgacaatcagcaacctgcaggccgaggatgtggccatctattactgccagcagtattacagcaccctgaccttcggcggcggcaccaaggtggagatcaag

SEQ ID NO: 225 - protein, 112 aa, synthetic construct
DIVMTQSPDSLAVSLGERATINCRSSQSVLYSSNNKNYLAWYQQKPGQPPKVLMYWASTRESGVPDRFSGSGSGAEFTLTISNLQAEDVAIYYCQQYYSTLTFGGGTKVEIK

SEQ ID NO: 226 - DNA, 723 bp, synthetic construct
  source 1..723: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaggtctagccagagcgtgctgtattcctctaacaataagaattatctggcatggtaccagcagaagccaggacagccacccaaggtgctgatgtactgggcatctacccgggagagcggagtgcctgaccgcttctctggcagcggctccggagcagagtttaccctgacaatcagcaacctgcaggccgaggatgtggccatctattactgccagcagtattacagcaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataa

SEQ ID NO: 227 - protein, 239 aa, synthetic construct
MVLQTQVFISLLLWISGAYGDIVMTQSPDSLAVSLGERATINCRSSQSVLYSSNNKNYLAWYQQKPGQPPKVLMYWASTRESGVPDRFSGSGSGAEFTLTISNLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 228 - DNA, 36 bp, synthetic construct
  source 1..36: mol_type = other DNA
cagagcgtgttctattcctctaacaataagaattat

SEQ ID NO: 229 - protein, 12 aa, synthetic construct
QSVFYSSNNKNY

SEQ ID NO: 230 - DNA, 336 bp, synthetic construct
  source 1..336: mol_type = other DNA
gacatcgtgatgacccagtctcctgatagcctggccgtgtctctgggagagagggcaacaatcaactgtaagtctagccagagcgtgttctattcctctaacaataagaattatctggcatggtaccagcagaagccaggacagccaccccgggtgctgatgtactgggcatctacccgggagagcggagtgcctgaccgcttctctggcagcggctccggagcagagtttaccctgacaatcagctccctgcaggccgaggatgtggccatctattactgccagcagtattacaccaccctgaccttcggcggcggcaccaaggtggagatcaag

SEQ ID NO: 231 - protein, 112 aa, synthetic construct
DIVMTQSPDSLAVSLGERATINCKSSQSVFYSSNNKNYLAWYQQKPGQPPRVLMYWASTRESGVPDRFSGSGSGAEFTLTISSLQAEDVAIYYCQQYYTTLTFGGGTKVEIK

SEQ ID NO: 232 - DNA, 723 bp, synthetic construct
  source 1..723: mol_type = other DNA
atggtgctgcagacccaggtgtttatctccctgctgctgtggatctctggcgcctacggcgacatcgtgatgacccagtctcctgatagcctggccgtgtctctgggagagagggcaacaatcaactgtaagtctagccagagcgtgttctattcctctaacaataagaattatctggcatggtaccagcagaagccaggacagccaccccgggtgctgatgtactgggcatctacccgggagagcggagtgcctgaccgcttctctggcagcggctccggagcagagtttaccctgacaatcagctccctgcaggccgaggatgtggccatctattactgccagcagtattacaccaccctgaccttcggcggcggcaccaaggtggagatcaagaggacagtggccgcccctagcgtgttcatctttcctccaagcgacgagcagctgaagtccggcaccgcctctgtggtgtgcctgctgaacaacttctacccaagagaggccaaggtgcagtggaaggtggataacgccctgcagtccggcaattctcaggagagcgtgaccgagcaggactccaaggattctacatactctctgagcagcaccctgacactgagcaaggccgactatgagaagcacaaggtgtacgcctgcgaagtgacccaccaggggctgagcagtccagtgaccaagtctttcaatcggggagaatgctgataa

SEQ ID NO: 233 - protein, 239 aa, synthetic construct
MVLQTQVFISLLLWISGAYGDIVMTQSPDSLAVSLGERATINCKSSQSVFYSSNNKNYLAWYQQKPGQPPRVLMYWASTRESGVPDRFSGSGSGAEFTLTISSLQAEDVAIYYCQQYYTTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 234 - DNA, 1425 bp, synthetic construct
  source 1..1425: mol_type = other DNA
atggactggacctggagaatcctgttcctggtggcagcagcaaccggaacacacgcacaggtgcagctggtgcagtccggagcagaggtgaagaagcctggagcctctgtgaaggtgagctgcaaggcctccggctacacctttacatcttatggaatcagctgggtgaggcaggcaccaggacagggactggagtggatgggctggatcagcgcctacaacggcaatacaaactatgcccagaagctgcagggcagagtgaccatgaccacagacaccagcacatccaccgcctacatggagctgaggtctctgagaagcgacgatacagccgtgtactattgcgcccgggactatacccgcggcgcctggttcggagagtctctgatcggcggatttgataattggggccagggcacactggtgaccgtgtctgccagcacaaagggaccaagcgtgttcccactggcacccagctccaagtccacatctggcggcaccgccgccctgggatgtctggtgaaggattacttcccagagcccgtgaccgtgtcctggaattctggcgccctgacaagcggcgtgcacacctttccagccgtgctgcagtctagcggcctgtactccctgtcctctgtggtgacagtgcccagctcctctctgggcacacagacctatatctgcaatgtgaaccacaagccaagcaacaccaaggtggacaagaaggtggagcccaagtcctgtgataagacacacacctgccctccctgtcctgcaccagagctgctgggcggcccatccgtgttcctgtttccacccaagcctaaggacaccctgatgatctctcggacacccgaggtgacctgcgtggtggtggacgtgagccacgaggaccccgaggtgaagtttaattggtacgtggatggcgtggaggtgcacaacgccaagaccaagcccagggaggagcagtacaactccacatatagagtggtgtctgtgctgaccgtgctgcaccaggactggctgaatggcaaggagtataagtgcaaggtgtccaacaaggccctgcccgcccctatcgagaagacaatctctaaggcaaagggacagcctcgggagccacaggtgtacaccctgcctccatcccgcgacgagctgacaaagaatcaggtgtctctgacctgtctggtgaagggcttctatccttctgatatcgcagtggagtgggagagcaacggacagccagagaacaattacaagaccacaccccctgtgctggacagcgatggctccttctttctgtatagcaagctgacagtggataagtcccgctggcagcagggcaacgtgttcagctgttccgtgatgcacgaggccctgcacaaccactacacccagaagtctctgagcctgtcccctggcaag

SEQ ID NO: 235 - protein, 748 aa, synthetic construct
MDWTWRILFLVAAATGTHAEVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRGRKRRSGSGATNFSLLKQAGDVEENPGPMVLQTQVFISLLLWISGAYGDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSNTLTLSKADYEKHKVYACEVTHQGLSSPVTKSFNRGEC

(19) United States
(12) Patent Application Publication
(10) Pub. No.: US 2026/0365718 A1
(43) Pub. Date: Nov. 19, 2026

(54) MODIFIED ANTIBODIES AND METHODS OF USE THEREOF

(71) Applicant: THE WISTAR INSTITUTE OF ANATOMY AND BIOLOGY, Philadelphia, PA (US)

(72) Inventors: Daniel Kulp, Moorestown, NJ (US); Michaela Helble, Philadelphia, PA (US); Ami Patel, Philadelphia, PA (US)

(21) Appl. No.: 19/102,888

(22) PCT Filed: Aug. 9, 2023
(86) PCT No.: PCT/US2023/071919

§ 371 Date: Feb. 11, 2025

Related U.S. Application Data
(60) Provisional application No. 63/371,142, filed on Aug. 11, 2022.
(60) Provisional application No. 63/489,807, filed on Mar. 13, 2023.

(51) Int. Cl. C07K 16/104

(52) U.S. Cl. CPC C07K 16/104; C07K 2317/24; C07K 2317/41; C07K 2317/52; C07K 2317/565

(57) ABSTRACT

Disclosed herein are antibodies having variations for increased expression and recombinant nucleic acid sequences that encode the antibodies. The disclosure also provides methods of preventing and/or treating a disease or disorder in a subject using said compositions and methods.

### CROSS REFERENCE TO RELATED APPLICATIONS

This application claims priority to U.S. Provisional Application No. 63/371,142, filed on Aug. 11, 2022 and to U.S. Provisional Application No. 63/489,807, filed Mar. 13, 2023, each which is hereby incorporated by reference herein in its entirety.

### STATEMENT REGARDING FEDERALLY SPONSORED RESEARCH OR DEVELOPMENT

This invention was made with government support under N66001-20-1-4049 awarded by the Defense Advanced Research Projects Agency (DARPA). The government has certain rights in the invention.

### TECHNICAL FIELD

The present invention relates to optimized synthetic antibodies, optimized recombinant nucleic acid molecules for generating one or more synthetic antibodies, functional fragments thereof, and compositions comprising the optimized synthetic antibodies, and optimized recombinant nucleic acid molecules, as well as methods of use for treating or preventing a disease or disorder in a subject by administering said composition.

### BACKGROUND

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, OC43, NL63, and HKU1 are endemic in the human population.

COVID-19, known previously as 2019-nCoV pneumonia or disease, has rapidly emerged as a global public health crisis, joining severe acute respiratory syndrome (SARS) and Middle East respiratory syndrome (MERS) in a growing number of coronavirus-associated illnesses which have jumped from animals to people. There are at least seven identified coronaviruses that infect humans. In December 2019 the city of Wuhan in China became the epicenter for an outbreak of the novel coronavirus, SARS-CoV-2. SARS-CoV-2 was isolated and sequenced from human airway epithelial cells from infected patients (Zhu et al., 2020 N Engl J Med, 382:727-733; Wu et al., 2020, Nature, 579:265-269). Disease symptoms can range from mild flu-like to severe cases with life-threatening pneumonia (Huang et al., 2020, Lancet, 395:497-506). The global situation is dynamically evolving, and on Jan. 30, 2020 the World Health Organization declared COVID-19 as a public health emergency of international concern (PHEIC) and on Mar. 11, 2020 it was declared a global pandemic. As of Apr. 1, 2020 there were 932,605 people infected and 46,809 deaths (gisaid.org/epiflu-applications/global-cases-covid-19). Infections have spread to multiple continents. Human-to-human transmission has been observed in multiple countries, and a shortage of disposal personal protective equipment, and prolonged survival times of coronaviruses on inanimate surfaces (Hulkower et al., 2011, Am J Infect Control 39, 401-407), have compounded this already delicate situation and heightened the risk of nosocomial infections. Advanced research activities must be pursued in parallel to push forward protective modalities in an effort to protect billions of vulnerable individuals worldwide.

There remains a need in the art for therapeutics that prevent and/or treat diseases included COVID-19. The current invention satisfies this need.

### SUMMARY OF THE INVENTION

In one embodiment, the invention relates to an anti-SARS-CoV-2 antibody or fragment thereof comprising a heavy chain sequence comprising at least the constant heavy chain sequence of the full-length heavy chain sequence of SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88. In one embodiment, the invention relates to an anti-SARS-CoV-2 antibody or fragment thereof comprising a heavy chain sequence comprising a fragment comprising at least the variable heavy chain sequence of the full-length heavy chain sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, or SEQ ID NO:86.

In one embodiment, the invention relates to an anti-SARS-CoV-2 antibody or fragment thereof comprising a light chain sequence comprising at least the constant light chain sequence of the full-length light chain sequence selected from the group consisting of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, and SEQ ID NO:112.

In one embodiment, the invention relates to an anti-SARS-CoV-2 antibody or fragment thereof comprising a LCDR1 comprising SEQ ID NO:138, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:148, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:156, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:162, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:168, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:138, a LCDR2 comprising SEQ ID NO:140 and a LCDR3 comprising SEQ ID NO:183.

In one embodiment, the invention relates to an anti-SARS-CoV-2 antibody or fragment thereof comprising a LCDR1 comprising SEQ ID NO:189, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:198, a LCDR2 comprising SEQ ID NO:140 and a LCDR3 comprising SEQ ID NO:142.

In one embodiment, the invention relates to an anti-SARS-CoV-2 antibody or fragment thereof comprising a LCDR1 comprising SEQ ID NO:205, a LCDR2 comprising SEQ ID NO:207 and a LCDR3 comprising SEQ ID NO:209.

In one embodiment, the invention relates to an anti-SARS-CoV-2 antibody or fragment thereof comprising a LCDR1 comprising SEQ ID NO:215, a LCDR2 comprising SEQ ID NO:207 and a LCDR3 comprising SEQ ID NO:217.

In one embodiment, the invention relates to an anti-SARS-CoV-2 antibody or fragment thereof comprising a LCDR1 comprising SEQ ID NO:223, a LCDR2 comprising SEQ ID NO:207 and a LCDR3 comprising SEQ ID NO:209.

In one embodiment, the invention relates to an anti-SARS-CoV-2 antibody or fragment thereof comprising a LCDR1 comprising SEQ ID NO:229, a LCDR2 comprising SEQ ID NO:207 and a LCDR3 comprising SEQ ID NO:217.

In one embodiment, the invention relates to an anti-SARS-CoV-2 antibody or fragment thereof comprising a light chain comprising a variable region of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, SEQ ID NO:201, SEQ ID NO:211, SEQ ID NO:219, SEQ ID NO:225 and SEQ ID NO:231.

In one embodiment, the invention relates to an anti-SARS-CoV-2 antibody or fragment thereof comprising a fragment comprising at least the variable light chain sequence of the full-length light chain sequence selected of SEQ ID NO:22, SEQ ID NO:118, SEQ ID NO:120, SEQ ID NO:122, SEQ ID NO:124, SEQ ID NO:126, SEQ ID NO:128, SEQ ID NO:130, or SEQ ID NO:132.

In one embodiment, the invention relates to an anti-SARS-CoV-2 antibody or fragment thereof comprising a heavy chain comprising at least the constant heavy chain sequence of the full-length heavy chain sequence of SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88; or a fragment comprising at least the variable heavy chain sequence of the full-length heavy chain sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, or SEQ ID NO:86; and a light chain amino acid sequence comprising at least the constant light chain sequence of the full-length light chain sequence of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, or SEQ ID NO:112; or a light chain comprising a set of CDR sequences of a LCDR1 comprising SEQ ID NO:138, a LCDR2 comprising SEQ ID NO:140 and a LCDR3 comprising SEQ ID NO:142; a LCDR1 comprising SEQ ID NO:148, a LCDR2 comprising SEQ ID NO:140 and a LCDR3 comprising SEQ ID NO:142; a LCDR1 comprising SEQ ID NO:156, a LCDR2 comprising SEQ ID NO:140 and a LCDR3 comprising SEQ ID NO:142; a LCDR1 comprising SEQ ID NO:162, a LCDR2 comprising SEQ ID NO:140 and a LCDR3 comprising SEQ ID NO:142; a LCDR1 comprising SEQ ID NO:168, a LCDR2 comprising SEQ ID NO:140 and a LCDR3 comprising SEQ ID NO:142; a LCDR1 comprising SEQ ID NO:138, a LCDR2 comprising SEQ ID NO:140 and a LCDR3 comprising SEQ ID NO:183; a LCDR1 comprising SEQ ID NO:189, a LCDR2 comprising SEQ ID NO:140 and a LCDR3 comprising SEQ ID NO:142; a LCDR1 comprising SEQ ID NO:198, a LCDR2 comprising SEQ ID NO:140 and a LCDR3 comprising SEQ ID NO:142; a light chain comprising a variable region of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, or SEQ ID NO:201; or a fragment comprising at least the variable light chain sequence of the full-length light chain sequence of SEQ ID NO:22, SEQ ID NO:118, SEQ ID NO:120, SEQ ID NO:122, SEQ ID NO:124, SEQ ID NO:126, SEQ ID NO:128, SEQ ID NO:130, or SEQ ID NO:132.

In one embodiment, the invention relates to an anti-SARS-CoV-2 antibody or fragment thereof comprising a heavy chain amino acid sequence of: SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88; and a light chain amino acid sequence of: SEQ ID NO:22, SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, SEQ ID NO:112; SEQ ID NO:146, SEQ ID NO:154, SEQ ID NO:160, SEQ ID NO:166, SEQ ID NO:172, SEQ ID NO:177, SEQ ID NO:181, SEQ ID NO:187, SEQ ID NO:193, SEQ ID NO:197, or SEQ ID NO:203.

In one embodiment, the invention relates to an anti-SARS-CoV-2 antibody or fragment thereof comprising a heavy chain sequence comprising SEQ ID NO:235; and a light chain sequence comprising a LCDR1 comprising SEQ ID NO:205, a LCDR2 comprising SEQ ID NO:207 and a LCDR3 comprising SEQ ID NO:209; a LCDR1 comprising SEQ ID NO:215, a LCDR2 comprising SEQ ID NO:207 and a LCDR3 comprising SEQ ID NO:217; a LCDR1 comprising SEQ ID NO:223, a LCDR2 comprising SEQ ID NO:207 and a LCDR3 comprising SEQ ID NO:209; or a LCDR1 comprising SEQ ID NO:229, a LCDR2 comprising SEQ ID NO:207 and a LCDR3 comprising SEQ ID NO:217; or a light chain comprising a variable region of SEQ ID NO:211, SEQ ID NO:219, SEQ ID NO:225 or SEQ ID NO:231.

In one embodiment, the invention relates to an anti-SARS-CoV-2 antibody or fragment thereof comprising a heavy chain sequence comprising SEQ ID NO:235; and a light chain sequence of: SEQ ID NO:213, SEQ ID NO:221, SEQ ID NO:227, or SEQ ID NO:233.

In one embodiment, the anti-SARS-CoV-2 antibody or fragment thereof is a humanized antibody, a chimeric antibody, a fully human antibody, or an antibody mimetic.

In one embodiment, the invention relates to a nucleic acid molecule, or combination of nucleic acid molecules, comprising at least one nucleotide sequence encoding at least one of a heavy chain or a light chain of an anti-SARS-CoV-2 antibody, or fragment thereof.

In one embodiment, the invention relates to a nucleic acid molecule comprising a nucleotide sequence encoding at least the constant heavy chain sequence of the full-length heavy chain sequence of, wherein the nucleotide sequence comprises SEQ ID NO:3, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the invention relates to a nucleic acid molecule comprising a nucleotide sequence encoding at least the variable heavy chain sequence of the full-length heavy chain sequence, wherein the nucleotide sequence comprises SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:63, SEQ ID NO:75, SEQ ID NO:83, our SEQ ID NO:85.

In one embodiment, the invention relates to a nucleic acid molecule comprising a nucleotide sequence encoding at least the constant light chain sequence of the full-length light chain sequence, wherein the nucleotide sequence comprises SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, or SEQ ID NO:111.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:137, a LCDR2 encoding sequence comprising SEQ ID NO:139 and a LCDR3 encoding sequence comprising SEQ ID NO:141.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:147, a LCDR2 encoding sequence comprising SEQ ID NO:139 and a LCDR3 encoding sequence comprising SEQ ID NO:141.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:155, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:161, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:167, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:173, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:173, a LCDR2 encoding sequence comprising SEQ ID NO:149 and a LCDR3 encoding sequence comprising SEQ ID NO:182.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:188, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:198, a LCDR2 encoding sequence comprising SEQ ID NO:149 and a LCDR3 encoding sequence comprising SEQ ID NO:150.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:204, a LCDR2 encoding sequence comprising SEQ ID NO:206 and a LCDR3 encoding sequence comprising SEQ ID NO:208.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:214, a LCDR2 encoding sequence comprising SEQ ID NO:206 and a LCDR3 encoding sequence comprising SEQ ID NO:216.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:222, a LCDR2 encoding sequence comprising SEQ ID NO:206 and a LCDR3 encoding sequence comprising SEQ ID NO:208.

In one embodiment, the invention relates to a nucleic acid molecule comprising a nucleotide sequence encoding at least a set of CDRs, wherein the nucleotide sequence comprises a LCDR1 encoding sequence comprising SEQ ID NO:228, a LCDR2 encoding sequence comprising SEQ ID NO:206 and a LCDR3 encoding sequence comprising SEQ ID NO:216.

In one embodiment, the invention relates to a nucleic acid molecule comprising a nucleotide sequence encoding at least a light chain variable region, wherein the nucleotide sequence comprises SEQ ID NO:143, SEQ ID NO:151, SEQ ID NO:159, SEQ ID NO:163, SEQ ID NO:169, SEQ ID NO:174, SEQ ID NO:178, SEQ ID NO:184, SEQ ID NO:190, SEQ ID NO:194, SEQ ID NO:200, SEQ ID NO:210, SEQ ID NO:218, SEQ ID NO:224 and SEQ ID NO:230.

In one embodiment, the invention relates to a nucleic acid molecule comprising a nucleotide sequence encoding at least the variable light chain sequence of the full-length light chain sequence, wherein the full length nucleotide sequence comprises SEQ ID NO:21, SEQ ID NO:117, SEQ ID NO:119, SEQ ID NO:121, SEQ ID NO:123, SEQ ID NO:125, SEQ ID NO:127, SEQ ID NO:129, and SEQ ID NO:131.

In one embodiment, the invention relates to a nucleic acid molecule comprising a nucleotide sequence encoding a heavy chain amino acid sequence, wherein the nucleotide sequence comprises SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the invention relates to a nucleic acid molecule comprising a nucleotide sequence encoding a light chain amino acid sequence, wherein the nucleotide sequence comprises SEQ ID NO:21, SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, SEQ ID NO:111, SEQ ID NO:145, SEQ ID NO:153, SEQ ID NO:159, SEQ ID NO:165, SEQ ID NO:171, SEQ ID NO:176, SEQ ID NO:180, SEQ ID NO:186, SEQ ID NO:192, SEQ ID NO:196, or SEQ ID NO:202.

In one embodiment, the invention relates to a combination of nucleic acid molecules comprising a first nucleic acid molecule comprising a nucleotide sequence encoding a heavy chain amino acid sequence, wherein the nucleotide sequence comprises SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87; and a second nucleic acid molecule comprising a nucleotide sequence encoding a light chain amino acid sequence, wherein the nucleotide sequence comprises SEQ ID NO:21, SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, SEQ ID NO:111, SEQ ID NO:145, SEQ ID NO:153, SEQ ID NO:159, SEQ ID NO:165, SEQ ID NO:171, SEQ ID NO:176, SEQ ID NO:180, SEQ ID NO:186, SEQ ID NO:192, SEQ ID NO:196, or SEQ ID NO:202.

In one embodiment, the invention relates to a nucleic acid molecule comprising a nucleotide sequence encoding a light chain amino acid sequence, wherein the nucleotide sequence comprises SEQ ID NO:212, SEQ ID NO:220, SEQ ID NO:226, or SEQ ID NO:232.

In one embodiment, the invention relates to a combination of nucleic acid molecules comprising a first nucleic acid molecule comprising a nucleotide sequence encoding a heavy chain amino acid sequence, wherein the nucleotide sequence comprises SEQ ID NO:234; and a second nucleic acid molecule comprising a nucleotide sequence encoding a light chain amino acid sequence, wherein the nucleotide sequence comprises SEQ ID NO:212, SEQ ID NO:220, SEQ ID NO:226, and SEQ ID NO:232.

In one embodiment, the nucleotide sequence encodes a leader sequence. In one embodiment, the nucleic acid molecule comprises an expression vector.

In one embodiment, the invention relates to a composition comprising at least one anti-SARS-CoV-2 antibody or fragment thereof. In one embodiment, the invention relates to a composition comprising at least one nucleic acid molecule encoding at least one anti-SARS-CoV-2 antibody or fragment thereof. In one embodiment, the invention relates to a composition comprising a combination of nucleic acid molecules encoding at least one anti-SARS-CoV-2 antibody or fragment thereof.

In one embodiment, the composition further comprises a pharmaceutically acceptable excipient.

In one embodiment, the invention relates to a method of preventing or treating a disease in a subject, the method comprising administering to the subject a composition comprising at least one anti-SARS-CoV-2 antibody or fragment thereof, a composition comprising at least one nucleic acid molecule encoding at least one anti-SARS-CoV-2 antibody or fragment thereof, or a composition comprising a combination of nucleic acid molecules encoding at least one anti-SARS-CoV-2 antibody or fragment thereof. In one embodiment, the disease is COVID-19.

In one embodiment, the invention relates to a glycan-modified binding molecule, or fragment thereof.

In one embodiment, the glycan-modified binding molecule or heavy chain fragment thereof, comprises at least one glycan-modification in the CH region corresponding to the CH region modifications as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88.

In one embodiment, the glycan-modified binding molecule, or light chain fragment thereof, comprises at least one glycan-modification in the CL region corresponding to the CL region modifications as set forth in SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

In one embodiment, the glycan-modified binding molecule, or heavy chain fragment thereof, comprises a heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88.

In one embodiment, the glycan-modified binding molecule, or light chain fragment thereof, comprises a light chain as set forth in SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

In one embodiment, the invention relates to a composition comprising at least one glycan-modified binding molecule, or fragment thereof.

In one embodiment, the glycan-modified binding molecule is incorporated into a nanoparticle.

In one embodiment, the composition further comprises a pharmaceutically acceptable excipient. In one embodiment, the composition further comprises an adjuvant.

In one embodiment, the invention relates to a nucleic acid molecule, or combination of nucleic acid molecules, encoding a glycan-modified binding molecule or fragment thereof.

In one embodiment, the nucleic acid molecule comprises a sequence encoding a heavy chain of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the nucleic acid molecule comprises a sequence encoding a light chain sequence of: SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

In one embodiment, the invention relates to a combination of nucleic acid molecules, comprising a first nucleic acid molecule comprising a sequence encoding a heavy chain of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87, and a second nucleic acid molecule comprising a sequence encoding a light chain of: SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

In one embodiment, the invention relates to a method of treating a disease or disorder in a subject in need thereof, the method comprising administering a glycan-modified binding molecule, a composition comprising a glycan-modified binding molecule, or a nucleic acid molecule encoding a glycan-modified binding molecule or a combination of nucleic acid molecules encoding a glycan-modified binding molecule to the subject.

In one embodiment, the invention relates to a method of protecting a subject in need thereof from COVID-19, the method comprising administering a glycan-modified binding molecule, or heavy chain or light chain thereof to the subject.

In one embodiment, the method comprises administering at least one of:
a) a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88;
b) a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112; or
c) a combination of a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

In one embodiment, the invention relates to a method of treating a subject in need thereof against SARS-CoV-2, the method comprising administering a glycan-modified binding molecule, or heavy chain or light chain thereof to the subject.

In one embodiment, the method comprises administering at least one of:
a) a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88;
b) a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112; or
c) a combination of a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

In one embodiment, the invention relates to a method of protecting a subject in need thereof from a disease or disorder associated with HIV infection, the method comprising administering a glycan-modified binding molecule, or heavy chain or light chain thereof to the subject.

In one embodiment, the method comprises administering at least one of:
a) a glycan-modified heavy chain as set forth in SEQ ID NO:48;
b) a glycan-modified light chain as set forth in SEQ ID NO:52 or SEQ ID NO:54; or
c) a combination of a glycan-modified heavy chain as set forth in SEQ ID NO:48, and a glycan-modified light chain as set forth in SEQ ID NO:52 or SEQ ID NO:54.

In one embodiment, the invention relates to a method of treating a subject in need thereof against a disease or disorder associated with HIV infection, the method comprising administering a glycan-modified binding molecule, or heavy chain or light chain thereof to the subject.

In one embodiment, the method comprises administering at least one of:
a) a glycan-modified heavy chain as set forth in SEQ ID NO:48;
b) a glycan-modified light chain as set forth in SEQ ID NO:52 or SEQ ID NO:54; or
c) a combination of a glycan-modified heavy chain as set forth in SEQ ID NO:48, and a glycan-modified light chain as set forth in SEQ ID NO:52 or SEQ ID NO:54.

### BRIEF DESCRIPTION OF THE DRAWINGS

FIG. 1 depicts an overview of glycosylation pipeline. Our lab built a method to identify all possible glycosylation sites on a given protein. The program (CWG) takes a target structure and looks for potential new glycosylation sites, models the glycan, and then determines potential clashes, folding energy and sugar energy to output sets of mutations that are amenable to the introduction of glycosylation sites. CWG was run on SARS-COV-2 antibody 2196 (PDB 7L7E) to identify sites that would be amenable to glycosylation. Single glycans in either the CH1 and CL were identified, and combinations of glycans in the CH1 and CL were created from the single glycan hits.

FIG. 2 depicts an overview of the aaScan process. aaScan interrogates local protein structure for stabilization opportunities. The program scans an input structure five residues at a time looking for close structural matches in the PDB. The amino acid identity of the middle residue in all the matches from diverse proteins is compared to the middle residue of the input structure to identify how frequently each amino acid is observed in a five amino acid structural motif.

FIG. 3A and FIG. 3B depict data demonstrating that CH1 or CL glycan modification to improve expression. (A) Relative expression (AUC/AUC WT) of single, triple or quintuple glycan modifications to the CH1 or CL region of a SARS-CoV2 antibody, paired with the WT opposite chain. Automated Western Blots were run to probe for the heavy chain and AUC corresponding to the heavy chain peak was assessed. (B) Relative expression (AUC/AUC WT) of combined CH1 and CL glycan modifications using the same methodology as in (A). Combinations assessed were picked from prior rounds of screening.

FIG. 4A through FIG. 4D depict data demonstrating large scale binding and expression of lead glycan modified variants. (A) Relative expression (yield/WT yield) of promising combinations of glycan modifications as identified by small scale screening across three separate experiments. Modifications to the CH1 region were paired with modifications to the CL region. Expi293 cells were transfected with the indicated combinations and protein A columns were used to purify the antibodies. Yield was then quantified (B) Binding of each large-scale variant to WT receptor-binding domain (RBD) of SARS-CoV2. ELISA was used to assess the impact of glycan modifications to binding. (C) Model depicting the glycan combination that resulted in the largest increase in expression as compared to WT (CH1: G3.4 and CL: G3.2) (D) Relative expression (yield/WT yield) of the top glycan modifications to the CH1 and CL region of an unrelated HIV antibody.

FIG. 5 depicts data demonstrating that aaScan was applied to SARS-COV2 antibody 2196 (PDB 7L7E). Results from aaScan show positions that frequently have a different central amino acid in a five amino acid structural motif.

FIG. 6A and FIG. 6B depicts data demonstrating that the pentapeptide modifications to improve expression. (A) Relative expression (AUC/AUC WT) of triple, quadruple, or quintuple combinations of point mutations to the heavy chain paired with either the WT light chain or a light chain with a single mutation (pos 9). Automated Western Blots were run to probe for the heavy chain and AUC corresponding to the heavy chain peak was assessed. (B) Total rosetta energy of notable combinations. FastRelax was run on the WT structure, and FastDesign was used to model the mutation combinations. Energy was subsequently determined.

FIG. 7 depicts a diagram of different methods of chain swapping.

FIG. 8 depicts a diagram of the experimental design.

FIG. 9A through FIG. 9D depicts an analysis of chain swapped expression boosts. (FIG. 9A) Average in vivo expression of 2196 WT heavy chain paired with 2072 or alphamod1 light chain as measured by IgG quantification ELISA. n=5 mice/group. Nomenclature is HC_ID+LC_ID. For (FIG. 3B-FIG. 3D): Structure of 2196 VH/VL bound to SARs-CoV-2 receptor binding domain (RBD). VH (gray ribbon), VL (green ribbon), SARS-CoV-2 RBD (pink ribbon) (FIG. 9B) Location of mutations unique to 2072 (purple sphere) map to the antibody FRM2 region (FIG. 9C) Location of mutations unique to alphamod1 (cyan spheres) map mainly to CDRL1 (FIG. 9D) Mutations shared between 2072 or alphamod1 map to the CDRL3 (red spheres). There is one deletion relative to 2196 light chain (black sphere).

FIG. 10A through FIG. 10D depict the rational design of antibody CDRLs. (FIG. 10A-FIG. 10D) color schemes are as outlined above. (FIG. 10A) New variant ‘P’ incorporates a deletion and a mutation to proline at positions within the CDRL3 loop. (FIG. 10B) New variant ‘GP’ incorporates the same proline as in (FIG. 10A) plus an additional glycine mutation to maintain CDRL3 loop length. (FIG. 10C) New variant ‘cluster’ incorporates the mutations found in the CDRL1 of alphamod1. (FIG. 10D) New variant ‘Cluster_rev2’ has the same mutations as (FIG. 10C) but reverts two antigen-contacting mutations back to WT to preserve binding.

FIG. 11A through FIG. 11B depict the affinity and expression of designed variants. (FIG. 11A) Affinity of rationally designed antibodies to WT RBD as determined by Surface Plasmon Resonance. 2196 WT HC was paired with the designated rationally designed LC. Each antibody was captured at 2 μg/mL on a Protein A chip. RBD was injected as the analyte in a 4×dilution series, starting at 1000 nM. The data were fit by a 1:1 Langmuir model. (FIG. 11B) In vivo expression of variants as in (FIG. 11A) as measured by IgG quantification ELISA at day 14 post injection. n=5 mice/group. Nomenclature is HC_ID+LC_ID.

FIG. 12A and FIG. 12B depict exemplary experimental results demonstrating a CDRL3 proline analysis. (FIG. 12A) Amino acid identity at position 97. The OAS database was used to identify the amino acid identity at position 97 of the CDRL3 in all IGKV3-20 antibodies with CDRL3s of length 10, as in 2196. (FIG. 12B) Analysis of potential codons at position 97. In order to get an in-frame sequence, two nucleotides must be inserted before the J gene, which begins with guanine, meaning the codon must take the form X-X-G. The number of X-X-G codons the encode each amino acid is plotted. The enrichment of proline in overall number of sequences as in (FIG. 12B) despite only having one X-X-G codon that specifies it suggests that proline enrichment may be selected for.

FIG. 13 depicts in vivo DMAb expression of designed variants as measured by quantification ELISA on D14 post injection. Variants 2196_CDRL3_GP and 2196_CDRL1_alpha_rev show 5 and 6-fold improvement in expression over WT, respectively. Nomenclature is HC_ID+LC_ID.

FIG. 14 depicts an overview of the frequency alignment process. The observed antibody space (OAS) database collates data from studies sequencing antibody repertoires. After sorting by healthy individuals, the database has sequences from 291 million unpaired LCs. We wrote a script that is capable of searching through these sequences by light chain V and J gene, and then tabulates the frequency of each amino acid at every position in the antibody, similar to WebLogo plots.

FIG. 15 depicts example antibody frequency score (AFS) workflow. The distribution of every amino acid at a particular position (here, position 28) was analyzed. Germline was most frequent, followed by isoleucine, the AA found in high-expressing CDRL1_alpha_rev. As we were interested in changes to germline sequence, we filtered out germline sequences and recalculated the frequency of every amino acid expressed as % non-germline sequences. The most frequent non-germline amino acid (I) was termed s1 and the second most frequent termed s2. The difference in s1−s2 represented AFS, which would be indicative of potential preference for a given non-germline amino acid.

FIG. 16 depicts AFS for 2196-like IGKV3-20 LCs by position. OAS was searched for full length sequences with VK3-20 germline. The higher the AFS, the more a particular amino acid is enriched at that position across the millions of light chains searched. Residue 95A (striped purple bar) at the VJ junction has no corresponding germline amino acid and so AFS is represented as most frequent overall amino acid-second most frequent amino acid.

FIG. 17 depicts mutations identified in a given LC germline using AFS with V and J gene specified.

FIG. 18 depicts mutations identified in a given LC germline using AFS with truncated V.

FIG. 19A depicts an analysis of amino acids encoded by Int changes to germline codon at position 27A. The most frequent non-germline AA at that position (T) is colored red. Germline amino acid(S) is the striped bar. FIG. 19B depicts codon adjusted AFS (cAFS) for CDRL1 positions. A false positive signal for AFS might occur due to codon bias (Preference for a given non-germline mutation could be due to random chance rather than selection) Therefore, AFS was adjusted by the number of codons encoding for each amino acid at a given position, using information as in A

FIG. 20 depicts an example of full VK4-1s with J gene incorporated, which was used to identify new mutations in VK4-1 germline that could boost expression in vivo.

FIG. 21 depicts an example of trimmed VK4-1s with J gene incorporated, which was used to identify new mutations in VK4-1 germline that could boost expression in vivo.

FIG. 22 depicts data demonstrating chain swapping. Heavy chain of 2196 was paired with light chains from clonally related anti-SARS-COV-2 antibodies and clonally-unrelated antibodies (influenza, SARS-COV-2, S. aureas alpha-hemolysin).

FIG. 23 depicts data demonstrating that the LC CDR loop length and composition may drive expression.

FIG. 24 depicts data demonstrating that chain swap mutations impact RBD binding.

FIG. 25 depicts data demonstrating that rational design of CDR1 can improve binding affinity.

FIG. 26 depicts data demonstrating that rational design of CDR3 can improve binding affinity.

FIG. 27 depicts data demonstrating that rationally designed variants improve binding affinity.

FIG. 28A and FIG. 28B depict data demonstrating that DNA-launched antibody variants improve expression 5-6X in vivo.

### DETAILED DESCRIPTION

The present invention relates to compositions comprising a recombinant nucleic acid sequence encoding an antibody, a fragment thereof, a variant thereof, or a combination thereof. The composition can be administered to a subject in need thereof to facilitate in vivo expression and formation of a synthetic antibody.

In particular, the heavy chain and light chain polypeptides expressed from the recombinant nucleic acid sequences can assemble into the synthetic antibody. The heavy chain polypeptide and the light chain polypeptide can interact with one another such that assembly results in the synthetic antibody being capable of binding the antigen, being more immunogenic as compared to an antibody not assembled as described herein, and being capable of eliciting or inducing an immune response against the antigen.

Additionally, these synthetic antibodies are generated more rapidly in the subject than antibodies that are produced in response to antigen induced immune response. The synthetic antibodies are able to effectively bind and neutralize a range of antigens. The synthetic antibodies are also able to effectively protect against and/or promote survival of disease.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Antibody” may mean an antibody of classes IgG, IgM, IgA, IgD or IgE, or fragments, fragments or derivatives thereof, including Fab, F(ab′)2, Fd, and single chain antibodies, and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

“Antibody fragment” or “fragment of an antibody” as used interchangeably herein refers to a portion of an intact antibody comprising the antigen-binding site or variable region. The portion does not include the constant heavy chain domains (i.e. CH2, CH3, or CH4, depending on the antibody isotype) of the Fc region of the intact antibody. Examples of antibody fragments include, but are not limited to, Fab fragments, Fab′ fragments, Fab′-SH fragments, F(ab′)2 fragments, Fd fragments, Fv fragments, diabodies, single-chain Fv (scFv) molecules, single-chain polypeptides containing only one light chain variable domain, single-chain polypeptides containing the three CDRs of the light-chain variable domain, single-chain polypeptides containing only one heavy chain variable region, and single-chain polypeptides containing the three CDRs of the heavy chain variable region.

“Antigen” refers to proteins that have the ability to generate an immune response in a host. An antigen may be recognized and bound by an antibody. An antigen may originate from within the body or from the external environment.

“Coding sequence” or “encoding nucleic acid” as used herein may mean refers to the nucleic acid (RNA or DNA molecule) that comprise a nucleotide sequence which encodes an antibody as set forth herein. The coding sequence may also comprise a DNA sequence which encodes an RNA sequence. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

“Constant current” as used herein to define a current that is received or experienced by a tissue, or cells defining said tissue, over the duration of an electrical pulse delivered to same tissue. The electrical pulse is delivered from the electroporation devices described herein. This current remains at a constant amperage in said tissue over the life of an electrical pulse because the electroporation device provided herein has a feedback element, preferably having instantaneous feedback. The feedback element can measure the resistance of the tissue (or cells) throughout the duration of the pulse and cause the electroporation device to alter its electrical energy output (e.g., increase voltage) so current in same tissue remains constant throughout the electrical pulse (on the order of microseconds), and from pulse to pulse. In some embodiments, the feedback element comprises a controller.

“Current feedback” or “feedback” as used herein may be used interchangeably and may mean the active response of the provided electroporation devices, which comprises measuring the current in tissue between electrodes and altering the energy output delivered by the EP device accordingly in order to maintain the current at a constant level. This constant level is preset by a user prior to initiation of a pulse sequence or electrical treatment. The feedback may be accomplished by the electroporation component, e.g., controller, of the electroporation device, as the electrical circuit therein is able to continuously monitor the current in tissue between electrodes and compare that monitored current (or current within tissue) to a preset current and continuously make energy-output adjustments to maintain the monitored current at preset levels. The feedback loop may be instantaneous as it is an analog closed-loop feedback.

“Decentralized current” as used herein may mean the pattern of electrical currents delivered from the various needle electrode arrays of the electroporation devices described herein, wherein the patterns minimize, or preferably eliminate, the occurrence of electroporation related heat stress on any area of tissue being electroporated.

“Electroporation,” “electro-permeabilization,” or “electro-kinetic enhancement” (“EP”) as used interchangeably herein may refer to the use of a transmembrane electric field pulse to induce microscopic pathways (pores) in a bio-membrane; their presence allows biomolecules such as plasmids, oligonucleotides, siRNA, drugs, ions, and water to pass from one side of the cellular membrane to the other.

“Endogenous antibody” as used herein may refer to an antibody that is generated in a subject that is administered an effective dose of an antigen for induction of a humoral immune response.

“Feedback mechanism” as used herein may refer to a process performed by either software or hardware (or firmware), which process receives and compares the impedance of the desired tissue (before, during, and/or after the delivery of pulse of energy) with a present value, preferably current, and adjusts the pulse of energy delivered to achieve the preset value. A feedback mechanism may be performed by an analog closed loop circuit.

“Fragment” may mean a polypeptide fragment of an antibody that is function, i.e., can bind to desired target and have the same intended effect as a full length antibody. A fragment of an antibody may be 100% identical to the full length except missing at least one amino acid from the N and/or C terminal, in each case with or without signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antibody, excluding any heterologous signal peptide added. The fragment may comprise a fragment of a polypeptide that is 95% or more, 96% or more, 97% or more, 98% or more or 99% or more identical to the antibody and additionally comprise an N terminal methionine or heterologous signal peptide which is not included when calculating percent identity. Fragments may further comprise an N terminal methionine and/or a signal peptide such as an immunoglobulin signal peptide, for example an IgE or IgG signal peptide. The N terminal methionine and/or signal peptide may be linked to a fragment of an antibody.

A fragment of a nucleic acid sequence that encodes an antibody may be 100% identical to the full length except missing at least one nucleotide from the 5′ and/or 3′ end, in each case with or without sequences encoding signal peptides and/or a methionine at position 1. Fragments may comprise 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may comprise a fragment that encode a polypeptide that is 95% or more, 96% or more, 97% or more, 98% or more or 99% or more identical to the antibody and additionally optionally comprise sequence encoding an N terminal methionine or heterologous signal peptide which is not included when calculating percent identity. Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

“Genetic construct” as used herein refers to the DNA or RNA molecules that comprise a nucleotide sequence which encodes a protein, such as an antibody. The genetic construct may also refer to a DNA molecule which transcribes an RNA. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed.

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Impedance” as used herein may be used when discussing the feedback mechanism and can be converted to a current value according to Ohm's law, thus enabling comparisons with the preset current.

“Immune response” as used herein may mean the activation of a host's immune system, e.g., that of a mammal, in response to the introduction of one or more nucleic acids and/or peptides. The immune response can be in the form of a cellular or humoral response, or both.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

A “peptide,” “protein,” or “polypeptide” as used herein can mean a linked sequence of amino acids and can be natural, synthetic, or a modification or combination of natural and synthetic.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, RSV-LTR promoter, tac promoter, SV40 early promoter or SV40 late promoter and the CMV IE promoter.

“Sample” or “biological sample” as used herein means a biological material isolated from an individual. The biological sample may contain any biological material suitable for detecting the desired biomarkers, and may comprise cellular and/or non-cellular material obtained from the individual.

“Signal peptide” and “leader sequence” are used interchangeably herein and refer to an amino acid sequence that can be linked at the amino terminus of a protein set forth herein. Signal peptides/leader sequences typically direct localization of a protein. Signal peptides/leader sequences used herein preferably facilitate secretion of the protein from the cell in which it is produced. Signal peptides/leader sequences are often cleaved from the remainder of the protein, often referred to as the mature protein, upon secretion from the cell. Signal peptides/leader sequences are linked at the N terminus of the protein.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence dependent and will be different in different circumstances. Stringent conditions may be selected to be about 5-10 °C lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30 °C for short probes (e.g., about 10-50 nucleotides) and at least about 60 °C for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42 °C, or, 5×SSC, 1% SDS, incubating at 65 °C, with wash in 0.2×SSC, and 0.1% SDS at 65 °C.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc) and a human). In some embodiments, the subject may be a human or a non-human. The subject or patient may be undergoing other forms of treatment.

“Substantially complementary” as used herein may mean that a first sequence is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that a first and second sequence are at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 or more nucleotides or amino acids, or with respect to nucleic acids, if the first sequence is substantially complementary to the complement of the second sequence.

“Synthetic antibody” as used herein refers to an antibody that is encoded by the recombinant nucleic acid sequence described herein and is generated in a subject.

“Treatment” or “treating,” as used herein can mean protecting of a subject from a disease through means of preventing, suppressing, repressing, or completely eliminating the disease. Preventing the disease involves administering a vaccine of the present invention to a subject prior to onset of the disease. Suppressing the disease involves administering a vaccine of the present invention to a subject after induction of the disease but before its clinical appearance. Repressing the disease involves administering a vaccine of the present invention to a subject after clinical appearance of the disease.

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

A variant may be a nucleic acid sequence that is substantially identical over the full length of the full gene sequence or a fragment thereof. The nucleic acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the gene sequence or a fragment thereof. A variant may be an amino acid sequence that is substantially identical over the full length of the amino acid sequence or fragment thereof. The amino acid sequence may be 80%, 81%, 82%, 83%, 84%, 85%, 86%, 87%, 88%, 89%, 90%, 91%, 92%, 93%, 94%, 95%, 96%, 97%, 98%, 99%, or 100% identical over the full length of the amino acid sequence or a fragment thereof.

“Vector” as used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### 2. GLYCAN-MODIFIED BINDING MOLECULES

The invention is based, in part, on the development of glycan-modified binding molecules or nucleic acid molecules encoding the glycan-modified binding molecules. In one embodiment, the glycan-modified binding molecules or nucleic acid molecules encoding the glycan-modified binding molecules comprise at least one glycan modification in a constant region of an antibody. In one embodiment, the glycan-modified binding molecules or nucleic acid molecules encoding the glycan-modified binding molecules comprise 1, 2, 3, 4, 5, 6, 7, 8, 9, or 10 glycan modifications in a constant heavy (CH) region of an antibody. In one embodiment, the glycan-modified binding molecules or nucleic acid molecules encoding the glycan-modified binding molecules comprise 1, 2, 3, 4, 5, 6, 7, 8, 9, or 10 glycan modifications in a constant light (CL) region of an antibody.

In one embodiment, the glycan-modified binding molecules or nucleic acid molecules encoding the glycan-modified binding molecules comprise 1, 2, 3, 4, 5, 6, 7, 8, 9, or 10 glycan modifications in a constant heavy (CH) region of an antibody and further comprise 1, 2, 3, 4, 5, 6, 7, 8, 9, or 10 glycan modifications in a constant light (CL) region of an antibody.

In one embodiment, the invention relates to a binding molecule comprising at least one glycan-modification in the CH region corresponding to the CH region modifications as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88. In one embodiment, the invention relates to a nucleic acid molecule encoding a CH of a binding molecule, comprising at least one glycan-modification in the sequence encoding the CH corresponding to the CH modifications as set forth in SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the invention relates to a binding molecule comprising at least one glycan-modification in the CL region corresponding to the CL region modifications as set forth in SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112. In one embodiment, the invention relates to a nucleic acid molecule encoding a CL of a binding molecule, comprising at least one glycan-modification in the sequence encoding the CL corresponding to the CL modifications as set forth in SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

In one embodiment, the invention relates to a glycan-modified binding molecule comprising a heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88. In one embodiment, the invention relates to a nucleic acid molecule encoding glycan-modified binding molecule comprising SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the invention relates to a glycan-modified binding molecule comprising a light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112. In one embodiment, the invention relates to a nucleic acid molecule encoding glycan-modified binding molecule comprising SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

For example, provided herein are glycan-modified SARS-CoV-2 antibodies and nucleic acid molecules encoding the same which can be used to treat pathologies relating to SARS-CoV-2 infection. In one embodiment, the pathology relating to SARS-CoV-2 infection is COVID-19. Exemplary antibodies that can be used for the treatment or prevention of pathologies relating to SARS-CoV-2 infection include but are not limited to, antibodies comprising a heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, a light chain as set forth in SEQ ID NO:22, 36, 38, 40, 42, 44, 90, 92, 94, 96, 98, 100, 102, 104, 106, 108, 110, or 112 or a combination of a heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and a light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

Also provided herein are glycan-modified Human Immunodeficiency Virus (HIV) antibodies and nucleic acid molecules encoding the same which can be used to treat pathologies relating to HIV infection. In one embodiment, the pathology relating to HIV infection is AIDS. Exemplary antibodies that can be used for the treatment or prevention of pathologies relating to SARS-CoV-2 infection include but are not limited to, antibodies comprising a heavy chain as set forth in SEQ ID NO:48, a light chain as set forth in SEQ ID NO:52 or 54, or a combination of a heavy chain as set forth in SEQ ID NO:48, and a light chain as set forth in SEQ ID NO:52 or 54.

However, the invention should not be considered as being limited to these specific embodiments, as the constant region of antibody targeting any antigen of interest can be modified to contain the glycan-modifications of the CH and CL of the invention.

### 3. FRAMEWORK MODIFICATION OF BINDING MOLECULES

The invention is based, in part, on the development of framework modified binding molecules or nucleic acid molecules encoding the framework modified binding molecules. In one embodiment, the framework modified binding molecules or nucleic acid molecules encoding the framework modified binding molecules comprise at least one modification in a constant region of an antibody. In one embodiment, the framework modified binding molecules or nucleic acid molecules encoding the framework modified binding molecules comprise 1, 2, 3, 4, 5, 6, 7, 8, 9, or 10 framework modifications in a constant heavy (CH) region of an antibody. In one embodiment, the framework modified binding molecules or nucleic acid molecules encoding the framework modified binding molecules comprise 1, 2, 3, 4, 5, 6, 7, 8, 9, or 10 framework modifications in a variable heavy chain region of an antibody, wherein the framework modifications are in a region outside of the CDRs. In one embodiment, framework modified binding molecules or nucleic acid molecules encoding the framework modified binding molecules comprise 1, 2, 3, 4, 5, 6, 7, 8, 9, or 10 framework modifications in a constant light (CL) region of an antibody. In one embodiment, the framework modified binding molecules or nucleic acid molecules encoding the framework modified binding molecules comprise 1, 2, 3, 4, 5, 6, 7, 8, 9, or 10 framework modifications in a variable light chain region of an antibody, wherein the framework modifications are in a region outside of the CDRs.

In one embodiment, the framework modified binding molecules or nucleic acid molecules encoding the framework modified binding molecules comprise 1, 2, 3, 4, 5, 6, 7, 8, 9, or 10 framework modifications in a heavy chain of the antibody and further comprise 1, 2, 3, 4, 5, 6, 7, 8, 9, or 10 framework modifications in a light chain of the antibody.

Exemplary framework modification that can be incorporated into the binding molecules of the invention include, but are not limited to, framework modification in the light chain region (FWk) as set forth in Table 1, Table 2 or Table 3, or any combination thereof.

For example, in some embodiments, the framework modified binding molecule of the invention comprises a D at position 1 of the light chain as defined by Kabat numbering. In some embodiments, the framework modified binding molecule of the invention comprises a V at position 2 of the light chain as defined by Kabat numbering. In some embodiments, the framework modified binding molecule of the invention comprises a V at position 11 of the light chain as defined by Kabat numbering. In some embodiments, the framework modified binding molecule of the invention comprises a D at position 17 of the light chain as defined by Kabat numbering. In some embodiments, the framework modified binding molecule of the invention comprises a G at position 18 of the light chain as defined by Kabat numbering. In some embodiments, the framework modified binding molecule of the invention comprises an A at position 19 of the light chain as defined by Kabat numbering. In some embodiments, the framework modified binding molecule of the invention comprises a V at position 19 of the light chain as defined by Kabat numbering. In some embodiments, the framework modified binding molecule of the invention comprises a T at position 25 of the light chain as defined by Kabat numbering. In some embodiments, the framework modified binding molecule of the invention comprises a F at position 36 of the light chain as defined by Kabat numbering. In some embodiments, the framework modified binding molecule of the invention comprises a R at position 37 of the light chain as defined by Kabat numbering. In some embodiments, the framework modified binding molecule of the invention comprises a H at position 38 of the light chain as defined by Kabat numbering. In some embodiments, the framework modified binding molecule of the invention comprises a R at position 39 of the light chain as defined by Kabat numbering. In some embodiments, the framework modified binding molecule of the invention comprises a D at position 41 of the light chain as defined by Kabat numbering. In some embodiments, the framework modified binding molecule of the invention comprises a P at position 43 of the light chain as defined by Kabat numbering. In some embodiments, the framework modified binding molecule of the invention comprises a M at position 48 of the light chain as defined by Kabat numbering. In some embodiments, the framework modified binding molecule of the invention comprises a F at position 49 of the light chain as defined by Kabat numbering. In some embodiments, the framework modified binding molecule of the invention comprises a V at position 55 of the light chain as defined by Kabat numbering. In some embodiments, the framework modified binding molecule of the invention comprises an A at position 56 of the light chain as defined by Kabat numbering. In some embodiments, the framework modified binding molecule of the invention comprises a V at position 58 of the light chain as defined by Kabat numbering. In some embodiments, the framework modified binding molecule of the invention comprises a S at position 59 of the light chain as defined by Kabat numbering. In some embodiments, the framework modified binding molecule of the invention comprises an A at position 64 of the light chain as defined by Kabat numbering. In some embodiments, the framework modified binding molecule of the invention comprises an E at position 70 of the light chain as defined by Kabat numbering. In some embodiments, the framework modified binding molecule of the invention comprises a S at position 80 of the light chain as defined by Kabat numbering. In some embodiments, the framework modified binding molecule of the invention comprises a D at position 81 of the light chain as defined by Kabat numbering. In some embodiments, the framework modified binding molecule of the invention comprises a S at position 83 of the light chain as defined by Kabat numbering. In some embodiments, the framework modified binding molecule of the invention comprises a F at position 87 of the light chain as defined by Kabat numbering. In some embodiments, the framework modified binding molecule of the invention comprises a R at position 103 of the light chain as defined by Kabat numbering. In some embodiments, the framework modified binding molecule of the invention comprises a L at position 104 of the light chain as defined by Kabat numbering. In some embodiments, the framework modified binding molecule of the invention comprises a D at position 105 of the light chain as defined by Kabat numbering.

In some embodiments, the framework modified binding molecule of the invention comprises a combination of at least 2, 3, 4, 5, 6, 7, 8, 9, or 10 framework modifications as outlined in any one of Table 1, Table 2 and Table 3.

TABLE 1
Listing of positions with AFS > 20

| Antibody position | AA mutation | |
|---|---|---|
| (Kabat numbering) | suggested by AFS | Location |
| 1 | D | FWK |
| 2 | V | FWK |
| 11 | V | FWK |
| 17 | D | FWK |
| 18 | G | FWK |
| 19 | A, V | FWK |
| 25 | T | FWK |
| 27A | T | CDRL1 |
| 28 | I | CDRL1 |
| 29 | T | CDRL1 |
| 30 | N | CDRL1 |
| 31 | N | CDRL1 |
| 32 | F | CDRL1 |
| 36 | F | FWk |
| 37 | R | FWk |
| 38 | H | FWk |
| 39 | R | FWk |
| 41 | D | FWk |
| 43 | P | FWk |
| 48 | M | FWk |
| 49 | F | FWk |
| 50 | A | CDRL2 |
| 51 | T | CDRL2 |
| 55 | V | FWK |
| 56 | A | FWK |
| 58 | V | FWK |
| 59 | S | FWK |
| 64 | A | FWK |
| 70 | E | FWK |
| 74 | S | FWK |
| 77 | S | FWK |
| 79 | Q | FWK |
| 80 | S | FWK |
| 81 | D | FWK |
| 83 | S | FWK |
| 87 | F | FWK |
| 89 | H | CDRL3 |
| 90 | H | CDRL3 |
| 91 | F | CDRL3 |
| 95A | P | CDRL3 (VJ junction) |
| 97 | A | CDRL3 |
| 103 | R | FWk |
| 104 | L | FWk |
| 105 | D | FWk |

TABLE 2
AFS cutoff >20 with kabat #

| Position | Came from | | AA | location |
|---|---|---|---|---|
| 1 | VK320 full no J | | D | FWk |
| 2 | VK320 full no J | | V | FWk |
| 11 | VK320 full no J | | V | FWk |
| 17 | VK320 full no J | | D | FWk |
| 18 | | | | |
| 19 | VK320 full no J | | V | FWk |
| 25 | VK320 full no J | | T | FWk |
| 27A | VK320 full no J | VK320 full no | T | CDRL1 |
| | | J (cAFS) + | | |
| | | germ cutoff | | |
| 28 | VK320 full no J | VK320 full no | I | CDRL1 |
| | | J (cAFS) + | | |
| | | germ cutoff | | |
| 29 | | | | |
| 30 | VK320 full no J | VK320 full no | N | CDRL1 |
| | | J (cAFS) + | | |
| | | germ cutoff | | |
| 31 | VK320 full no J | VK320 full no | N | CDRL1 |
| | | J (cAFS) + | | |
| | | germ cutoff | | |
| 32 | | | | CDRL1 |
| 36 | VK320 full no J | | F | FWk |
| 37 | | | | |
| 38 | VK320 full no J | | H | FWk |
| 39 | VK320 full no J | VK320 full no | R | FWk |
| | | J (cAFS) + | | |
| | | germ cutoff | | |
| 41 | | | | |
| 43 | | | | |
| 48 | VK320 full no J | | M | FWk |
| 49 | VK320 full no J | VK320 full no | F | FWk |
| | | J (cAFS) + | | |
| | | germ cutoff | | |
| 50 | | | | CDRL2 |
| 51 | | | | CDRL2 |
| 55 | | | | FWK |
| 56 | | VK320 full no | A | |
| | | J (cAFS) + | | |
| | | germ cutoff | | |
| 58 | VK320 full no J | | V | FWk |
| 59 | | | | FWK |
| 64 | VK320 full no J | | A | FWk |
| 70 | | | | FWK |
| 74 | | | | |
| 77 | VK320 full no J | | S | FWk |
| 79 | | | | FWk |
| 80 | | | | FWk |
| 81 | VK320 full no J | | D | FWk |
| 83 | | | | |
| 87 | VK320 full no J | VK320 full no | F | FWk |
| | | J (cAFS) + | | |
| | | germ cutoff | | |
| 89 | VK320 full no J | | H | FWk |
| 90 | VK320 full no J | | H | FWk |
| 91 | VK320 full no J | VK320 full no | F | FWk |
| | | J (cAFS) + | | |
| | | germ cutoff | | |
| 95A | VK320 full no J | VK320 full no | P | CDRL3 (VJ |
| | | J (cAFS) + | | junction) |
| | | germ cutoff | | |
| 97 | | | | |
| 103 | VK320 full no J | | R | FWk |
| 104 | VK320 full no J | | L | FWk |
| 105 | VK320 full no J | | D | FWk |

TABLE 3
AFS cutoff >20 with kabat #

| Position | Came from | | AA | location |
|---|---|---|---|---|
| 1 | VK320 full with J | | D | FWk |
| 2 | VK320 full with J | | V | FWk |
| 11 | VK320 full with J | | V | FWk |
| 17 | VK320 full with J | | D | FWk |
| 18 | VK320 full with J | | G | |
| 19 | VK320 full with J | | A | FWk |
| 25 | | | | FWk |
| 27A | VK320 full with J | VK320 full w | T | CDRL1 |
| | | J (cAFS) + | | |
| | | germ cutoff | | |
| 28 | VK320 full with J | VK320 full w | I | CDRL1 |
| | | J (cAFS) + | | |
| | | germ cutoff | | |
| 29 | | VK320 full w | T | |
| | | J (cAFS) + | | |
| | | germ cutoff | | |
| 30 | | | | CDRL1 |
| 31 | VK320 full with J | VK320 full w | N | CDRL1 |
| | | J (cAFS) + | | |
| | | germ cutoff | | |
| 32 | VK320 full with J | VK320 full w | F | CDRL1 |
| | | J (cAFS) + | | |
| | | germ cutoff | | |
| 36 | VK320 full with J | | F | FWk |
| 37 | VK320 full with J | | R | |
| 38 | VK320 full with J | | H | FWk |
| 39 | VK320 full with J | | R | FWk |
| 41 | | VK320 full w | D | |
| | | J (cAFS) + | | |
| | | germ cutoff | | |
| 43 | VK320 full with J | | P | |
| 48 | VK320 full with J | | M | FWk |
| 49 | | | | FWk |
| 50 | VK320 full with J | VK320 full w | A | CDRL2 |
| | | J (cAFS) + | | |
| | | germ cutoff | | |
| 51 | VK320 full with J | VK320 full w | T | CDRL2 |
| | | J (cAFS) + | | |
| | | germ cutoff | | |
| 55 | VK320 full with J | | V | FWK |
| 56 | | | | |
| 58 | VK320 full with J | | V | FWk |
| 59 | VK320 full with J | | S | FWK |
| 64 | VK320 full with J | | A | FWk |
| 70 | VK320 full with J | VK320 full w | E | FWK |
| | | J (cAFS) + | | |
| | | germ cutoff | | |
| 74 | VK320 full with J | | S | |
| 77 | VK320 full with J | | S | FWk |
| 79 | VK320 full with J | | Q | FWk |
| 80 | VK320 full with J | | S | FWk |
| 81 | VK320 full with J | | D | FWk |
| 83 | VK320 full with J | VK320 full w | S | |
| | | J (cAFS) + | | |
| | | germ cutoff | | |
| 87 | VK320 full with J | | F | FWk |
| 89 | VK320 full with J | VK320 full w | H | FWk |
| | | J (cAFS) + | | |
| | | germ cutoff | | |
| 90 | VK320 full with J | | H | FWk |
| 91 | VK320 full with J | VK320 full w | F | FWk |
| | | J (cAFS) + | | |
| | | germ cutoff | | |
| 95A | VK320 full with J | VK320 full w | P | CDRL3 (VJ |
| | | J (cAFS) + | | junction) |
| | | germ cutoff | | |
| 97 | VK320 full with J | | A | |
| 103 | VK320 full with J | | R | FWk |
| 104 | | | L | FWk |
| 105 | | | | FWk |

### 4. SARS-COV-2 ANTIBODIES

In some embodiments, the invention provides compositions that bind to SARS-CoV-2 antigen, including, but not limited to, a SARS-CoV-2 spike protein. In some embodiments, the composition that binds to SARS-CoV-2 spike protein is an antibody.

The instant invention relates to the design and development of a synthetic DNA plasmid-encoding human anti-SARS-CoV-2 monoclonal antibody sequences as a novel approach to immunotherapy of SARS-CoV-2 infection, or COVID-19. A single inoculation with this anti-SARS-CoV-2-DMAb generates functional anti-SARS-CoV-2 activity for several weeks in the serum of inoculated animals. Anti-SARS-CoV-2 DMAbs can function as an immune-prophylaxis strategy for SARS-CoV-2 infection, or COVID-19.

The present invention relates to a composition comprising a recombinant nucleic acid sequence encoding an antibody, a fragment thereof, a variant thereof, or a combination thereof. The composition, when administered to a subject in need thereof, can result in the generation of a synthetic antibody in the subject. The synthetic antibody can bind a target molecule (i.e., an antigen) present in the subject. Such binding can neutralize the antigen, block recognition of the antigen by another molecule, for example, a protein or nucleic acid, and elicit or induce an immune response to the antigen.

In one embodiment, the composition comprises a nucleotide sequence encoding a synthetic antibody. In one embodiment, the composition comprises a nucleic acid molecule comprising a first nucleotide sequence encoding a first synthetic antibody and a second nucleotide sequence encoding a second synthetic antibody. In one embodiment, the nucleic acid molecule comprises a nucleotide sequence encoding a cleavage domain.

In one embodiment, the nucleic acid molecule comprises a nucleotide sequence encoding an antibody to the receptor binding domain (RBD) or the Spike protein of the SARS-CoV-2 virus (anti-SARS-CoV-2).

In one embodiment, the nucleic acid molecule comprises a nucleotide sequence encoding a variable heavy chain region and a nucleotide sequence encoding a variable light chain region of an anti-SARS-CoV-2 antibody.

In one embodiment, the invention provides a composition comprising a first nucleic acid molecule comprising a nucleotide sequence encoding a variable heavy chain region of an anti-SARS-CoV-2 antibody and a second nucleic acid molecule comprising a nucleotide sequence encoding a variable light chain region of an anti-SARS-CoV-2 antibody.

Antibodies, including SARS-CoV-2 spike protein fragments, of the present invention include, in certain embodiments, antibody amino acid sequences disclosed herein encoded by any suitable polynucleotide, or any isolated or formulated antibody. Further, antibodies of the present disclosure comprise antibodies having the structural and/or functional features of anti-SARS-CoV-2 spike protein antibodies described herein. In one embodiment, the anti-SARS-CoV-2 spike protein antibody binds SARS-CoV-2 spike protein and, thereby partially or substantially alters at least one biological activity of SARS-CoV-2 spike protein (e.g., receptor binding activity).

In one embodiment, anti-SARS-CoV-2 spike protein antibodies of the invention immunospecifically bind at least one specified epitope specific to the SARS-CoV-2 spike protein and do not specifically bind to other polypeptides. The at least one epitope can comprise at least one antibody binding region that comprises at least one portion of the SARS-CoV-2 spike protein. The term “epitope” as used herein refers to a protein determinant capable of binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents.

In some embodiments, the invention includes compositions comprising an antibody that specifically binds to SARS-CoV-2 spike protein (e.g., binding portion of an antibody). In one embodiment, the anti-SARS-CoV-2 spike protein antibody is a polyclonal antibody. In another embodiment, the anti-SARS-CoV-2 spike protein antibody is a monoclonal antibody. In some embodiments, the anti-SARS-CoV-2 spike protein antibody is a chimeric antibody. In further embodiments, the anti-SARS-CoV-2 spike protein antibody is a humanized antibody.

The binding portion of an antibody comprises one or more fragments of an antibody that retain the ability to specifically bind to binding partner molecule (e.g., SARS-CoV-2 spike protein). It has been shown that the binding function of an antibody can be performed by fragments of a full-length antibody. Examples of binding fragments encompassed within the term “binding portion” of an antibody include (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “binding portion” of an antibody. These antibody fragments are obtained using conventional techniques known to those with skill in the art, and the fragments are screened for utility in the same manner as are intact antibodies. Binding portions can be produced by recombinant DNA techniques, or by enzymatic or chemical cleavage of intact immunoglobulins.

An antibody that binds to SARS-CoV-2 spike protein of the invention is an antibody that inhibits, blocks, or interferes with at least one SARS-CoV-2 spike protein activity (e.g., receptor binding activity), in vitro, in situ and/or in vivo.

In one embodiment, the SARS-CoV-2 antibody comprises a heavy chain comprising an amino acid sequence of SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88. In one embodiment, the SARS-CoV-2 antibody comprises at least the variable heavy chain sequence of the full-length heavy chain sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, or SEQ ID NO:86.

In one embodiment, the SARS-CoV-2 antibody comprises a light chain comprising at least the constant light chain sequence of the full-length light chain sequence of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, or SEQ ID NO:112.

In one embodiment, the SARS-CoV-2 antibody comprises a light chain comprising a LCDR1 comprising SEQ ID NO:138, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:148, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:156, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:162, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:168, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:138, a LCDR2 comprising SEQ ID NO:140 and a LCDR3 comprising SEQ ID NO:183. In one embodiment, the SARS-CoV-2 antibody comprises a light chain comprising a LCDR1 comprising SEQ ID NO:189, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:198, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:205, a LCDR2 comprising SEQ ID NO:207 and a LCDR3 comprising SEQ ID NO:209. In one embodiment, the SARS-CoV-2 antibody comprises a light chain comprising a LCDR1 comprising SEQ ID NO:215, a LCDR2 comprising SEQ ID NO:207 and a LCDR3 comprising SEQ ID NO:217. In one embodiment, the SARS-CoV-2 antibody comprises a light chain comprising a LCDR1 comprising SEQ ID NO:223, a LCDR2 comprising SEQ ID NO:207 and a LCDR3 comprising SEQ ID NO:209. In one embodiment, the SARS-CoV-2 antibody comprises a light chain comprising a LCDR1 comprising SEQ ID NO:229, a LCDR2 comprising SEQ ID NO:207 and a LCDR3 comprising SEQ ID NO:217.

In one embodiment, the SARS-CoV-2 antibody comprises a light chain comprising at least the variable light chain sequence of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, SEQ ID NO:201, SEQ ID NO:211, SEQ ID NO:219, SEQ ID NO:225 or SEQ ID NO:23.

In one embodiment, the SARS-CoV-2 antibody comprises a fragment comprising at least the variable light chain sequence of the full-length light chain sequence of SEQ ID NO:22, SEQ ID NO:118, SEQ ID NO:120, SEQ ID NO:122, SEQ ID NO:124, SEQ ID NO:126, SEQ ID NO:128, SEQ ID NO:130, or SEQ ID NO:132.

Given that certain of the monoclonal antibodies can bind to the SARS-CoV-2 spike protein, the VH and VL sequences can be “mixed and matched” to create other anti-SARS-CoV-2 spike protein binding molecules of this disclosure. Binding of such “mixed and matched” antibodies can be tested using standard binding assays known in the art (e.g., immunoblot etc.). In some embodiments, when VH and VL chains are mixed and matched, a VH sequence from a particular VH/VL pairing is replaced with a structurally similar VH sequence. Likewise, preferably a VL sequence from a particular VH/VL pairing is replaced with a structurally similar VL sequence.

In one embodiment, the heavy chain of the anti-SARS-CoV-2 antibody comprises SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and the light chain of the anti-SARS-CoV-2 antibody comprises SEQ ID NO:22, SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, SEQ ID NO:112; SEQ ID NO:146, SEQ ID NO:154, SEQ ID NO:160, SEQ ID NO:166, SEQ ID NO:172, SEQ ID NO:177, SEQ ID NO:181, SEQ ID NO:187, SEQ ID NO:193, SEQ ID NO:197, or SEQ ID NO:203 or a fragment thereof.

In one embodiment, the heavy chain of the anti-SARS-CoV-2 antibody comprises SEQ ID NO:235, and the light chain of the anti-SARS-CoV-2 antibody comprises SEQ ID NO:212, SEQ ID NO:220, SEQ ID NO:226, or SEQ ID NO:232 or a fragment thereof.

In one embodiment, the invention relates to a nucleic acid molecule comprising a nucleotide sequence encoding a heavy chain or light chain of an anti-SARS-CoV-2 antibody. In one embodiment, the invention relates to a combination of nucleic acid molecules comprising a nucleotide sequence encoding a heavy chain and a nucleotide sequence encoding a light chain of an anti-SARS-CoV-2 antibody.

In some embodiments, the nucleic acid molecule comprises a nucleic acid sequence encoding a heavy chain comprising an amino acid sequence of SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88. In some embodiments, the nucleic acid molecule comprises a nucleic acid sequence encoding at least the variable heavy chain sequence of the full-length heavy chain sequence of SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, or SEQ ID NO:86.

In some embodiments, the nucleic acid molecule comprises a nucleic acid sequence encoding at least the constant light chain sequence of the full-length light chain sequence of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, or SEQ ID NO:112.

In some embodiments, the nucleic acid molecule comprises a nucleic acid sequence encoding the LCDR1, LCDR2 and LCDR3 of the SARS-CoV-2 antibody. In one embodiment, the SARS-CoV-2 antibody comprises a light chain comprising a LCDR1 comprising SEQ ID NO:138, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:148, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:156, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:162, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:168, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:138, a LCDR2 comprising SEQ ID NO:140 and a LCDR3 comprising SEQ ID NO:183. In one embodiment, the SARS-CoV-2 antibody comprises a light chain comprising a LCDR1 comprising SEQ ID NO:189, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:198, a LCDR2 comprising SEQ ID NO:140 and a LCDR3 comprising SEQ ID NO:142. In one embodiment, the SARS-CoV-2 antibody comprises a light chain comprising a LCDR1 comprising SEQ ID NO:205, a LCDR2 comprising SEQ ID NO:207 and a LCDR3 comprising SEQ ID NO:209. In one embodiment, the SARS-CoV-2 antibody comprises a light chain comprising a LCDR1 comprising SEQ ID NO:215, a LCDR2 comprising SEQ ID NO:207 and a LCDR3 comprising SEQ ID NO:217. In one embodiment, the SARS-CoV-2 antibody comprises a light chain comprising a LCDR1 comprising SEQ ID NO:223, a LCDR2 comprising SEQ ID NO:207 and a LCDR3 comprising SEQ ID NO:209. In one embodiment, the SARS-CoV-2 antibody comprises a light chain comprising a LCDR1 comprising SEQ ID NO:229, a LCDR2 comprising SEQ ID NO:207 and a LCDR3 comprising SEQ ID NO:217.

In some embodiments, the nucleic acid molecule comprises a nucleic acid sequence encoding a light chain comprising at least the variable light chain sequence of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, SEQ ID NO:201, SEQ ID NO:211, SEQ ID NO:219, SEQ ID NO:225 or SEQ ID NO:231.

In some embodiments, the nucleic acid molecule comprises a nucleic acid sequence at least the variable light chain sequence of the full-length light chain sequence of SEQ ID NO:22, SEQ ID NO:118, SEQ ID NO:120, SEQ ID NO:122, SEQ ID NO:124, SEQ ID NO:126, SEQ ID NO:128, SEQ ID NO:130, or SEQ ID NO:132.

In one embodiment, the invention relates to a nucleic acid molecule comprising a nucleotide sequence encoding a heavy chain of an anti-SARS-CoV-2 antibody comprising a nucleotide sequence of SEQ ID NO:3, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

In one embodiment, the invention relates to a nucleic acid molecule comprising a nucleotide sequence encoding a variable heavy chain sequence of the full-length heavy chain sequence selected from the group consisting of SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:63, SEQ ID NO:75, SEQ ID NO:83, or SEQ ID NO:85.

In one embodiment, the invention relates to a nucleic acid molecule comprising a nucleotide sequence encoding at least the constant light chain sequence of the full-length light chain sequence of SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, or SEQ ID NO:111.

In one embodiment, the invention relates to a nucleic acid molecule comprising a LCDR1 encoding sequence comprising SEQ ID NO:137, a LCDR2 encoding sequence comprising SEQ ID NO:139 and a LCDR3 encoding sequence comprising SEQ ID NO:141. In one embodiment, the invention relates to a nucleic acid molecule comprising a LCDR1 encoding sequence comprising SEQ ID NO:147, a LCDR2 encoding sequence comprising SEQ ID NO:139 and a LCDR3 encoding sequence comprising SEQ ID NO:141. In one embodiment, the invention relates to a nucleic acid molecule comprising a LCDR1 encoding sequence comprising SEQ ID NO:155, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:161, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:167, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:173, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:173, a LCDR2 encoding sequence comprising SEQ ID NO:149 and a LCDR3 encoding sequence comprising SEQ ID NO:182. In one embodiment, the invention relates to a nucleic acid molecule comprising a LCDR1 encoding sequence comprising SEQ ID NO:188, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:198, a LCDR2 encoding sequence comprising SEQ ID NO:149 and a LCDR3 encoding sequence comprising SEQ ID NO:150. In one embodiment, the invention relates to a nucleic acid molecule comprising a LCDR1 encoding sequence comprising SEQ ID NO:204, a LCDR2 encoding sequence comprising SEQ ID NO:206 and a LCDR3 encoding sequence comprising SEQ ID NO:208. In one embodiment, the invention relates to a nucleic acid molecule comprising a LCDR1 encoding sequence comprising SEQ ID NO:214, a LCDR2 encoding sequence comprising SEQ ID NO:206 and a LCDR3 encoding sequence comprising SEQ ID NO:216. In one embodiment, the invention relates to a nucleic acid molecule comprising a LCDR1 encoding sequence comprising SEQ ID NO:222, a LCDR2 encoding sequence comprising SEQ ID NO:206 and a LCDR3 encoding sequence comprising SEQ ID NO:208. In one embodiment, the invention relates to a nucleic acid molecule comprising a LCDR1 encoding sequence comprising SEQ ID NO:228, a LCDR2 encoding sequence comprising SEQ ID NO:206 and a LCDR3 encoding sequence comprising SEQ ID NO:216.

In one embodiment, the invention relates to a nucleic acid molecule comprising a nucleotide sequence encoding a light chain variable region of SEQ ID NO:143, SEQ ID NO:151, SEQ ID NO:159, SEQ ID NO:163, SEQ ID NO:169, SEQ ID NO:174, SEQ ID NO:178, SEQ ID NO:184, SEQ ID NO:190, SEQ ID NO:194, SEQ ID NO:200, SEQ ID NO:210, SEQ ID NO:218, SEQ ID NO:224 or SEQ ID NO:230.

In one embodiment, the invention relates to a nucleic acid molecule comprising a nucleotide sequence encoding at least the variable light chain sequence of the full-length light chain sequence of: SEQ ID NO:21, SEQ ID NO:117, SEQ ID NO:119, SEQ ID NO:121, SEQ ID NO:123, SEQ ID NO:125, SEQ ID NO:127, SEQ ID NO:129, or SEQ ID NO:131.

In one embodiment, the nucleotide sequence encoding an anti-SARS-CoV-2 antibody comprises an RNA sequence transcribed from a DNA sequence encoding a light chain amino acid sequence or a fragment thereof. In one embodiment, the nucleotide sequence encoding an anti-SARS-CoV-2 antibody comprises an RNA sequence transcribed from a DNA sequence encoding a heavy chain amino acid sequence or a fragment thereof.

In one embodiment, the invention relates to a combination of a first nucleic acid molecule encoding a heavy chain of an anti-SARS-CoV-2 antibody, and a second nucleic acid molecule encoding a light chain of an anti-SARS-CoV-2 antibody. In one embodiment, the first nucleic acid molecule is a first plasmid comprising a nucleotide sequence encoding a heavy chain of an anti-SARS-CoV-2 antibody and the second nucleic acid molecule is a second plasmid encoding a light chain of an anti-SARS-CoV-2 antibody.

In one embodiment, the first nucleic acid molecule encoding a heavy chain of an anti-SARS-CoV-2 antibody encodes SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and the second nucleic acid molecule encoding a light chain of an anti-SARS-CoV-2 antibody encodes SEQ ID NO:22, SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, SEQ ID NO:112; SEQ ID NO:146, SEQ ID NO:154, SEQ ID NO:160, SEQ ID NO:166, SEQ ID NO:172, SEQ ID NO:177, SEQ ID NO:181, SEQ ID NO:187, SEQ ID NO:193, SEQ ID NO:197, or SEQ ID NO:203 or a fragment thereof.

In one embodiment, the first nucleic acid molecule encoding a heavy chain of an anti-SARS-CoV-2 antibody encodes SEQ ID NO:235, and the second nucleic acid molecule encoding a light chain of an anti-SARS-CoV-2 antibody encodes SEQ ID NO:212, SEQ ID NO:220, SEQ ID NO:226, or SEQ ID NO:232 or a fragment thereof.

In one embodiment, the first nucleic acid molecule comprises SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87, encoding the heavy chain of the anti-SARS-CoV-2 antibody, and the second nucleic acid molecule comprises a nucleotide sequence of SEQ ID NO:21, SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, SEQ ID NO:111, SEQ ID NO:145, SEQ ID NO:153, SEQ ID NO:159, SEQ ID NO:165, SEQ ID NO:171, SEQ ID NO:176, SEQ ID NO:180, SEQ ID NO:186, SEQ ID NO:192, SEQ ID NO:196, or SEQ ID NO:202 encoding the light chain of the anti-SARS-CoV-2 antibody.

In one embodiment, the first nucleic acid molecule comprises SEQ ID NO:235 encoding the heavy chain of the anti-SARS-CoV-2 antibody, and the second nucleic acid molecule comprises a nucleotide sequence of SEQ ID NO:212, SEQ ID NO:220, SEQ ID NO:226, or SEQ ID NO:232 encoding the light chain of the anti-SARS-CoV-2 antibody.

In one embodiment, at least one of the first nucleic acid molecule and the second nucleic acid molecule is a DNA molecule. Therefore, in some embodiments, the invention provides a combination of DNA molecules encoding an anti-SARS-CoV-2 antibody of the invention.

In one embodiment, at least one of the first nucleic acid molecule and the second nucleic acid molecule is an RNA molecule. Therefore, in some embodiments, the invention provides a combination of RNA molecules encoding an anti-SARS-CoV-2 antibody of the invention.

The composition of the invention can treat, prevent and/or protect against any disease, disorder, or condition associated with SARS-CoV-2 infection. In certain embodiments, the composition can treat, prevent, and or/protect against viral infection. In certain embodiments, the composition can treat, prevent, and or/protect against condition associated with SARS-CoV-2 infection. In certain embodiments, the composition can treat, prevent, and or/protect against COVID-19.

The composition can result in the generation of the synthetic antibody in the subject within at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 20 hours, 25 hours, 30 hours, 35 hours, 40 hours, 45 hours, 50 hours, or 60 hours of administration of the composition to the subject. The composition can result in generation of the synthetic antibody in the subject within at least about 1 day, 2 days, 3 days, 4 days, 5 days, 6 days, 7 days, 8 days, 9 days, or 10 days of administration of the composition to the subject. The composition can result in generation of the synthetic antibody in the subject within about 1 hour to about 6 days, about 1 hour to about 5 days, about 1 hour to about 4 days, about 1 hour to about 3 days, about 1 hour to about 2 days, about 1 hour to about 1 day, about 1 hour to about 72 hours, about 1 hour to about 60 hours, about 1 hour to about 48 hours, about 1 hour to about 36 hours, about 1 hour to about 24 hours, about 1 hour to about 12 hours, or about 1 hour to about 6 hours of administration of the composition to the subject.

The composition, when administered to the subject in need thereof, can result in the generation of the synthetic antibody in the subject more quickly than the generation of an endogenous antibody in a subject who is administered an antigen to induce a humoral immune response. The composition can result in the generation of the synthetic antibody at least about 1 day, 2 days, 3 days, 4 days, 5 days, 6 days, 7 days, 8 days, 9 days, or 10 days before the generation of the endogenous antibody in the subject who was administered an antigen to induce a humoral immune response.

The composition of the present invention can have features required of effective compositions such as being safe so that the composition does not cause illness or death; being protective against illness; and providing ease of administration, few side effects, biological stability and low cost per dose.

In some embodiments, the SARS-CoV-2 spike protein binding molecules (e.g., antibodies, etc.) of the present invention, exhibit a high capacity to detect and bind SARS-CoV-2 spike protein in a complex mixture of salts, compounds and other polypeptides, e.g., as assessed by any one of several in vitro and in vivo assays known in the art. The skilled artisan will understand that the SARS-CoV-2 spike protein binding molecules (e.g., antibodies, etc.) described herein as useful in the methods of diagnosis and treatment and prevention of disease, are also useful in procedures and methods of the invention that include, but are not limited to, an immunochromatography assay, an immunodot assay, a Luminex assay, an ELISA assay, an ELISPOT assay, a protein microarray assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, a protein chip assay, separation and purification processes, and affinity chromatography (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, the SARS-CoV-2 spike protein binding molecules (e.g., antibodies, etc.) of the present invention, exhibit a high capacity to reduce or to neutralize SARS-CoV-2 spike protein activity (e.g., receptor binding activity, etc.) as assessed by any one of several in vitro and in vivo assays known in the art. For example, these SARS-CoV-2 spike protein binding molecules (e.g., antibodies, etc.) neutralize SARS-CoV-2-associated or SARS-CoV-2-mediated disease or disorder.

As used herein, a SARS-CoV-2 antigen binding molecule (e.g., antibody, etc.) that “specifically binds to a SARS-CoV-2 antigen” binds to a SARS-CoV-2 spike protein with a KD of 1×10⁻⁶ M or less, more preferably 1×10⁻⁷ M or less, more preferably 1×10⁻⁸ M or less, more preferably 5×10⁻⁹ M or less, more preferably 1×10⁻⁹ M or less or even more preferably 3×10⁻¹⁰ M or less. The term “does not substantially bind” to a protein or cells, as used herein, means does not bind or does not bind with a high affinity to the protein or cells, i.e., binds to the protein or cells with a KD of greater than 1×10⁶ M or more, more preferably 1×10⁵ M or more, more preferably 1×10⁴ M or more, more preferably 1×10³ M or more, even more preferably 1×10² M or more. The term “KD”, as used herein, is intended to refer to the dissociation constant, which is obtained from the ratio of Kd to Ka (i.e., Kd/Ka) and is expressed as a molar concentration (M). KD values for a SARS-CoV-2 spike protein binding molecule (e.g., antibody, etc.) can be determined using methods well established in the art. A preferred method for determining the KD of a binding molecule (e.g., antibody, etc.) is by using surface plasmon resonance, preferably using a biosensor system such as a Biacore® system.

As used herein, the term “high affinity” for an IgG antibody refers to an antibody having a KD of 1×10⁻⁷ M or less, more preferably 5×10⁻⁸ M or less, even more preferably 1×10⁻⁸ M or less, even more preferably 5×10⁻⁹M or less and even more preferably 1×10⁻⁹ M or less for a target binding partner molecule. However, “high affinity” binding can vary for other antibody isotypes. For example, “high affinity” binding for an IgM isotype refers to an antibody having a KD of 10⁻⁶ M or less, more preferably 10⁻⁷ M or less, even more preferably 10⁻⁸ M or less.

In certain embodiments, the antibody comprises a heavy chain constant region, such as an IgG1, IgG2, IgG3, IgG4, IgA, IgE, IgM or IgD constant region. Preferably, the heavy chain constant region is an IgG1 heavy chain constant region or an IgG4 heavy chain constant region. Furthermore, the antibody can comprise a light chain constant region, either a kappa light chain constant region or a lambda light chain constant region. Preferably, the antibody comprises a kappa light chain constant region. Alternatively, the antibody portion can be, for example, a Fab fragment or a single chain Fv fragment.

### Recombinant Nucleic Acid Sequence

As described above, the composition can comprise a recombinant nucleic acid sequence. The recombinant nucleic acid sequence can encode the antibody, a fragment thereof, a variant thereof, or a combination thereof. The antibody is described in more detail below.

The recombinant nucleic acid sequence can be a heterologous nucleic acid sequence. The recombinant nucleic acid sequence can include one or more heterologous nucleic acid sequences.

The recombinant nucleic acid sequence can be an optimized nucleic acid sequence. Such optimization can increase or alter the immunogenicity of the antibody. Optimization can also improve transcription and/or translation. Optimization can include one or more of the following: low GC content leader sequence to increase transcription; mRNA stability and codon optimization; addition of a kozak sequence for increased translation; addition of an immunoglobulin (Ig) leader sequence encoding a signal peptide; addition of an internal IRES sequence and eliminating to the extent possible cis-acting sequence motifs (i.e., internal TATA boxes).

### Recombinant Nucleic Acid Sequence Construct

The recombinant nucleic acid sequence can include one or more recombinant nucleic acid sequence constructs. The recombinant nucleic acid sequence construct can include one or more components, which are described in more detail below.

The recombinant nucleic acid sequence construct can include a heterologous nucleic acid sequence that encodes a heavy chain polypeptide, a fragment thereof, a variant thereof, or a combination thereof. The recombinant nucleic acid sequence construct can include a heterologous nucleic acid sequence that encodes a light chain polypeptide, a fragment thereof, a variant thereof, or a combination thereof. The recombinant nucleic acid sequence construct can also include a heterologous nucleic acid sequence that encodes a protease or peptidase cleavage site. The recombinant nucleic acid sequence construct can also include a heterologous nucleic acid sequence that encodes an internal ribosome entry site (IRES). An IRES may be either a viral IRES or an eukaryotic IRES. The recombinant nucleic acid sequence construct can include one or more leader sequences, in which each leader sequence encodes a signal peptide. The recombinant nucleic acid sequence construct can include one or more promoters, one or more introns, one or more transcription termination regions, one or more initiation codons, one or more termination or stop codons, and/or one or more polyadenylation signals. The recombinant nucleic acid sequence construct can also include one or more linker or tag sequences. The tag sequence can encode a hemagglutinin (HA) tag.

### (1) Heavy Chain Polypeptide

The recombinant nucleic acid sequence construct can include the heterologous nucleic acid encoding the heavy chain polypeptide, a fragment thereof, a variant thereof, or a combination thereof. The heavy chain polypeptide can include a variable heavy chain (VH) region and/or at least one constant heavy chain (CH) region. The at least one constant heavy chain region can include a constant heavy chain region 1 (CH1), a constant heavy chain region 2 (CH2), and a constant heavy chain region 3 (CH3), and/or a hinge region.

In some embodiments, the heavy chain polypeptide can include a VH region and a CH1 region. In other embodiments, the heavy chain polypeptide can include a VH region, a CH1 region, a hinge region, a CH2 region, and a CH3 region.

The heavy chain polypeptide can include a complementarity determining region (“CDR”) set. The CDR set can contain three hypervariable regions of the VH region. Proceeding from N-terminus of the heavy chain polypeptide, these CDRs are denoted “CDR1,” “CDR2,” and “CDR3,” respectively. CDR1, CDR2, and CDR3 of the heavy chain polypeptide can contribute to binding or recognition of the antigen.

### (2) Light Chain Polypeptide

The recombinant nucleic acid sequence construct can include the heterologous nucleic acid sequence encoding the light chain polypeptide, a fragment thereof, a variant thereof, or a combination thereof. The light chain polypeptide can include a variable light chain (VL) region and/or a constant light chain (CL) region.

The light chain polypeptide can include a complementarity determining region (“CDR”) set. The CDR set can contain three hypervariable regions of the VL region. Proceeding from N-terminus of the light chain polypeptide, these CDRs are denoted “CDR1,” “CDR2,” and “CDR3,” respectively. CDR1, CDR2, and CDR3 of the light chain polypeptide can contribute to binding or recognition of the antigen.

### (3) Protease Cleavage Site

The recombinant nucleic acid sequence construct can include heterologous nucleic acid sequence encoding a protease cleavage site. The protease cleavage site can be recognized by a protease or peptidase. The protease can be an endopeptidase or endoprotease, for example, but not limited to, furin, elastase, HtrA, calpain, trypsin, chymotrypsin, trypsin, and pepsin. The protease can be furin. In other embodiments, the protease can be a serine protease, a threonine protease, cysteine protease, aspartate protease, metalloprotease, glutamic acid protease, or any protease that cleaves an internal peptide bond (i.e., does not cleave the N-terminal or C-terminal peptide bond).

The protease cleavage site can include one or more amino acid sequences that promote or increase the efficiency of cleavage. The one or more amino acid sequences can promote or increase the efficiency of forming or generating discrete polypeptides. The one or more amino acids sequences can include a 2A peptide sequence.

### (4) Linker Sequence

The recombinant nucleic acid sequence construct can include one or more linker sequences. The linker sequence can spatially separate or link the one or more components described herein. In other embodiments, the linker sequence can encode an amino acid sequence that spatially separates or links two or more polypeptides.

### (5) Promoter

The recombinant nucleic acid sequence construct can include one or more promoters. The one or more promoters may be any promoter that is capable of driving gene expression and regulating gene expression. Such a promoter is a cis-acting sequence element required for transcription via a DNA dependent RNA polymerase. Selection of the promoter used to direct gene expression depends on the particular application. The promoter may be positioned about the same distance from the transcription start in the recombinant nucleic acid sequence construct as it is from the transcription start site in its natural setting. However, variation in this distance may be accommodated without loss of promoter function.

The promoter may be operably linked to the heterologous nucleic acid sequence encoding the heavy chain polypeptide and/or light chain polypeptide. The promoter may be a promoter shown effective for expression in eukaryotic cells. The promoter operably linked to the coding sequence may be a CMV promoter, a promoter from simian virus 40 (SV40), such as SV40 early promoter and SV40 later promoter, a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, human polyhedrin, or human metalothionein.

The promoter can be a constitutive promoter or an inducible promoter, which initiates transcription only when the host cell is exposed to some particular external stimulus. In the case of a multicellular organism, the promoter can also be specific to a particular tissue or organ or stage of development. The promoter may also be a tissue specific promoter, such as a muscle or skin specific promoter, natural or synthetic. Examples of such promoters are described in US patent application publication no. US20040175727, the contents of which are incorporated herein in its entirety.

The promoter can be associated with an enhancer. The enhancer can be located upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

### (6) Intron

The recombinant nucleic acid sequence construct can include one or more introns. Each intron can include functional splice donor and acceptor sites. The intron can include an enhancer of splicing. The intron can include one or more signals required for efficient splicing.

### (7) Transcription Termination Region

The recombinant nucleic acid sequence construct can include one or more transcription termination regions. The transcription termination region can be downstream of the coding sequence to provide for efficient termination. The transcription termination region can be obtained from the same gene as the promoter described above or can be obtained from one or more different genes.

### (8) Initiation Codon

The recombinant nucleic acid sequence construct can include one or more initiation codons. The initiation codon can be located upstream of the coding sequence. The initiation codon can be in frame with the coding sequence. The initiation codon can be associated with one or more signals required for efficient translation initiation, for example, but not limited to, a ribosome binding site.

### (9) Termination Codon

The recombinant nucleic acid sequence construct can include one or more termination or stop codons. The termination codon can be downstream of the coding sequence. The termination codon can be in frame with the coding sequence. The termination codon can be associated with one or more signals required for efficient translation termination.

### (10) Polyadenylation Signal

The recombinant nucleic acid sequence construct can include one or more polyadenylation signals. The polyadenylation signal can include one or more signals required for efficient polyadenylation of the transcript. The polyadenylation signal can be positioned downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

### (11) Leader Sequence

The recombinant nucleic acid sequence construct can include one or more leader sequences. The leader sequence can encode a signal peptide. The signal peptide can be an immunoglobulin (Ig) signal peptide, for example, but not limited to, an IgG signal peptide and a IgE signal peptide.

### Arrangement of the Recombinant Nucleic Acid Sequence Construct

As described above, the recombinant nucleic acid sequence can include one or more recombinant nucleic acid sequence constructs, in which each recombinant nucleic acid sequence construct can include one or more components. The one or more components are described in detail above. The one or more components, when included in the recombinant nucleic acid sequence construct, can be arranged in any order relative to one another. In some embodiments, the one or more components can be arranged in the recombinant nucleic acid sequence construct as described below.

### (12) Arrangement 1

In one arrangement, a first recombinant nucleic acid sequence construct can include the heterologous nucleic acid sequence encoding the heavy chain polypeptide and a second recombinant nucleic acid sequence construct can include the heterologous nucleic acid sequence encoding the light chain polypeptide. The first recombinant nucleic acid sequence construct can be placed in a vector. The second recombinant nucleic acid sequence construct can be placed in a second or separate vector. Placement of the recombinant nucleic acid sequence construct into the vector is described in more detail below.

The first recombinant nucleic acid sequence construct can also include the promoter, intron, transcription termination region, initiation codon, termination codon, and/or polyadenylation signal. The first recombinant nucleic acid sequence construct can further include the leader sequence, in which the leader sequence is located upstream (or 5′) of the heterologous nucleic acid sequence encoding the heavy chain polypeptide. Accordingly, the signal peptide encoded by the leader sequence can be linked by a peptide bond to the heavy chain polypeptide.

The second recombinant nucleic acid sequence construct can also include the promoter, initiation codon, termination codon, and polyadenylation signal. The second recombinant nucleic acid sequence construct can further include the leader sequence, in which the leader sequence is located upstream (or 5′) of the heterologous nucleic acid sequence encoding the light chain polypeptide. Accordingly, the signal peptide encoded by the leader sequence can be linked by a peptide bond to the light chain polypeptide.

Accordingly, one example of arrangement 1 can include the first vector (and thus first recombinant nucleic acid sequence construct) encoding the heavy chain polypeptide that includes VH and CH1, and the second vector (and thus second recombinant nucleic acid sequence construct) encoding the light chain polypeptide that includes VL and CL. A second example of arrangement 1 can include the first vector (and thus first recombinant nucleic acid sequence construct) encoding the heavy chain polypeptide that includes VH, CH1, hinge region, CH2, and CH3, and the second vector (and thus second recombinant nucleic acid sequence construct) encoding the light chain polypeptide that includes VL and CL.

### (13) Arrangement 2

In a second arrangement, the recombinant nucleic acid sequence construct can include the heterologous nucleic acid sequence encoding the heavy chain polypeptide and the heterologous nucleic acid sequence encoding the light chain polypeptide. The heterologous nucleic acid sequence encoding the heavy chain polypeptide can be positioned upstream (or 5′) of the heterologous nucleic acid sequence encoding the light chain polypeptide. Alternatively, the heterologous nucleic acid sequence encoding the light chain polypeptide can be positioned upstream (or 5′) of the heterologous nucleic acid sequence encoding the heavy chain polypeptide.

The recombinant nucleic acid sequence construct can be placed in the vector as described in more detail below.

The recombinant nucleic acid sequence construct can include the heterologous nucleic acid sequence encoding the protease cleavage site and/or the linker sequence. If included in the recombinant nucleic acid sequence construct, the heterologous nucleic acid sequence encoding the protease cleavage site can be positioned between the heterologous nucleic acid sequence encoding the heavy chain polypeptide and the heterologous nucleic acid sequence encoding the light chain polypeptide. Accordingly, the protease cleavage site allows for separation of the heavy chain polypeptide and the light chain polypeptide into distinct polypeptides upon expression. In other embodiments, if the linker sequence is included in the recombinant nucleic acid sequence construct, then the linker sequence can be positioned between the heterologous nucleic acid sequence encoding the heavy chain polypeptide and the heterologous nucleic acid sequence encoding the light chain polypeptide.

The recombinant nucleic acid sequence construct can also include the promoter, intron, transcription termination region, initiation codon, termination codon, and/or polyadenylation signal. The recombinant nucleic acid sequence construct can include one or more promoters. The recombinant nucleic acid sequence construct can include two promoters such that one promoter can be associated with the heterologous nucleic acid sequence encoding the heavy chain polypeptide and the second promoter can be associated with the heterologous nucleic acid sequence encoding the light chain polypeptide. In still other embodiments, the recombinant nucleic acid sequence construct can include one promoter that is associated with the heterologous nucleic acid sequence encoding the heavy chain polypeptide and the heterologous nucleic acid sequence encoding the light chain polypeptide.

The recombinant nucleic acid sequence construct can further include two leader sequences, in which a first leader sequence is located upstream (or 5′) of the heterologous nucleic acid sequence encoding the heavy chain polypeptide and a second leader sequence is located upstream (or 5′) of the heterologous nucleic acid sequence encoding the light chain polypeptide. Accordingly, a first signal peptide encoded by the first leader sequence can be linked by a peptide bond to the heavy chain polypeptide and a second signal peptide encoded by the second leader sequence can be linked by a peptide bond to the light chain polypeptide.

Accordingly, one example of arrangement 2 can include the vector (and thus recombinant nucleic acid sequence construct) encoding the heavy chain polypeptide that includes VH and CH1, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A second example of arrangement of 2 can include the vector (and thus recombinant nucleic acid sequence construct) encoding the heavy chain polypeptide that includes VH and CH1, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

A third example of arrangement 2 can include the vector (and thus recombinant nucleic acid sequence construct) encoding the heavy chain polypeptide that includes VH, CH1, hinge region, CH2, and CH3, and the light chain polypeptide that includes VL and CL, in which the linker sequence is positioned between the heterologous nucleic acid sequence encoding the heavy chain polypeptide and the heterologous nucleic acid sequence encoding the light chain polypeptide.

A forth example of arrangement of 2 can include the vector (and thus recombinant nucleic acid sequence construct) encoding the heavy chain polypeptide that includes VH, CH1, hinge region, CH2, and CH3, and the light chain polypeptide that includes VL and CL, in which the heterologous nucleic acid sequence encoding the protease cleavage site is positioned between the heterologous nucleic acid sequence encoding the heavy chain polypeptide and the heterologous nucleic acid sequence encoding the light chain polypeptide.

Expression from the Recombinant Nucleic Acid Sequence Construct

As described above, the recombinant nucleic acid sequence construct can include, amongst the one or more components, the heterologous nucleic acid sequence encoding the heavy chain polypeptide and/or the heterologous nucleic acid sequence encoding the light chain polypeptide. Accordingly, the recombinant nucleic acid sequence construct can facilitate expression of the heavy chain polypeptide and/or the light chain polypeptide.

When arrangement 1 as described above is utilized, the first recombinant nucleic acid sequence construct can facilitate the expression of the heavy chain polypeptide and the second recombinant nucleic acid sequence construct can facilitate expression of the light chain polypeptide. When arrangement 2 as described above is utilized, the recombinant nucleic acid sequence construct can facilitate the expression of the heavy chain polypeptide and the light chain polypeptide.

Upon expression, for example, but not limited to, in a cell, organism, or mammal, the heavy chain polypeptide and the light chain polypeptide can assemble into the synthetic antibody. In particular, the heavy chain polypeptide and the light chain polypeptide can interact with one another such that assembly results in the synthetic antibody being capable of binding the antigen. In other embodiments, the heavy chain polypeptide and the light chain polypeptide can interact with one another such that assembly results in the synthetic antibody being more immunogenic as compared to an antibody not assembled as described herein. In still other embodiments, the heavy chain polypeptide and the light chain polypeptide can interact with one another such that assembly results in the synthetic antibody being capable of eliciting or inducing an immune response against the antigen.

### Vector

The recombinant nucleic acid sequence construct described above can be placed in one or more vectors. The one or more vectors can contain an origin of replication. The one or more vectors can be a plasmid, bacteriophage, bacterial artificial chromosome or yeast artificial chromosome. The one or more vectors can be either a self-replication extra chromosomal vector, or a vector which integrates into a host genome.

Vectors include, but are not limited to, plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and include both the expression and non-expression plasmids. In some embodiments, the vector includes linear DNA, enzymatic DNA or synthetic DNA. Where a recombinant microorganism or cell culture is described as hosting an “expression vector” this includes both extra-chromosomal circular and linear DNA and DNA that has been incorporated into the host chromosome(s). Where a vector is being maintained by a host cell, the vector may either be stably replicated by the cells during mitosis as an autonomous structure, or is incorporated within the host's genome.

The one or more vectors can be a heterologous expression construct, which is generally a plasmid that is used to introduce a specific gene into a target cell. Once the expression vector is inside the cell, the heavy chain polypeptide and/or light chain polypeptide that are encoded by the recombinant nucleic acid sequence construct is produced by the cellular-transcription and translation machinery ribosomal complexes. The one or more vectors can express large amounts of stable messenger RNA, and therefore proteins.

### (14) Expression Vector

The one or more vectors can be a circular plasmid or a linear nucleic acid. The circular plasmid and linear nucleic acid are capable of directing expression of a particular nucleotide sequence in an appropriate subject cell. The one or more vectors comprising the recombinant nucleic acid sequence construct may be chimeric, meaning that at least one of its components is heterologous with respect to at least one of its other components.

### (15) Plasmid

The one or more vectors can be a plasmid. The plasmid may be useful for transfecting cells with the recombinant nucleic acid sequence construct. The plasmid may be useful for introducing the recombinant nucleic acid sequence construct into the subject. The plasmid may also comprise a regulatory sequence, which may be well suited for gene expression in a cell into which the plasmid is administered.

The plasmid may also comprise a mammalian origin of replication in order to maintain the plasmid extra-chromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may be pSE420 (Invitrogen, San Diego, Calif.), which may be used for protein production in Escherichia coli (E. coli). The plasmid may also be pYES2 (Invitrogen, San Diego, Calif.), which may be used for protein production in Saccharomyces cerevisiae strains of yeast. The plasmid may also be of the MAXBAC™ complete baculovirus expression system (Invitrogen, San Diego, Calif.), which may be used for protein production in insect cells. The plasmid may also be pcDNAI or pcDNA3 (Invitrogen, San Diego, Calif.), which may be used for protein production in mammalian cells such as Chinese hamster ovary (CHO) cells.

### (16) RNA

In one embodiment, the nucleic acid is an RNA molecule. In one embodiment, the RNA molecule is transcribed from a DNA sequence described herein. Accordingly, in one embodiment, the invention provides an RNA molecule encoding one or more of the DMAbs. The RNA may be plus-stranded. Accordingly, in some embodiments, the RNA molecule can be translated by cells without needing any intervening replication steps such as reverse transcription. A RNA molecule useful with the invention may have a 5′ cap (e.g. a 7-methylguanosine). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. A RNA molecule useful with the invention may be single-stranded. A RNA molecule useful with the invention may comprise synthetic RNA. In some embodiments, the RNA molecule is a naked RNA molecule. In one embodiment, the RNA molecule is comprised within a vector.

In one embodiment, the RNA has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of RNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many RNAs is known in the art. In other embodiments, the 5′ UTR can be derived from an RNA virus whose RNA genome is stable in cells. In other embodiments, various nucleotide analogues can be used in the 3′ or 5′ UTR to impede exonuclease degradation of the RNA.

In one embodiment, the RNA has both a cap on the 5′ end and a 3′ poly(A) tail which determine ribosome binding, initiation of translation and stability of RNA in the cell.

In one embodiment, the RNA is a nucleoside-modified RNA. Nucleoside-modified RNA have particular advantages over non-modified RNA, including for example, increased stability, low or absent innate immunogenicity, and enhanced translation.

### (17) Circular and Linear Vector

The one or more vectors may be circular plasmid, which may transform a target cell by integration into the cellular genome or exist extra-chromosomally (e.g., autonomous replicating plasmid with an origin of replication). The vector can be pVAX, pcDNA3.0, or provax, or any other expression vector capable of expressing the heavy chain polypeptide and/or light chain polypeptide encoded by the recombinant nucleic acid sequence construct.

Also provided herein is a linear nucleic acid, or linear expression cassette (“LEC”), that is capable of being efficiently delivered to a subject via electroporation and expressing the heavy chain polypeptide and/or light chain polypeptide encoded by the recombinant nucleic acid sequence construct. The LEC may be any linear DNA devoid of any phosphate backbone. The LEC may not contain any antibiotic resistance genes and/or a phosphate backbone. The LEC may not contain other nucleic acid sequences unrelated to the desired gene expression.

The LEC may be derived from any plasmid capable of being linearized. The plasmid may be capable of expressing the heavy chain polypeptide and/or light chain polypeptide encoded by the recombinant nucleic acid sequence construct. The plasmid can be pNP (Puerto Rico/34) or pM2 (New Caledonia/99). The plasmid may be WLV009, pVAX, pcDNA3.0, or provax, or any other expression vector capable of expressing the heavy chain polypeptide and/or light chain polypeptide encoded by the recombinant nucleic acid sequence construct.

The LEC can be pcrM2. The LEC can be pcrNP. pcrNP and pcrMR can be derived from pNP (Puerto Rico/34) and pM2 (New Caledonia/99), respectively.

### (18) Viral Vectors

In one embodiment, viral vectors are provided herein which are capable of delivering a nucleic acid of the invention to a cell. The expression vector may be provided to a cell in the form of a viral vector. Viral vector technology is well known in the art and is described, for example, in Sambrook et al. (2001), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

### (19) Method of Preparing the Vector

Provided herein is a method for preparing the one or more vectors in which the recombinant nucleic acid sequence construct has been placed. After the final subcloning step, the vector can be used to inoculate a cell culture in a large scale fermentation tank, using known methods in the art.

In other embodiments, after the final subcloning step, the vector can be used with one or more electroporation (EP) devices. The EP devices are described below in more detail.

The one or more vectors can be formulated or manufactured using a combination of known devices and techniques, but preferably they are manufactured using a plasmid manufacturing technique that is described in a licensed, co-pending U.S. provisional application U.S. Ser. No. 60/939,792, which was filed on May 23, 2007. In some examples, the DNA plasmids described herein can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### 5. ANTIBODY

As described above, the recombinant nucleic acid sequence can encode the antibody, a fragment thereof, a variant thereof, or a combination thereof. The antibody can bind or react with the antigen, which is described in more detail below.

The antibody may comprise a heavy chain and a light chain complementarity determining region (“CDR”) set, respectively interposed between a heavy chain and a light chain framework (“FR”) set which provide support to the CDRs and define the spatial relationship of the CDRs relative to each other. The CDR set may contain three hypervariable regions of a heavy or light chain V region. Proceeding from the N-terminus of a heavy or light chain, these regions are denoted as “CDR1,” “CDR2,” and “CDR3,” respectively. An antigen-binding site, therefore, may include six CDRs, comprising the CDR set from each of a heavy and a light chain V region.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)₂ fragment, which comprises both antigen-binding sites. Accordingly, the antibody can be the Fab or F(ab′)₂. The Fab can include the heavy chain polypeptide and the light chain polypeptide. The heavy chain polypeptide of the Fab can include the VH region and the CH1 region. The light chain of the Fab can include the VL region and CL region.

The antibody can be an immunoglobulin (Ig). The Ig can be, for example, IgA, IgM, IgD, IgE, and IgG. The immunoglobulin can include the heavy chain polypeptide and the light chain polypeptide. The heavy chain polypeptide of the immunoglobulin can include a VH region, a CH1 region, a hinge region, a CH2 region, and a CH3 region. The light chain polypeptide of the immunoglobulin can include a VL region and CL region.

The antibody can be a polyclonal or monoclonal antibody. The antibody can be a chimeric antibody, a single chain antibody, an affinity matured antibody, a human antibody, a humanized antibody, or a fully human antibody. The humanized antibody can be an antibody from a non-human species that binds the desired antigen having one or more complementarity determining regions (CDRs) from the non-human species and framework regions from a human immunoglobulin molecule.

The antibody can be a bispecific antibody as described below in more detail. The antibody can be a bifunctional antibody as also described below in more detail.

As described above, the antibody can be generated in the subject upon administration of the composition to the subject. The antibody may have a half-life within the subject. In some embodiments, the antibody may be modified to extend or shorten its half-life within the subject. Such modifications are described below in more detail.

The antibody can be defucosylated as described in more detail below.

In one embodiment, the antibody binds a SARS-CoV-2 antigen. In one embodiment, the antibody binds at least one epitope of a SARS-CoV-2 Spike protein. In one embodiment, the antibody binds a SARS-CoV-2 RBD.

The antibody may be modified to reduce or prevent antibody-dependent enhancement (ADE) of disease associated with the antigen as described in more detail below.

### Bispecific Antibody

The recombinant nucleic acid sequence can encode a bispecific antibody, a fragment thereof, a variant thereof, or a combination thereof. The bispecific antibody can bind or react with two antigens, for example, two of the antigens described below in more detail. The bispecific antibody can be comprised of fragments of two of the antibodies described herein, thereby allowing the bispecific antibody to bind or react with two desired target molecules, which may include the antigen, which is described below in more detail, a ligand, including a ligand for a receptor, a receptor, including a ligand-binding site on the receptor, a ligand-receptor complex, and a marker.

The invention provides novel bispecific antibodies comprising a first antigen-binding site that specifically binds to a first target and a second antigen-binding site that specifically binds to a second target, with particularly advantageous properties such as producibility, stability, binding affinity, biological activity, specific targeting of certain T cells, targeting efficiency and reduced toxicity. In some instances, there are bispecific antibodies, wherein the bispecific antibody binds to the first target with high affinity and to the second target with low affinity. In other instances, there are bispecific antibodies, wherein the bispecific antibody binds to the first target with low affinity and to the second target with high affinity. In other instances, there are bispecific antibodies, wherein the bispecific antibody binds to the first target with a desired affinity and to the second target with a desired affinity.

In one embodiment, the bispecific antibody is a bivalent antibody comprising a) a first light chain and a first heavy chain of an antibody specifically binding to a first antigen, and b) a second light chain and a second heavy chain of an antibody specifically binding to a second antigen.

A bispecific antibody molecule according to the invention may have two binding sites of any desired specificity. In some embodiments one of the binding sites is capable of binding a tumor associated antigen. In some embodiments, the binding site included in the Fab fragment is a binding site specific for a SARS-CoV-2 antigen. In some embodiments, the binding site included in the single chain Fv fragment is a binding site specific for a SARS-CoV-2 antigen such as a SARS-CoV-2 spike antigen.

In some embodiments, one of the binding sites of a bispecific antibody according to the invention is able to bind a T-cell specific receptor molecule and/or a natural killer cell (NK cell) specific receptor molecule. A T-cell specific receptor is the so called “T-cell receptor” (TCRs), which allows a T cell to bind to and, if additional signals are present, to be activated by and respond to an epitope/antigen presented by another cell called the antigen-presenting cell or APC. The T cell receptor is known to resemble a Fab fragment of a naturally occurring immunoglobulin. It is generally monovalent, encompassing α- and β-chains, in some embodiments it encompasses γ-chains and δ-chains. Accordingly, in some embodiments the TCR is TCR (alpha/beta) and in some embodiments it is TCR (gamma/delta). The T cell receptor forms a complex with the CD3 T-Cell co-receptor. CD3 is a protein complex and is composed of four distinct chains. In mammals, the complex contains a CD3γ chain, a CD36 chain, and two CD3E chains. These chains associate with a molecule known as the T cell receptor (TCR) and the ζ-chain to generate an activation signal in T lymphocytes. Hence, in some embodiments a T-cell specific receptor is the CD3 T-Cell co-receptor. In some embodiments, a T-cell specific receptor is CD28, a protein that is also expressed on T cells. CD28 can provide co-stimulatory signals, which are required for T cell activation. CD28 plays important roles in T-cell proliferation and survival, cytokine production, and T-helper type-2 development. Yet a further example of a T-cell specific receptor is CD134, also termed Ox40. CD134/Ox40 is being expressed after 24 to 72 hours following activation and can be taken to define a secondary costimulatory molecule. Another example of a T-cell receptor is 4-1 BB capable of binding to 4-1 BB-Ligand on antigen presenting cells (APCs), whereby a costimulatory signal for the T cell is generated. Another example of a receptor predominantly found on T-cells is CD5, which is also found on B cells at low levels. A further example of a receptor modifying T cell functions is CD95, also known as the Fas receptor, which mediates apoptotic signaling by Fas-ligand expressed on the surface of other cells. CD95 has been reported to modulate TCR/CD3-driven signaling pathways in resting T lymphocytes.

An example of a NK cell specific receptor molecule is CD16, a low affinity Fc receptor and NKG2D. An example of a receptor molecule that is present on the surface of both T cells and natural killer (NK) cells is CD2 and further members of the CD2-superfamily. CD2 is able to act as a co-stimulatory molecule on T and NK cells.

In some embodiments, the first binding site of the bispecific antibody molecule binds a SARS-CoV-2 antigen and the second binding site binds a T cell specific receptor molecule and/or a natural killer (NK) cell specific receptor molecule.

In some embodiments, the first binding site of the antibody molecule binds the SARS-CoV-2 spike antigen, and the second binding site binds a T cell specific receptor molecule and/or a natural killer (NK) cell specific receptor molecule. In some embodiments, the first binding site of the antibody molecule binds a SARS-CoV-2 spike antigen and the second binding site binds one of CD3, the T cell receptor (TCR), CD28, CD16, NKG2D, Ox40, 4-1BB, CD2, CD5 and CD95.

In some embodiments, the first binding site of the antibody molecule binds a T cell specific receptor molecule and/or a natural killer (NK) cell specific receptor molecule and the second binding site binds a SARS-CoV-2 antigen. In some embodiments, the first binding site of the antibody binds a T cell specific receptor molecule and/or a natural killer (NK) cell specific receptor molecule and the second binding site binds the SARS-CoV-2 spike antigen. In some embodiments, the first binding site of the antibody binds one of CD3, the T cell receptor (TCR), CD28, CD16, NKG2D, Ox40, 4-1BB, CD2, CD5 and CD95, and the second binding site binds the SARS-CoV-2 spike antigen.

### CAR Molecules

In one embodiment, the invention provides a chimeric antigen receptor (CAR) comprising a binding domain comprising a SARS-CoV-2 antibody of the invention. In one embodiment, the CAR comprises an antigen binding domain. In one embodiment, the antigen binding domain is a targeting domain, wherein the targeting domain directs the T cell expressing the CAR to a SARS-CoV-2 viral particle. For example, in one embodiment, the targeting domain comprises an antibody, antibody fragment, or peptide that specifically binds to a SARS-CoV-2 antigen.

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD32 signaling domain. Preclinical studies have indicated that “Second Generation” CARs can improve the anti-tumor activity of T cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD32 signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above. In a particular non-limiting embodiment, the antigen-binding domain is a SARS-CoV-2 antibody of the invention or a variant thereof, such as an scFV fragment of a SARS-CoV-2 antibody of the invention specific for binding to a surface antigen of SARS-CoV-2.

### Bifunctional Antibody

The recombinant nucleic acid sequence can encode a bifunctional antibody, a fragment thereof, a variant thereof, or a combination thereof. The bifunctional antibody can bind or react with the antigen described below. The bifunctional antibody can also be modified to impart an additional functionality to the antibody beyond recognition of and binding to the antigen. Such a modification can include, but is not limited to, coupling to factor H or a fragment thereof. Factor His a soluble regulator of complement activation and thus, may contribute to an immune response via complement-mediated lysis (CML).

### Immune Cells

In various embodiments, the invention relates to a composition comprising an immune cell engineered for expression or endogenous secretion of an anti-SARS-CoV-2 antibody of the invention. In one embodiment, the anti-SARS-CoV-2 antibody is a bi-specific T cell engaging antibody comprising a domain for binding to a SARS-CoV-2 antigen and a domain for activating an immune cell. Examples of immune cells that can be engineered for expression or secretion of an anti-SARS-CoV-2 antibody of the invention include, but are not limited to, T cells, B cells, natural killer (NK) cells, or macrophages. In some embodiments, the immune cell further comprises a chimeric antigen receptor (CAR). Therefore, in some embodiments, the invention relates to the use of CAR T-cells for expression or delivery of an anti-SARS-CoV-2 antibody of the invention.

In various embodiments, the invention relates to compositions for endogenous secretion of a T cell-redirecting bispecific antibody (T-bsAb) by engineered T cells (STAb-T cells), which have been engineered to express the anti-SARS-CoV-2 antibody of the invention. In various embodiments, the method comprises administering to a subject in need thereof a composition comprising a STAb-T cell, wherein the STAb-T cell has been engineered to express a bispecific immune cell engaging anti-SARS-CoV-2 antibody of the invention. In some embodiments, the STAb-T cell further comprises a chimeric antigen receptor (CAR). Therefore, in some embodiments, the invention relates to the use of CAR T-cells for expression or delivery of an anti-SARS-CoV-2 antibody of the invention.

### Extension of Antibody Half-Life

As described above, the antibody may be modified to extend or shorten the half-life of the antibody in the subject. The modification may extend or shorten the half-life of the antibody in the serum of the subject.

The modification may be present in a constant region of the antibody. The modification may be one or more amino acid substitutions in a constant region of the antibody that extend the half-life of the antibody as compared to a half-life of an antibody not containing the one or more amino acid substitutions. The modification may be one or more amino acid substitutions in the CH2 domain of the antibody that extend the half-life of the antibody as compared to a half-life of an antibody not containing the one or more amino acid substitutions.

In some embodiments, the one or more amino acid substitutions in the constant region may include replacing a methionine residue in the constant region with a tyrosine residue, a serine residue in the constant region with a threonine residue, a threonine residue in the constant region with a glutamate residue, or any combination thereof, thereby extending the half-life of the antibody.

In other embodiments, the one or more amino acid substitutions in the constant region may include replacing a methionine residue in the CH2 domain with a tyrosine residue, a serine residue in the CH2 domain with a threonine residue, a threonine residue in the CH2 domain with a glutamate residue, or any combination thereof, thereby extending the half-life of the antibody.

### Defucosylation

The recombinant nucleic acid sequence can encode an antibody that is not fucosylated (i.e., a defucosylated antibody or a non-fucosylated antibody), a fragment thereof, a variant thereof, or a combination thereof. Fucosylation includes the addition of the sugar fucose to a molecule, for example, the attachment of fucose to N-glycans, O-glycans and glycolipids. Accordingly, in a defucosylated antibody, fucose is not attached to the carbohydrate chains of the constant region. In turn, this lack of fucosylation may improve FcγRIIIa binding and antibody directed cellular cytotoxic (ADCC) activity by the antibody as compared to the fucosylated antibody. Therefore, in some embodiments, the non-fucosylated antibody may exhibit increased ADCC activity as compared to the fucosylated antibody.

The antibody may be modified so as to prevent or inhibit fucosylation of the antibody. In some embodiments, such a modified antibody may exhibit increased ADCC activity as compared to the unmodified antibody. The modification may be in the heavy chain, light chain, or a combination thereof. The modification may be one or more amino acid substitutions in the heavy chain, one or more amino acid substitutions in the light chain, or a combination thereof.

### Reduced ADE Response

The antibody may be modified to reduce or prevent antibody-dependent enhancement (ADE) of disease associated with the antigen, but still neutralize the antigen.

In some embodiments, the antibody may be modified to include one or more amino acid substitutions that reduce or prevent binding of the antibody to FcγR1a. The one or more amino acid substitutions may be in the constant region of the antibody. The one or more amino acid substitutions may include replacing a leucine residue with an alanine residue in the constant region of the antibody, i.e., also known herein as LA, LA mutation or LA substitution. The one or more amino acid substitutions may include replacing two leucine residues, each with an alanine residue, in the constant region of the antibody and also known herein as LALA, LALA mutation, or LALA substitution. The presence of the LALA substitutions may prevent or block the antibody from binding to FcγR1a, and thus, the modified antibody does not enhance or cause ADE of disease associated with the antigen, but still neutralizes the antigen.

### 6. ANTIGEN

The synthetic antibody is directed to the antigen or fragment or variant thereof. The antigen can be a nucleic acid sequence, an amino acid sequence, a polysaccharide or a combination thereof. The nucleic acid sequence can be DNA, RNA, cDNA, a variant thereof, a fragment thereof, or a combination thereof. The amino acid sequence can be a protein, a peptide, a variant thereof, a fragment thereof, or a combination thereof. The polysaccharide can be a nucleic acid encoded polysaccharide.

The antigen can be from a virus. The antigen can be associated with viral infection. In one embodiment, the antigen can be associated with SARS-CoV-2 infection, or COVID-19. In one embodiment, the antigen can be associated with human immunodeficiency virus (HIV) infection.

In one embodiment, the antigen can be a fragment of a SARS-CoV-2 antigen. For example, in one embodiment, the antigen is a fragment of a SARS-CoV-2 spike protein. In one embodiment, the antigen is the receptor binding domain (RBD) of the SARS-CoV-2 spike protein.

In one embodiment, a synthetic antibody of the invention targets two or more antigens. In one embodiment, at least one antigen of a bispecific antibody is selected from the antigens described herein. In one embodiment, the two or more antigens are selected from the antigens described herein.

### Viral Antigens

The viral antigen can be a viral antigen or fragment or variant thereof. The virus can be a disease-causing virus. The virus can be a coronavirus. The virus can be SARS or the SARS-CoV-2 virus. The virus can be human immunodeficiency virus (HIV).

The antigen may be a SARS-CoV-2 viral antigen, or fragment thereof, or variant thereof. The SARS-CoV-2 antigen can be from a factor that allows the virus to replicate, infect or survive. Factors that allow a SARS-CoV-2 virus to replicate or survive include, but are not limited to, structural proteins and non-structural proteins. Such a protein can be a spike protein.

### 7. EXCIPIENTS AND OTHER COMPONENTS OF THE COMPOSITION

The composition may further comprise a pharmaceutically acceptable excipient. The pharmaceutically acceptable excipient can be functional molecules such as vehicles, carriers, or diluents. The pharmaceutically acceptable excipient can be a transfection facilitating agent, which can include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and the poly-L-glutamate may be present in the composition at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the composition. The composition may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. The transfection facilitating agent is a polyanion, polycation, including poly-L-glutamate (LGS), or lipid. Concentration of the transfection agent in the vaccine is less than 4 mg/ml, less than 2 mg/ml, less than 1 mg/ml, less than 0.750 mg/ml, less than 0.500 mg/ml, less than 0.250 mg/ml, less than 0.100 mg/ml, less than 0.050 mg/ml, or less than 0.010 mg/ml.

The composition may further comprise a genetic facilitator agent as described in U.S. Ser. No. 021,579 filed Apr. 1, 1994, which is fully incorporated by reference.

In some embodiments, the composition comprises hyaluronidase. In some embodiments, the composition comprises recombinant human hyaluronidase.

The composition may comprise DNA at quantities of from about 1 nanogram to 100 milligrams; about 1 microgram to about 10 milligrams; or preferably about 0.1 microgram to about 10 milligrams; or more preferably about 1 milligram to about 2 milligrams. In some preferred embodiments, composition according to the present invention comprises about 5 nanograms to about 1000 micrograms of DNA. In some preferred embodiments, composition can contain about 10 nanograms to about 800 micrograms of DNA. In some preferred embodiments, the composition can contain about 0.1 to about 500 micrograms of DNA. In some preferred embodiments, the composition can contain about 1 to about 350 micrograms of DNA. In some preferred embodiments, the composition can contain about 25 to about 250 micrograms, from about 100 to about 200 microgram, from about 1 nanogram to 100 milligrams; from about 1 microgram to about 10 milligrams; from about 0.1 microgram to about 10 milligrams; from about 1 milligram to about 2 milligrams, from about 5 nanograms to about 1000 micrograms, from about 10 nanograms to about 800 micrograms, from about 0.1 to about 500 micrograms, from about 1 to about 350 micrograms, from about 25 to about 250 micrograms, from about 100 to about 200 microgram of DNA.

The composition can be formulated according to the mode of administration to be used. An injectable pharmaceutical composition can be sterile, pyrogen free and particulate free. An isotonic formulation or solution can be used. Additives for isotonicity can include sodium chloride, dextrose, mannitol, sorbitol, and lactose. The composition can comprise a vasoconstriction agent. The isotonic solutions can include phosphate buffered saline. The composition can further comprise stabilizers including gelatin and albumin. The stabilizers can allow the formulation to be stable at room or ambient temperature for extended periods of time, including LGS or polycations or polyanions.

In some embodiments, the composition can be formulated for administration of a dosage of 0.5 mg of DNA. In some embodiments, the composition can be formulated for administration of a dosage of 1.0 mg of DNA.

### 8. NANOPARTICLE FORMULATIONS

In one embodiment, the immunogenic composition of the invention may comprise a nanoparticle, including but not limited to a lipid nanoparticle (LNP), comprising a binding molecule of the invention, or a LNP comprising a nucleic acid encoding a binding molecule of the invention. In some embodiments, the composition comprises or encodes all or part of an antigen binding molecule of the invention, or an immunogenically functional equivalent thereof. In some embodiments, the composition comprises an mRNA molecule that encodes all or part of an antigen binding molecule of the invention.

In one embodiment, the immunogenic composition of the invention may comprise a composition comprising one or more glycan modified antibodies of the invention, or a LNP comprising one or more nucleic acid molecules encoding a glycan modified antibody of the invention.

In one embodiment, the immunogenic composition of the invention may comprise a composition comprising one or more framework modified antibodies of the invention, or a LNP comprising one or more nucleic acid molecules encoding a framework modified antibody of the invention.

In one embodiment, the immunogenic composition of the invention may comprise a composition comprising one or more SARS-CoV-2 antibodies of the invention, or a LNP comprising one or more nucleic acid molecules encoding a SARS-CoV-2 antibody of the invention.

In one embodiment, the immunogenic composition of the invention may comprise a composition comprising one or more HIV antibodies of the invention, or a LNP comprising one or more nucleic acid molecules encoding an HIV antibody of the invention.

In one embodiment, the immunogenic composition of the invention may comprise a composition comprising a combination of SARS-CoV-2 antibodies of the invention, or a LNP comprising one or more nucleic acid molecules encoding a combination of SARS-CoV-2 antibodies of the invention. In one embodiment, the immunogenic composition of the invention may comprise a composition comprising a combination of LNP, wherein the combination of LNP comprises one or more nucleic acid molecules encoding a combination of SARS-CoV-2 antibodies of the invention.

In one embodiment, the invention relates to a combination of LNPs comprising or encapsulating a combination of at least two RNA molecules encoding the combination of the heavy chain and light chain of the synthetic antibody of the invention or fragments or variants thereof.

In one embodiment, the composition further comprises one or more additional immunostimulatory agents. Immunostimulatory agents include, but are not limited to, an additional antigen or antigen binding molecule, an immunomodulator, or an adjuvant.

### 9. GENERATION OF ANTIBODIES

The present invention also relates a method of generating the synthetic antibody. The method can include administering the composition to the subject in need thereof by using the method of delivery described in more detail below. Accordingly, the synthetic antibody is generated in the subject or in vivo upon administration of the composition to the subject.

The method can also include introducing the composition into one or more cells, and therefore, the synthetic antibody can be generated or produced in the one or more cells. The method can further include introducing the composition into one or more tissues, for example, but not limited to, skin and muscle, and therefore, the synthetic antibody can be generated or produced in the one or more tissues.

### 10. METHODS OF DIAGNOSING A DISEASE OR DISORDER

The present invention further relates to a method of diagnosing a subject as having a disease or disorder using an antibody, fragment thereof, or nucleic acid molecule encoding the same as described herein. In some embodiments, the present invention features methods for identifying subjects who are at risk of spreading SARS-CoV-2 infection or COVID-19, including those subjects who are asymptomatic or only exhibit non-specific indicators of SARS-CoV-2 infection or COVID-19. In some embodiments, the present invention is also useful for monitoring subjects undergoing treatments and therapies for SARS-CoV-2 infection or COVID-19, and for selecting or modifying therapies and treatments that would be efficacious in subjects having SARS-CoV-2 infection or COVID-19, wherein selection and use of such treatments and therapies promote immunity to SARS-CoV-2, or prevent infection by SARS-CoV-2.

In one embodiment, the antibody, fragment thereof, or nucleic acid molecule encoding the same can be used in an immunoassay for diagnosing a subject as having an active SARS-CoV-2 infection, having COVID-19, or having immunity to SARS-CoV-2 infection, or for monitoring subjects undergoing treatments and therapies for SARS-CoV-2 infection or COVID-19. Non-limiting exemplary immunoassays include, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, New York; Harlow et al., 1999, Using Antibodies: A Laboratory Manual, Cold Spring Harbor Laboratory Press, NY.

In some embodiments the methods include obtaining a sample from a subject and contacting the sample with an antibody of the invention or a cell expressing an antibody of the invention and detecting binding of the antibody to an antigen present in the sample.

In some embodiments, samples can be provided from a subject undergoing treatment regimens or therapeutic interventions, e.g., drug treatments, vaccination, etc. for SARS-CoV-2 infection or COVID-19. Samples can be obtained from the subject at various time points before, during, or after treatment.

The SARS-CoV-2 antibodies of the present invention, or nucleic acid molecules encoding the same, can thus be used to generate a risk profile or signature of subjects: (i) who are expected to have immunity to SARS-CoV-2 infection or COVID-19 and/or (ii) who are at risk of developing SARS-CoV-2 infection or COVID-19. The antibody profile of a subject can be compared to a predetermined or reference antibody profile to diagnose or identify subjects at risk for developing SARS-CoV-2 infection or COVID-19, to monitor the progression of disease, as well as the rate of progression of disease, and to monitor the effectiveness of SARS-CoV-2 infection or COVID-19 treatments. Data concerning the antibodies of the present invention can also be combined or correlated with other data or test results for SARS-CoV-2 infection or COVID-19, including but not limited to age, weight, BMI, imaging data, medical history, smoking status and any relevant family history.

The present invention also provides methods for identifying agents for treating SARS-CoV-2 infection or COVID-19 that are appropriate or otherwise customized for a specific subject. In this regard, a test sample from a subject, exposed to a therapeutic agent, drug, or other treatment regimen, can be taken and the level of one or more SARS-CoV-2 antibody can be determined. The level of one or more SARS-CoV-2 antibody can be compared to a sample derived from the subject before and after treatment, or can be compared to samples derived from one or more subjects who have shown improvements in risk factors as a result of such treatment or exposure.

In one embodiment, the invention is a method of diagnosing SARS-CoV-2 infection or COVID-19. In one embodiment, the method includes determining immunity to infection or reinfection by SARS-CoV-2. In some embodiments, these methods may utilize at least one biological sample (such as urine, saliva, blood, serum, plasma, amniotic fluid, or tears), for the detection of one or more SARS-CoV-2 antibody of the invention in the sample. Frequently the sample is a “clinical sample” which is a sample derived from a patient. In one embodiment, the biological sample is a blood sample.

In one embodiment, the method comprises detecting one or more SARS-CoV-2 antigen in at least one biological sample of the subject. In various embodiments, the level of one or more SARS-CoV-2 antigen of the invention in the biological sample of the subject is compared to a comparator. Non-limiting examples of comparators include, but are not limited to, a negative control, a positive control, an expected normal background value of the subject, a historical normal background value of the subject, an expected normal background value of a population that the subject is a member of, or a historical normal background value of a population that the subject is a member of.

### 11. METHODS OF DELIVERY OF THE COMPOSITION

The present invention also relates to a method of delivering the composition to the subject in need thereof. The method of delivery can include, administering the composition to the subject. Administration can include, but is not limited to, DNA injection with and without in vivo electroporation, liposome mediated delivery, and nanoparticle facilitated delivery.

The mammal receiving delivery of the composition may be human, primate, non-human primate, cow, cattle, sheep, goat, antelope, bison, water buffalo, bison, bovids, deer, hedgehogs, elephants, llama, alpaca, mice, rats, and chicken.

The composition may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

### 12. METHODS OF TREATMENT

Also provided herein is a method of treating, protecting against, and/or preventing disease in a subject in need thereof by generating the synthetic antibody in the subject. The method can include administering the composition to the subject. Administration of the composition to the subject can be done using the method of delivery described above.

The method can include administering a combination of antibodies or nucleic acid molecules encoding the same to the subject. In one embodiment, the method of the invention provides for administration of an antibody cocktail to the subject. In one embodiment, the cocktail is administered as a single formulation comprising multiple antibodies or nucleic acid molecules encoding the same. In one embodiment, the cocktail is administered as multiple formulations, either sequentially or concurrently. Administration of the composition to the subject can be done using the method of delivery described above. In one embodiment, the method of administration is intramuscular administration.

In certain embodiments, the invention provides a method of treating protecting against, and/or preventing a SARS-CoV-2 virus infection. In one embodiment, the method treats, protects against, and/or prevents a disease or disorder associated with SARS-CoV-2 virus infection. In one embodiment, the method treats, protects against, and/or prevents COVID-19. In one embodiment the subject has, or is at risk of, SARS-CoV-2 virus infection.

In certain embodiments, the invention provides a method of treating protecting against, and/or preventing a HIV infection. In one embodiment, the method treats, protects against, and/or prevents a disease or disorder associated with HIV infection. In one embodiment, the method treats, protects against, and/or prevents AIDS. In one embodiment the subject has, or is at risk of, HIV infection.

Upon generation of one or more synthetic antibody in the subject, the one or more synthetic antibody can bind to or react with the antigen. Such binding can neutralize the antigen, block recognition of the antigen by another molecule, for example, a protein or nucleic acid, and elicit or induce an immune response to the antigen, thereby treating, protecting against, and/or preventing the disease associated with the antigen in the subject.

The one or more synthetic antibody can treat, prevent, and/or protect against disease in the subject administered the composition. The one or more synthetic antibody by binding the antigen can treat, prevent, and/or protect against disease in the subject administered the composition. The synthetic antibody can promote survival of the disease in the subject administered the composition. In one embodiment, the synthetic antibody can provide increased survival of the disease in the subject over the expected survival of a subject having the disease who has not been administered the synthetic antibody. In various embodiments, the synthetic antibody can provide at least about a 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or a 100% increase in survival of the disease in subjects administered the composition over the expected survival in the absence of the composition. In one embodiment, the synthetic antibody can provide increased protection against the disease in the subject over the expected protection of a subject who has not been administered the synthetic antibody. In various embodiments, the synthetic antibody can protect against disease in at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or 100% of subjects administered the composition over the expected protection in the absence of the composition.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

In one embodiment, immunotherapy with the binding molecule of the invention will have a direct therapeutic effect. In one embodiment, immunotherapy with the binding molecule of the invention can be used as immune “adjuvant” treatment to reduce viral protein load, in order to provide host immunity and optimize the effect of antiviral drugs.

### 13. GENERATION OF SYNTHETIC ANTIBODIES IN VITRO AND EX VIVO

In one embodiment, one or more synthetic antibody is generated in vitro or ex vivo. For example, in one embodiment, a nucleic acid encoding a synthetic antibody can be introduced and expressed in an in vitro or ex vivo cell. Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome or lipid nanoparticle. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

The present invention has multiple aspects, illustrated by the following non-limiting examples.

### 14. EXAMPLES

The present invention is further illustrated in the following Examples. It should be understood that these Examples, while demonstrating some embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Glycan Modified Binding Molecules

Glycosylation is a quality control mechanism in protein synthesis which can promote protein stability and expression. Introducing glycosylation sites has successfully been used to improve protein expression in eukaryotic cells (5,6).

Computational analyses show preferences for AA to be in certain secondary structures (7). Probing the protein data bank (PDB) may give structural information on how nature ‘solves’ particular folds.

The experiments presented herein demonstrate that key locations to introduce glycosylation sites were identified computationally. Multiple rounds of small and large scale screening identified key sites. Introduction of glycosylation sites can improved antibody expression 1.5-2× over WT. Antibody binding to target remains largely intact.

Combinations of up to six additional glycans are functional and improve expression. Lead glycan combinations also boost expression of unrelated HIV antibody.

aaScan identified proteins with similar structural motifs. Combinations of mutations boost small scale expression. Expression may correlate with design energy

### Cloaking With Glycans (CWG)

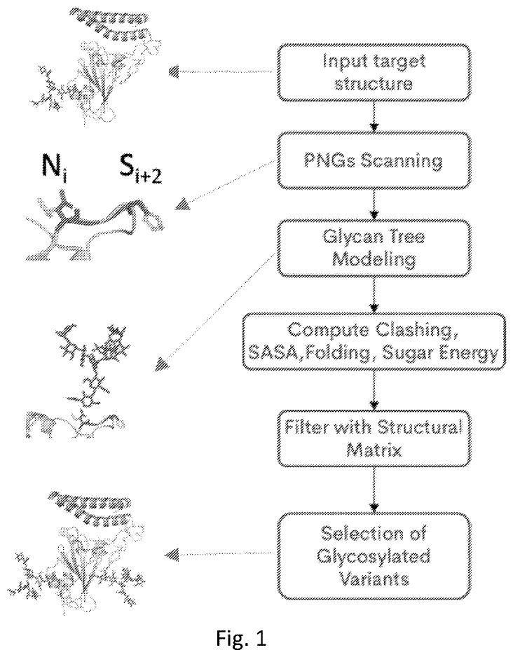

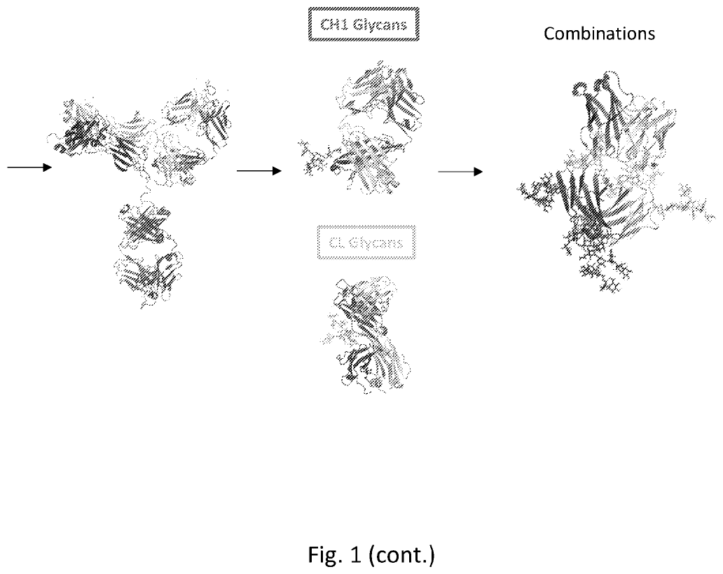

A method has been built to identify all possible glycosylation sites on a given protein. The program takes a target structure and looks for potential new glycosylation sites, models the glycan, and then determines potential clashes, folding energy and sugar energy to output sets of mutations that are amenable to the introduction of glycosylation sites (FIG. 1; Konrath et al. 2022, Cell Rep. 2022; 38(5):110318).

Here, CWG was applied to the CH1 and CL regions of an IgG1 antibody for SARS-CoV-2.

Pentapeptide Scan (aaScan)

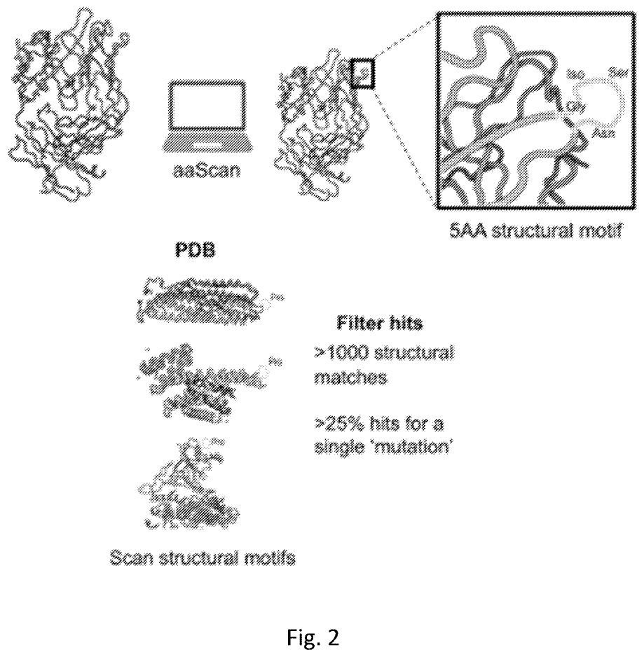

A code was written in MSL (Kulp et al., 2012, J Comput Chem. 33(20):1645-1661) called aaScan that interrogates local protein structure for stabilization opportunities. The program scans an input structure five residues at a time looking for close structural matches in the PDB. The amino acid identity of the middle residue in all the matches from diverse proteins is compared to the middle residue of the input structure to identify how frequently each amino acid is observed in a five amino acid structural motif (FIG. 2).

### Glycosylation to Boost Expression

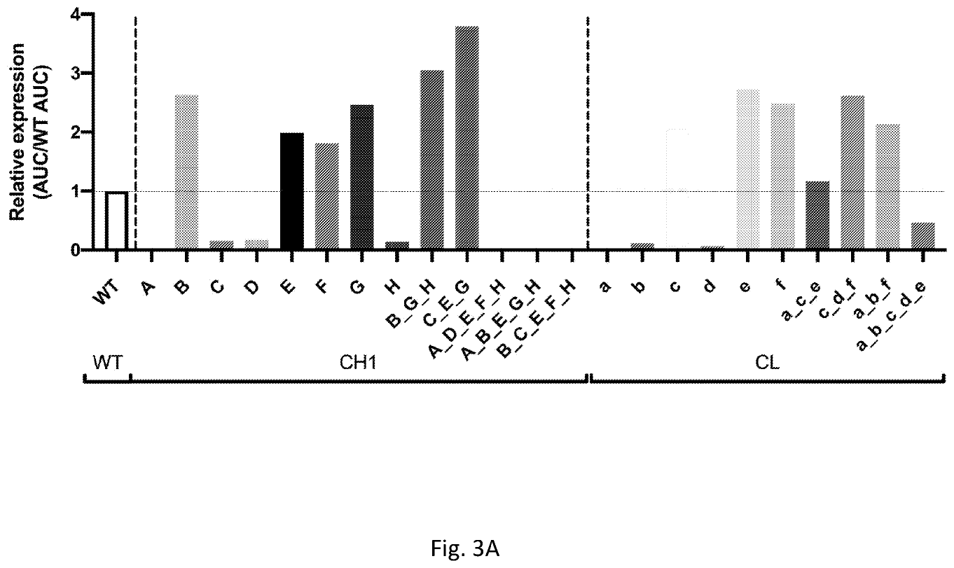

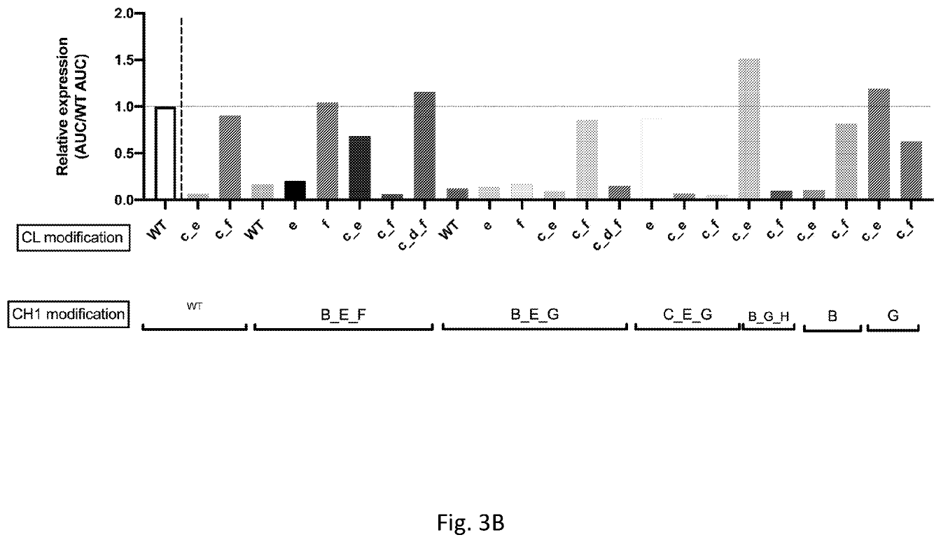

CH1 or CL glycan modifications were made to improve expression. FIG. 3A shows the relative expression (AUC/AUC WT) of single, triple or quintuple glycan modifications to the CH1 or CL region of a SARS-CoV2 antibody, paired with the WT opposite chain. Automated Western Blots were run to probe for the heavy chain and AUC corresponding to the heavy chain peak was assessed. FIG. 3B shows the relative expression (AUC/AUC WT) of combined CH1 and CL glycan modifications using the same methodology as in FIG. 3A.

### Large Scale Binding and Expression of Lead Glycan Modified Variants.

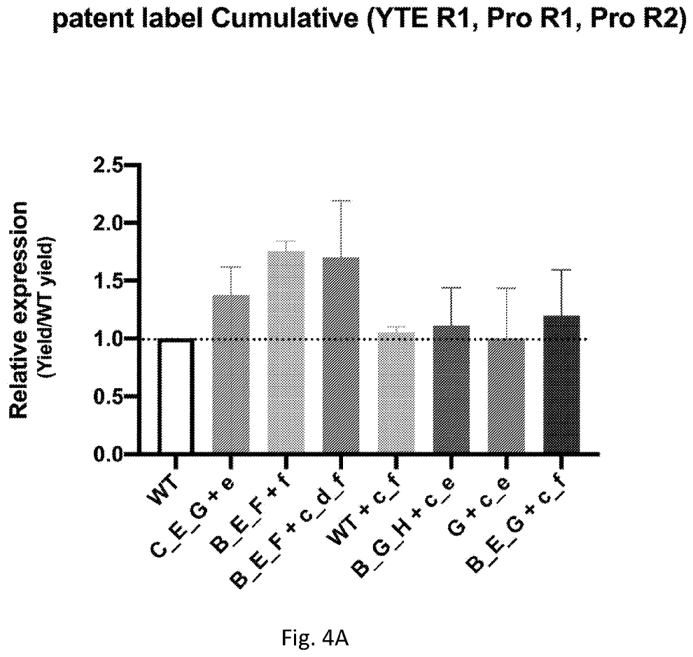

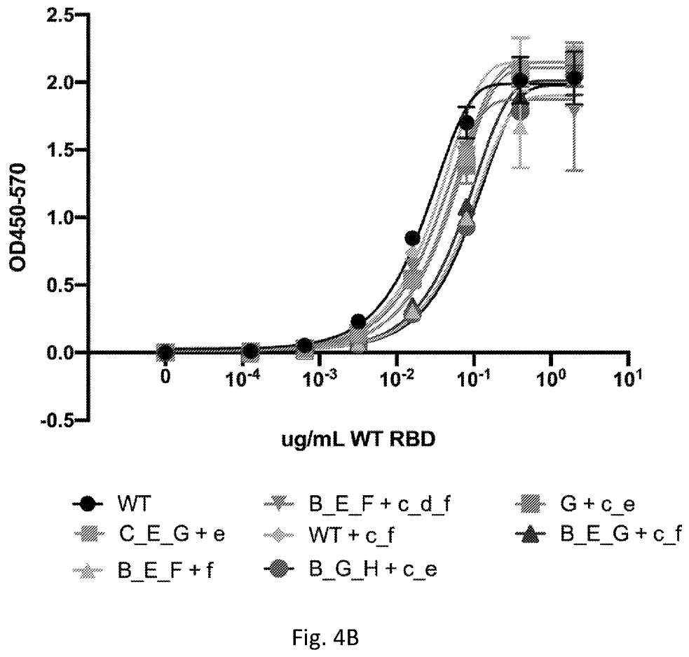

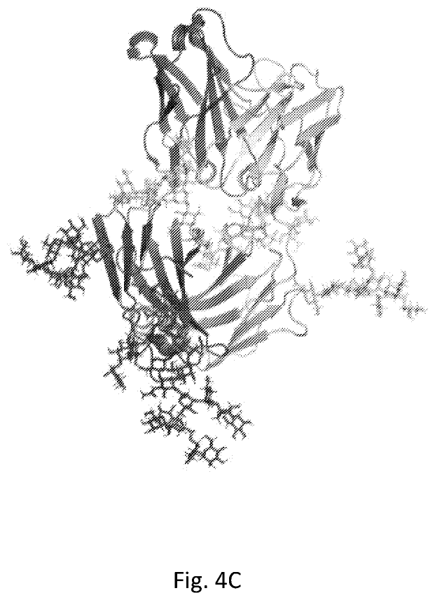

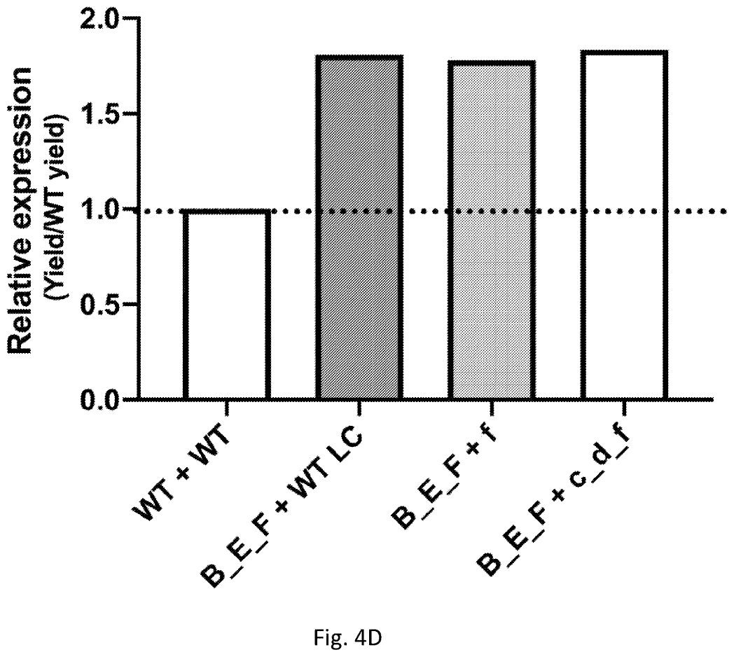

FIG. 4A shows the relative expression (yield/WT yield) of promising combinations of glycan modifications as identified by small scale screening across three separate experiments. Modifications to the CH1 region were paired with modifications to the CL region. Expi293 cells were transfected with the indicated combinations and protein A columns were used to purify the antibodies. Yield was then quantified (FIG. 4B). Binding of each largescale variant to WT receptor-binding domain (RBD) of SARS-CoV2. ELISA was used to assess the impact of glycan modifications to binding. FIG. 4C shows a model depicting the glycan combination that resulted in the largest increase in expression as compared to WT (CH1: B_E_F and CL: c_d_f) FIG. 4D shows the relative expression (yield/WT yield) of the top glycan modifications to the CH1 and CL region of an unrelated HIV antibody. Combinations assessed were picked from prior rounds of screening.

### Pentapeptide Scan to Boost Expression

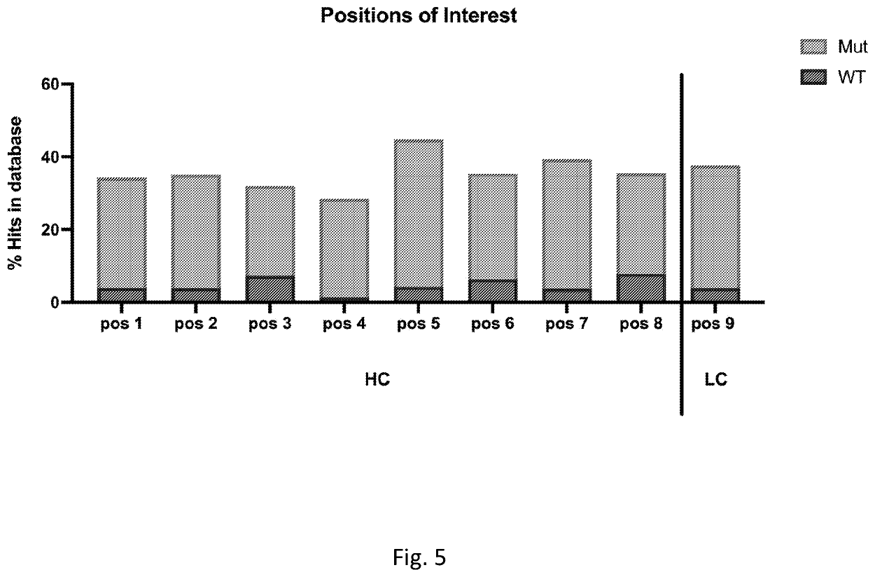

Mutations of interest were then identified. Percentage that a given mutation is found in a five amino acid structural motif in comparison to the wild type amino acid from the SARS-COV2 antibody. Hits from aaScan were filtered to only include >1000 structural matches, with over 25% of the hits at a position comprised of a single amino acid residue (FIG. 5).

### Pentapeptide Modifications to Improve Expression.

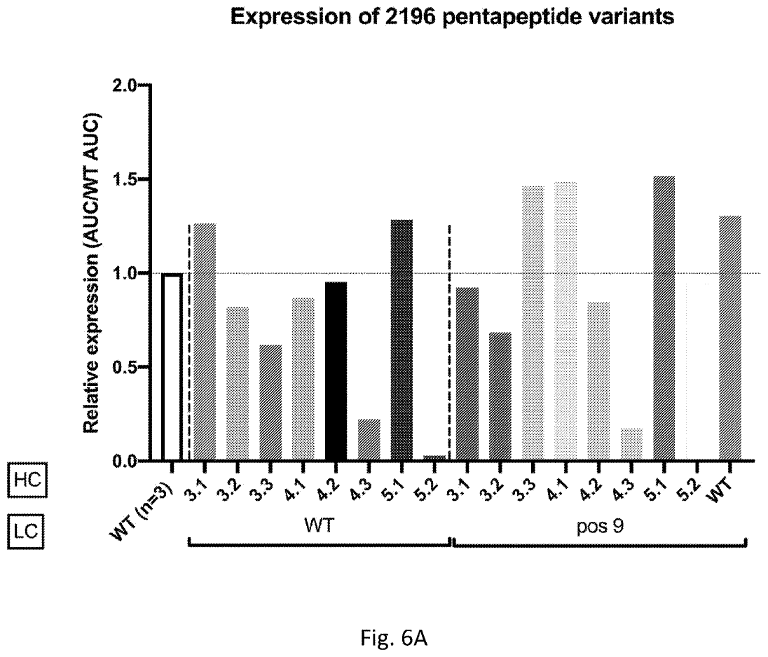

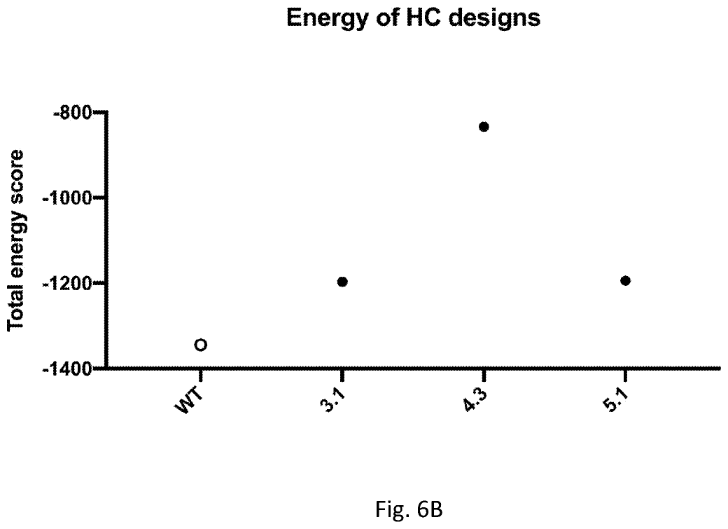

FIG. 6A shows the relative expression (AUC/AUC WT) of triple, quadruple, or quintuple combinations of point mutations to the heavy chain paired with either the WT light chain or a light chain with a single mutation (pos 9). Automated Western Blots were run to probe for the heavy chain and AUC corresponding to the heavy chain peak was assessed. FIG. 6B shows the total rosetta energy of notable combinations. FastRelax was run on the WT structure, and FastDesign was used to model the mutation combinations. Energy was subsequently determined.

### Pentapeptide Sequences:

Secretion tag
Heavy chains:
VH region (mutations in VH)
Constant region (CH1-CH2-CH3) (mutations in CH regions)
Light chains:
VL region (mutations in VL)
CL region
Prototype: Glycan Modification of 2196 antibody (targeting SARS-CoV-2)
SEQ ID NO: 1-2196_HC_WT_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACAAGCCTAGCAACACCAAAGTTGATAAGAAGGTGGAGC
CTAAGAGCTGCGATAAAACACATACATGTCCTCCGTGCCCTGCTCCTGAGCT
GCTGGGCGGCCCAAGCGTGTTCCTTTTTCCTCCTAAGCCCAAGGACACCCTG
ATGATCAGTAGAACCCCAGAGGTGACCTGTGTGGTGGTGGACGTGTCCCAC
GAGGACCCTGAGGTGAAGTTCAACTGGTACGTGGATGGCGTGGAAGTTCAT
AATGCCAAGACAAAACCTAGGGAAGAGCAGTACAACAGCACCTATAGAGTG
GTCTCCGTCCTGACCGTGCTGCACCAGGACTGGCTGAACGGCAAGGAATAC
AAGTGCAAGGTGAGCAACAAGGCCCTGCCTGCCCCTATCGAGAAGACCATT
AGCAAGGCTAAGGGCCAGCCTAGAGAGCCCCAGGTGTACACCCTGCCCCCC
AGCAGAGATGAGCTGACAAAGAACCAGGTGAGTCTGACATGCCTGGTCAAA
GGCTTCTACCCATCCGATATCGCCGTGGAGTGGGAGAGCAATGGCCAGCCTG
AAAACAACTACAAGACCACCCCTCCTGTGCTGGACAGCGACGGCAGCTTCTT
CCTGTACAGCAAGCTGACAGTGGACAAGTCCCGGTGGCAACAGGGCAACGT
GTTCAGCTGTAGCGTGATGCACGAGGCCCTGCACAACCACTACACACAGAA
AAGCCTCTCCCTGAGCCCTGGAAAGTGATGA
SEQ ID NO: 2-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 3-2196_HC_3.1_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGACATCGGCAGCGGCAATACCAACTACGCCCAGAAGTTTCAGGAGCGG
GTGACCATCACAAGAGACATGAGCACCAGCACCGCCTATATGGAACTGTCC
AGCCTGAGAAGTGAAGATACCGCCGTGTACTACTGTGCCGCCCCATACTGCA
GCAGCATCAGCTGCAACGACGGCTTCGACATCTGGGGACAAGGCACCATGG
TGACCGTGTCAAGCGCCTCTACCAAGGGCCCCAGCGTGTTCCCTCTTGCTCC
TTCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCCCCTCTTCTCTGGGCACCCAGACCTACATC
TGCAATGTGAACCACCCCCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 4-
MDWTWRILFLVAAATGTHAQMQLVQSGPEVKKPGTSVKVSCKASGFTFMSSAVQ
WVRQARGQRLEWIGWIDIGSGNTNYAQKFQERVTITRDMSTSTAYMELSSLRS
EDTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPPS
SLGTQTYICNVNHPPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 5-2196_HC_3.2_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGCCCGGCAGCGGCAATACCAACTACGCCCAGAAGTTTCAGGAGCGG
GTGACCATCACAAGAGACATGAGCACCAGCACCGCCTATATGGAACTGTCC
AGCCTGACCAGTGAAGATACCGCCGTGTACTACTGTGCCGCCCCATACTGC
AGCAGCATCAGCTGCAACGACGGCTTCGACATCTGGACCCAAGGCACCATG
GTGACCGTGTCAAGCGCCTCTACCAAGGGCCCCAGCGTGTTCCCTCTTGCTC
CTTCCTCCAAAAGCACCAGCGGAGGCACCGCCGCCCTGGGATGTCTGGTTAA
AGACTACTTCCCCGAACCTGTGACAGTGTCTTGGAACAGCGGTGCTCTGACC
TCCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACAAGCCTAGCAACACCAAAGTTGATAAGAAGGTGGAGC
CTAAGAGCTGCGATAAAACACATACATGTCCTCCGTGCCCTGCTCCTGAGCT
GCTGGGCGGCCCAAGCGTGTTCCTTTTTCCTCCTAAGCCCAAGGACACCCTG
ATGATCAGTAGAACCCCAGAGGTGACCTGTGTGGTGGTGGACGTGTCCCAC
GAGGACCCTGAGGTGAAGTTCAACTGGTACGTGGATGGCGTGGAAGTTCAT
AATGCCAAGACAAAACCTAGGGAAGAGCAGTACAACAGCACCTATAGAGTG
GTCTCCGTCCTGACCGTGCTGCACCAGGACTGGCTGAACGGCAAGGAATAC
AAGTGCAAGGTGAGCAACAAGGCCCTGCCTGCCCCTATCGAGAAGACCATT
AGCAAGGCTAAGGGCCAGCCTAGAGAGCCCCAGGTGTACACCCTGCCCCCC
AGCAGAGATGAGCTGACAAAGAACCAGGTGAGTCTGACATGCCTGGTCAAA
GGCTTCTACCCATCCGATATCGCCGTGGAGTGGGAGAGCAATGGCCAGCCTG
AAAACAACTACAAGACCACCCCTCCTGTGCTGGACAGCGACGGCAGCTTCTT
CCTGTACAGCAAGCTGACAGTGGACAAGTCCCGGTGGCAACAGGGCAACGT
GTTCAGCTGTAGCGTGATGCACGAGGCCCTGCACAACCACTACACACAGAA
AAGCCTCTCCCTGAGCCCTGGAAAGTGATGA
SEQ ID NO: 6-
MDWTWRILFLVAAATGTHAQMQLVQSGPEVKKPGTSVKVSCKASGFTFMSSAVQ
WVRQARGQRLEWIGWIVPGSGNTNYAQKFQERVTITRDMSTSTAYMELSSLTS
EDTAVYYCAAPYCSSISCNDGFDIWTQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 7-2196_HC_3.3_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGACATCGGCAGCGGCAATACCAACTACGCCCAGAAGTTTCAGGAGCGG
GTGACCATCACAAGAGACCCCAGCACCAGCACCGCCTATATGGAACTGTCC
AGCCTGACCAGTGAAGATACCGCCGTGTACTACTGTGCCGCCCCATACTGC
AGCAGCATCAGCTGCAACGACGGCTTCGACATCTGGGGACAAGGCACCATG
GTGACCGTGTCAAGCGCCTCTACCAAGGGCCCCAGCGTGTTCCCTCTTGCTC
CTTCCTCCAAAAGCACCAGCGGAGGCACCGCCGCCCTGGGATGTCTGGTTAA
AGACTACTTCCCCGAACCTGTGACAGTGTCTTGGAACAGCGGTGCTCTGACC
TCCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACAAGCCTAGCAACACCAAAGTTGATAAGAAGGTGGAGC
CTAAGAGCTGCGATAAAACACATACATGTCCTCCGTGCCCTGCTCCTGAGCT
GCTGGGCGGCCCAAGCGTGTTCCTTTTTCCTCCTAAGCCCAAGGACACCCTG
ATGATCAGTAGAACCCCAGAGGTGACCTGTGTGGTGGTGGACGTGTCCCAC
GAGGACCCTGAGGTGAAGTTCAACTGGTACGTGGATGGCGTGGAAGTTCAT
AATGCCAAGACAAAACCTAGGGAAGAGCAGTACAACAGCACCTATAGAGTG
GTCTCCGTCCTGACCGTGCTGCACCAGGACTGGCTGAACGGCAAGGAATAC
AAGTGCAAGGTGAGCAACAAGGCCCTGCCTGCCCCTATCGAGAAGACCATT
AGCAAGGCTAAGGGCCAGCCTAGAGAGCCCCAGGTGTACACCCTGCCCCCC
AGCAGAGATGAGCTGACAAAGAACCAGGTGAGTCTGACATGCCTGGTCAAA
GGCTTCTACCCATCCGATATCGCCGTGGAGTGGGAGAGCAATGGCCAGCCTG
AAAACAACTACAAGACCACCCCTCCTGTGCTGGACAGCGACGGCAGCTTCTT
CCTGTACAGCAAGCTGACAGTGGACAAGTCCCGGTGGCAACAGGGCAACGT
GTTCAGCTGTAGCGTGATGCACGAGGCCCTGCACAACCACTACACACAGAA
AAGCCTCTCCCTGAGCCCTGGAAAGTGATGA
SEQ ID NO: 8-
MDWTWRILFLVAAATGTHAQMQLVQSGPEVKKPGTSVKVSCKASGFTFMSSAVQ
WVRQARGQRLEWIGWIDIGSGNTNYAQKFQERVTITRDPSTSTAYMELSSLTS
EDTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 9-2196_HC_4.1_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACAGCCAGAAGTTTCAGGAGCGG
GTGACCATCACAAGAGACCCCAGCACCAGCACCGCCTATATGGAACTGTCC
AGCCTGACCAGTGAAGATACCGCCGTGTACTACTGTGCCGCCCCATACTGC
AGCAGCATCAGCTGCAACGACGGCTTCGACATCTGGGGACAAGGCACCATG
GTGACCGTGTCAAGCGCCTCTACCAAGGGCCCCAGCGTGTTCCCTCTTGCTC
CTTCCTCCAAAAGCACCAGCGGAGGCACCGCCGCCCTGGGATGTCTGGTTAA
AGACTACTTCCCCGAACCTGTGACAGTGTCTTGGAACAGCGGTGCTCTGACC
TCCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACCCCCCTAGCAACACCAAAGTTGATAAGAAGGTGGAG
CCTAAGAGCTGCGATAAAACACATACATGTCCTCCGTGCCCTGCTCCTGAGC
TGCTGGGCGGCCCAAGCGTGTTCCTTTTTCCTCCTAAGCCCAAGGACACCCT
GATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 10-
MDWTWRILFLVAAATGTHAQMQLVQSGPEVKKPGTSVKVSCKASGFTFMSSAVQ
WVRQARGQRLEWIGWIVIGSGNTNYSQKFQERVTITRDPSTSTAYMELSSLTSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHPPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 11-2196_HC_4.2_p Vax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGACATCGGCAGCGGCAATACCAACTACAGCCAGAAGTTTCAGGAGCGG
GTGACCATCACAAGAGACATGAGCACCAGCACCGCCTATATGGAACTGTCC
AGCCTGAGAAGTGAAGATACCGCCGTGTACTACTGTGCCGCCCCATACTGCA
GCAGCATCAGCTGCAACGACGGCTTCGACATCTGGACCCAAGGCACCATGG
TGACCGTGTCAAGCGCCTCTACCAAGGGCCCCAGCGTGTTCCCTCTTGCTCC
TTCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCCCCTCTTCTCTGGGCACCCAGACCTACATC
TGCAATGTGAACCACAAGCCTAGCAACACCAAAGTTGATAAGAAGGTGGAG
CCTAAGAGCTGCGATAAAACACATACATGTCCTCCGTGCCCTGCTCCTGAGC
TGCTGGGCGGCCCAAGCGTGTTCCTTTTTCCTCCTAAGCCCAAGGACACCCT
GATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 12-
MDWTWRILFLVAAATGTHAQMQLVQSGPEVKKPGTSVKVSCKASGFTFMSSAVQ
WVRQARGQRLEWIGWIDIGSGNTNYSQKFQERVTITRDMSTSTAYMELSSLRS
EDTAVYYCAAPYCSSISCNDGFDIWTQGTMVTVSSASTKGPSVFPLAPSSKSTSG
GTAALGCLVKDYFPEPVTVSWNSGALTSGVHTFPAVLQSSGLYSLSSVVTVPPS
SLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 13-2196_HC_4.3_p Vax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGCCCGGCAGCGGCAATACCAACTACGCCCAGAAGTTTCAGGAGCGG
GTGACCATCACAAGAGACCCCAGCACCAGCACCGCCTATATGGAACTGTCC
AGCCTGAGAAGTGAAGATACCGCCGTGTACTACTGTGCCGCCCCATACTGCA
GCAGCATCAGCTGCAACGACGGCTTCGACATCTGGACCCAAGGCACCATGG
TGACCGTGTCAAGCGCCTCTACCAAGGGCCCCAGCGTGTTCCCTCTTGCTCC
TTCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCCCCTCTTCTCTGGGCACCCAGACCTACATC
TGCAATGTGAACCACAAGCCTAGCAACACCAAAGTTGATAAGAAGGTGGAG
CCTAAGAGCTGCGATAAAACACATACATGTCCTCCGTGCCCTGCTCCTGAGC
TGCTGGGCGGCCCAAGCGTGTTCCTTTTTCCTCCTAAGCCCAAGGACACCCT
GATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 14-
MDWTWRILFLVAAATGTHAQMQLVQSGPEVKKPGTSVKVSCKASGFTFMSSAVQ
WVRQARGQRLEWIGWIVPGSGNTNYAQKFQERVTITRDPSTSTAYMELSSLRS
EDTAVYYCAAPYCSSISCNDGFDIWTQGTMVTVSSASTKGPSVFPLAPSSKSTSG
GTAALGCLVKDYFPEPVTVSWNSGALTSGVHTFPAVLQSSGLYSLSSVVTVPPS
SLGTQTYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 15-2196_HC_5.1_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGACATCGGCAGCGGCAATACCAACTACGCCCAGAAGTTTCAGGAGCGG
GTGACCATCACAAGAGACCCCAGCACCAGCACCGCCTATATGGAACTGTCC
AGCCTGACCAGTGAAGATACCGCCGTGTACTACTGTGCCGCCCCATACTGC
AGCAGCATCAGCTGCAACGACGGCTTCGACATCTGGGGACAAGGCACCATG
GTGACCGTGTCAAGCGCCTCTACCAAGGGCCCCAGCGTGTTCCCTCTTGCTC
CTTCCTCCAAAAGCACCAGCGGAGGCACCGCCGCCCTGGGATGTCTGGTTAA
AGACTACTTCCCCGAACCTGTGACAGTGTCTTGGAACAGCGGTGCTCTGACC
TCCGGCGTGCACACATTCCCCGCTGTTCTGCAGTCTTCTGGACTGTACAGCCT
GTCTTCTGTGGTGACAGTCCCCCCCTCTTCTCTGGGCACCCAGACCTACATC
TGCAATGTGAACCACCCCCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 16-
MDWTWRILFLVAAATGTHAQMQLVQSGPEVKKPGTSVKVSCKASGFTFMSSAVQ
WVRQARGQRLEWIGWIDIGSGNTNYAQKFQERVTITRDPSTSTAYMELSSLTS
EDTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPPS
SLGTQTYICNVNHPPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 17-2196_HC_5.2_p Vax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGCCCGGCAGCGGCAATACCAACTACAGCCAGAAGTTTCAGGAGCGG
GTGACCATCACAAGAGACCCCAGCACCAGCACCGCCTATATGGAACTGTCC
AGCCTGAGAAGTGAAGATACCGCCGTGTACTACTGTGCCGCCCCATACTGCA
GCAGCATCAGCTGCAACGACGGCTTCGACATCTGGGGACAAGGCACCATGG
TGACCGTGTCAAGCGCCTCTACCAAGGGCCCCAGCGTGTTCCCTCTTGCTCC
TTCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCCCCTCTTCTCTGGGCACCCAGACCTACATC
TGCAATGTGAACCACCCCCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 18-
MDWTWRILFLVAAATGTHAQMQLVQSGPEVKKPGTSVKVSCKASGFTFMSSAVQ
WVRQARGQRLEWIGWIVPGSGNTNYSQKFQERVTITRDPSTSTAYMELSSLRS
EDTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPPS
SLGTQTYICNVNHPPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 19-2196_LC_WT_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
SEQ ID NO: 20-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVT
HQGLSSPVTKSFNRGEC**
SEQ ID NO: 21-2196_LC_G101S_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCAGCCA
AGGCACAAAGGTCGAGATCAAGCGGACCGTGGCCGCCCCTAGCGTGTTTAT
CTTTCCTCCATCCGACGAGCAGCTGAAGAGCGGCACCGCCAGCGTGGTGTGC
CTGCTGAACAACTTCTACCCCCGGGAAGCCAAGGTGCAGTGGAAGGTGGAC
AACGCCCTGCAGAGCGGAAATAGCCAGGAGAGCGTTACAGAGCAGGATAGC
AAGGACTCCACCTACAGCCTGAGCAACACCCTGACTCTGTCTAAGGCCGACT
ATGAGAAGCACAAGGTCTATGCCTGCGAGGTGACACACCAGGGCCTGTCAA
GCCCCGTGACCAAAAGCTTCAACAGAGGAGAATGTTGATGA
SEQ ID NO: 22-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFSQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVT
HQGLSSPVTKSFNRGEC**
CH1_CL_glycans Sequences
SEQ ID NO: 23-2196p_HC_G.2_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACACCCCTAGCAACACCAAAGTTGATAAGAAGGTGGAG
CCTAAGAGCTGCGATAAAACACATACATGTCCTCCGTGCCCTGCTCCTGAGC
TGCTGGGCGGCCCAAGCGTGTTCCTTTTTCCTCCTAAGCCCAAGGACACCCT
GATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 24-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHTPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 25-2196p_HC_G3.2_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTAACACC
TCCGGCGTGCACACATTCCCCGCTGTTCTGCAGTCTTCTGGACTGTACAGCCT
GTCTTCTGTGGTGACAGTCCCCAGCTCTTCTCTGGGCACCCAGACCTACATCT
GCAATGTGAACCACACCCCTAGCAACACCAAAAACGATACCAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 26-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SLGTQTYICNVNHTPSNTKNDTKVEPKSCDKTHTCPPCPAPELLGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 27-2196p_HC_G3.3_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTCTGACCT
CCGGCGTGCACACATTCCCCGCTGTTCTGAACTCTTCTGGACTGTACAGCCT
GTCTTCTGTGGTGACAGTCCCCAGCTCTTCTAACGGCACCCAGACCTACATC
TGCAATGTGAACCACACCCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 28-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLNSSGLYSLSSVVTVPSS
SNGTQTYICNVNHTPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 29-2196p_HC_G3.4_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTAACACC
TCCGGCGTGCACACATTCCCCGCTGTTCTGCAGTCTTCTGGACTGTACAGCCT
GTCTTCTGTGGTGACAGTCCCCAGCTCTTCTAACGGCACCCAGACCTACATC
TGCAATGTGAGCCACAAGCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 30-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SNGTQTYICNVSHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 31-2196p_HC_G3.5_pVax1
ATGGACTGGACCTGGAGAATCCTGTTCCTGGTGGCTGCTGCTACAGGCACCCAC
GCCCAAATGCAGCTGGTCCAGAGCGGCCCTGAGGTGAAAAAGCCTGGCACA
TCTGTGAAGGTGTCCTGCAAGGCCAGCGGGTTTACATTCATGAGCTCTGCCG
TGCAGTGGGTGCGGCAGGCCAGAGGCCAGAGACTGGAATGGATCGGCTGGA
TCGTGATCGGCAGCGGCAATACCAACTACGCCCAGAAGTTTCAGGAGCGGG
TGACCATCACAAGAGACATGAGCACCAGCACCGCCTATATGGAACTGTCCA
GCCTGAGAAGTGAAGATACCGCCGTGTACTACTGTGCCGCCCCATACTGCAG
CAGCATCAGCTGCAACGACGGCTTCGACATCTGGGGACAAGGCACCATGGT
GACCGTGTCAAGCGCCTCTACCAAGGGCCCCAGCGTGTTCCCTCTTGCTCCT
TCCTCCAAAAGCACCAGCGGAGGCACCGCCGCCCTGGGATGTCTGGTTAAA
GACTACTTCCCCGAACCTGTGACAGTGTCTTGGAACAGCGGTGCTAACACC
TCCGGCGTGCACACATTCCCCGCTGTTCTGCAGTCTTCTGGACTGTACAGCCT
GTCTTCTGTGGTGACAGTCCCCAGCTCTTCTAACGGCACCCAGACCTACATC
TGCAATGTGAACCACACCCCTAGCAACACCAAAGTTGATAAGAAGGTGGA
GCCTAAGAGCTGCGATAAAACACATACATGTCCTCCGTGCCCTGCTCCTGAG
CTGCTGGGCGGCCCAAGCGTGTTCCTTTTTCCTCCTAAGCCCAAGGACACCC
TGATGATCAGTAGAACCCCAGAGGTGACCTGTGTGGTGGTGGACGTGTCCCA
CGAGGACCCTGAGGTGAAGTTCAACTGGTACGTGGATGGCGTGGAAGTTCA
TAATGCCAAGACAAAACCTAGGGAAGAGCAGTACAACAGCACCTATAGAGT
GGTCTCCGTCCTGACCGTGCTGCACCAGGACTGGCTGAACGGCAAGGAATA
CAAGTGCAAGGTGAGCAACAAGGCCCTGCCTGCCCCTATCGAGAAGACCAT
TAGCAAGGCTAAGGGCCAGCCTAGAGAGCCCCAGGTGTACACCCTGCCCCC
CAGCAGAGATGAGCTGACAAAGAACCAGGTGAGTCTGACATGCCTGGTCAA
AGGCTTCTACCCATCCGATATCGCCGTGGAGTGGGAGAGCAATGGCCAGCCT
GAAAACAACTACAAGACCACCCCTCCTGTGCTGGACAGCGACGGCAGCTTC
TTCCTGTACAGCAAGCTGACAGTGGACAAGTCCCGGTGGCAACAGGGCAAC
GTGTTCAGCTGTAGCGTGATGCACGAGGCCCTGCACAACCACTACACACAG
AAAAGCCTCTCCCTGAGCCCTGGAAAGTGATGA
SEQ ID NO: 32-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SNGTQTYICNVNHTPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 33-2196_LC_WT_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
SEQ ID NO: 34-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVT
HQGLSSPVTKSFNRGEC**
SEQ ID NO: 35-2196p_LC_G.5_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCAACGTGACACACCAGGGCCTGTCAAG
CCCCGTGACCAAAAGCTTCAACAGAGGAGAATGTTGATGA
SEQ ID NO: 36-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACNVT
HQGLSSPVTKSFNRGEC**
SEQ ID NO: 37-2196p_LC_G.6_p Vax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGAACCACAGCGGCCTGTCAAG
CCCCGTGACCAAAAGCTTCAACAGAGGAGAATGTTGATGA
SEQ ID NO: 38-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVN
HSGLSSPVTKSFNRGEC**
SEQ ID NO: 39-2196p_LC_G2.1_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGAACAGCA
CCGACTCCACCTACAGCCTGAGCAACACCCTGACTCTGTCTAAGGCCGACT
ATGAGAAGCACAAGGTCTATGCCTGCAACGTGACACACCAGGGCCTGTCAA
GCCCCGTGACCAAAAGCTTCAACAGAGGAGAATGTTGATGA
SEQ ID NO: 40-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSNTLTLSKADYEKHKVYACNVT
HQGLSSPVTKSFNRGEC**
SEQ ID NO: 41-2196p_LC_G2.2_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGAACAGCA
CCGACTCCACCTACAGCCTGAGCAACACCCTGACTCTGTCTAAGGCCGACT
ATGAGAAGCACAAGGTCTATGCCTGCGAGGTGAACCACAGCGGCCTGTCA
AGCCCCGTGACCAAAAGCTTCAACAGAGGAGAATGTTGATGA
SEQ ID NO: 42-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSNTLTLSKADYEKHKVYACEVN
HSGLSSPVTKSFNRGEC**
SEQ ID NO: 43-2196p_LC_G3.2_pVax1
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGAACAGCA
CCGACTCCACCTACAGCCTGAGCAACACCCTGAACCTGTCTAAGGCCGACT
ATGAGAAGCACAAGGTCTATGCCTGCGAGGTGAACCACAGCGGCCTGTCA
AGCCCCGTGACCAAAAGCTTCAACAGAGGAGAATGTTGATGA
SEQ ID NO: 44-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSNTLNLSKADYEKHKVYACEVN
HSGLSSPVTKSFNRGEC**
118mat variants (antibodies targeting HIV)
SEQ ID NO: 45-118mat_WT_HC_pVax
ATGGACTGGACCTGGAGAATCCTGTTCCTGGTGGCTGCTGCTACAGGCACCCAC
GCCCAGGGCAGACTGTTCCAGAGTGGCGCAGAGGTGAAGAGACCAGGAGCA
AGCGTGAGGATTAGTTGTCGGGCAGATGATGACCCTTACACCGACGATGAC
ACCTTCACAAAGTACTGGACACACTGGATCAGGCAGGCACCAGGACAGCGC
CCTGAGTGGCTGGGCGTGATCTCTCCACACTTCGCCAGGCCCATCTACAGCT
ATAAGTTTAGGGATAGACTGACCCTGACACGCGACAGCTCCCTGACCGCCGT
GTACCTGGAGCTGAAGGGCCTGCAGCCTGATGACAGCGGCATCTATTTCTGC
GCCAGGGACCCCTTCGGCGACAGGGCCCCACACTATAACTACCACATGGAT
GTCTGGGGAGGAGGAACCGCAGTCATTGTCTCTTCTGCCTCTACCAAGGGCC
CCAGCGTGTTCCCTCTTGCTCCTTCCTCCAAAAGCACCAGCGGAGGCACCGC
CGCCCTGGGATGTCTGGTTAAAGACTACTTCCCCGAACCTGTGACAGTGTCT
TGGAACAGCGGTGCTCTGACCTCCGGCGTGCACACATTCCCCGCTGTTCTGC
AGTCTTCTGGACTGTACAGCCTGTCTTCTGTGGTGACAGTCCCCAGCTCTTCT
CTGGGCACCCAGACCTACATCTGCAATGTGAACCACAAGCCTAGCAACACC
AAAGTTGATAAGAAGGTGGAGCCTAAGAGCTGCGATAAAACACATACATGT
CCTCCGTGCCCTGCTCCTGAGCTGCTGGGCGGCCCAAGCGTGTTCCTTTTTCC
TCCTAAGCCCAAGGACACCCTGATGATCAGTAGAACCCCAGAGGTGACCTG
TGTGGTGGTGGACGTGTCCCACGAGGACCCTGAGGTGAAGTTCAACTGGTAC
GTGGATGGCGTGGAAGTTCATAATGCCAAGACAAAACCTAGGGAAGAGCAG
TACAACAGCACCTATAGAGTGGTCTCCGTCCTGACCGTGCTGCACCAGGACT
GGCTGAACGGCAAGGAATACAAGTGCAAGGTGAGCAACAAGGCCCTGCCTG
CCCCTATCGAGAAGACCATTAGCAAGGCTAAGGGCCAGCCTAGAGAGCCCC
AGGTGTACACCCTGCCCCCCAGCAGAGATGAGCTGACAAAGAACCAGGTGA
GTCTGACATGCCTGGTCAAAGGCTTCTACCCATCCGATATCGCCGTGGAGTG
GGAGAGCAATGGCCAGCCTGAAAACAACTACAAGACCACCCCTCCTGTGCT
GGACAGCGACGGCAGCTTCTTCCTGTACAGCAAGCTGACAGTGGACAAGTC
CCGGTGGCAACAGGGCAACGTGTTCAGCTGTAGCGTGATGCACGAGGCCCT
GCACAACCACTACACACAGAAAAGCCTCTCCCTGAGCCCTGGAAAGTGATG
A
SEQ ID NO: 46-
MDWTWRILFLVAAATGTHAQGRLFQSGAEVKRPGASVRISCRADDDPYTDDDTF
TKYWTHWIRQAPGQRPEWLGVISPHFARPIYSYKFRDRLTLTRDSSLTAVYLEL
KGLQPDDSGIYFCARDPFGDRAPHYNYHMDVWGGGTAVIVSSASTKGPSVFPL
APSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK**
SEQ ID NO: 47-118mat_HC_G3.4_pVax
ATGGACTGGACCTGGAGAATCCTGTTCCTGGTGGCTGCTGCTACAGGCACCCAC
GCCCAGGGCAGACTGTTCCAGAGTGGCGCAGAGGTGAAGAGACCAGGAGCA
AGCGTGAGGATTAGTTGTCGGGCAGATGATGACCCTTACACCGACGATGAC
ACCTTCACAAAGTACTGGACACACTGGATCAGGCAGGCACCAGGACAGCGC
CCTGAGTGGCTGGGCGTGATCTCTCCACACTTCGCCAGGCCCATCTACAGCT
ATAAGTTTAGGGATAGACTGACCCTGACACGCGACAGCTCCCTGACCGCCGT
GTACCTGGAGCTGAAGGGCCTGCAGCCTGATGACAGCGGCATCTATTTCTGC
GCCAGGGACCCCTTCGGCGACAGGGCCCCACACTATAACTACCACATGGAT
GTCTGGGGAGGAGGAACCGCAGTCATTGTCTCTTCTGCCTCTACCAAGGGCC
CCAGCGTGTTCCCTCTTGCTCCTTCCTCCAAAAGCACCAGCGGAGGCACCGC
CGCCCTGGGATGTCTGGTTAAAGACTACTTCCCCGAACCTGTGACAGTGTCT
TGGAACAGCGGTGCTAACACCTCCGGCGTGCACACATTCCCCGCTGTTCTG
CAGTCTTCTGGACTGTACAGCCTGTCTTCTGTGGTGACAGTCCCCAGCTCTTC
TAACGGCACCCAGACCTACATCTGCAATGTGAGCCACAAGCCTAGCAACA
CCAAAGTTGATAAGAAGGTGGAGCCTAAGAGCTGCGATAAAACACATACAT
GTCCTCCGTGCCCTGCTCCTGAGCTGCTGGGCGGCCCAAGCGTGTTCCTTTTT
CCTCCTAAGCCCAAGGACACCCTGATGATCAGTAGAACCCCAGAGGTGACC
TGTGTGGTGGTGGACGTGTCCCACGAGGACCCTGAGGTGAAGTTCAACTGGT
ACGTGGATGGCGTGGAAGTTCATAATGCCAAGACAAAACCTAGGGAAGAGC
AGTACAACAGCACCTATAGAGTGGTCTCCGTCCTGACCGTGCTGCACCAGGA
CTGGCTGAACGGCAAGGAATACAAGTGCAAGGTGAGCAACAAGGCCCTGCC
TGCCCCTATCGAGAAGACCATTAGCAAGGCTAAGGGCCAGCCTAGAGAGCC
CCAGGTGTACACCCTGCCCCCCAGCAGAGATGAGCTGACAAAGAACCAGGT
GAGTCTGACATGCCTGGTCAAAGGCTTCTACCCATCCGATATCGCCGTGGAG
TGGGAGAGCAATGGCCAGCCTGAAAACAACTACAAGACCACCCCTCCTGTG
CTGGACAGCGACGGCAGCTTCTTCCTGTACAGCAAGCTGACAGTGGACAAG
TCCCGGTGGCAACAGGGCAACGTGTTCAGCTGTAGCGTGATGCACGAGGCC
CTGCACAACCACTACACACAGAAAAGCCTCTCCCTGAGCCCTGGAAAGTGA
TGA
SEQ ID NO: 48-
MDWTWRILFLVAAATGTHAQGRLFQSGAEVKRPGASVRISCRADDDPYTDDDTF
TKYWTHWIRQAPGQRPEWLGVISPHFARPIYSYKFRDRLTLTRDSSLTAVYLEL
KGLQPDDSGIYFCARDPFGDRAPHYNYHMDVWGGGTAVIVSSASTKGPSVFPL
APSSKSTSGGTAALGCLVKDYFPEPVTVSWNSGANTSGVHTFPAVLQSSGLYSL
SSVVTVPSSSNGTQTYICNVSHKPSNTKVDKKVEPKSCDKTHTCPPCPAPELLG
GPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPGK**
SEQ ID NO: 49-118mat_WT_LC_pVax
ATGGTGCTGCAAACACAGGTGTTCATTAGCCTGCTCCTGTGGATCTCCGGCGCCT
ACGGCGAGGTGGTGCTGACCCAGAGCCCCGCCATTCTGAGCGTGAGCCCAG
GAGACCGAGTGATTCTGAGTTGTAGAGCAAGCCAGGGACTGGACAGCTCCC
ACCTGGCCTGGTACCGGTTCAAGCGCGGCCAGATCCCCACCCTGGTGATCTT
TGGCACATCTAACAGGGCCAGAGGCACCCCTGACAGGTTCTCTGGCAGCGG
CTCCGGAGCAGACTTCACCCTGACAATCAGCCGGGTGGAGCCCGAGGATTTC
GCCACATACTATTGTCAGAGATACGGAGGCACACCCATCACCTTTGGCGGCG
GCACAACCCTGGATAAAAAACGGACCGTGGCCGCCCCTAGCGTGTTTATCTT
TCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
SEQ ID NO: 50-
MVLQTQVFISLLLWISGAYGEVVLTQSPAILSVSPGDRVILSCRASQGLDSSHLAW
YRFKRGQIPTLVIFGTSNRARGTPDRFSGSGSGADFTLTISRVEPEDFATYYCQRY
GGTPITFGGGTTLDKKRTVAAPSVFIFPPSDEQLKSGTASVVCLLNNFYPREAKV
QWKVDNALQSGNSQESVTEQDSKDSTYSLSNTLTLSKADYEKHKVYACEVTH
QGLSSPVTKSFNRGEC**
SEQ ID NO: 51-118mat_LC_G.6_pVax
ATGGTGCTGCAAACACAGGTGTTCATTAGCCTGCTCCTGTGGATCTCCGGCGCCT
ACGGCGAGGTGGTGCTGACCCAGAGCCCCGCCATTCTGAGCGTGAGCCCAG
GAGACCGAGTGATTCTGAGTTGTAGAGCAAGCCAGGGACTGGACAGCTCCC
ACCTGGCCTGGTACCGGTTCAAGCGCGGCCAGATCCCCACCCTGGTGATCTT
TGGCACATCTAACAGGGCCAGAGGCACCCCTGACAGGTTCTCTGGCAGCGG
CTCCGGAGCAGACTTCACCCTGACAATCAGCCGGGTGGAGCCCGAGGATTTC
GCCACATACTATTGTCAGAGATACGGAGGCACACCCATCACCTTTGGCGGCG
GCACAACCCTGGATAAAAAACGGACCGTGGCCGCCCCTAGCGTGTTTATCTT
TCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGAACCACAGCGGCCTGTCAAG
CCCCGTGACCAAAAGCTTCAACAGAGGAGAATGTTGATGA
SEQ ID NO: 52-
MVLQTQVFISLLLWISGAYGEVVLTQSPAILSVSPGDRVILSCRASQGLDSSHLAW
YRFKRGQIPTLVIFGTSNRARGTPDRFSGSGSGADFTLTISRVEPEDFATYYCQRY
GGTPITFGGGTTLDKKRTVAAPSVFIFPPSDEQLKSGTASVVCLLNNFYPREAKV
QWKVDNALQSGNSQESVTEQDSKDSTYSLSNTLTLSKADYEKHKVYACEVNH
SGLSSPVTKSFNRGEC**
SEQ ID NO: 53-118mat_LC_G3.2_pVax
ATGGTGCTGCAAACACAGGTGTTCATTAGCCTGCTCCTGTGGATCTCCGGCGCCT
ACGGCGAGGTGGTGCTGACCCAGAGCCCCGCCATTCTGAGCGTGAGCCCAG
GAGACCGAGTGATTCTGAGTTGTAGAGCAAGCCAGGGACTGGACAGCTCCC
ACCTGGCCTGGTACCGGTTCAAGCGCGGCCAGATCCCCACCCTGGTGATCTT
TGGCACATCTAACAGGGCCAGAGGCACCCCTGACAGGTTCTCTGGCAGCGG
CTCCGGAGCAGACTTCACCCTGACAATCAGCCGGGTGGAGCCCGAGGATTTC
GCCACATACTATTGTCAGAGATACGGAGGCACACCCATCACCTTTGGCGGCG
GCACAACCCTGGATAAAAAACGGACCGTGGCCGCCCCTAGCGTGTTTATCTT
TCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGAACAGCA
CCGACTCCACCTACAGCCTGAGCAACACCCTGAACCTGTCTAAGGCCGACT
ATGAGAAGCACAAGGTCTATGCCTGCGAGGTGAACCACAGCGGCCTGTCA
AGCCCCGTGACCAAAAGCTTCAACAGAGGAGAATGTTGATGA
SEQ ID NO: 54-
MVLQTQVFISLLLWISGAYGEVVLTQSPAILSVSPGDRVILSCRASQGLDSSHLAW
YRFKRGQIPTLVIFGTSNRARGTPDRFSGSGSGADFTLTISRVEPEDFATYYCQRY
GGTPITFGGGTTLDKKRTVAAPSVFIFPPSDEQLKSGTASVVCLLNNFYPREAKV
QWKVDNALQSGNSQESVTEQNSTDSTYSLSNTLNLSKADYEKHKVYACEVNH
SGLSSPVTKSFNRGEC**
AZ sequences (targeting SARS-CoV-2)
(CLIN + YTE mods)
SEQ ID NO: 55-2196modAZ_VH_YTE_pVax (WT HC)
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCCTGTACAGCCTGA
GCTCCGTGGTGACCGTGCCATCTAGCTCCCTGGGCACCCAGACATATATCTG
TAACGTGAATCACAAGCCTTCCAATACAAAGGTGGACAAGAGGGTGGAGCC
AAAGTCTTGCGATAAGACCCACACATGCCCTCCCTGTCCAGCACCTGAGCTG
CTGGGCGGCCCAAGCGTGTTCCTGTTTCCACCCAAGCCCAAGGACACCCTGT
ACATCACCAGGGAGCCAGAGGTGACATGCGTGGTGGTGGACGTGTCCCACG
AGGACCCCGAGGTGAAGTTTAACTGGTACGTGGATGGCGTGGAGGTGCACA
ATGCCAAGACCAAGCCCAGAGAGGAGCAGTACAATTCCACCTATCGGGTGG
TGTCTGTGCTGACAGTGCTGCACCAGGACTGGCTGAACGGCAAGGAGTACA
AGTGTAAGGTGTCTAATAAGGCCCTGCCAGCCCCCATCGAGAAGACCATCA
GCAAGGCAAAGGGACAGCCACGCGAGCCACAGGTGTATACACTGCCTCCAT
CTAGGGAGGAGATGACCAAGAACCAGGTGAGCCTGACATGCCTGGTGAAGG
GCTTCTACCCCAGCGATATCGCCGTGGAGTGGGAGTCCAATGGCCAGCCTGA
GAACAATTACAAGACCACACCCCCTGTGCTGGACTCTGATGGCAGCTTCTTT
CTGTATTCTAAGCTGACCGTGGACAAGAGCAGATGGCAGCAGGGCAACGTG
TTTTCTTGTAGCGTGATGCACGAGGCCCTGCACAATCACTACACCCAGAAGT
CCCTGTCTCTGAGCCCTGGCAAGTGATAA
SEQ ID NO: 56-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 57-96HC_YTE_G.1_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCCTGTACAGCCTGA
GCTCCGTGGTGACCGTGCCATCTAGCTCCCTGGGCACCCAGACATATATCTG
TAACGTGAGCCACAAGCCTTCCAATACAAAGGTGGACAAGAGGGTGGAGC
CAAAGTCTTGCGATAAGACCCACACATGCCCTCCCTGTCCAGCACCTGAGCT
GCTGGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 58-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVSHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 59-96HC_YTE_G.2_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCCTGTACAGCCTGA
GCTCCGTGGTGACCGTGCCATCTAGCTCCCTGGGCACCCAGACATATATCTG
TAACGTGAATCACACCCCTTCCAATACAAAGGTGGACAAGAGGGTGGAGC
CAAAGTCTTGCGATAAGACCCACACATGCCCTCCCTGTCCAGCACCTGAGCT
GCTGGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 60-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHTPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 61-96HC_YTE_G.3_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCCTGTACAGCCTGA
GCTCCGTGGTGACCGTGCCATCTAGCTCCCTGGGCACCCAGACATATATCTG
TAACGTGAATCACAAGCCTTCCAATACAAAGAACGACACCAGGGTGGAGC
CAAAGTCTTGCGATAAGACCCACACATGCCCTCCCTGTCCAGCACCTGAGCT
GCTGGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 62-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKNDTRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 63-96HC_YTE_G.4_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAACTCCAGCTCTACCAAGGGCCCAAGCGTGTTTCCCCTGGCCCCTT
CTAGCAAGAGCACCTCCGGCGGCACAGCCGCCCTGGGCTGTCTGGTGAAGG
ACTACTTCCCTGAGCCAGTGACAGTGAGCTGGAACTCCGGCGCCCTGACCTC
CGGAGTGCACACATTTCCTGCCGTGCTGCAGTCCTCTGGCCTGTACAGCCTG
AGCTCCGTGGTGACCGTGCCATCTAGCTCCCTGGGCACCCAGACATATATCT
GTAACGTGAATCACAAGCCTTCCAATACAAAGGTGGACAAGAGGGTGGAGC
CAAAGTCTTGCGATAAGACCCACACATGCCCTCCCTGTCCAGCACCTGAGCT
GCTGGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 64-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVNSSSTKGPSVFPLAPSSKSTSG
GTAALGCLVKDYFPEPVTVSWNSGALTSGVHTFPAVLQSSGLYSLSSVVTVPSS
SLGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 65-96HC_YTE_G.5_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCAACACCTC
CGGAGTGCACACATTTCCTGCCGTGCTGCAGTCCTCTGGCCTGTACAGCCTG
AGCTCCGTGGTGACCGTGCCATCTAGCTCCCTGGGCACCCAGACATATATCT
GTAACGTGAATCACAAGCCTTCCAATACAAAGGTGGACAAGAGGGTGGAGC
CAAAGTCTTGCGATAAGACCCACACATGCCCTCCCTGTCCAGCACCTGAGCT
GCTGGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 66-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SLGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 67-96HC_YTE_G.6_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGAACTCCTCTGGCCTGTACAGCCTGA
GCTCCGTGGTGACCGTGCCATCTAGCTCCCTGGGCACCCAGACATATATCTG
TAACGTGAATCACAAGCCTTCCAATACAAAGGTGGACAAGAGGGTGGAGCC
AAAGTCTTGCGATAAGACCCACACATGCCCTCCCTGTCCAGCACCTGAGCTG
CTGGGCGGCCCAAGCGTGTTCCTGTTTCCACCCAAGCCCAAGGACACCCTGT
ACATCACCAGGGAGCCAGAGGTGACATGCGTGGTGGTGGACGTGTCCCACG
AGGACCCCGAGGTGAAGTTTAACTGGTACGTGGATGGCGTGGAGGTGCACA
ATGCCAAGACCAAGCCCAGAGAGGAGCAGTACAATTCCACCTATCGGGTGG
TGTCTGTGCTGACAGTGCTGCACCAGGACTGGCTGAACGGCAAGGAGTACA
AGTGTAAGGTGTCTAATAAGGCCCTGCCAGCCCCCATCGAGAAGACCATCA
GCAAGGCAAAGGGACAGCCACGCGAGCCACAGGTGTATACACTGCCTCCAT
CTAGGGAGGAGATGACCAAGAACCAGGTGAGCCTGACATGCCTGGTGAAGG
GCTTCTACCCCAGCGATATCGCCGTGGAGTGGGAGTCCAATGGCCAGCCTGA
GAACAATTACAAGACCACACCCCCTGTGCTGGACTCTGATGGCAGCTTCTTT
CTGTATTCTAAGCTGACCGTGGACAAGAGCAGATGGCAGCAGGGCAACGTG
TTTTCTTGTAGCGTGATGCACGAGGCCCTGCACAATCACTACACCCAGAAGT
CCCTGTCTCTGAGCCCTGGCAAGTGATAA
SEQ ID NO: 68-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLNSSGLYSLSSVVTVPSS
SLGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 69-96HC_YTE_G.7_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCTACAGCCTGAGCT
CCGTGGTGACCGTGCCATCTAGCTCCCTGGGCACCCAGACATATATCTGTAA
CGTGAATCACAAGCCTTCCAATACAAAGGTGGACAAGAGGGTGGAGCCAAA
GTCTTGCGATAAGACCCACACATGCCCTCCCTGTCCAGCACCTGAGCTGCTG
AACGGCGGCCCAAGCGTGTTCCTGTTTCCACCCAAGCCCAAGGACACCCTG
TACATCACCAGGGAGCCAGAGGTGACATGCGTGGTGGTGGACGTGTCCCAC
GAGGACCCCGAGGTGAAGTTTAACTGGTACGTGGATGGCGTGGAGGTGCAC
AATGCCAAGACCAAGCCCAGAGAGGAGCAGTACAATTCCACCTATCGGGTG
GTGTCTGTGCTGACAGTGCTGCACCAGGACTGGCTGAACGGCAAGGAGTAC
AAGTGTAAGGTGTCTAATAAGGCCCTGCCAGCCCCCATCGAGAAGACCATC
AGCAAGGCAAAGGGACAGCCACGCGAGCCACAGGTGTATACACTGCCTCCA
TCTAGGGAGGAGATGACCAAGAACCAGGTGAGCCTGACATGCCTGGTGAAG
GGCTTCTACCCCAGCGATATCGCCGTGGAGTGGGAGTCCAATGGCCAGCCTG
AGAACAATTACAAGACCACACCCCCTGTGCTGGACTCTGATGGCAGCTTCTT
TCTGTATTCTAAGCTGACCGTGGACAAGAGCAGATGGCAGCAGGGCAACGT
GTTTTCTTGTAGCGTGATGCACGAGGCCCTGCACAATCACTACACCCAGAAG
TCCCTGTCTCTGAGCCCTGGCAAGTGATAA
SEQ ID NO: 70-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGNYSLSSVVTVPSS
SLGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 71-96HC_YTE_G.8_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGCAGTCCTCTGGCCTGTACAGCCTGA
GCTCCGTGGTGACCGTGCCATCTAGCTCCAACGGCACCCAGACATATATCTG
TAACGTGAATCACAAGCCTTCCAATACAAAGGTGGACAAGAGGGTGGAGCC
AAAGTCTTGCGATAAGACCCACACATGCCCTCCCTGTCCAGCACCTGAGCTG
CTGGGCGGCCCAAGCGTGTTCCTGTTTCCACCCAAGCCCAAGGACACCCTGT
ACATCACCAGGGAGCCAGAGGTGACATGCGTGGTGGTGGACGTGTCCCACG
AGGACCCCGAGGTGAAGTTTAACTGGTACGTGGATGGCGTGGAGGTGCACA
ATGCCAAGACCAAGCCCAGAGAGGAGCAGTACAATTCCACCTATCGGGTGG
TGTCTGTGCTGACAGTGCTGCACCAGGACTGGCTGAACGGCAAGGAGTACA
AGTGTAAGGTGTCTAATAAGGCCCTGCCAGCCCCCATCGAGAAGACCATCA
GCAAGGCAAAGGGACAGCCACGCGAGCCACAGGTGTATACACTGCCTCCAT
CTAGGGAGGAGATGACCAAGAACCAGGTGAGCCTGACATGCCTGGTGAAGG
GCTTCTACCCCAGCGATATCGCCGTGGAGTGGGAGTCCAATGGCCAGCCTGA
GAACAATTACAAGACCACACCCCCTGTGCTGGACTCTGATGGCAGCTTCTTT
CTGTATTCTAAGCTGACCGTGGACAAGAGCAGATGGCAGCAGGGCAACGTG
TTTTCTTGTAGCGTGATGCACGAGGCCCTGCACAATCACTACACCCAGAAGT
CCCTGTCTCTGAGCCCTGGCAAGTGATAA
SEQ ID NO: 72-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLQSSGLYSLSSVVTVPSS
SNGTQTYICNVNHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 73-96HC_YTE_G3.1_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAACTCCAGCTCTACCAAGGGCCCAAGCGTGTTTCCCCTGGCCCCTT
CTAGCAAGAGCACCTCCGGCGGCACAGCCGCCCTGGGCTGTCTGGTGAAGG
ACTACTTCCCTGAGCCAGTGACAGTGAGCTGGAACTCCGGCGCCCTGACCTC
CGGAGTGCACACATTTCCTGCCGTGCTGCAGTCCTCTGGCCTGTACAGCCTG
AGCTCCGTGGTGACCGTGCCATCTAGCTCCAACGGCACCCAGACATATATC
TGTAACGTGAGCCACAAGCCTTCCAATACAAAGGTGGACAAGAGGGTGGA
GCCAAAGTCTTGCGATAAGACCCACACATGCCCTCCCTGTCCAGCACCTGAG
CTGCTGGGCGGCCCAAGCGTGTTCCTGTTTCCACCCAAGCCCAAGGACACCC
TGTACATCACCAGGGAGCCAGAGGTGACATGCGTGGTGGTGGACGTGTCCC
ACGAGGACCCCGAGGTGAAGTTTAACTGGTACGTGGATGGCGTGGAGGTGC
ACAATGCCAAGACCAAGCCCAGAGAGGAGCAGTACAATTCCACCTATCGGG
TGGTGTCTGTGCTGACAGTGCTGCACCAGGACTGGCTGAACGGCAAGGAGT
ACAAGTGTAAGGTGTCTAATAAGGCCCTGCCAGCCCCCATCGAGAAGACCA
TCAGCAAGGCAAAGGGACAGCCACGCGAGCCACAGGTGTATACACTGCCTC
CATCTAGGGAGGAGATGACCAAGAACCAGGTGAGCCTGACATGCCTGGTGA
AGGGCTTCTACCCCAGCGATATCGCCGTGGAGTGGGAGTCCAATGGCCAGC
CTGAGAACAATTACAAGACCACACCCCCTGTGCTGGACTCTGATGGCAGCTT
CTTTCTGTATTCTAAGCTGACCGTGGACAAGAGCAGATGGCAGCAGGGCAA
CGTGTTTTCTTGTAGCGTGATGCACGAGGCCCTGCACAATCACTACACCCAG
AAGTCCCTGTCTCTGAGCCCTGGCAAGTGATAA
SEQ ID NO: 74-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVNSSSTKGPSVFPLAPSSKSTSG
GTAALGCLVKDYFPEPVTVSWNSGALTSGVHTFPAVLQSSGLYSLSSVVTVPSS
SNGTQTYICNVSHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 75-96HC_YTE_G3.2_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCAACACCTC
CGGAGTGCACACATTTCCTGCCGTGCTGCAGTCCTCTGGCCTGTACAGCCTG
AGCTCCGTGGTGACCGTGCCATCTAGCTCCCTGGGCACCCAGACATATATCT
GTAACGTGAATCACACCCCTTCCAATACAAAGAACGACACCAGGGTGGA
GCCAAAGTCTTGCGATAAGACCCACACATGCCCTCCCTGTCCAGCACCTGAG
CTGCTGGGCGGCCCAAGCGTGTTCCTGTTTCCACCCAAGCCCAAGGACACCC
TGTACATCACCAGGGAGCCAGAGGTGACATGCGTGGTGGTGGACGTGTCCC
ACGAGGACCCCGAGGTGAAGTTTAACTGGTACGTGGATGGCGTGGAGGTGC
ACAATGCCAAGACCAAGCCCAGAGAGGAGCAGTACAATTCCACCTATCGGG
TGGTGTCTGTGCTGACAGTGCTGCACCAGGACTGGCTGAACGGCAAGGAGT
ACAAGTGTAAGGTGTCTAATAAGGCCCTGCCAGCCCCCATCGAGAAGACCA
TCAGCAAGGCAAAGGGACAGCCACGCGAGCCACAGGTGTATACACTGCCTC
CATCTAGGGAGGAGATGACCAAGAACCAGGTGAGCCTGACATGCCTGGTGA
AGGGCTTCTACCCCAGCGATATCGCCGTGGAGTGGGAGTCCAATGGCCAGC
CTGAGAACAATTACAAGACCACACCCCCTGTGCTGGACTCTGATGGCAGCTT
CTTTCTGTATTCTAAGCTGACCGTGGACAAGAGCAGATGGCAGCAGGGCAA
CGTGTTTTCTTGTAGCGTGATGCACGAGGCCCTGCACAATCACTACACCCAG
AAGTCCCTGTCTCTGAGCCCTGGCAAGTGATAA
SEQ ID NO: 76-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SLGTQTYICNVNHTPSNTKNDTRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 77-96HC_YTE_G3.3_p Vax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCCTGACCTCC
GGAGTGCACACATTTCCTGCCGTGCTGAACTCCTCTGGCCTGTACAGCCTGA
GCTCCGTGGTGACCGTGCCATCTAGCTCCAACGGCACCCAGACATATATCT
GTAACGTGAATCACACCCCTTCCAATACAAAGGTGGACAAGAGGGTGGAG
CCAAAGTCTTGCGATAAGACCCACACATGCCCTCCCTGTCCAGCACCTGAGC
TGCTGGGCGGCCCAAGCGTGTTCCTGTTTCCACCCAAGCCCAAGGACACCCT
GTACATCACCAGGGAGCCAGAGGTGACATGCGTGGTGGTGGACGTGTCCCA
CGAGGACCCCGAGGTGAAGTTTAACTGGTACGTGGATGGCGTGGAGGTGCA
CAATGCCAAGACCAAGCCCAGAGAGGAGCAGTACAATTCCACCTATCGGGT
GGTGTCTGTGCTGACAGTGCTGCACCAGGACTGGCTGAACGGCAAGGAGTA
CAAGTGTAAGGTGTCTAATAAGGCCCTGCCAGCCCCCATCGAGAAGACCAT
CAGCAAGGCAAAGGGACAGCCACGCGAGCCACAGGTGTATACACTGCCTCC
ATCTAGGGAGGAGATGACCAAGAACCAGGTGAGCCTGACATGCCTGGTGAA
GGGCTTCTACCCCAGCGATATCGCCGTGGAGTGGGAGTCCAATGGCCAGCCT
GAGAACAATTACAAGACCACACCCCCTGTGCTGGACTCTGATGGCAGCTTCT
TTCTGTATTCTAAGCTGACCGTGGACAAGAGCAGATGGCAGCAGGGCAACG
TGTTTTCTTGTAGCGTGATGCACGAGGCCCTGCACAATCACTACACCCAGAA
GTCCCTGTCTCTGAGCCCTGGCAAGTGATAA
SEQ ID NO: 78-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGALTSGVHTFPAVLNSSGLYSLSSVVTVPSS
SNGTQTYICNVNHTPSNTKVDKRVEPKSCDKTHTCPPCPAPELLGGPSVFLFPP
KPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 79-96HC_YTE_G3.4_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCAACACCTC
CGGAGTGCACACATTTCCTGCCGTGCTGCAGTCCTCTGGCCTGTACAGCCTG
AGCTCCGTGGTGACCGTGCCATCTAGCTCCAACGGCACCCAGACATATATC
TGTAACGTGAGCCACAAGCCTTCCAATACAAAGGTGGACAAGAGGGTGGA
GCCAAAGTCTTGCGATAAGACCCACACATGCCCTCCCTGTCCAGCACCTGAG
CTGCTGGGCGGCCCAAGCGTGTTCCTGTTTCCACCCAAGCCCAAGGACACCC
TGTACATCACCAGGGAGCCAGAGGTGACATGCGTGGTGGTGGACGTGTCCC
ACGAGGACCCCGAGGTGAAGTTTAACTGGTACGTGGATGGCGTGGAGGTGC
ACAATGCCAAGACCAAGCCCAGAGAGGAGCAGTACAATTCCACCTATCGGG
TGGTGTCTGTGCTGACAGTGCTGCACCAGGACTGGCTGAACGGCAAGGAGT
ACAAGTGTAAGGTGTCTAATAAGGCCCTGCCAGCCCCCATCGAGAAGACCA
TCAGCAAGGCAAAGGGACAGCCACGCGAGCCACAGGTGTATACACTGCCTC
CATCTAGGGAGGAGATGACCAAGAACCAGGTGAGCCTGACATGCCTGGTGA
AGGGCTTCTACCCCAGCGATATCGCCGTGGAGTGGGAGTCCAATGGCCAGC
CTGAGAACAATTACAAGACCACACCCCCTGTGCTGGACTCTGATGGCAGCTT
CTTTCTGTATTCTAAGCTGACCGTGGACAAGAGCAGATGGCAGCAGGGCAA
CGTGTTTTCTTGTAGCGTGATGCACGAGGCCCTGCACAATCACTACACCCAG
AAGTCCCTGTCTCTGAGCCCTGGCAAGTGATAA
SEQ ID NO: 80-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SNGTQTYICNVSHKPSNTKVDKRVEPKSCDKTHTCPPCPAPELLGGPSVFLFPPK
PKDTLYITREPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPP
SREEMTKNQVSLTCLVKGFYPSDIAVEWESNGQPENNYKTTPPVLDSDGSFFLY
SKLTVDKSRWQQGNVFSCSVMHEALHNHYTQKSLSLSPGK**
SEQ ID NO: 81-96HC_YTE_G3.5_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCAACACCTC
CGGAGTGCACACATTTCCTGCCGTGCTGCAGTCCTCTGGCCTGTACAGCCTG
AGCTCCGTGGTGACCGTGCCATCTAGCTCCAACGGCACCCAGACATATATC
TGTAACGTGAATCACACCCCTTCCAATACAAAGGTGGACAAGAGGGTGGA
GCCAAAGTCTTGCGATAAGACCCACACATGCCCTCCCTGTCCAGCACCTGAG
CTGCTGGGCGGCCCAAGCGTGTTCCTGTTTCCACCCAAGCCCAAGGACACCC
TGTACATCACCAGGGAGCCAGAGGTGACATGCGTGGTGGTGGACGTGTCCC
ACGAGGACCCCGAGGTGAAGTTTAACTGGTACGTGGATGGCGTGGAGGTGC
ACAATGCCAAGACCAAGCCCAGAGAGGAGCAGTACAATTCCACCTATCGGG
TGGTGTCTGTGCTGACAGTGCTGCACCAGGACTGGCTGAACGGCAAGGAGT
ACAAGTGTAAGGTGTCTAATAAGGCCCTGCCAGCCCCCATCGAGAAGACCA
TCAGCAAGGCAAAGGGACAGCCACGCGAGCCACAGGTGTATACACTGCCTC
CATCTAGGGAGGAGATGACCAAGAACCAGGTGAGCCTGACATGCCTGGTGA
AGGGCTTCTACCCCAGCGATATCGCCGTGGAGTGGGAGTCCAATGGCCAGC
CTGAGAACAATTACAAGACCACACCCCCTGTGCTGGACTCTGATGGCAGCTT
CTTTCTGTATTCTAAGCTGACCGTGGACAAGAGCAGATGGCAGCAGGGCAA
CGTGTTTTCTTGTAGCGTGATGCACGAGGCCCTGCACAATCACTACACCCAG
AAGTCCCTGTCTCTGAGCCCTGGCAAGTGATAA
SEQ ID NO: 82-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLQSSGLYSLSSVVTVPSS
SNGTQTYICNVNHTPSNTKVDKRVEPKSCDKTHTCPPCPAPELLGGPSVFLFPP
KPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 83-96HC_YTE_G5.1_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAACTCCAGCTCTACCAAGGGCCCAAGCGTGTTTCCCCTGGCCCCTT
CTAGCAAGAGCACCTCCGGCGGCACAGCCGCCCTGGGCTGTCTGGTGAAGG
ACTACTTCCCTGAGCCAGTGACAGTGAGCTGGAACTCCGGCGCCCTGACCTC
CGGAGTGCACACATTTCCTGCCGTGCTGCAGTCCTCTGGCAACTACAGCCT
GAGCTCCGTGGTGACCGTGCCATCTAGCTCCAACGGCACCCAGACATATAT
CTGTAACGTGAGCCACAAGCCTTCCAATACAAAGAACGACACCAGGGTG
GAGCCAAAGTCTTGCGATAAGACCCACACATGCCCTCCCTGTCCAGCACCTG
AGCTGCTGGGCGGCCCAAGCGTGTTCCTGTTTCCACCCAAGCCCAAGGACAC
CCTGTACATCACCAGGGAGCCAGAGGTGACATGCGTGGTGGTGGACGTGTC
CCACGAGGACCCCGAGGTGAAGTTTAACTGGTACGTGGATGGCGTGGAGGT
GCACAATGCCAAGACCAAGCCCAGAGAGGAGCAGTACAATTCCACCTATCG
GGTGGTGTCTGTGCTGACAGTGCTGCACCAGGACTGGCTGAACGGCAAGGA
GTACAAGTGTAAGGTGTCTAATAAGGCCCTGCCAGCCCCCATCGAGAAGAC
CATCAGCAAGGCAAAGGGACAGCCACGCGAGCCACAGGTGTATACACTGCC
TCCATCTAGGGAGGAGATGACCAAGAACCAGGTGAGCCTGACATGCCTGGT
GAAGGGCTTCTACCCCAGCGATATCGCCGTGGAGTGGGAGTCCAATGGCCA
GCCTGAGAACAATTACAAGACCACACCCCCTGTGCTGGACTCTGATGGCAGC
TTCTTTCTGTATTCTAAGCTGACCGTGGACAAGAGCAGATGGCAGCAGGGCA
ACGTGTTTTCTTGTAGCGTGATGCACGAGGCCCTGCACAATCACTACACCCA
GAAGTCCCTGTCTCTGAGCCCTGGCAAGTGATAA
SEQ ID NO: 84-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVNSSSTKGPSVFPLAPSSKSTSG
GTAALGCLVKDYFPEPVTVSWNSGALTSGVHTFPAVLQSSGNYSLSSVVTVPSS
SNGTQTYICNVSHKPSNTKNDTRVEPKSCDKTHTCPPCPAPELLGGPSVFLFPP
KPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 85-96HC_YTE_G5.2_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAACTCCAGCTCTACCAAGGGCCCAAGCGTGTTTCCCCTGGCCCCTT
CTAGCAAGAGCACCTCCGGCGGCACAGCCGCCCTGGGCTGTCTGGTGAAGG
ACTACTTCCCTGAGCCAGTGACAGTGAGCTGGAACTCCGGCGCCAACACCT
CCGGAGTGCACACATTTCCTGCCGTGCTGCAGTCCTCTGGCCTGTACAGCCT
GAGCTCCGTGGTGACCGTGCCATCTAGCTCCAACGGCACCCAGACATATAT
CTGTAACGTGAATCACACCCCTTCCAATACAAAGAACGACACCAGGGTG
GAGCCAAAGTCTTGCGATAAGACCCACACATGCCCTCCCTGTCCAGCACCTG
AGCTGCTGGGCGGCCCAAGCGTGTTCCTGTTTCCACCCAAGCCCAAGGACAC
CCTGTACATCACCAGGGAGCCAGAGGTGACATGCGTGGTGGTGGACGTGTC
CCACGAGGACCCCGAGGTGAAGTTTAACTGGTACGTGGATGGCGTGGAGGT
GCACAATGCCAAGACCAAGCCCAGAGAGGAGCAGTACAATTCCACCTATCG
GGTGGTGTCTGTGCTGACAGTGCTGCACCAGGACTGGCTGAACGGCAAGGA
GTACAAGTGTAAGGTGTCTAATAAGGCCCTGCCAGCCCCCATCGAGAAGAC
CATCAGCAAGGCAAAGGGACAGCCACGCGAGCCACAGGTGTATACACTGCC
TCCATCTAGGGAGGAGATGACCAAGAACCAGGTGAGCCTGACATGCCTGGT
GAAGGGCTTCTACCCCAGCGATATCGCCGTGGAGTGGGAGTCCAATGGCCA
GCCTGAGAACAATTACAAGACCACACCCCCTGTGCTGGACTCTGATGGCAGC
TTCTTTCTGTATTCTAAGCTGACCGTGGACAAGAGCAGATGGCAGCAGGGCA
ACGTGTTTTCTTGTAGCGTGATGCACGAGGCCCTGCACAATCACTACACCCA
GAAGTCCCTGTCTCTGAGCCCTGGCAAGTGATAA
SEQ ID NO: 86-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVNSSSTKGPSVFPLAPSSKSTSG
GTAALGCLVKDYFPEPVTVSWNSGANTSGVHTFPAVLQSSGLYSLSSVVTVPSS
SNGTQTYICNVNHTPSNTKNDTRVEPKSCDKTHTCPPCPAPELLGGPSVFLFPP
KPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 87-96HC_YTE_G5.3_pVax
ATGGACTGGACATGGAGAATCCTGTTCCTGGTCGCCGCCGCAACTGGCACTCAC
GCTCAGATGCAGCTGGTGCAGTCTGGACCCGAGGTGAAGAAGCCCGGCACC
AGCGTGAAGGTGTCCTGTAAGGCCTCTGGCTTCACCTTTATGAGCTCCGCCG
TGCAGTGGGTGAGGCAGGCCAGAGGCCAGCGGCTGGAGTGGATCGGATGGA
TCGTGATCGGCTCCGGAAACACCAATTACGCCCAGAAGTTCCAGGAGCGCG
TGACCATCACAAGGGACATGTCCACCTCTACAGCCTATATGGAGCTGTCTAG
CCTGCGGTCCGAGGATACAGCCGTGTACTATTGCGCCGCCCCTTACTGTTCC
TCTATCAGCTGCAACGACGGCTTCGATATCTGGGGCCAGGGCACCATGGTGA
CAGTGAGCTCCGCCTCTACCAAGGGCCCAAGCGTGTTTCCCCTGGCCCCTTC
TAGCAAGAGCACCTCCGGCGGCACAGCCGCCCTGGGCTGTCTGGTGAAGGA
CTACTTCCCTGAGCCAGTGACAGTGAGCTGGAACTCCGGCGCCAACACCTC
CGGAGTGCACACATTTCCTGCCGTGCTGAACTCCTCTGGCCTGTACAGCCTG
AGCTCCGTGGTGACCGTGCCATCTAGCTCCAACGGCACCCAGACATATATC
TGTAACGTGAGCCACAAGCCTTCCAATACAAAGAACGACACCAGGGTGG
AGCCAAAGTCTTGCGATAAGACCCACACATGCCCTCCCTGTCCAGCACCTGA
GCTGCTGGGCGGCCCAAGCGTGTTCCTGTTTCCACCCAAGCCCAAGGACACC
CTGTACATCACCAGGGAGCCAGAGGTGACATGCGTGGTGGTGGACGTGTCC
CACGAGGACCCCGAGGTGAAGTTTAACTGGTACGTGGATGGCGTGGAGGTG
CACAATGCCAAGACCAAGCCCAGAGAGGAGCAGTACAATTCCACCTATCGG
GTGGTGTCTGTGCTGACAGTGCTGCACCAGGACTGGCTGAACGGCAAGGAG
TACAAGTGTAAGGTGTCTAATAAGGCCCTGCCAGCCCCCATCGAGAAGACC
ATCAGCAAGGCAAAGGGACAGCCACGCGAGCCACAGGTGTATACACTGCCT
CCATCTAGGGAGGAGATGACCAAGAACCAGGTGAGCCTGACATGCCTGGTG
AAGGGCTTCTACCCCAGCGATATCGCCGTGGAGTGGGAGTCCAATGGCCAG
CCTGAGAACAATTACAAGACCACACCCCCTGTGCTGGACTCTGATGGCAGCT
TCTTTCTGTATTCTAAGCTGACCGTGGACAAGAGCAGATGGCAGCAGGGCAA
CGTGTTTTCTTGTAGCGTGATGCACGAGGCCCTGCACAATCACTACACCCAG
AAGTCCCTGTCTCTGAGCCCTGGCAAGTGATAA
SEQ ID NO: 88-
MDWTWRILFLVAAATGTHAQMQLVQSGPEVKKPGTSVKVSCKASGFTFMSSAVQ
WVRQARGQRLEWIGWIVIGSGNTNYAQKFQERVTITRDMSTSTAYMELSSLRSE
DTAVYYCAAPYCSSISCNDGFDIWGQGTMVTVSSASTKGPSVFPLAPSSKSTSG
GTAALGCLVKDYFPEPVTVSWNSGANTSGVHTFPAVLNSSGLYSLSSVVTVPSS
SNGTQTYICNVSHKPSNTKNDTRVEPKSCDKTHTCPPCPAPELLGGPSVFLFPP
KPKDTLYITREPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLP
PSREEMTKNQVSLTCLVKGFYPSDIAVEWESNGQPENNYKTTPPVLDSDGSFFL
YSKLTVDKSRWQQGNVFSCSVMHEALHNHYTQKSLSLSPGK**
SEQ ID NO: 89-96_LG.1_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGAACAAGAGCGGCACAGCCTCCGTGGTGTGC
CTGCTGAACAATTTCTATCCCCGCGAGGCCAAGGTGCAGTGGAAGGTGGAT
AACGCCCTGCAGTCCGGCAATTCTCAGGAGAGCGTGACCGAGCAGGACTCC
AAGGATTCTACATACTCCCTGTCTAGCACCCTGACACTGAGCAAGGCCGATT
ACGAGAAGCACAAGGTGTATGCCTGCGAGGTGACCCATCAGGGGCTGTCTT
CTCCAGTGACCAAATCCTTCAATCGCGGGGAATGTTGATAA
SEQ ID NO: 90-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQNKSGTASVVCLLNNFYPREA
KVQWKVDNALQSGNSQESVTEQDSKDSTYSLSSTLTLSKADYEKHKVYACEVT
HQGLSSPVTKSFNRGEC**
SEQ ID NO: 91-96_LG.2_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTAGCGAGAGCGTGACCGAGCAGGACTCCA
AGGATTCTACATACTCCCTGTCTAGCACCCTGACACTGAGCAAGGCCGATTA
CGAGAAGCACAAGGTGTATGCCTGCGAGGTGACCCATCAGGGGCTGTCTTCT
CCAGTGACCAAATCCTTCAATCGCGGGGAATGTTGATAA
SEQ ID NO: 92-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSSESVTEQDSKDSTYSLSSTLTLSKADYEKHKVYACEVTH
QGLSSPVTKSFNRGEC**
SEQ ID NO: 93-96_LG.3_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGAACTCC
ACCGATTCTACATACTCCCTGTCTAGCACCCTGACACTGAGCAAGGCCGAT
TACGAGAAGCACAAGGTGTATGCCTGCGAGGTGACCCATCAGGGGCTGTCT
TCTCCAGTGACCAAATCCTTCAATCGCGGGGAATGTTGATAA
SEQ ID NO: 94-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSSTLTLSKADYEKHKVYACEVT
HQGLSSPVTKSFNRGEC**
SEQ ID NO: 95-96_LG.4_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGGACTCCA
AGGATTCTACATACTCCCTGTCTAGCACCCTGAACCTGAGCAAGGCCGATT
ACGAGAAGCACAAGGTGTATGCCTGCGAGGTGACCCATCAGGGGCTGTCTT
CTCCAGTGACCAAATCCTTCAATCGCGGGGAATGTTGATAA
SEQ ID NO: 96-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSSTLNLSKADYEKHKVYACEVT
HQGLSSPVTKSFNRGEC**
SEQ ID NO: 97-96_LG.5_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGGACTCCA
AGGATTCTACATACTCCCTGTCTAGCACCCTGACACTGAGCAAGGCCGATTA
CGAGAAGCACAAGGTGTATGCCTGCAACGTGACCCATCAGGGGCTGTCTTC
TCCAGTGACCAAATCCTTCAATCGCGGGGAATGTTGATAA
SEQ ID NO: 98-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACNVT
HQGLSSPVTKSFNRGEC**
SEQ ID NO: 99-96_LG.6_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGGACTCCA
AGGATTCTACATACTCCCTGTCTAGCACCCTGACACTGAGCAAGGCCGATTA
CGAGAAGCACAAGGTGTATGCCTGCGAGGTGAACCATAGCGGGCTGTCTT
CTCCAGTGACCAAATCCTTCAATCGCGGGGAATGTTGATAA
SEQ ID NO: 100-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACEVN
HSGLSSPVTKSFNRGEC**
SEQ ID NO: 101-96_LG2.1_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGAACTCC
ACCGATTCTACATACTCCCTGTCTAGCACCCTGACACTGAGCAAGGCCGAT
TACGAGAAGCACAAGGTGTATGCCTGCAACGTGACCCATCAGGGGCTGTCT
TCTCCAGTGACCAAATCCTTCAATCGCGGGGAATGTTGATAA
SEQ ID NO: 102-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSSTLTLSKADYEKHKVYACNVT
HQGLSSPVTKSFNRGEC**
SEQ ID NO: 103-96_LG2.2_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGAACTCC
ACCGATTCTACATACTCCCTGTCTAGCACCCTGACACTGAGCAAGGCCGAT
TACGAGAAGCACAAGGTGTATGCCTGCGAGGTGAACCATAGCGGGCTGTC
TTCTCCAGTGACCAAATCCTTCAATCGCGGGGAATGTTGATAA
SEQ ID NO: 104-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSSTLTLSKADYEKHKVYACEVN
HSGLSSPVTKSFNRGEC**
SEQ ID NO: 105-96_LG3.1_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGAACAAGAGCGGCACAGCCTCCGTGGTGTGC
CTGCTGAACAATTTCTATCCCCGCGAGGCCAAGGTGCAGTGGAAGGTGGAT
AACGCCCTGCAGTCCGGCAATTCTCAGGAGAGCGTGACCGAGCAGAACTCC
ACCGATTCTACATACTCCCTGTCTAGCACCCTGACACTGAGCAAGGCCGAT
TACGAGAAGCACAAGGTGTATGCCTGCAACGTGACCCATCAGGGGCTGTCT
TCTCCAGTGACCAAATCCTTCAATCGCGGGGAATGTTGATAA
SEQ ID NO: 106-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQNKSGTASVVCLLNNFYPREA
KVQWKVDNALQSGNSQESVTEQNSTDSTYSLSSTLTLSKADYEKHKVYACNV
THQGLSSPVTKSFNRGEC**
SEQ ID NO: 107-96_LG3.2_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGCTGAAGAGCGGCACAGCCTCCGTGGTGTGCC
TGCTGAACAATTTCTATCCCCGCGAGGCCAAGGTGCAGTGGAAGGTGGATA
ACGCCCTGCAGTCCGGCAATTCTCAGGAGAGCGTGACCGAGCAGAACTCC
ACCGATTCTACATACTCCCTGTCTAGCACCCTGAACCTGAGCAAGGCCGAT
TACGAGAAGCACAAGGTGTATGCCTGCGAGGTGAACCATAGCGGGCTGTC
TTCTCCAGTGACCAAATCCTTCAATCGCGGGGAATGTTGATAA
SEQ ID NO: 108-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQNSTDSTYSLSSTLNLSKADYEKHKVYACEVN
HSGLSSPVTKSFNRGEC**
SEQ ID NO: 109-96_LG3.3_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGAACAAGAGCGGCACAGCCTCCGTGGTGTGC
CTGCTGAACAATTTCTATCCCCGCGAGGCCAAGGTGCAGTGGAAGGTGGAT
AACGCCCTGCAGTCCGGCAATTCTAGCGAGAGCGTGACCGAGCAGGACTCC
AAGGATTCTACATACTCCCTGTCTAGCACCCTGACACTGAGCAAGGCCGATT
ACGAGAAGCACAAGGTGTATGCCTGCGAGGTGAACCATAGCGGGCTGTCT
TCTCCAGTGACCAAATCCTTCAATCGCGGGGAATGTTGATAA
SEQ ID NO: 110-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQNKSGTASVVCLLNNFYPREA
KVQWKVDNALQSGNSSESVTEQDSKDSTYSLSSTLTLSKADYEKHKVYACEV
NHSGLSSPVTKSFNRGEC**
SEQ ID NO: 111-96_LG5.1_pVax1
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGAGCGTGTCTAGCTCCTA
CCTGGCCTGGTATCAGCAGAAGCCTGGCCAGGCCCCAAGACTGCTGATCTAC
GGAGCCTCTAGCCGCGCCACCGGCATCCCCGACAGGTTCTCCGGCTCTGGCA
GCGGCACAGACTTCACCCTGACAATCTCCCGGCTGGAGCCTGAGGACTTCGC
CGTGTACTACTGTCAGCACTATGGCTCCTCTCGGGGCTGGACCTTTGGCCAG
GGCACAAAGGTGGAGATCAAGAGGACCGTGGCAGCACCATCCGTGTTCATC
TTTCCACCCTCTGACGAGCAGAACAAGAGCGGCACAGCCTCCGTGGTGTGC
CTGCTGAACAATTTCTATCCCCGCGAGGCCAAGGTGCAGTGGAAGGTGGAT
AACGCCCTGCAGTCCGGCAATTCTAGCGAGAGCGTGACCGAGCAGAACTC
CACCGATTCTACATACTCCCTGTCTAGCACCCTGAACCTGAGCAAGGCCGA
TTACGAGAAGCACAAGGTGTATGCCTGCAACGTGACCCATCAGGGGCTGTC
TTCTCCAGTGACCAAATCCTTCAATCGCGGGGAATGTTGATAA
SEQ ID NO: 112-
MVLQTQVFISLLLWISGAYGEIVLTQSPGTLSLSPGERATLSCRASQSVSSSYLAWY
QQKPGQAPRLLIYGASSRATGIPDRFSGSGSGTDFTLTISRLEPEDFAVYYCQHY
GSSRGWTFGQGTKVEIKRTVAAPSVFIFPPSDEQNKSGTASVVCLLNNFYPREA
KVQWKVDNALQSGNSSESVTEQNSTDSTYSLSSTLNLSKADYEKHKVYACNV
THQGLSSPVTKSFNRGEC**

### Example 2: Chain Swapping of Antibody CDRs for Enhanced In Vivo Expression

Coronaviruses (CoV) are a family of viruses that are common worldwide and cause a range of illnesses in humans from the common cold to severe acute respiratory syndrome (SARS). Coronaviruses can also cause a number of diseases in animals. Human coronaviruses 229E, OC43, NL63, and HKU are endemic in the human population.

The sudden rise and spread of severe acute respiratory syndrome coronavirus 2 (SARS-CoV-2), the causative agent of coronavirus disease 2019 (COVID-19), has resulted in a global pandemic with >230 million infections and claimed >4.7 million lives to date (Johns Hopkins Coronavirus Resource Center. No Title. COVID-19 Map; coronavirus.jhu.edu/map.html). Development of countermeasures, both prophylactic and therapeutic, are needed to combat the emergence of viral variants that threaten the efficacy of current approaches (Kuzmina et al., 2021, Cell Host Microbe, 29:522-528.e2; Wang et al., 2021, Nature, 592:616-622; Chen et al., 2021, Nat Med, 27:717-26). Antibody-based therapy is an important strategy for both prevention and treatment that could play a valuable role in protecting numerous populations.

Neutralizing Abs (nAbs) targeting the SARS-CoV-2 Spike(S) protein block viral attachment and invasion with the human ACE-2 receptor. Efforts to identify, characterize and develop SARS-CoV-2 nAbs for clinical translation resulted in the identification of many highly potent monoclonal candidates (Robbiani et al., 2020, Nature, 584:437-442; Barnes et al., 2020, Cell, 182:828-842.e16; Zost et al., 2020, Nature, 584:443-9; Cao et al., 2020, Cell, 182:73-84.e16; Liu et al., 2020, Nature, 584:450-6; Pinto et al., 2020, Nature, 583:290-5; Kreye et al., 2020, Cell, 183 (4): 1058-1069.e19; Hansen et al., 2020, Science, 369:1010-4; Brouwer et al., 2020, Science, 369:643-50; Baum et al., 2020, Science, 369:1014-8; Zost et al., 2020, Nat Med, 26:1422-7). Several of these advanced through the clinic and have been granted emergency use authorization (EUA) for the treatment of mild-to-moderate disease, including dual cocktails REGN-COV2 (Weinreich et al., 2021, Engl J Med, 384:238-51) (Regeneron), LY3832479 (Gottlieb et al., 2021, JAMA, 325:632-44) (Eli Lilly/AbCellera) and monotherapy Sotrovimab/VIR-7831 (Cathcart et al., 2021, bioRxiv, 434607) (Vir Biotechnology/GlaxoSmithKline). Additional candidates are showing promising Phase III trials, including cocktails AZD7442 (Dong et al., 2021, Nat Microbiol 6, 1233-1244) (AstraZeneca) and C144-LS/C135-LS (Rompay et al., 2021, PLOS Pathog, 17: e1009688) (Bristol-Myers Squibb/Rockefeller). These important biologics thus far appear to remain largely effective against emerging SARS-CoV-2 variants of concern, especially multi-component formulations containing non-redundant mAbs that simultaneously bind RBD at separate epitopes (Gottlieb et al., 2021, JAMA, 325:632-44; Copin et al., 2021, Cell, 184(15):3949-3961.e11; Chen et al., 2021, NatureCom, 27:717-26; Chen et al., 2021, Nature, 596:103-8; Starr et al., 2021, Cell Reports Med, Volume 2, Issue 4, 20 Apr. 2021, 100255; Wang et al., 2021, Nature, 593:130-135).

DNA-encoded monoclonal antibodies (DMAbs) are DNA-vectored antibodies that have demonstrated strong protective efficacy in animal models, are simple to manufacture, are cold chain resistant and have no anti-vector immunity, thus are an important alternative to traditional biologics. However, ensuring consistent, high levels of DMAb expression is a major barrier to clinical translation.

The Experiments presented herein demonstrate the generation of DMAbs having a high level of expression using chain swapping. Chain Swapping is the deliberate mismatch of antibody heavy and light chains.

Chain swap improves expression: pairing heavy chain from antibody of interest with clonally related or same-germline light chains from other antibodies results in boosts to in vivo expression. Chains from the antibody of interest are paired with chains from clonally related antibodies, or antibodies with the same VH or VK/VL germline gene.

The materials and methods are now described.

Light chain swapping was performed on 2196, a VK3-20 IgG1 antibody for SARS-CoV-2 (Zost et al., 2020, Nature, 584(7821):443-449).

The light chains from clonally related anti-SARS-CoV-2 antibody 2072 and germline-matched anti-Ebola antibody alphamod1 were paired with the heavy chain of 2196 (Zost et al., 2020, Nature, 584(7821):443-449). Structural analysis was performed to identify which sites might boost expression while maintaining binding. In vivo expression of selected variants was assessed.

Sequence information related to antibodies of interest was extracted from the Observed Antibody Space (OAS) (Kovaltsuk et al., 2018, J Immunol, 201:2502-2509; Olsen et al., 2022, Protein Science, 31:141-146). The OAS is database with over 230 million light chains identified from studies where sequences was taken from healthy individuals. The frequency of amino acids of interest in the designed constructs was compared to naturally occurring antibodies from the database to determine whether natural bias towards certain amino acids might indicate preferences for antibodies with better expression.

The experimental results are now described.

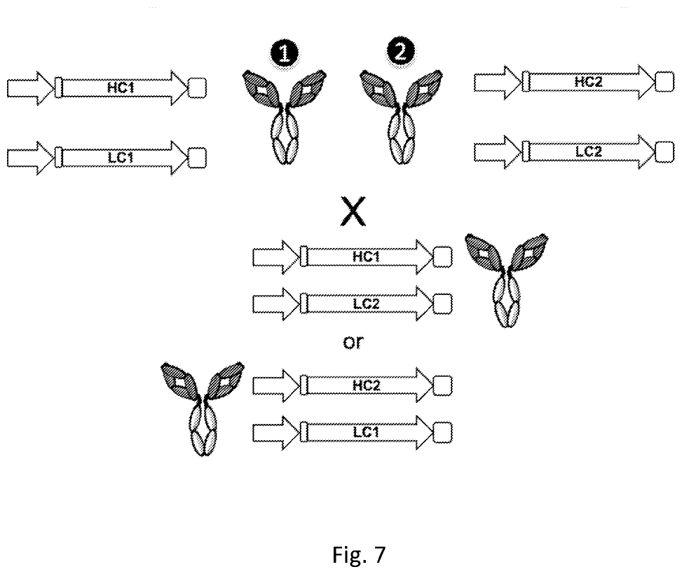

FIG. 7 depicts a diagram of different methods of chain swapping.

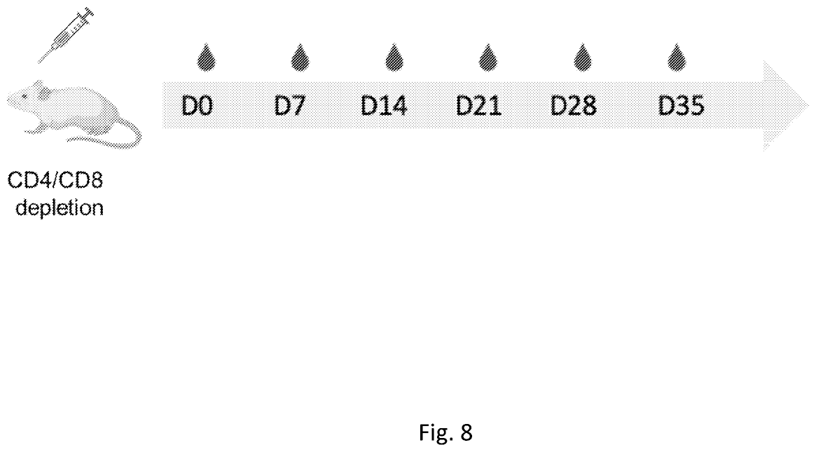

FIG. 8 depicts a diagram of the experimental design.

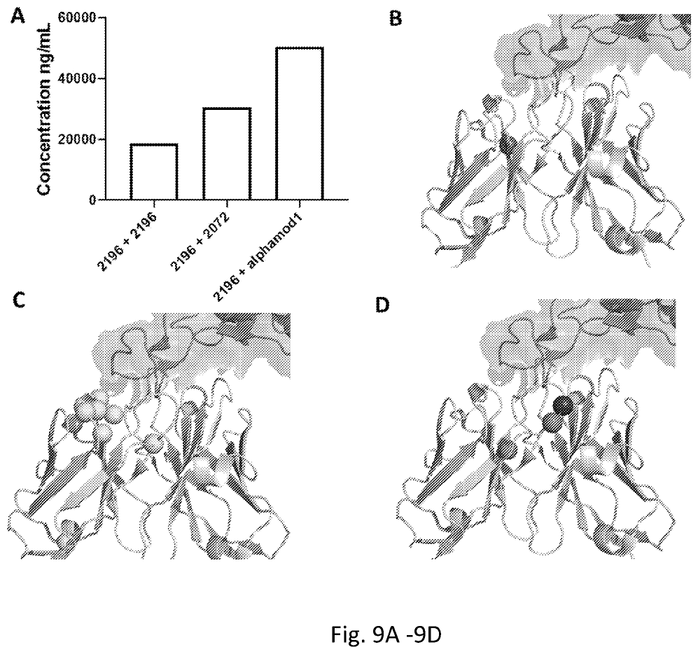

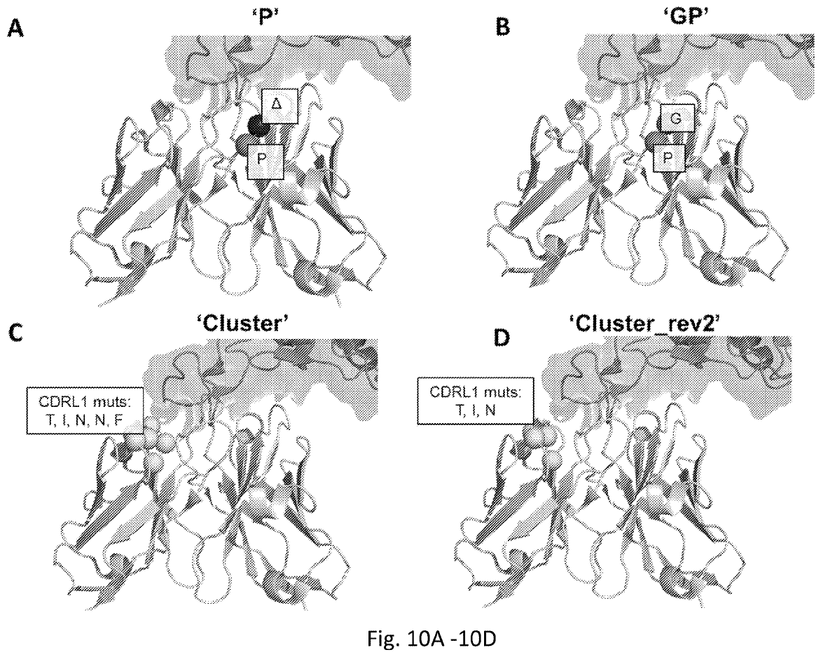

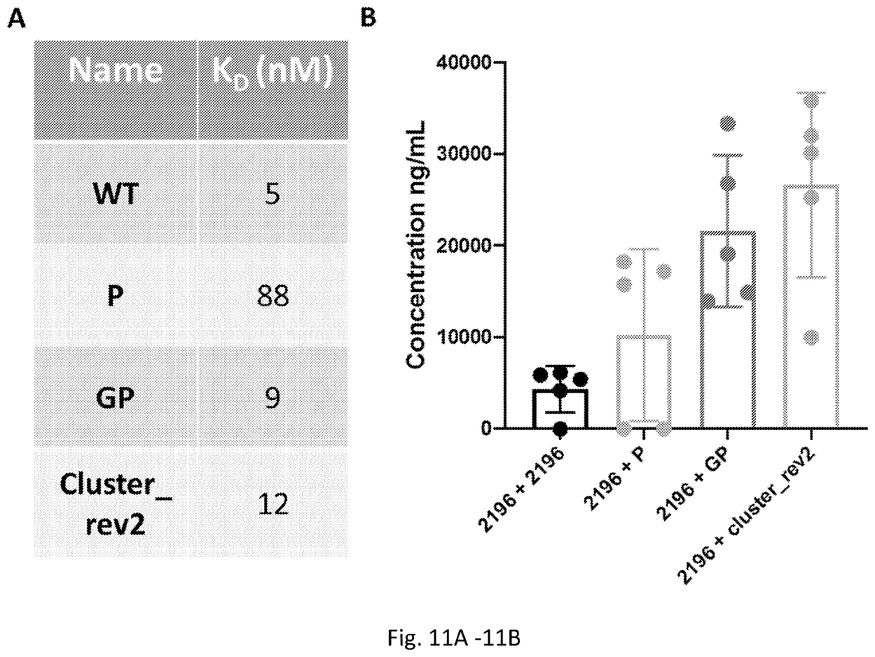

Structural analysis revealed mutations that might be responsible for expression boosts locate to antibody CDR loops (FIG. 9). Second generation designs based on structural analysis preserved binding while boosting in vivo expression (FIG. 10). Expression improvements of designed variants are 5-6× higher than WT (FIG. 11).

Analysis of proline in CDRL3 shows enrichment across all VK3-20 antibodies found in OAS database.

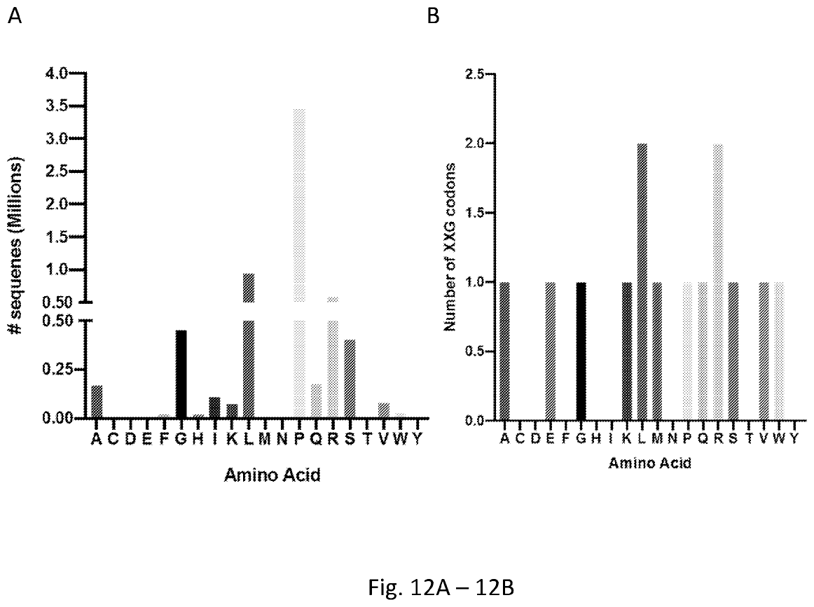

Codon analysis suggests that enrichment for proline is selected for, and may provide support for this position as key for in vivo expression improvements (FIG. 12).

### Sequences

Key: Leader-Variable light chain sequence-Constant light chain sequence
Leader sequence in italics
Variants from WT within the variable light chain sequence are indicated in bold
Constant chain underlined

2196_LC_WT_pVax1
SEQ ID NO: 113
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGAGCGTGTCC
AGCAGCTACCTGGCTTGGTACCAGCAGAAACCTGGCCAGGCCCCTAGACT
GCTGATCTACGGCGCTAGCTCTAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCAGAGG
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 114
MVLQTQVFISLLLWISGAYGEIVLTQSPGTLSLSPGERATLSCRASQSVS
SSYLAWYQQKPGQAPRLLIYGASSRATGIPDRFSGSGSGTDFTLTISRLE
PEDFAVYYCQHYGSSRGWTFGQGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC**
Alphamod1_LC_pVax1
SEQ ID NO: 115
ATGGTGCTGCAGACCCAGGTGTTCATCAGCCTGCTGCTGTGGATCTCCGG
CGCCTACGGCGAGATCGTGCTGACCCAGTCCCCAGGCACACTGAGCCTGT
CCCCTGGAGAGAGAGCCACCCTGAGCTGCAGGGCATCTCAGACCATTTCT
AATAATTTTGTTGCCTGGTATCAGCAGAAGCCTGGCCAGGCCCCAAGACT
GCTGATCTACGGAGCCAGCACCCGCGCCACCGGCATCCCCGACAGGTTCT
CCGGCTCTGGCAGCGGCACAGACTTCACCCTGACAATCTCCCGGCTGGAG
CCTGAGGACTTCGCCGTGTACTACTGTCAGCAGTATGGATCCTCTCCCTA
CACCTTTGGCCAGGGCACAAAGCTGGAGATCAAGCGTACAGTGGCCGCCC
CCAGCGTGTTCATCTTTCCACCCAGCGACGAGCAGCTGAAGTCCGGCACC
GCCTCTGTGGTGTGCCTGCTGAACAATTTCTACCCTCGGGAGGCCAAGGT
GCAGTGGAAGGTGGATAACGCCCTGCAGTCCGGCAATTCTCAGGAGAGCG
TGACCGAGCAGGACTCCAAGGATTCTACATATAGCCTGAGCTCCACCCTG
ACACTGAGCAAGGCCGACTACGAGAAGCACAAGGTGTATGCCTGTGAGGT
CACCCACCAGGGGCTGTCAAGTCCAGTCACTAAAAGTTTCAATAGGGGAG
AATGTTGATAA
SEQ ID NO: 116
MVLQTQVFISLLLWISGAYGEIVLTQSPGTLSLSPGERATLSCRASQTIS
NNFVAWYQQKPGQAPRLLIYGASTRATGIPDRFSGSGSGTDFTLTISRLE
PEDFAVYYCQQYGSSPYTFGQGTKLEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC*
2196_LC_H91Q_pVax1
SEQ ID NO: 117
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGAGCGTGTCC
AGCAGCTACCTGGCTTGGTACCAGCAGAAACCTGGCCAGGCCCCTAGACT
GCTGATCTACGGCGCTAGCTCTAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCAGTACGGCAGCTCCAGAGG
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 118
MVLQTQVFISLLLWISGAYGEIVLTQSPGTLSLSPGERATLSCRASQSVS
SSYLAWYQQKPGQAPRLLIYGASSRATGIPDRFSGSGSGTDFTLTISRLE
PEDFAVYYCQQYGSSRGWTFGQGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC**
2196_LC_P_pVax1
SEQ ID NO: 119
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGAGCGTGTCC
AGCAGCTACCTGGCTTGGTACCAGCAGAAACCTGGCCAGGCCCCTAGACT
GCTGATCTACGGCGCTAGCTCTAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCCCCTG
GACCTTCGGCCAAGGCACAAAGGTCGAGATCAAGCGGACCGTGGCCGCCC
CTAGCGTGTTTATCTTTCCTCCATCCGACGAGCAGCTGAAGAGCGGCACC
GCCAGCGTGGTGTGCCTGCTGAACAACTTCTACCCCCGGGAAGCCAAGGT
GCAGTGGAAGGTGGACAACGCCCTGCAGAGCGGAAATAGCCAGGAGAGCG
TTACAGAGCAGGATAGCAAGGACTCCACCTACAGCCTGAGCAACACCCTG
ACTCTGTCTAAGGCCGACTATGAGAAGCACAAGGTCTATGCCTGCGAGGT
GACACACCAGGGCCTGTCAAGCCCCGTGACCAAAAGCTTCAACAGAGGAG
AATGTTGATGA
SEQ ID NO: 120
MVLQTQVFISLLLWISGAYGEIVLTQSPGTLSLSPGERATLSCRASQSVS
SSYLAWYQQKPGQAPRLLIYGASSRATGIPDRFSGSGSGTDFTLTISRLE
PEDFAVYYCQHYGSSPWTFGQGTKVEIKRTVAAPSVFIFPPSDEQLKSGT
ASVVCLLNNFYPREAKVQWKVDNALQSGNSQESVTEQDSKDSTYSLSNTL
TLSKADYEKHKVYACEVTHQGLSSPVTKSFNRGEC**
2196_LC_RP_pVax1
SEQ ID NO: 121
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGAGCGTGTCC
AGCAGCTACCTGGCTTGGTACCAGCAGAAACCTGGCCAGGCCCCTAGACT
GCTGATCTACGGCGCTAGCTCTAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCAGACC
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 122
MVLQTQVFISLLLWISGAYGEIVLTQSPGTLSLSPGERATLSCRASQSVS
SSYLAWYQQKPGQAPRLLIYGASSRATGIPDRFSGSGSGTDFTLTISRLE
PEDFAVYYCQHYGSSRPWTFGQGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC**
2196_LC_GP_pVax1
SEQ ID NO: 123
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGAGCGTGTCC
AGCAGCTACCTGGCTTGGTACCAGCAGAAACCTGGCCAGGCCCCTAGACT
GCTGATCTACGGCGCTAGCTCTAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCGGCCC
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 124
MVLQTQVFISLLLWISGAYGEIVLTQSPGTLSLSPGERATLSCRASQSVS
SSYLAWYQQKPGQAPRLLIYGASSRATGIPDRFSGSGSGTDFTLTISRLE
PEDFAVYYCQHYGSSGPWTFGQGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC**
2196_LC_cluster_pVax1
SEQ ID NO: 125
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGACCATTTCC
AATAATTTTGTTGCTTGGTACCAGCAGAAACCTGGCCAGGCCCCTAGACT
GCTGATCTACGGCGCTAGCACCAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCAGAGG
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 126
MVLQTQVFISLLLWISGAYGEIVLTQSPGTLSLSPGERATLSCRASQTIS
NNFVAWYQQKPGQAPRLLIYGASTRATGIPDRFSGSGSGTDFTLTISRLE
PEDFAVYYCQHYGSSRGWTFGQGTKVEIKRTVAAPSVFIFPPSDEQLKSG
TASVVCLLNNFYPREAKVOWKVDNALOSGNSQESVTEQDSKDSTYSLSNT
LTLSKADYEKHKVYACEVTHQGLSSPVTKSFNRGEC**
2196_LC_cluster_rev2_pVax1
SEQ ID NO: 127
ATGGTGCTGCAAACACAGGTGTTCATTAGCCTGCTCCTGTGGATCTCCGG
CGCCTACGGCGAAATCGTGCTGACCCAGTCTCCTGGAACACTGAGCCTGT
CTCCCGGCGAACGGGCCACACTGAGCTGCAGAGCTTCTCAGACCATTTCC
AATAGCTACGTTGCTTGGTACCAGCAGAAACCTGGCCAGGCCCCTAGACT
GCTGATCTACGGCGCTAGCACCAGAGCCACCGGCATCCCTGATAGATTCA
GCGGCTCTGGATCTGGCACCGATTTCACCCTGACCATCAGCCGCCTGGAA
CCAGAGGACTTCGCCGTGTACTACTGTCAGCACTACGGCAGCTCCAGAGG
CTGGACCTTCGGCCAAGGCACAAAGGTCGAGATCAAGCGGACCGTGGCCG
CCCCTAGCGTGTTTATCTTTCCTCCATCCGACGAGCAGCTGAAGAGCGGC
ACCGCCAGCGTGGTGTGCCTGCTGAACAACTTCTACCCCCGGGAAGCCAA
GGTGCAGTGGAAGGTGGACAACGCCCTGCAGAGCGGAAATAGCCAGGAGA
GCGTTACAGAGCAGGATAGCAAGGACTCCACCTACAGCCTGAGCAACACC
CTGACTCTGTCTAAGGCCGACTATGAGAAGCACAAGGTCTATGCCTGCGA
GGTGACACACCAGGGCCTGTCAAGCCCCGTGACCAAAAGCTTCAACAGAG
GAGAATGTTGATGA
SEQ ID NO: 128
MVLQTQVFISLLLWISGAYGEIVLTQSPGTLSLSPGERATLSCRASQTIS
NSYVAWYQQKPGQAPRLLIYGASTRATGIPDRFSGSGSGTDFTLTISRLE
PEDFAVYYCQHYGSSRGWTFGQGTKVEIKRTVAAPSVFIFPPSDEQLKSG
TASVVCLLNNFYPREAKVQWKVDNALQSGNSQESVTEQDSKDSTYSLSNT
LTLSKADYEKHKVYACEVTHQGLSSPVTKSFNRGEC**
alphamod1_LC_rev1_pVax1
SEQ ID NO: 129
ATGGTGCTGCAGACCCAGGTGTTCATCAGCCTGCTGCTGTGGATCTCCGG
CGCCTACGGCGAGATCGTGCTGACCCAGTCCCCAGGCACACTGAGCCTGT
CCCCTGGAGAGAGAGCCACCCTGAGCTGCAGGGCATCTCAGACCATTTCT
AATAGCTACGTTGCCTGGTATCAGCAGAAGCCTGGCCAGGCCCCAAGACT
GCTGATCTACGGAGCCAGCACCCGCGCCACCGGCATCCCCGACAGGTTCT
CCGGCTCTGGCAGCGGCACAGACTTCACCCTGACAATCTCCCGGCTGGAG
CCTGAGGACTTCGCCGTGTACTACTGTCAGCAGTATGGATCCTCTCCCTA
CACCTTTGGCCAGGGCACAAAGCTGGAGATCAAGCGTACAGTGGCCGCCC
CCAGCGTGTTCATCTTTCCACCCAGCGACGAGCAGCTGAAGTCCGGCACC
GCCTCTGTGGTGTGCCTGCTGAACAATTTCTACCCTCGGGAGGCCAAGGT
GCAGTGGAAGGTGGATAACGCCCTGCAGTCCGGCAATTCTCAGGAGAGCG
TGACCGAGCAGGACTCCAAGGATTCTACATATAGCCTGAGCTCCACCCTG
ACACTGAGCAAGGCCGACTACGAGAAGCACAAGGTGTATGCCTGTGAGGT
CACCCACCAGGGGCTGTCAAGTCCAGTCACTAAAAGTTTCAATAGGGGAG
AATGTTGATAA
SEQ ID NO: 130
MVLQTQVFISLLLWISGAYGEIVLTQSPGTLSLSPGERATLSCRASQTIS
NSYVAWYQQKPGQAPRLLIYGASTRATGIPDRFSGSGSGTDFTLTISRLE
PEDFAVYYCQQYGSSPYTFGQGTKLEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC**
alphamod1_LC_rev2_pVax1
SEQ ID NO: 131
ATGGTGCTGCAGACCCAGGTGTTCATCAGCCTGCTGCTGTGGATCTCCGG
CGCCTACGGCGAGATCGTGCTGACCCAGTCCCCAGGCACACTGAGCCTGT
CCCCTGGAGAGAGAGCCACCCTGAGCTGCAGGGCATCTCAGACCATTTCT
AATAGCTACGTTGCCTGGTATCAGCAGAAGCCTGGCCAGGCCCCAAGACT
GCTGATCTACGGAGCCAGCACCCGCGCCACCGGCATCCCCGACAGGTTCT
CCGGCTCTGGCAGCGGCACAGACTTCACCCTGACAATCTCCCGGCTGGAG
CCTGAGGACTTCGCCGTGTACTACTGTCAGCAGTATGGATCCTCTCCCTG
GACCTTTGGCCAGGGCACAAAGCTGGAGATCAAGCGTACAGTGGCCGCCC
CCAGCGTGTTCATCTTTCCACCCAGCGACGAGCAGCTGAAGTCCGGCACC
GCCTCTGTGGTGTGCCTGCTGAACAATTTCTACCCTCGGGAGGCCAAGGT
GCAGTGGAAGGTGGATAACGCCCTGCAGTCCGGCAATTCTCAGGAGAGCG
TGACCGAGCAGGACTCCAAGGATTCTACATATAGCCTGAGCTCCACCCTG
ACACTGAGCAAGGCCGACTACGAGAAGCACAAGGTGTATGCCTGTGAGGT
CACCCACCAGGGGCTGTCAAGTCCAGTCACTAAAAGTTTCAATAGGGGAG
AATGTTGATAA
SEQ ID NO: 132
MVLQTQVFISLLLWISGAYGEIVLTQSPGTLSLSPGERATLSCRASQTIS
NSYVAWYQQKPGQAPRLLIYGASTRATGIPDRFSGSGSGTDFTLTISRLE
PEDFAVYYCQQYGSSPWTFGQGTKLEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC**
2072_LC_WT_pVax1
SEQ ID NO: 133
ATGGTGCTGCAGACCCAGGTGTTCATCAGCCTGCTGCTGTGGATCTCCGG
CGCCTACGGCGAGATCGTGCTGACCCAGTCCCCAGGCACACTGAGCCTGT
CCCCTGGAGAGAGAGCCACCCTGAGCTGCAGGGCATCTCAGAGCGTGTCT
AGCTCCTACCTGGGCTGGTATCAGCAGAAGCCTGGCCAGGCCCCAAGACT
GCTGATCTACGGAGCCTCTAGCCGCGCCACCGGCATCCCCGACAGGTTCT
CCGGCTCTGGCAGCGGCACAGACTTCACCCTGACAATCTCCCGGCTGGAG
CCTGAGGACTTCGCCGTGTACTACTGTCAGCAGTATGGCTCCTCTCCCTG
GACCTTTGGCCAGGGCACAAAGGTGGAGATCAAGCGTACAGTGGCCGCCC
CCAGCGTGTTCATCTTTCCACCCAGCGACGAGCAGCTGAAGTCCGGCACC
GCCTCTGTGGTGTGCCTGCTGAACAATTTCTACCCTCGGGAGGCCAAGGT
GCAGTGGAAGGTGGATAACGCCCTGCAGTCCGGCAATTCTCAGGAGAGCG
TGACCGAGCAGGACTCCAAGGATTCTACATATAGCCTGAGCTCCACCCTG
ACACTGAGCAAGGCCGACTACGAGAAGCACAAGGTGTATGCCTGTGAGGT
CACCCACCAGGGGCTGTCAAGTCCAGTCACTAAAAGTTTCAATAGGGGAG
AATGTTGATAA
SEQ ID NO: 134
MVLQTQVFISLLLWISGAYGEIVLTQSPGTLSLSPGERATLSCRASQSVS
SSYLGWYQQKPGQAPRLLIYGASSRATGIPDRFSGSGSGTDFTLTISRLE
PEDFAVYYCQQYGSSPWTFGQGTKVEIKRTVAAPSVFIFPPSDEQLKSGT
ASVVCLLNNFYPREAKVQWKVDNALQSGNSQESVTEQDSKDSTYSLSSTL
TLSKADYEKHKVYACEVTHQGLSSPVTKSFNRGEC**
2196_HC_WT_pVax1
Key: Leader-Variable heavy chain sequence-
Constant heavy chain sequence
SEQ ID NO: 135
ATGGACTGGACCTGGAGAATCCTGTTCCTGGTGGCTGCTGCTACAGGCAC
CCACGCCCAAATGCAGCTGGTCCAGAGCGGCCCTGAGGTGAAAAAGCCTG
GCACATCTGTGAAGGTGTCCTGCAAGGCCAGCGGGTTTACATTCATGAGC
TCTGCCGTGCAGTGGGTGCGGCAGGCCAGAGGCCAGAGACTGGAATGGAT
CGGCTGGATCGTGATCGGCAGCGGCAATACCAACTACGCCCAGAAGTTTC
AGGAGCGGGTGACCATCACAAGAGACATGAGCACCAGCACCGCCTATATG
GAACTGTCCAGCCTGAGAAGTGAAGATACCGCCGTGTACTACTGTGCCGC
CCCATACTGCAGCAGCATCAGCTGCAACGACGGCTTCGACATCTGGGGAC
AAGGCACCATGGTGACCGTGTCAAGCGCCTCTACCAAGGGCCCCAGCGTG
TTCCCTCTTGCTCCTTCCTCCAAAAGCACCAGCGGAGGCACCGCCGCCCT
GGGATGTCTGGTTAAAGACTACTTCCCCGAACCTGTGACAGTGTCTTGGA
ACAGCGGTGCTCTGACCTCCGGCGTGCACACATTCCCCGCTGTTCTGCAG
TCTTCTGGACTGTACAGCCTGTCTTCTGTGGTGACAGTCCCCAGCTCTTC
TCTGGGCACCCAGACCTACATCTGCAATGTGAACCACAAGCCTAGCAACA
CCAAAGTTGATAAGAAGGTGGAGCCTAAGAGCTGCGATAAAACACATACA
TGTCCTCCGTGCCCTGCTCCTGAGCTGCTGGGCGGCCCAAGCGTGTTCCT
TTTTCCTCCTAAGCCCAAGGACACCCTGATGATCAGTAGAACCCCAGAGG
TGACCTGTGTGGTGGTGGACGTGTCCCACGAGGACCCTGAGGTGAAGTTC
AACTGGTACGTGGATGGCGTGGAAGTTCATAATGCCAAGACAAAACCTAG
GGAAGAGCAGTACAACAGCACCTATAGAGTGGTCTCCGTCCTGACCGTGC
TGCACCAGGACTGGCTGAACGGCAAGGAATACAAGTGCAAGGTGAGCAAC
AAGGCCCTGCCTGCCCCTATCGAGAAGACCATTAGCAAGGCTAAGGGCCA
GCCTAGAGAGCCCCAGGTGTACACCCTGCCCCCCAGCAGAGATGAGCTGA
CAAAGAACCAGGTGAGTCTGACATGCCTGGTCAAAGGCTTCTACCCATCC
GATATCGCCGTGGAGTGGGAGAGCAATGGCCAGCCTGAAAACAACTACAA
GACCACCCCTCCTGTGCTGGACAGCGACGGCAGCTTCTTCCTGTACAGCA
AGCTGACAGTGGACAAGTCCCGGTGGCAACAGGGCAACGTGTTCAGCTGT
AGCGTGATGCACGAGGCCCTGCACAACCACTACACACAGAAAAGCCTCTC
CCTGAGCCCTGGAAAGTGATGA
SEQ ID NO: 136
MDWTWRILFLVAAATGTHAQMQLVQSGPEVKKPGTSVKVSCKASGFTFMS
SAVQWVRQARGQRLEWIGWIVIGSGNTNYAQKFQERVTITRDMSTSTAYM
ELSSLRSEDTAVYYCAAPYCSSISCNDGFDIWGQGTMVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHODWLNGKEYKCKVSN
KALPAPIEKTISKAKGOPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK**

### Example 3: Structure-Based Design of Antibody CDRs for Enhanced In Vivo Expression

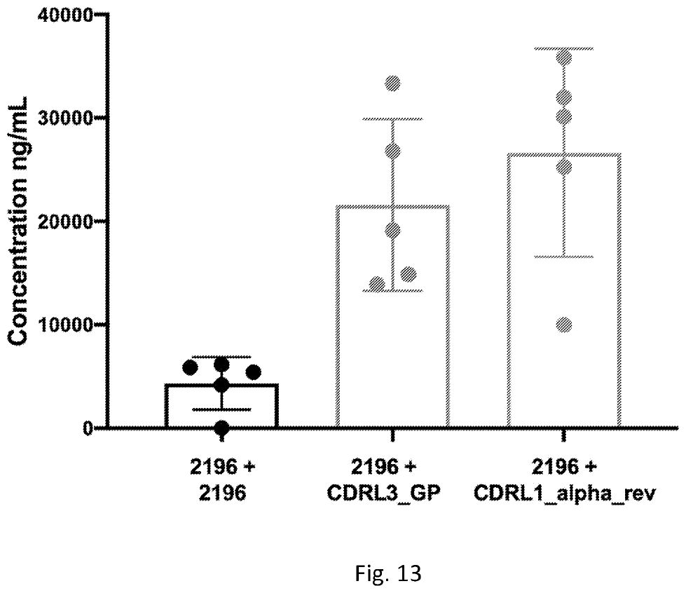

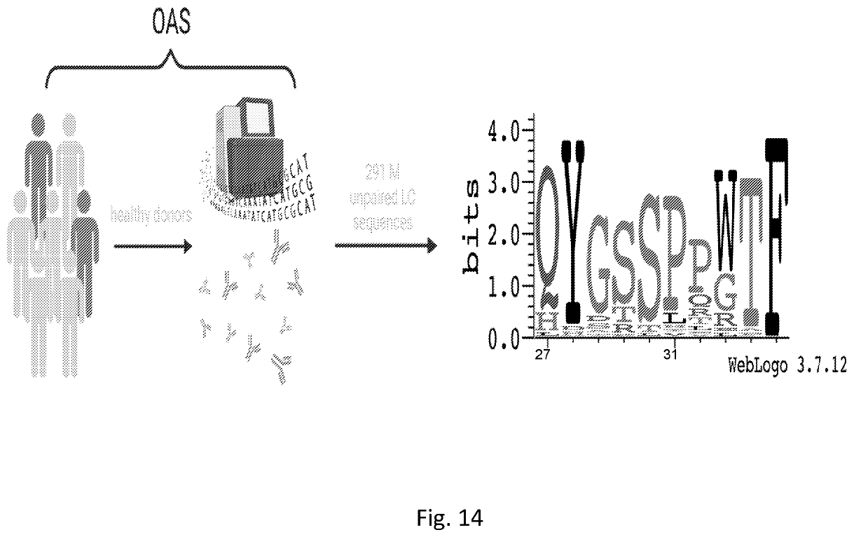

Some LC sequences demonstrated improved expression in vivo (FIG. 13). An analysis was performed to see if any of those mutations were commonly enriched in naturally occurring antibodies. In order to do this comparison, antibody sequences were downloaded from the OAS and the frequencies of the amino acids were determined at every position in naturally occurring antibodies of a given antibody V and J gene germline (see the overview in FIG. 14).

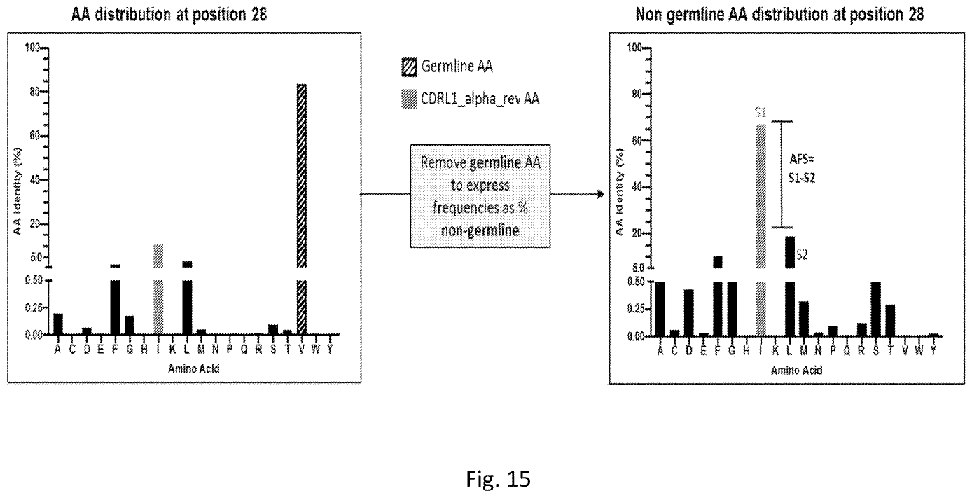

The AA found in high expressing CDRL1_alpha_rev (also referred to as 2196_LC_cluster_rev2) was compared to enrichments found in naturally occurring antibodies in the OAS. To do this, an antibody frequency score (AFS) was developed following the workflow shown in FIG. 15.

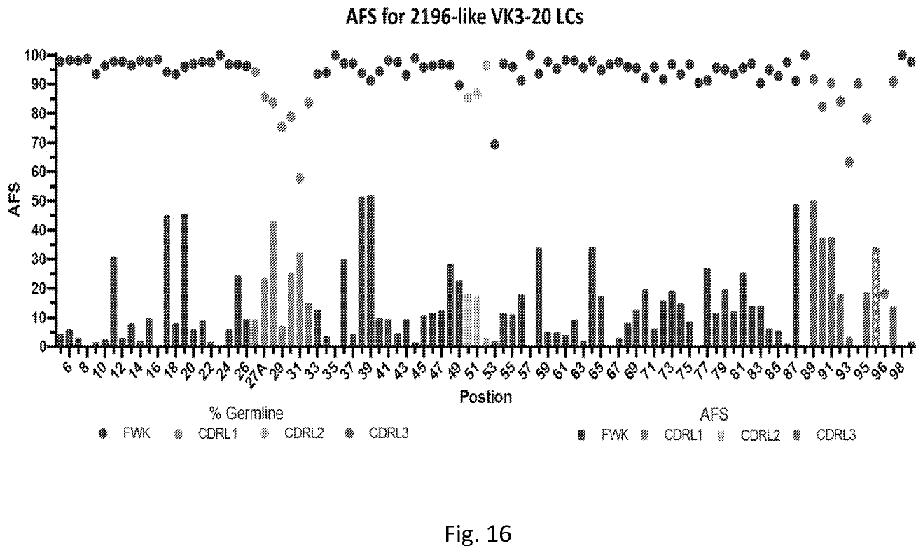

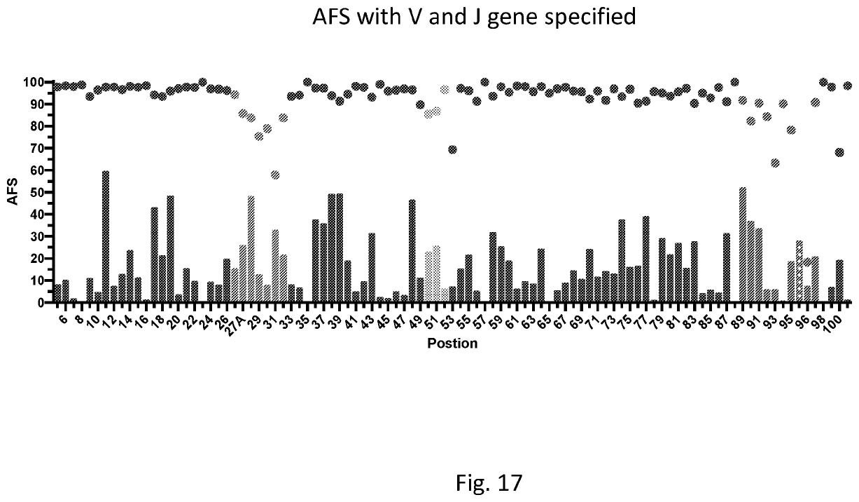

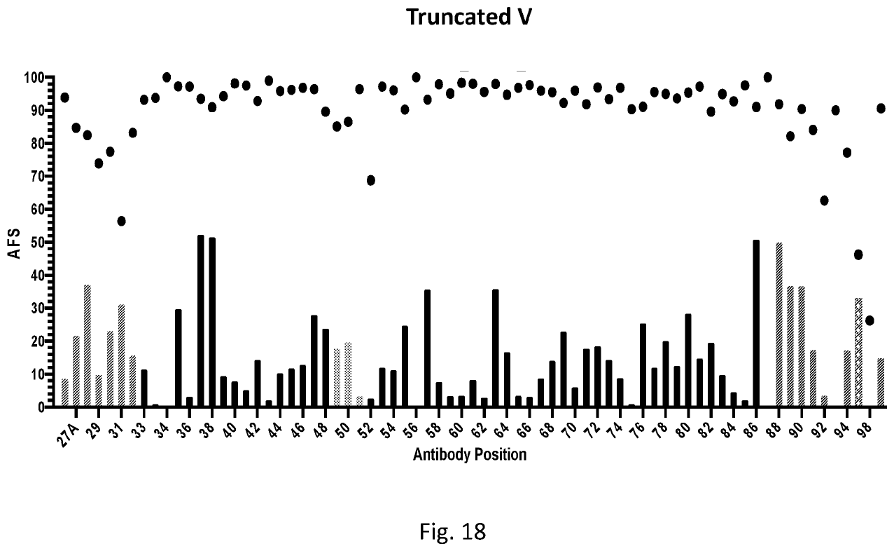

The AFS across the whole VK3-20 LCs. VK3-20 LCs (with identical CDR lengths to 2196) were searched to determine additional positions that show particular AA enrichments (FIG. 16). Spots with higher AFS show preference for a particular AA (and so such positions and the associated AA identified through AFS) might boost in vivo expression. Spots with a low % germline were also selected as this means that overall there are more non-germline sequences i.e., more naturally occurring sequences that contain the mutation of interest.

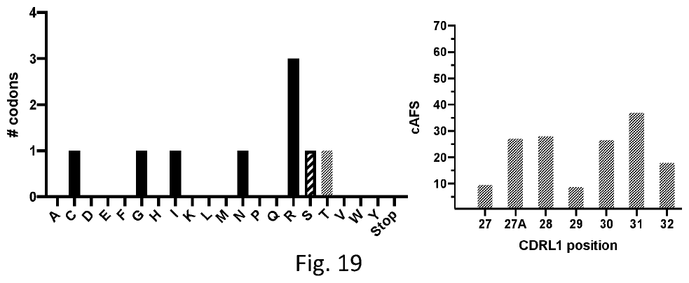

Codon bias was incorporated into AFS to create a codon adjusted AFS (cAFS). This scales scores based on whether a certain mutation is more likely to pop up simply because it is easiest to mutate to from the germline codon (i.e., an artificial high AFS may not be due to selection, but just due to random codon mutations.) This scaling makes AFS better at predicting good mutations (FIG. 19).

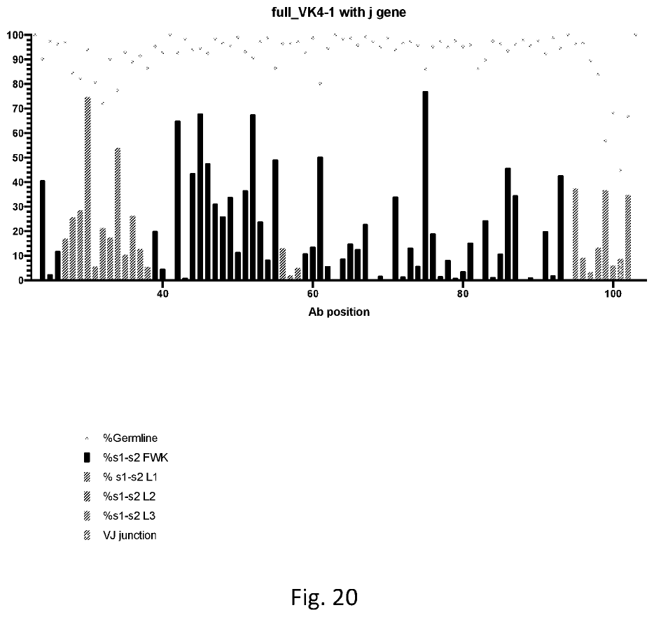

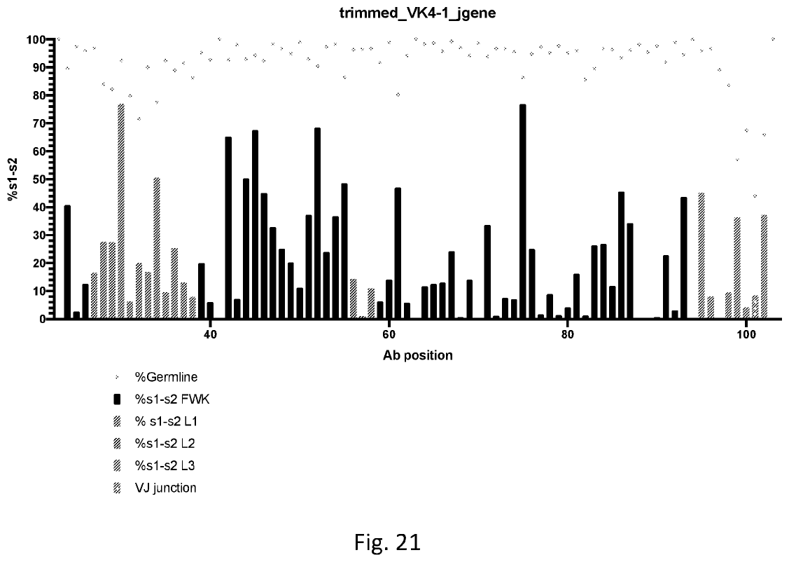

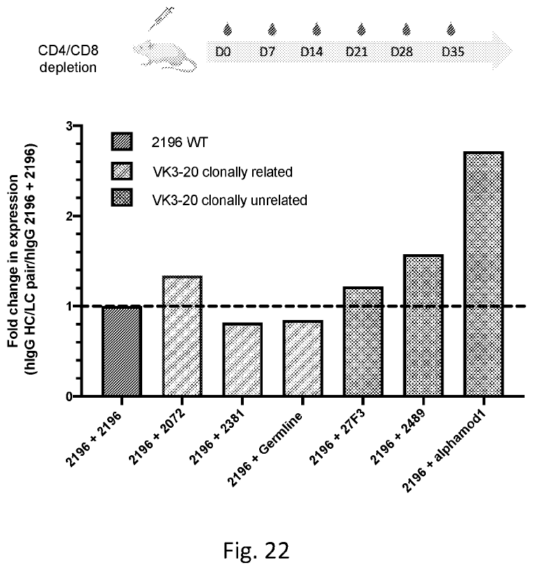

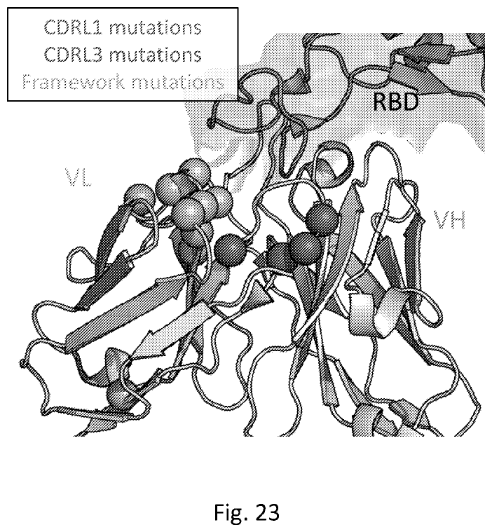

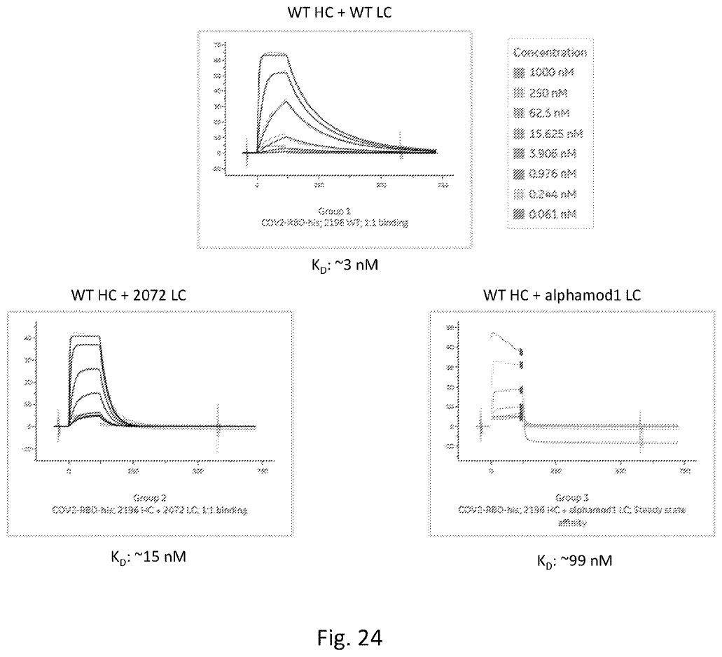

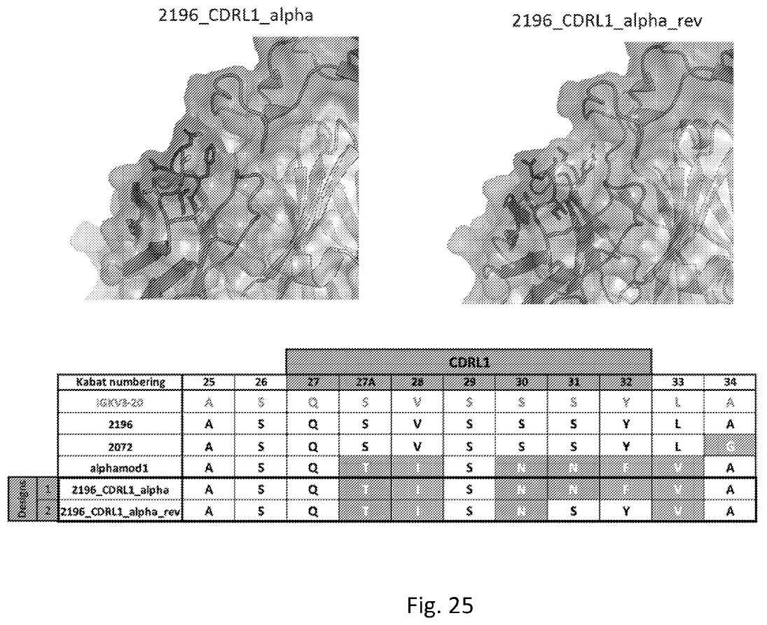

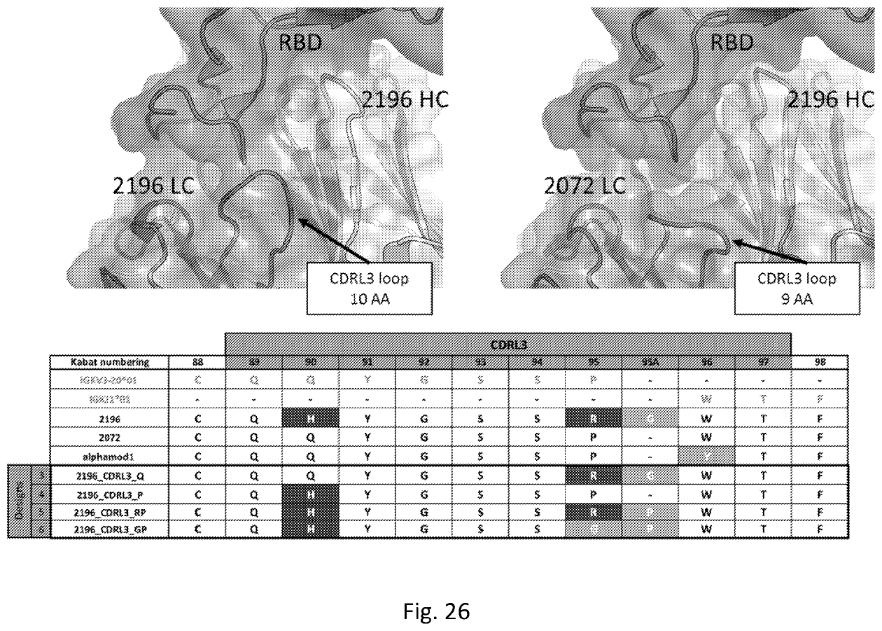

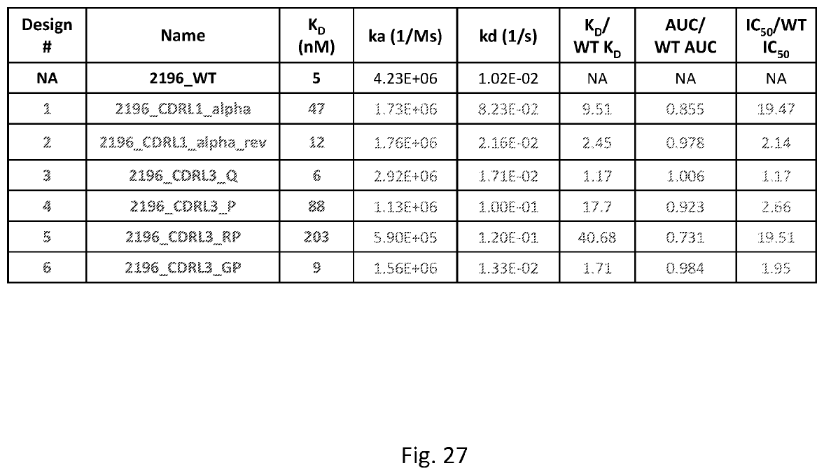

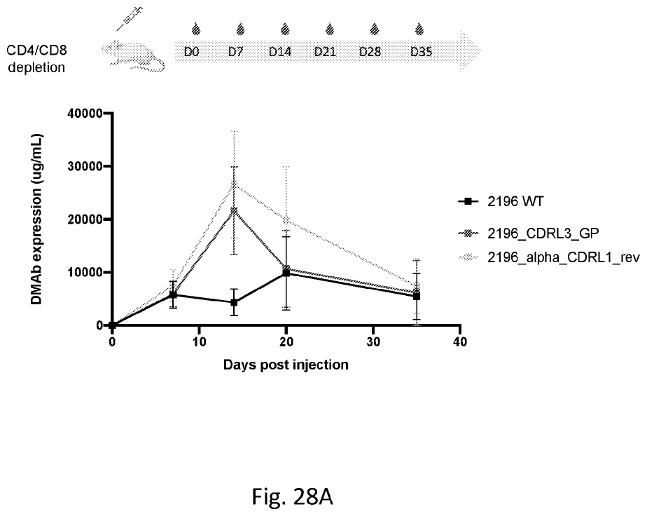

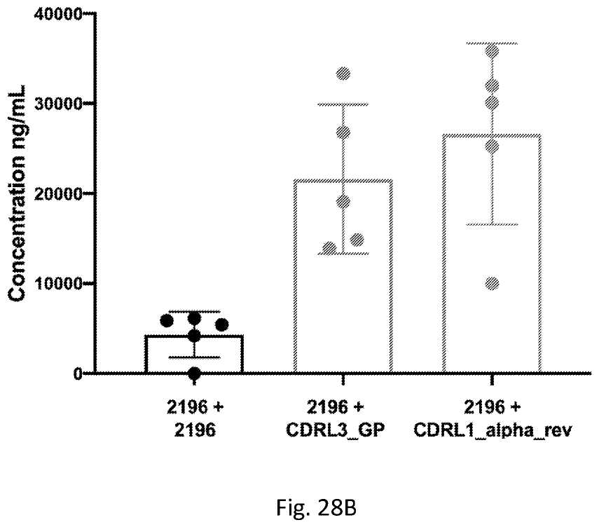

The same workflow to search for 2130-like LCs (VK4-1s) w/or without the relevant J gene (FIG. 20 and FIG. 21.)

2196_LC_clin_cluster_rev2_pVax1_CDR1
(SEQ ID NO: 137)
CAGACCATTTCTAATTCCTAC
(SEQ ID NO: 138)
QTISNSY
2196_LC_clin_cluster_rev2_pVax1_CDR2
(SEQ ID NO: 139)
GGAGCCTCT
(SEQ ID NO: 140)
GAS
2196_LC_clin_cluster_rev2_pVax1_CDR3
(SEQ ID NO: 141)
CAGCACTATGGCTCCTCTCGGGGCTGGACC
(SEQ ID NO: 142)
QHYGSSRGWT
2196_LC_clin_cluster_rev2_pVax1 VL
(SEQ ID NO: 143)
GAGATCGTGCTGACCCAGTCCCCAGGCACACTGAGCCTGTCCCCTGGAGAG
AGAGCCACCCTGAGCTGCAGGGCATCTCAGACCATTTCTAATTCCTACGT
TGCCTGGTATCAGCAGAAGCCTGGCCAGGCCCCAAGACTGCTGATCTACGG
AGCCTCTACCCGCGCCACCGGCATCCCCGACAGGTTCTCCGGCTCTGGCAG
CGGCACAGACTTCACCCTGACAATCTCCCGGCTGGAGCCTGAGGACTTCGCC
GTGTACTACTGTCAGCACTATGGCTCCTCTCGGGGCTGGACCTTTGGCCAGG
GCACAAAGGTGGAGATCAAG
(SEQ ID NO: 144)
EIVLTQSPGTLSLSPGERATLSCRASQTISNSYVAWYQQKPGQAPRLLIYGAST
RATGIPDRFSGSGSGTDFTLTISRLEPEDFAVYYCQHYGSSRGWTFGQGTKVEIK
2196_LC_clin_cluster_rev2_pVax1
(SEQ ID NO: 145)
ATGGTGCTGCAGACCCAGGTGTTTATCAGCCTGCTGCTGTGGATCTCCGGAGCAT
ATGGAGAGATCGTGCTGACCCAGTCCCCAGGCACACTGAGCCTGTCCCCTGG
AGAGAGAGCCACCCTGAGCTGCAGGGCATCTCAGACCATTTCTAATTCCT
ACGTTGCCTGGTATCAGCAGAAGCCTGGCCAGGCCCCAAGACTGCTGATCT
ACGGAGCCTCTACCCGCGCCACCGGCATCCCCGACAGGTTCTCCGGCTCTG
GCAGCGGCACAGACTTCACCCTGACAATCTCCCGGCTGGAGCCTGAGGACTT
CGCCGTGTACTACTGTCAGCACTATGGCTCCTCTCGGGGCTGGACCTTTGGC
CAGGGCACAAAGGTGGAGATCAAGAGGACCGTGGCAGCACCATCCGTGTTC
ATCTTTCCACCCTCTGACGAGCAGCTGAAGAGCGGCACAGCCTCCGTGGTGT
GCCTGCTGAACAATTTCTATCCCCGCGAGGCCAAGGTGCAGTGGAAGGTGG
ATAACGCCCTGCAGTCCGGCAATTCTCAGGAGAGCGTGACCGAGCAGGACT
CCAAGGATTCTACATACTCCCTGTCTAGCACCCTGACACTGAGCAAGGCCGA
TTACGAGAAGCACAAGGTGTATGCCTGCGAGGTGACCCATCAGGGGCTGTC
TTCTCCAGTGACCAAATCCTTCAATCGCGGGGAATGTTGATAA
(SEQ ID NO: 146)
MVLQTQVFISLLLWISGAYGEIVLTQSPGTLSLSPGERATLSCRASQTISNSYVAW
YQQKPGQAPRLLIYGASTRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSSTLTLSKADYEKHKVYACEVT
HQGLSSPVTKSFNRGEC**
2196_LC_CDRL1_pos2_pVax1 LC CDR1
(SEQ ID NO: 147)
CAGACCGTGTCCAGCAGCTAC
(SEQ ID NO: 148)
QTVSSSY
2196_LC_CDRL1_pos2_pVax1 LC CDR2
(SEQ ID NO: 149)
GGCGCTAGC
(SEQ ID NO: 140)
GAS
2196_LC_CDRL1_pos2_pVax1 LC CDR3
(SEQ ID NO: 150)
CAGCACTACGGCAGCTCCAGAGGCTGGACC
(SEQ ID NO: 142)
QHYGSSRGWT
2196_LC_CDRL1_pos2_pVax1 VL
(SEQ ID NO: 151)
GAAATCGTGCTGACCCAGTCTCCTGGAACACTGAGCCTGTCTCCCGGCGAAC
GGGCCACACTGAGCTGCAGAGCTTCTCAGACCGTGTCCAGCAGCTACCTGG
CTTGGTACCAGCAGAAACCTGGCCAGGCCCCTAGACTGCTGATCTACGGCGC
TAGCTCTAGAGCCACCGGCATCCCTGATAGATTCAGCGGCTCTGGATCTGGC
ACCGATTTCACCCTGACCATCAGCCGCCTGGAACCAGAGGACTTCGCCGTGT
ACTACTGTCAGCACTACGGCAGCTCCAGAGGCTGGACCTTCGGCCAAGGCA
CAAAGGTCGAGATCAAG
(SEQ ID NO: 152)
EIVLTQSPGTLSLSPGERATLSCRASQTVSSSYLAWYQQKPGQAPRLLIYGASSR
ATGIPDRFSGSGSGTDFTLTISRLEPEDFAVYYCQHYGSSRGWTFGQGTKVEIK
2196_LC_CDRL1_pos2_pVax1
(SEQ ID NO: 153)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGACCGTGTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
(SEQ ID NO: 154)
MVLQTQVFISLLLWISGAYGEIVLTQSPGTLSLSPGERATLSCRASQTVSSSYLAW
YQQKPGQAPRLLIYGASSRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2196_LC_CDRL1_pos3_pVax1 LC CDR1
(SEQ ID NO: 155)
CAGAGCATTTCCAGCAGCTAC
(SEQ ID NO: 156)
QSISSSY
2196_LC_CDRL1_pos3_pVax1 LC CDR2
(SEQ ID NO: 149)
GGCGCTAGC
(SEQ ID NO: 140)
GAS
2196_LC_CDRL1_pos3_pVax1 LC CDR3
(SEQ ID NO: 150)
CAGCACTACGGCAGCTCCAGAGGCTGGACC
(SEQ ID NO: 142)
QHYGSSRGWT
2196_LC_CDRL1_pos3_pVax1 VL
(SEQ ID NO: 157)
GAAATCGTGCTGACCCAGTCTCCTGGAACACTGAGCCTGTCTCCCGGCGAAC
GGGCCACACTGAGCTGCAGAGCTTCTCAGAGCATTTCCAGCAGCTACCTGG
CTTGGTACCAGCAGAAACCTGGCCAGGCCCCTAGACTGCTGATCTACGGCGC
TAGCTCTAGAGCCACCGGCATCCCTGATAGATTCAGCGGCTCTGGATCTGGC
ACCGATTTCACCCTGACCATCAGCCGCCTGGAACCAGAGGACTTCGCCGTGT
ACTACTGTCAGCACTACGGCAGCTCCAGAGGCTGGACCTTCGGCCAAGGCA
CAAAGGTCGAGATCAAG
(SEQ ID NO: 158)
EIVLTQSPGTLSLSPGERATLSCRASQSISSSYLAWYQQKPGQAPRLLIYGASSRA
TGIPDRFSGSGSGTDFTLTISRLEPEDFAVYYCQHYGSSRGWTFGQGTKVEIK
2196_LC_CDRL1_pos3_pVax1
(SEQ ID NO: 159)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCATTTCCAGCAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
(SEQ ID NO: 160)
MVLQTQVFISLLLWISGAYGEIVLTQSPGTLSLSPGERATLSCRASQSISSSYLAW
YQQKPGQAPRLLIYGASSRATGIPDRFSGSGSGTDFTLTISRLEPEDFAVYYCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVT
HQGLSSPVTKSFNRGEC**
2196_LC_CDRL1_pos5_pVax1 LC CDR1
(SEQ ID NO: 161)
CAGAGCGTGTCCAATAGCTAC
(SEQ ID NO: 162)
QSVSNSY
2196_LC_CDRL1_pos5_pVax1 LC CDR2
(SEQ ID NO: 149)
GGCGCTAGC
(SEQ ID NO: 140)
GAS
2196_LC_CDRL1_pos5_pVax1 LC CDR3
(SEQ ID NO: 150)
CAGCACTACGGCAGCTCCAGAGGCTGGACC
(SEQ ID NO: 142)
QHYGSSRGWT
2196_LC_CDRL1_pos5_pVax1 VL
(SEQ ID NO: 163)
GAAATCGTGCTGACCCAGTCTCCTGGAACACTGAGCCTGTCTCCCGGCGAAC
GGGCCACACTGAGCTGCAGAGCTTCTCAGAGCGTGTCCAATAGCTACCTGG
CTTGGTACCAGCAGAAACCTGGCCAGGCCCCTAGACTGCTGATCTACGGCGC
TAGCTCTAGAGCCACCGGCATCCCTGATAGATTCAGCGGCTCTGGATCTGGC
ACCGATTTCACCCTGACCATCAGCCGCCTGGAACCAGAGGACTTCGCCGTGT
ACTACTGTCAGCACTACGGCAGCTCCAGAGGCTGGACCTTCGGCCAAGGCA
CAAAGGTCGAGATCAAG
(SEQ ID NO: 164)
EIVLTQSPGTLSLSPGERATLSCRASQSVSNSYLAWYQQKPGQAPRLLIYGASSR
ATGIPDRFSGSGSGTDFTLTISRLEPEDFAVYYCQHYGSSRGWTFGQGTKVEIK
2196_LC_CDRL1_pos5_pVax1
(SEQ ID NO: 165)
ATGGTGCTGCAAACACAGGTGTTCATTAGCCTGCTCCTGTGGATCTCCGGCGCC
TACGGCGAAATCGTGCTGACCCAGTCTCCTGGAACACTGAGCCTGTCTCCCGG
CGAACGGGCCACACTGAGCTGCAGAGCTTCTCAGAGCGTGTCCAATAGCTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
(SEQ ID NO: 166)
MVLQTQVFISLLLWISGAYGEIVLTQSPGTLSLSPGERATLSCRASQSVSNSYLAW
YQQKPGQAPRLLIYGASSRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2196_LC_CDRL1_pos6_pVax1 LC CDR1
(SEQ ID NO: 167)
CAGAGCGTGTCCAGCAATTAC
(SEQ ID NO: 168)
QSVSSNY
2196_LC_CDRL1_pos6_pVax1 LC CDR2
(SEQ ID NO: 149)
GGCGCTAGC
(SEQ ID NO: 140)
GAS
2196_LC_CDRL1_pos6_pVax1 LC CDR3
(SEQ ID NO: 150)
CAGCACTACGGCAGCTCCAGAGGCTGGACC
(SEQ ID NO: 142)
QHYGSSRGWT
2196_LC_CDRL1_pos6_pVax1 VL
(SEQ ID NO: 169)
GAAATCGTGCTGACCCAGTCTCCTGGAACACTGAGCCTGTCTCCCGGCGAAC
GGGCCACACTGAGCTGCAGAGCTTCTCAGAGCGTGTCCAGCAATTACCTGG
CTTGGTACCAGCAGAAACCTGGCCAGGCCCCTAGACTGCTGATCTACGGCGC
TAGCTCTAGAGCCACCGGCATCCCTGATAGATTCAGCGGCTCTGGATCTGGC
ACCGATTTCACCCTGACCATCAGCCGCCTGGAACCAGAGGACTTCGCCGTGT
ACTACTGTCAGCACTACGGCAGCTCCAGAGGCTGGACCTTCGGCCAAGGCA
CAAAGGTCGAGATCAAG
(SEQ ID NO: 170)
EIVLTQSPGTLSLSPGERATLSCRASQSVSSNYLAWYQQKPGQAPRLLIYGASSR
ATGIPDRFSGSGSGTDFTLTISRLEPEDFAVYYCQHYGSSRGWTFGQGTKVEIK
2196_LC_CDRL1_pos6_pVax1
(SEQ ID NO: 171)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCGTGTCCAGCAATTA
CCTGGCTTGGTACCAGCAGAAACCTGGCCAGGCCCCTAGACTGCTGATCTAC
GGCGCTAGCTCTAGAGCCACCGGCATCCCTGATAGATTCAGCGGCTCTGGAT
CTGGCACCGATTTCACCCTGACCATCAGCCGCCTGGAACCAGAGGACTTCGC
CGTGTACTACTGTCAGCACTACGGCAGCTCCAGAGGCTGGACCTTCGGCCAA
GGCACAAAGGTCGAGATCAAGCGGACCGTGGCCGCCCCTAGCGTGTTTATCT
TTCCTCCATCCGACGAGCAGCTGAAGAGCGGCACCGCCAGCGTGGTGTGCCT
GCTGAACAACTTCTACCCCCGGGAAGCCAAGGTGCAGTGGAAGGTGGACAA
CGCCCTGCAGAGCGGAAATAGCCAGGAGAGCGTTACAGAGCAGGATAGCAA
GGACTCCACCTACAGCCTGAGCAACACCCTGACTCTGTCTAAGGCCGACTAT
GAGAAGCACAAGGTCTATGCCTGCGAGGTGACACACCAGGGCCTGTCAAGC
CCCGTGACCAAAAGCTTCAACAGAGGAGAATGTTGATGA
(SEQ ID NO: 172)
MVLQTQVFISLLLWISGAYGEIVLTQSPGTLSLSPGERATLSCRASQSVSSNYLAW
YQQKPGQAPRLLIYGASSRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2196_LC_cluster_rev2_noFWK_pVax1 LC CDR1
(SEQ ID NO: 173)
CAGACCATTTCCAATAGCTAC
(SEQ ID NO: 138)
QTISNSY
2196_LC_cluster_rev2_noFWK_pVax1 LC CDR2
(SEQ ID NO: 149)
GGCGCTAGC
(SEQ ID NO: 140)
GAS
2196_LC_cluster_rev2_noFWK_pVax1 LC CDR3
(SEQ ID NO: 150)
CAGCACTACGGCAGCTCCAGAGGCTGGACC
(SEQ ID NO: 142)
QHYGSSRGWT
2196_LC_cluster_rev2_noFWK_pVax1 VL
(SEQ ID NO: 174)
GAAATCGTGCTGACCCAGTCTCCTGGAACACTGAGCCTGTCTCCCGGCGAAC
GGGCCACACTGAGCTGCAGAGCTTCTCAGACCATTTCCAATAGCTACCTG
GCTTGGTACCAGCAGAAACCTGGCCAGGCCCCTAGACTGCTGATCTACGGCG
CTAGCTCTAGAGCCACCGGCATCCCTGATAGATTCAGCGGCTCTGGATCTGG
CACCGATTTCACCCTGACCATCAGCCGCCTGGAACCAGAGGACTTCGCCGTG
TACTACTGTCAGCACTACGGCAGCTCCAGAGGCTGGACCTTCGGCCAAGGCA
CAAAGGTCGAGATCAAG
(SEQ ID NO: 175)
EIVLTQSPGTLSLSPGERATLSCRASQTISNSYLAWYQQKPGQAPRLLIYGASSR
ATGIPDRFSGSGSGTDFTLTISRLEPEDFAVYYCQHYGSSRGWTFGQGTKVEIK
2196_LC_cluster_rev2_noFWK_pVax1
(SEQ ID NO: 176)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGACCATTTCCAATAGCT
ACCTGGCTTGGTACCAGCAGAAACCTGGCCAGGCCCCTAGACTGCTGATCTA
CGGCGCTAGCTCTAGAGCCACCGGCATCCCTGATAGATTCAGCGGCTCTGGA
TCTGGCACCGATTTCACCCTGACCATCAGCCGCCTGGAACCAGAGGACTTCG
CCGTGTACTACTGTCAGCACTACGGCAGCTCCAGAGGCTGGACCTTCGGCCA
AGGCACAAAGGTCGAGATCAAGCGGACCGTGGCCGCCCCTAGCGTGTTTAT
CTTTCCTCCATCCGACGAGCAGCTGAAGAGCGGCACCGCCAGCGTGGTGTGC
CTGCTGAACAACTTCTACCCCCGGGAAGCCAAGGTGCAGTGGAAGGTGGAC
AACGCCCTGCAGAGCGGAAATAGCCAGGAGAGCGTTACAGAGCAGGATAGC
AAGGACTCCACCTACAGCCTGAGCAACACCCTGACTCTGTCTAAGGCCGACT
ATGAGAAGCACAAGGTCTATGCCTGCGAGGTGACACACCAGGGCCTGTCAA
GCCCCGTGACCAAAAGCTTCAACAGAGGAGAATGTTGATGA
(SEQ ID NO: 177)
MVLQTQVFISLLLWISGAYGEIVLTQSPGTLSLSPGERATLSCRASQTISNSYLAW
YQQKPGQAPRLLIYGASSRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2196_LC_clrev2_plus_fwks_pVax1 LC CDR1
(SEQ ID NO: 173)
CAGACCATTTCCAATAGCTAC
(SEQ ID NO: 138)
QTISNSY
2196_LC_clrev2_plus_fwks_pVax1 LC CDR2
(SEQ ID NO: 149)
GGCGCTAGC
(SEQ ID NO: 140)
GAS
2196_LC_clrev2_plus_fwks_pVax1 LC CDR3
(SEQ ID NO: 150)
CAGCACTACGGCAGCTCCAGAGGCTGGACC
(SEQ ID NO: 142)
QHYGSSRGWT
2196_LC_clrev2_plus_fwks_pVax1 VL
(SEQ ID NO: 178)
GAAGTGGTGCTGACCCAGTCTCCTGGAACACTGAGCCTGTCTCCCGGCGAA
CGGGCCACACTGAGCTGCAGAGCTTCTCAGACCATTTCCAATAGCTACG
TTGCTTGGTACCAGCAGAGACCTGGCCAGGCCCCTAGACTGCTGATCTACG
GCGCTAGCACCAGAGCCGCCGGCATCCCTGATAGATTCAGCGGCTCTGGA
TCTGGCACCGATTTCACCCTGACCATCAGCCGCCTGGAACCAGAGGACTTCG
CCGTGTACTACTGTCAGCACTACGGCAGCTCCAGAGGCTGGACCTTCGGCCA
AGGCACAAAGGTCGAGATCAAG
(SEQ ID NO: 179)
EVVLTQSPGTLSLSPGERATLSCRASQTISNSYVAWYQQRPGQAPRLLIYGAS
TRAAGIPDRFSGSGSGTDFTLTISRLEPEDFAVYYCQHYGSSRGWTFGQGTKVEI
K
2196_LC_clrev2_plus_fwks_pVax1
(SEQ ID NO: 180)
ATGGTGCTGCAAACACAGGTGTTCATTAGCCTGCTCCTGTGGATCTCCGGCGC
CTACGGCGAAGTGGTGCTGACCCAGTCTCCTGGAACACTGAGCCTGTCTCCCG
GCGAACGGGCCACACTGAGCTGCAGAGCTTCTCAGACCATTTCCAATAGC
TACGTTGCTTGGTACCAGCAGAGACCTGGCCAGGCCCCTAGACTGCTGAT
CTACGGCGCTAGCACCAGAGCCGCCGGCATCCCTGATAGATTCAGCGGCT
CTGGATCTGGCACCGATTTCACCCTGACCATCAGCCGCCTGGAACCAGAGGA
CTTCGCCGTGTACTACTGTCAGCACTACGGCAGCTCCAGAGGCTGGACCTTC
GGCCAAGGCACAAAGGTCGAGATCAAGCGGACCGTGGCCGCCCCTAGCGTG
TTTATCTTTCCTCCATCCGACGAGCAGCTGAAGAGCGGCACCGCCAGCGTGG
TGTGCCTGCTGAACAACTTCTACCCCCGGGAAGCCAAGGTGCAGTGGAAGG
TGGACAACGCCCTGCAGAGCGGAAATAGCCAGGAGAGCGTTACAGAGCAGG
ATAGCAAGGACTCCACCTACAGCCTGAGCAACACCCTGACTCTGTCTAAGGC
CGACTATGAGAAGCACAAGGTCTATGCCTGCGAGGTGACACACCAGGGCCT
GTCAAGCCCCGTGACCAAAAGCTTCAACAGAGGAGAATGTTGATGA
(SEQ ID NO: 181)
MVLQTQVFISLLLWISGAYGEVVLTQSPGTLSLSPGERATLSCRASQTISNSYVA
WYQQRPGQAPRLLIYGASTRAAGIPDRFSGSGSGTDFTLTISRLEPEDFAVYYC
QHYGSSRGWTFGQGTKVEIKRTVAAPSVFIFPPSDEQLKSGTASVVCLLNNFYPR
EAKVQWKVDNALQSGNSQESVTEQDSKDSTYSLSNTLTLSKADYEKHKVYAC
EVTHQGLSSPVTKSFNRGEC**
2196_LC_cluster_rev2_GP_pVax1 LC CDR1
(SEQ ID NO: 173)
CAGACCATTTCCAATAGCTAC
(SEQ ID NO: 138)
QTISNSY
2196_LC_cluster_rev2_GP_pVax1 LC CDR2
(SEQ ID NO: 149)
GGCGCTAGC
(SEQ ID NO: 140)
GAS
2196_LC_cluster_rev2_GP_pVax1 CDR3
(SEQ ID NO: 182)
CAGCACTACGGCAGCTCCGGCCCCTGGACC
(SEQ ID NO: 183)
QHYGSSGPWT
2196_LC_cluster_rev2_GP_pVax1 VL
(SEQ ID NO: 184)
GAAATCGTGCTGACCCAGTCTCCTGGAACACTGAGCCTGTCTCCCGGCGAAC
GGGCCACACTGAGCTGCAGAGCTTCTCAGACCATTTCCAATAGCTACGT
TGCTTGGTACCAGCAGAAACCTGGCCAGGCCCCTAGACTGCTGATCTACGG
CGCTAGCACCAGAGCCACCGGCATCCCTGATAGATTCAGCGGCTCTGGATC
TGGCACCGATTTCACCCTGACCATCAGCCGCCTGGAACCAGAGGACTTCGCC
GTGTACTACTGTCAGCACTACGGCAGCTCCGGCCCCTGGACCTTCGGCCA
AGGCACAAAGGTCGAGATCAAG
(SEQ ID NO: 185)
EIVLTQSPGTLSLSPGERATLSCRASQTISNSYVAWYQQKPGQAPRLLIYGAST
RATGIPDRFSGSGSGTDFTLTISRLEPEDFAVYYCQHYGSSGPWTFGQGTKVEIK
2196_LC_cluster_rev2_GP_pVax1
(SEQ ID NO: 186)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGACCATTTCCAATAGCT
ACGTTGCTTGGTACCAGCAGAAACCTGGCCAGGCCCCTAGACTGCTGATCT
ACGGCGCTAGCACCAGAGCCACCGGCATCCCTGATAGATTCAGCGGCTCTG
GATCTGGCACCGATTTCACCCTGACCATCAGCCGCCTGGAACCAGAGGACTT
CGCCGTGTACTACTGTCAGCACTACGGCAGCTCCGGCCCCTGGACCTTCG
GCCAAGGCACAAAGGTCGAGATCAAGCGGACCGTGGCCGCCCCTAGCGTGT
TTATCTTTCCTCCATCCGACGAGCAGCTGAAGAGCGGCACCGCCAGCGTGGT
GTGCCTGCTGAACAACTTCTACCCCCGGGAAGCCAAGGTGCAGTGGAAGGT
GGACAACGCCCTGCAGAGCGGAAATAGCCAGGAGAGCGTTACAGAGCAGG
ATAGCAAGGACTCCACCTACAGCCTGAGCAACACCCTGACTCTGTCTAAGGC
CGACTATGAGAAGCACAAGGTCTATGCCTGCGAGGTGACACACCAGGGCCT
GTCAAGCCCCGTGACCAAAAGCTTCAACAGAGGAGAATGTTGATGA
(SEQ ID NO: 187)
MVLQTQVFISLLLWISGAYGEIVLTQSPGTLSLSPGERATLSCRASQTISNSYVAW
YQQKPGQAPRLLIYGASTRATGIPDRFSGSGSGTDFTLTISRLEPEDFAVYYCQH
YGSSGPWTFGQGTKVEIKRTVAAPSVFIFPPSDEQLKSGTASVVCLLNNFYPRE
AKVQWKVDNALQSGNSQESVTEQDSKDSTYSLSNTLTLSKADYEKHKVYACE
VTHQGLSSPVTKSFNRGEC**
2196_LC_lowGerm_FWK_pVax1 LC CDR1
(SEQ ID NO: 188)
CAGAGCGTGTCCAGCAGCTAC
(SEQ ID NO: 189)
QSVSSSY
2196_LC_lowGerm_FWK_pVax1 LC CDR2
(SEQ ID NO: 149)
GGCGCTAGC
(SEQ ID NO: 140)
GAS
2196_LC_lowGerm_FWK_pVax1 LC CDR3
(SEQ ID NO: 150)
CAGCACTACGGCAGCTCCAGAGGCTGGACC
(SEQ ID NO: 142)
QHYGSSRGWT
2196_LC_lowGerm_FWK_pVax1 VL
(SEQ ID NO: 190)
GAAGTGGTGCTGACCCAGTCTCCTGGAACACTGAGCCTGTCTCCCGGCGAA
CGGGCCACACTGAGCTGCAGAGCTTCTCAGAGCGTGTCCAGCAGCTACCTGG
CTTGGTACCAGCAGAAACCTGGCCAGGCCCCTAGACTGCTGATCTTCGGCG
CTAGCTCTAGAGCCACCGGCATCCCTGATAGATTCAGCGGCTCTGGATCTGG
CACCGATTTCACCCTGACCATCACCCGCCTGGAACCAGAGGACAGCGCCG
TGTACTACTGTCAGCACTACGGCAGCTCCAGAGGCTGGACCTTCGGCCAAGG
CACAAAGGTCGAGATCAAG
(SEQ ID NO: 191)
EVVLTQSPGTLSLSPGERATLSCRASQSVSSSYLAWYQQKPGQAPRLLIFGASSR
ATGIPDRFSGSGSGTDFTLTITRLEPEDSAVYYCQHYGSSRGWTFGQGTKVEIK
2196_LC_lowGerm_FWK_pVax1
(SEQ ID NO: 192)
ATGGTGCTGCAAACACAGGTGTTCATTAGCCTGCTCCTGTGGATCTCCGGCGCCT
ACGGCGAAGTGGTGCTGACCCAGTCTCCTGGAACACTGAGCCTGTCTCCCGG
CGAACGGGCCACACTGAGCTGCAGAGCTTCTCAGAGCGTGTCCAGCAGCTA
CCTGGCTTGGTACCAGCAGAAACCTGGCCAGGCCCCTAGACTGCTGATCTT
CGGCGCTAGCTCTAGAGCCACCGGCATCCCTGATAGATTCAGCGGCTCTGG
ATCTGGCACCGATTTCACCCTGACCATCACCCGCCTGGAACCAGAGGACA
GCGCCGTGTACTACTGTCAGCACTACGGCAGCTCCAGAGGCTGGACCTTCG
GCCAAGGCACAAAGGTCGAGATCAAGCGGACCGTGGCCGCCCCTAGCGTGT
TTATCTTTCCTCCATCCGACGAGCAGCTGAAGAGCGGCACCGCCAGCGTGGT
GTGCCTGCTGAACAACTTCTACCCCCGGGAAGCCAAGGTGCAGTGGAAGGT
GGACAACGCCCTGCAGAGCGGAAATAGCCAGGAGAGCGTTACAGAGCAGG
ATAGCAAGGACTCCACCTACAGCCTGAGCAACACCCTGACTCTGTCTAAGGC
CGACTATGAGAAGCACAAGGTCTATGCCTGCGAGGTGACACACCAGGGCCT
GTCAAGCCCCGTGACCAAAAGCTTCAACAGAGGAGAATGTTGATGA
(SEQ ID NO: 193)
MVLQTQVFISLLLWISGAYGEVVLTQSPGTLSLSPGERATLSCRASQSVSSSYLAW
YQQKPGQAPRLLIFGASSRATGIPDRFSGSGSGTDFTLTITRLEPEDSAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2196_LC_highSet_pVax1 LC CDR1
(SEQ ID NO: 155)
CAGAGCATTTCCAGCAGCTAC
(SEQ ID NO: 156)
QSISSSY
2196_LC_highSet_pVax1 LC CDR2
(SEQ ID NO: 149)
GGCGCTAGC
(SEQ ID NO: 140)
GAS
2196_LC_highSet_pVax1 LC CDR3
(SEQ ID NO: 150)
CAGCACTACGGCAGCTCCAGAGGCTGGACC
(SEQ ID NO: 142)
QHYGSSRGWT
2196_LC_highSet_pVax1 VL
(SEQ ID NO: 194)
GAAATCGTGCTGACCCAGTCTCCTGGAACACTGAGCCTGTCTCCCGGCGAAC
GGGCCACACTGAGCTGCAGAGCTTCTCAGAGCATTTCCAGCAGCTACCTGG
CTTGGTACCAGCAGAGACCTGACCAGGCCCCTAGACTGCTGATCTACGGC
GCTAGCTCTAGAGCCACCGGCATCCCTGATAGATTCAGCGGCTCTGGATCTG
GCACCGATTTCACCCTGACCATCAGCCGCCTGGAACCAGAGGACTTCGCCGT
GTACTTCTGTCAGCACTACGGCAGCTCCAGAGGCTGGACCTTCGGCCAAGG
CACAAAGGTCGAGATCAAG
(SEQ ID NO: 195)
EIVLTQSPGTLSLSPGERATLSCRASQSISSSYLAWYQQRPDQAPRLLIYGASSR
ATGIPDRFSGSGSGTDFTLTISRLEPEDFAVYFCQHYGSSRGWTFGQGTKVEIK
2196_LC_highSet_pVax1
(SEQ ID NO: 196)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGAGCATTTCCAGCAGCTA
CCTGGCTTGGTACCAGCAGAGACCTGACCAGGCCCCTAGACTGCTGATCT
ACGGCGCTAGCTCTAGAGCCACCGGCATCCCTGATAGATTCAGCGGCTCTGG
ATCTGGCACCGATTTCACCCTGACCATCAGCCGCCTGGAACCAGAGGACTTC
GCCGTGTACTTCTGTCAGCACTACGGCAGCTCCAGAGGCTGGACCTTCGGC
CAAGGCACAAAGGTCGAGATCAAGCGGACCGTGGCCGCCCCTAGCGTGTTT
ATCTTTCCTCCATCCGACGAGCAGCTGAAGAGCGGCACCGCCAGCGTGGTGT
GCCTGCTGAACAACTTCTACCCCCGGGAAGCCAAGGTGCAGTGGAAGGTGG
ACAACGCCCTGCAGAGCGGAAATAGCCAGGAGAGCGTTACAGAGCAGGATA
GCAAGGACTCCACCTACAGCCTGAGCAACACCCTGACTCTGTCTAAGGCCGA
CTATGAGAAGCACAAGGTCTATGCCTGCGAGGTGACACACCAGGGCCTGTC
AAGCCCCGTGACCAAAAGCTTCAACAGAGGAGAATGTTGATGA
(SEQ ID NO: 197)
MVLQTQVFISLLLWISGAYGEIVLTQSPGTLSLSPGERATLSCRASQSISSSYLAW
YQQRPDQAPRLLIYGASSRATGIPDRFSGSGSGTDFTLTISRLEPEDFAVYFCQHY
GSSRGWTFGQGTKVEIKRTVAAPSVFIFPPSDEQLKSGTASVVCLLNNFYPREAK
VQWKVDNALQSGNSQESVTEQDSKDSTYSLSNTLTLSKADYEKHKVYACEVT
HQGLSSPVTKSFNRGEC**
2196_LC_sharedSet_pVax1 LC CDR1
(SEQ ID NO: 198)
CAGACCATTTCCAGCAATTAC
(SEQ ID NO: 199)
QTISSNY
2196_LC_sharedSet_pVax1 LC CDR2
(SEQ ID NO: 149)
GGCGCTAGC
(SEQ ID NO: 140)
GAS
2196_LC_sharedSet_pVax1 LC CDR3
(SEQ ID NO: 150)
CAGCACTACGGCAGCTCCAGAGGCTGGACC
(SEQ ID NO: 142)
QHYGSSRGWT
2196_LC_sharedSet_pVax1 VL
(SEQ ID NO: 200)
GAAATCGTGCTGACCCAGTCTCCTGGAACACTGAGCCTGTCTCCCGGCGAAC
GGGCCACACTGAGCTGCAGAGCTTCTCAGACCATTTCCAGCAATTACCTG
GCTTGGTACCAGCAGAAACCTGGCCAGGCCCCTAGACTGCTGATCTACGGCG
CTAGCTCTAGAGCCACCGGCATCCCTGATAGATTCAGCGGCTCTGGATCTGG
CACCGATTTCACCCTGACCATCAGCCGCCTGGAACCAGAGGACTTCGCCGTG
TACTACTGTCAGCACTACGGCAGCTCCAGAGGCTGGACCTTCGGCCAAGGCA
CAAAGGTCGAGATCAAG
(SEQ ID NO: 201)
EIVLTQSPGTLSLSPGERATLSCRASQTISSNYLAWYQQKPGQAPRLLIYGASSR
ATGIPDRFSGSGSGTDFTLTISRLEPEDFAVYYCQHYGSSRGWTFGQGTKVEIK
2196_LC_sharedSet_pVax1
(SEQ ID NO: 202)
ATGGTGCTGCAAACACAGGTGTTCATTAGCCTGCTCCTGTGGATCTCCGGCGCCT
ACGGCGAAATCGTGCTGACCCAGTCTCCTGGAACACTGAGCCTGTCTCCCGG
CGAACGGGCCACACTGAGCTGCAGAGCTTCTCAGACCATTTCCAGCAATT
ACCTGGCTTGGTACCAGCAGAAACCTGGCCAGGCCCCTAGACTGCTGATCTA
CGGCGCTAGCTCTAGAGCCACCGGCATCCCTGATAGATTCAGCGGCTCTGGA
TCTGGCACCGATTTCACCCTGACCATCAGCCGCCTGGAACCAGAGGACTTCG
CCGTGTACTACTGTCAGCACTACGGCAGCTCCAGAGGCTGGACCTTCGGCCA
AGGCACAAAGGTCGAGATCAAGCGGACCGTGGCCGCCCCTAGCGTGTTTAT
CTTTCCTCCATCCGACGAGCAGCTGAAGAGCGGCACCGCCAGCGTGGTGTGC
CTGCTGAACAACTTCTACCCCCGGGAAGCCAAGGTGCAGTGGAAGGTGGAC
AACGCCCTGCAGAGCGGAAATAGCCAGGAGAGCGTTACAGAGCAGGATAGC
AAGGACTCCACCTACAGCCTGAGCAACACCCTGACTCTGTCTAAGGCCGACT
ATGAGAAGCACAAGGTCTATGCCTGCGAGGTGACACACCAGGGCCTGTCAA
GCCCCGTGACCAAAAGCTTCAACAGAGGAGAATGTTGATGA
(SEQ ID NO: 203)
MVLQTQVFISLLLWISGAYGEIVLTQSPGTLSLSPGERATLSCRASQTISSNYLAW
YQQKPGQAPRLLIYGASSRATGIPDRFSGSGSGTDFTLTISRLEPEDFAVYYCQH
YGSSRGWTFGQGTKVEIKRTVAAPSVFIFPPSDEQLKSGTASVVCLLNNFYPREA
KVQWKVDNALQSGNSQESVTEQDSKDSTYSLSNTLTLSKADYEKHKVYACEV
THQGLSSPVTKSFNRGEC**
2130_LC_clin_CDRL1_pVax1 LC CDR1
(SEQ ID NO: 204)
CAGACCATCTTCTATTCCTCTAACAGCAAGAATTAT
(SEQ ID NO: 205)
QTIFYSSNSKNY
2130_LC_clin_CDRL1_pVax1 LC CDR2
(SEQ ID NO: 206)
TGGGCATCT
(SEQ ID NO: 207)
WAS
2130_LC_clin_CDRL1_pVax1 LC CDR3
(SEQ ID NO: 208)
CAGCAGTATTACAGCACCCTGACCTTC
(SEQ ID NO: 209)
QQYYSTLT
2130_LC_clin_CDRL1_pVax1 VL
(SEQ ID NO: 210)
GACATCGTGATGACCCAGTCTCCTGATAGCCTGGCCGTGTCTCTGGGAGAGA
GGGCAACAATCAACTGTAAGTCTAGCCAGACCATCTTCTATTCCTCTAAC
AGCAAGAATTATCTGGCATGGTACCAGCAGAAGCCAGGACAGCCACCCAA
GCTGCTGATGTACTGGGCATCTACCCGGGAGAGCGGAGTGCCTGACCGCTTC
TCTGGCAGCGGCTCCGGAGCAGAGTTTACCCTGACAATCAGCTCCCTGCAGG
CCGAGGATGTGGCCATCTATTACTGCCAGCAGTATTACAGCACCCTGACCTT
CGGCGGCGGCACCAAGGTGGAGATCAAG
(SEQ ID NO: 211)
DIVMTQSPDSLAVSLGERATINCKSSQTIFYSSNSKNYLAWYQQKPGQPPKLL
MYWASTRESGVPDRFSGSGSGAEFTLTISSLQAEDVAIYYCQQYYSTLTFGGGT
KVEIK
2130_LC_clin_CDRL1_pVax1
(SEQ ID NO: 212)
ATGGTGCTGCAGACCCAGGTGTTTATCTCCCTGCTGCTGTGGATCTCTGGCGCCT
ACGGCGACATCGTGATGACCCAGTCTCCTGATAGCCTGGCCGTGTCTCTGGG
AGAGAGGGCAACAATCAACTGTAAGTCTAGCCAGACCATCTTCTATTCCT
CTAACAGCAAGAATTATCTGGCATGGTACCAGCAGAAGCCAGGACAGCCA
CCCAAGCTGCTGATGTACTGGGCATCTACCCGGGAGAGCGGAGTGCCTGAC
CGCTTCTCTGGCAGCGGCTCCGGAGCAGAGTTTACCCTGACAATCAGCTCCC
TGCAGGCCGAGGATGTGGCCATCTATTACTGCCAGCAGTATTACAGCACCCT
GACCTTCGGCGGCGGCACCAAGGTGGAGATCAAGAGGACAGTGGCCGCCCC
TAGCGTGTTCATCTTTCCTCCAAGCGACGAGCAGCTGAAGTCCGGCACCGCC
TCTGTGGTGTGCCTGCTGAACAACTTCTACCCAAGAGAGGCCAAGGTGCAGT
GGAAGGTGGATAACGCCCTGCAGTCCGGCAATTCTCAGGAGAGCGTGACCG
AGCAGGACTCCAAGGATTCTACATACTCTCTGAGCAGCACCCTGACACTGAG
CAAGGCCGACTATGAGAAGCACAAGGTGTACGCCTGCGAAGTGACCCACCA
GGGGCTGAGCAGTCCAGTGACCAAGTCTTTCAATCGGGGAGAATGCTGATA
A
(SEQ ID NO: 213)
MVLQTQVFISLLLWISGAYGDIVMTQSPDSLAVSLGERATINCKSSQTIFYSSNS
KNYLAWYQQKPGQPPKLLMYWASTRESGVPDRFSGSGSGAEFTLTISSLQAEDV
AIYYCQQYYSTLTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVY
ACEVTHQGLSSPVTKSFNRGEC**
2130_LC_clin_lowGerm_pVax1 LC CDR1
(SEQ ID NO: 214)
CAGAGCATCCTGTATTCCTCTAACAATAAGAATTAT
(SEQ ID NO: 215)
QSILYSSNNKNY
2130_LC_clin_lowGerm_pVax1 LC CDR2
(SEQ ID NO: 206)
TGGGCATCT
(SEQ ID NO: 207)
WAS
2130_LC_clin_lowGerm_pVax1 LC CDR3
(SEQ ID NO: 216)
CAGCAGTATTACACCACCCTGACCTTC
(SEQ ID NO: 217)
QQYYTTLT
2130_LC_clin_lowGerm_pVax1 VL
(SEQ ID NO: 218)
GACATCGTGATGACCCAGTCTCCTGATAGCCTGGCCGTGTCTCTGGGAGAGA
GGGCAACAATCAACTGTAAGTCTAGCCAGAGCATCCTGTATTCCTCTAACA
ATAAGAATTATCTGGCATGGTACCAGCAGAAGCCAGGACAGCCACCCAAGC
TGCTGATGTACTGGGCATCTACCCGGGAGAGCGGAGTGCCTGACCGCTTCTC
TGGCAGCGGCTCCGGAGCAGAGTTTACCCTGACAATCAGCAACCTGCAGGC
CGAGGATGTGGCCATCTATTACTGCCAGCAGTATTACACCACCCTGACCTTC
GGCGGCGGCACCAAGGTGGAGATCAAG
(SEQ ID NO: 219)
DIVMTQSPDSLAVSLGERATINCKSSQSILYSSNNKNYLAWYQQKPGQPPKLLM
YWASTRESGVPDRFSGSGSGAEFTLTISNLQAEDVAIYYCQQYYTTLTFGGGTK
VEIK
2130_LC_clin_lowGerm_pVax1
(SEQ ID NO: 220)
ATGGTGCTGCAGACCCAGGTGTTTATCTCCCTGCTGCTGTGGATCTCTGGCGCCT
ACGGC GACATCGTGATGACCCAGTCTCCTGATAGCCTGGCCGTGTCTCTGGG
AGAGAGGGCAACAATCAACTGTAAGTCTAGCCAGAGCATCCTGTATTCCTC
TAACAATAAGAATTATCTGGCATGGTACCAGCAGAAGCCAGGACAGCCACC
CAAGCTGCTGATGTACTGGGCATCTACCCGGGAGAGCGGAGTGCCTGACCG
CTTCTCTGGCAGCGGCTCCGGAGCAGAGTTTACCCTGACAATCAGCAACCT
GCAGGCCGAGGATGTGGCCATCTATTACTGCCAGCAGTATTACACCACCCT
GACCTTCGGCGGCGGCACCAAGGTGGAGATCAAGAGGACAGTGGCCGCCCC
TAGCGTGTTCATCTTTCCTCCAAGCGACGAGCAGCTGAAGTCCGGCACCGCC
TCTGTGGTGTGCCTGCTGAACAACTTCTACCCAAGAGAGGCCAAGGTGCAGT
GGAAGGTGGATAACGCCCTGCAGTCCGGCAATTCTCAGGAGAGCGTGACCG
AGCAGGACTCCAAGGATTCTACATACTCTCTGAGCAGCACCCTGACACTGAG
CAAGGCCGACTATGAGAAGCACAAGGTGTACGCCTGCGAAGTGACCCACCA
GGGGCTGAGCAGTCCAGTGACCAAGTCTTTCAATCGGGGAGAATGCTGATA
A
(SEQ ID NO: 221)
MVLQTQVFISLLLWISGAYGDIVMTQSPDSLAVSLGERATINCKSSQSILYSSNNKN
YLAWYQQKPGQPPKLLMYWASTRESGVPDRESGSGSGAEFTLTISNLQAEDVAI
YYCQQYYTTLTFGGGTKVEIKRTVAAPSVFIFPPSDEQLKSGTASVVCLLNNFYP
REAKVQWKVDNALQSGNSQESVTEQDSKDSTYSLSSTLTLSKADYEKHKVYAC
EVTHQGLSSPVTKSFNRGEC**
2130_LC_clin_FWKS_pVax1 LC CDR1
(SEQ ID NO: 222)
CAGAGCGTGCTGTATTCCTCTAACAATAAGAATTAT
(SEQ ID NO: 223)
QSVLYSSNNKNY
2130_LC_clin_FWKS_pVax1 LC CDR2
(SEQ ID NO: 206)
TGGGCATCT
(SEQ ID NO: 207)
WAS
2130_LC_clin_FWKS_pVax1 LC CDR3
(SEQ ID NO: 208)
CAGCAGTATTACAGCACCCTGACCTTC
(SEQ ID NO: 209)
QQYYSTLT
2130_LC_clin_FWKS_pVax1 VL
(SEQ ID NO: 224)
GACATCGTGATGACCCAGTCTCCTGATAGCCTGGCCGTGTCTCTGGGAGAGA
GGGCAACAATCAACTGTAGGTCTAGCCAGAGCGTGCTGTATTCCTCTAACA
ATAAGAATTATCTGGCATGGTACCAGCAGAAGCCAGGACAGCCACCCAAGG
TGCTGATGTACTGGGCATCTACCCGGGAGAGCGGAGTGCCTGACCGCTTCT
CTGGCAGCGGCTCCGGAGCAGAGTTTACCCTGACAATCAGCAACCTGCAGG
CCGAGGATGTGGCCATCTATTACTGCCAGCAGTATTACAGCACCCTGACCTT
CGGCGGCGGCACCAAGGTGGAGATCAAG
(SEQ ID NO: 225)
DIVMTQSPDSLAVSLGERATINCRSSQSVLYSSNNKNYLAWYQQKPGQPPKVL
MYWASTRESGVPDRFSGSGSGAEFTLTISNLQAEDVAIYYCQQYYSTLTFGGGT
KVEIK
2130_LC_clin_FWKS_pVax1
(SEQ ID NO: 226)
ATGGTGCTGCAGACCCAGGTGTTTATCTCCCTGCTGCTGTGGATCTCTGGCGCCT
ACGGCGACATCGTGATGACCCAGTCTCCTGATAGCCTGGCCGTGTCTCTGGG
AGAGAGGGCAACAATCAACTGTAGGTCTAGCCAGAGCGTGCTGTATTCCTC
TAACAATAAGAATTATCTGGCATGGTACCAGCAGAAGCCAGGACAGCCACC
CAAGGTGCTGATGTACTGGGCATCTACCCGGGAGAGCGGAGTGCCTGACC
GCTTCTCTGGCAGCGGCTCCGGAGCAGAGTTTACCCTGACAATCAGCAACC
TGCAGGCCGAGGATGTGGCCATCTATTACTGCCAGCAGTATTACAGCACCCT
GACCTTCGGCGGCGGCACCAAGGTGGAGATCAAGAGGACAGTGGCCGCCCC
TAGCGTGTTCATCTTTCCTCCAAGCGACGAGCAGCTGAAGTCCGGCACCGCC
TCTGTGGTGTGCCTGCTGAACAACTTCTACCCAAGAGAGGCCAAGGTGCAGT
GGAAGGTGGATAACGCCCTGCAGTCCGGCAATTCTCAGGAGAGCGTGACCG
AGCAGGACTCCAAGGATTCTACATACTCTCTGAGCAGCACCCTGACACTGAG
CAAGGCCGACTATGAGAAGCACAAGGTGTACGCCTGCGAAGTGACCCACCA
GGGGCTGAGCAGTCCAGTGACCAAGTCTTTCAATCGGGGAGAATGCTGATA
A
(SEQ ID NO: 227)
MVLQTQVFISLLLWISGAYGDIVMTQSPDSLAVSLGERATINCRSSQSVLYSSNNK
NYLAWYQQKPGQPPKVLMYWASTRESGVPDRESGSGSGAEFTLTISNLQAEDV
AIYYCQQYYSTLTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVY
ACEVTHQGLSSPVTKSFNRGEC**
2130_LC_clin_HighSet_pVax1 LC CDR1
(SEQ ID NO: 228)
CAGAGCGTGTTCTATTCCTCTAACAATAAGAATTAT
(SEQ ID NO: 229)
QSVFYSSNNKNY
2130_LC_clin_HighSet_pVax1 LC CDR2
(SEQ ID NO: 206)
TGGGCATCT
(SEQ ID NO: 207)
WAS
2130_LC_clin_HighSet_pVax1 LC CDR3
(SEQ ID NO: 216)
CAGCAGTATTACACCACCCTGACCTTC
(SEQ ID NO: 217)
QQYYTTLT
2130_LC_clin_HighSet_pVax1 VL
(SEQ ID NO: 230)
GACATCGTGATGACCCAGTCTCCTGATAGCCTGGCCGTGTCTCTGGGAGAGA
GGGCAACAATCAACTGTAAGTCTAGCCAGAGCGTGTTCTATTCCTCTAACA
ATAAGAATTATCTGGCATGGTACCAGCAGAAGCCAGGACAGCCACCCCGG
GTGCTGATGTACTGGGCATCTACCCGGGAGAGCGGAGTGCCTGACCGCTTC
TCTGGCAGCGGCTCCGGAGCAGAGTTTACCCTGACAATCAGCTCCCTGCAGG
CCGAGGATGTGGCCATCTATTACTGCCAGCAGTATTACACCACCCTGACCT
TCGGCGGCGGCACCAAGGTGGAGATCAAG
(SEQ ID NO: 231)
DIVMTQSPDSLAVSLGERATINCKSSQSVFYSSNNKNYLAWYQQKPGQPPRVL
MYWASTRESGVPDRFSGSGSGAEFTLTISSLQAEDVAIYYCQQYYTTLTFGGGT
KVEIK
2130_LC_clin_HighSet_pVax1
(SEQ ID NO: 232)
ATGGTGCTGCAGACCCAGGTGTTTATCTCCCTGCTGCTGTGGATCTCTGGCGCCT
ACGGCGACATCGTGATGACCCAGTCTCCTGATAGCCTGGCCGTGTCTCTGGG
AGAGAGGGCAACAATCAACTGTAAGTCTAGCCAGAGCGTGTTCTATTCCTC
TAACAATAAGAATTATCTGGCATGGTACCAGCAGAAGCCAGGACAGCCACC
CCGGGTGCTGATGTACTGGGCATCTACCCGGGAGAGCGGAGTGCCTGACC
GCTTCTCTGGCAGCGGCTCCGGAGCAGAGTTTACCCTGACAATCAGCTCCCT
GCAGGCCGAGGATGTGGCCATCTATTACTGCCAGCAGTATTACACCACCCT
GACCTTCGGCGGCGGCACCAAGGTGGAGATCAAGAGGACAGTGGCCGCCCC
TAGCGTGTTCATCTTTCCTCCAAGCGACGAGCAGCTGAAGTCCGGCACCGCC
TCTGTGGTGTGCCTGCTGAACAACTTCTACCCAAGAGAGGCCAAGGTGCAGT
GGAAGGTGGATAACGCCCTGCAGTCCGGCAATTCTCAGGAGAGCGTGACCG
AGCAGGACTCCAAGGATTCTACATACTCTCTGAGCAGCACCCTGACACTGAG
CAAGGCCGACTATGAGAAGCACAAGGTGTACGCCTGCGAAGTGACCCACCA
GGGGCTGAGCAGTCCAGTGACCAAGTCTTTCAATCGGGGAGAATGCTGATA
A
(SEQ ID NO: 233)
MVLQTQVFISLLLWISGAYGDIVMTQSPDSLAVSLGERATINCKSSQSVFYSSNNK
NYLAWYQQKPGQPPRVLMYWASTRESGVPDRFSGSGSGAEFTLTISSLQAEDV
AIYYCQQYYTTLTFGGGTKVEIKRTVAAPSVFIFPPSDEQLKSGTASVVCLLNNF
YPREAKVQWKVDNALQSGNSQESVTEQDSKDSTYSLSSTLTLSKADYEKHKVY
ACEVTHQGLSSPVTKSFNRGEC**
2130_HC (WT)
(SEQ ID NO: 234)
atggactggacctggagaatcctgttcctggtggcagcagcaaccggaac
acacgcacaggtgcagctggtgcagtccggagcagaggtgaagaagcctg
gagcctctgtgaaggtgagctgcaaggcctccggctacacctttacatct
tatggaatcagctgggtgaggcaggcaccaggacagggactggagtggat
gggctggatcagcgcctacaacggcaatacaaactatgcccagaagctgc
agggcagagtgaccatgaccacagacaccagcacatccaccgcctacatg
gagctgaggtctctgagaagcgacgatacagccgtgtactattgcgcccg
ggactatacccgcggcgcctggttcggagagtctctgatcggcggatttg
ataattggggccagggcacactggtgaccgtgtctgccagcacaaaggga
ccaagcgtgttcccactggcacccagctccaagtccacatctggcggcac
cgccgccctgggatgtctggtgaaggattacttcccagagcccgtgaccg
tgtcctggaattctggcgccctgacaagcggcgtgcacacctttccagcc
gtgctgcagtctagcggcctgtactccctgtcctctgtggtgacagtgcc
cagctcctctctgggcacacagacctatatctgcaatgtgaaccacaagc
caagcaacaccaaggtggacaagaaggtggagcccaagtcctgtgataag
acacacacctgccctccctgtcctgcaccagagctgctgggcggcccatc
cgtgttcctgtttccacccaagcctaaggacaccctgatgatctctcgga
cacccgaggtgacctgcgtggtggtggacgtgagccacgaggaccccgag
gtgaagtttaattggtacgtggatggcgtggaggtgcacaacgccaagac
caagcccagggaggagcagtacaactccacatatagagtggtgtctgtgc
tgaccgtgctgcaccaggactggctgaatggcaaggagtataagtgcaag
gtgtccaacaaggccctgcccgcccctatcgagaagacaatctctaaggc
aaagggacagcctcgggagccacaggtgtacaccctgcctccatcccgcg
acgagctgacaaagaatcaggtgtctctgacctgtctggtgaagggcttc
tatccttctgatatcgcagtggagtgggagagcaacggacagccagagaa
caattacaagaccacaccccctgtgctggacagcgatggctccttctttc
tgtatagcaagctgacagtggataagtcccgciggcagcagggcaacgtg
ttcagctgttccgtgatgcacgaggccctgcacaaccactacacccagaa
gtctctgagcctgtcccctggcaag
2130_HC (WT)
(SEQ ID NO: 235)
MDWTWRILFLVAAATGTHAEVQLVESGGGLVKPGGSLRLSCAASGFTFRD
VWMSWVRQAPGKGLEWVGRIKSKIDGGTTDYAAPVKGRFTISRDDSKNTL
YLQMNSLKTEDTAVYYCTTAGSYYYDTVGPGLPEGKFDYWGQGTLVTVSS
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGKRGRKRRSGSGATNFSLLKQA
GDVEENPGPMVLQTQVFISLLLWISGAYGDIVMTQSPDSLAVSLGERATI
NCKSSQSVLYSSNNKNYLAWYQQKPGQPPKLLMYWASTRESGVPDRFSGS
GSGAEFTLTISSLQAEDVAIYYCQQYYSTLTFGGGTKVEIKRTVAAPSVF
IFPPSDEQLKSGTASVVCLLNNFYPREAKVQWKVDNALQSGNSQESVTEQ
DSKDSTYSLSNTLTLSKADYEKHKVYACEVTHQGLSSPVTKSFNRGEC

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. An anti-SARS-CoV-2 antibody or fragment thereof, wherein the antibody comprises at least one of a heavy chain and a light chain selected from the group consisting of:
a heavy chain sequence comprising at least one selected from the group consisting of:
a) a fragment comprising at least the constant heavy chain sequence of the full-length heavy chain sequence selected from the group consisting of: SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88; and
b) a fragment comprising at least the variable heavy chain sequence of the full-length heavy chain sequence selected from the group consisting of: SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, SEQ ID NO:86;
and a light chain sequence comprising at least one selected from the group consisting of:
a′) a fragment comprising at least the constant light chain sequence of the full-length light chain sequence selected from the group consisting of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, and SEQ ID NO:112;
b′) a light chain comprising a set of CDR sequences selected from the group consisting of
b′1) a LCDR1 comprising SEQ ID NO:138, a LCDR2 comprising SEQ ID NO:140 and a LCDR3 comprising SEQ ID NO:142;
b′2) a LCDR1 comprising SEQ ID NO:148, a LCDR2 comprising SEQ ID NO:140 and a LCDR3 comprising SEQ ID NO:142;
b′3) a LCDR1 comprising SEQ ID NO:156, a LCDR2 comprising SEQ ID NO:140 and a LCDR3 comprising SEQ ID NO:142;
b′4) a LCDR1 comprising SEQ ID NO:162, a LCDR2 comprising SEQ ID NO:140 and a LCDR3 comprising SEQ ID NO:142;
b′5) a LCDR1 comprising SEQ ID NO:168, a LCDR2 comprising SEQ ID NO:140 and a LCDR3 comprising SEQ ID NO:142;
b′6) a LCDR1 comprising SEQ ID NO:138, a LCDR2 comprising SEQ ID NO:140 and a LCDR3 comprising SEQ ID NO:183;
b′7) a LCDR1 comprising SEQ ID NO:189, a LCDR2 comprising SEQ ID NO:140 and a LCDR3 comprising SEQ ID NO:142;
b′8) a LCDR1 comprising SEQ ID NO:198, a LCDR2 comprising SEQ ID NO:140 and a LCDR3 comprising SEQ ID NO:142;
b′9) a LCDR1 comprising SEQ ID NO:205, a LCDR2 comprising SEQ ID NO:207 and a LCDR3 comprising SEQ ID NO:209;
b′10) a LCDR1 comprising SEQ ID NO:215, a LCDR2 comprising SEQ ID NO:207 and a LCDR3 comprising SEQ ID NO:217;
b′11) a LCDR1 comprising SEQ ID NO:223, a LCDR2 comprising SEQ ID NO:207 and a LCDR3 comprising SEQ ID NO:209;
b′12) a LCDR1 comprising SEQ ID NO:229, a LCDR2 comprising SEQ ID NO:207 and a LCDR3 comprising SEQ ID NO:217;
c′ a light chain comprising a variable region selected from the group consisting of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, SEQ ID NO:201, SEQ ID NO:211, SEQ ID NO:219, SEQ ID NO:225 and SEQ ID NO:231; and
d′) a fragment comprising at least the variable light chain sequence of the full-length light chain sequence selected from the group consisting of: SEQ ID NO:22, SEQ ID NO:118, SEQ ID NO:120, SEQ ID NO:122, SEQ ID NO:124, SEQ ID NO:126, SEQ ID NO:128, SEQ ID NO:130, and SEQ ID NO:132.

2. The anti-SARS-CoV-2 antibody or fragment thereof of claim 1, comprising a heavy chain amino acid sequence comprising at least one selected from the group consisting of:
a) a fragment comprising at least the constant heavy chain sequence of the full-length heavy chain sequence selected from the group consisting of: SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, SEQ ID NO:88;
b) a fragment comprising at least the variable heavy chain sequence of the full-length heavy chain sequence selected from the group consisting of: SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:64, SEQ ID NO:74, SEQ ID NO:84, SEQ ID NO:86; and
and a light chain amino acid sequence comprising at least one selected from the group consisting of:
a′) a fragment comprising at least the constant light chain sequence of the full-length light chain sequence selected from the group consisting of SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, and SEQ ID NO:112;
b′) a light chain comprising a set of CDR sequences selected from the group consisting of
b′1) a LCDR1 comprising SEQ ID NO:138, a LCDR2 comprising SEQ ID NO:140 and a LCDR3 comprising SEQ ID NO:142;
b′2) a LCDR1 comprising SEQ ID NO:148, a LCDR2 comprising SEQ ID NO:140 and a LCDR3 comprising SEQ ID NO:142;
b′3) a LCDR1 comprising SEQ ID NO:156, a LCDR2 comprising SEQ ID NO:140 and a LCDR3 comprising SEQ ID NO:142;
b′4) a LCDR1 comprising SEQ ID NO:162, a LCDR2 comprising SEQ ID NO:140 and a LCDR3 comprising SEQ ID NO:142;
b′5) a LCDR1 comprising SEQ ID NO:168, a LCDR2 comprising SEQ ID NO:140 and a LCDR3 comprising SEQ ID NO:142;
b′6) a LCDR1 comprising SEQ ID NO:138, a LCDR2 comprising SEQ ID NO:140 and a LCDR3 comprising SEQ ID NO:183;
b′7) a LCDR1 comprising SEQ ID NO:189, a LCDR2 comprising SEQ ID NO:140 and a LCDR3 comprising SEQ ID NO:142;
b′8) a LCDR1 comprising SEQ ID NO:198, a LCDR2 comprising SEQ ID NO:140 and a LCDR3 comprising SEQ ID NO:142;
c′) a light chain comprising a variable region selected from the group consisting of SEQ ID NO:144, SEQ ID NO:152, SEQ ID NO:158, SEQ ID NO:164, SEQ ID NO:170, SEQ ID NO:175, SEQ ID NO:179, SEQ ID NO:185, SEQ ID NO:191, SEQ ID NO:195, and SEQ ID NO:201; and
d′) a fragment comprising at least the variable light chain sequence of the full-length light chain sequence selected from the group consisting of: SEQ ID NO:22, SEQ ID NO:118, SEQ ID NO:120, SEQ ID NO:122, SEQ ID NO:124, SEQ ID NO:126, SEQ ID NO:128, SEQ ID NO:130, and SEQ ID NO:132.

3. The anti-SARS-CoV-2 antibody or fragment thereof of claim 2, comprising
a) a heavy chain amino acid sequence comprising at least one selected from the group consisting of: SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, and SEQ ID NO:88;
b) a light chain amino acid sequence comprising at least one selected from the group consisting of: SEQ ID NO:22, SEQ ID NO:36, SEQ ID NO:38, SEQ ID NO:40, SEQ ID NO:42, SEQ ID NO:44, SEQ ID NO:90, SEQ ID NO:92, SEQ ID NO:94, SEQ ID NO:96, SEQ ID NO:98, SEQ ID NO:100, SEQ ID NO:102, SEQ ID NO:104; SEQ ID NO:106, SEQ ID NO:108, SEQ ID NO:110, SEQ ID NO:112; SEQ ID NO:146, SEQ ID NO:154, SEQ ID NO:160, SEQ ID NO:166, SEQ ID NO:172, SEQ ID NO:177, SEQ ID NO:181, SEQ ID NO:187, SEQ ID NO:193, SEQ ID NO:197, and SEQ ID NO:203.

4. The anti-SARS-CoV-2 antibody or fragment thereof of claim 1, wherein the antibody comprises a heavy chain sequence comprising SEQ ID NO:235;
and a light chain sequence comprising a set of CDR sequences selected from the group consisting of
a) a LCDR1 comprising SEQ ID NO:205, a LCDR2 comprising SEQ ID NO:207 and a LCDR3 comprising SEQ ID NO:209;
b) a LCDR1 comprising SEQ ID NO:215, a LCDR2 comprising SEQ ID NO:207 and a LCDR3 comprising SEQ ID NO:217;
c) a LCDR1 comprising SEQ ID NO:223, a LCDR2 comprising SEQ ID NO:207 and a LCDR3 comprising SEQ ID NO:209;
d) a LCDR1 comprising SEQ ID NO:229, a LCDR2 comprising SEQ ID NO:207 and a LCDR3 comprising SEQ ID NO:217; and
e) a light chain comprising a variable region selected from the group consisting of SEQ ID NO:211, SEQ ID NO:219, SEQ ID NO:225 and SEQ ID NO:231.

5. The anti-SARS-CoV-2 antibody or fragment thereof of claim 4, wherein the antibody comprises a heavy chain sequence comprising SEQ ID NO:235; and a light chain sequence selected from the group consisting of: SEQ ID NO:213, SEQ ID NO:221, SEQ ID NO:227, and SEQ ID NO:233.

6. The anti-SARS-CoV-2 antibody or fragment thereof of any one of claims 1-5, wherein the antibody is selected from the group consisting of a humanized antibody, a chimeric antibody, a fully human antibody, an antibody mimetic.

7. A nucleic acid molecule, or combination of nucleic acid molecules, comprising at least one nucleotide sequence encoding at least one of a heavy chain or a light chain of an anti-SARS-CoV-2 antibody, or fragment thereof, of any one of claims 1-6.

8. The nucleic acid molecule of claim 7, wherein at least one nucleotide sequence is selected from the group consisting of:
a nucleotide sequence encoding a heavy chain sequence comprising at least one selected from the group consisting of:
a) a fragment encoding at least the constant heavy chain sequence of the full-length heavy chain sequence selected from the group consisting of: SEQ ID NO:3, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, and SEQ ID NO:87; and
b) a fragment encoding at least the variable heavy chain sequence of the full-length heavy chain sequence selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:63, SEQ ID NO:75, SEQ ID NO:83, SEQ ID NO:85;
and a light chain sequence comprising at least one selected from the group consisting of:
a′) a fragment encoding at least the constant light chain sequence of the full-length light chain sequence selected from the group consisting of SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, and SEQ ID NO:111;
b′) a light chain comprising a set of CDR encoding sequences selected from the group consisting of
b′1) a LCDR1 encoding sequence comprising SEQ ID NO:137, a LCDR2 encoding sequence comprising SEQ ID NO:139 and a LCDR3 encoding sequence comprising SEQ ID NO:141;
b′2) a LCDR1 encoding sequence comprising SEQ ID NO:147, a LCDR2 encoding sequence comprising SEQ ID NO:139 and a LCDR3 encoding sequence comprising SEQ ID NO:141;
b′3) a LCDR1 encoding sequence comprising SEQ ID NO:155, a LCDR2 encoding sequence comprising SEQ ID NO:149 and a LCDR3 encoding sequence comprising SEQ ID NO:150;
b′4) a LCDR1 encoding sequence comprising SEQ ID NO:161, a LCDR2 encoding sequence comprising SEQ ID NO:149 and a LCDR3 encoding sequence comprising SEQ ID NO:150;
b′5) a LCDR1 encoding sequence comprising SEQ ID NO:167, a LCDR2 encoding sequence comprising SEQ ID NO:149 and a LCDR3 encoding sequence comprising SEQ ID NO:150;
b′6) a LCDR1 encoding sequence comprising SEQ ID NO:173, a LCDR2 encoding sequence comprising SEQ ID NO:149 and a LCDR3 encoding sequence comprising SEQ ID NO:150;
b′7) a LCDR1 encoding sequence comprising SEQ ID NO:173, a LCDR2 encoding sequence comprising SEQ ID NO:149 and a LCDR3 encoding sequence comprising SEQ ID NO:182;
b′8) a LCDR1 encoding sequence comprising SEQ ID NO:188, a LCDR2 encoding sequence comprising SEQ ID NO:149 and a LCDR3 encoding sequence comprising SEQ ID NO:150;
b′9) a LCDR1 encoding sequence comprising SEQ ID NO:198, a LCDR2 encoding sequence comprising SEQ ID NO:149 and a LCDR3 encoding sequence comprising SEQ ID NO:150;
b′10) a LCDR1 encoding sequence comprising SEQ ID NO:204, a LCDR2 encoding sequence comprising SEQ ID NO:206 and a LCDR3 encoding sequence comprising SEQ ID NO:208;
b′11) a LCDR1 encoding sequence comprising SEQ ID NO:214, a LCDR2 encoding sequence comprising SEQ ID NO:206 and a LCDR3 encoding sequence comprising SEQ ID NO:216;
b′12) a LCDR1 encoding sequence comprising SEQ ID NO:222, a LCDR2 encoding sequence comprising SEQ ID NO:206 and a LCDR3 encoding sequence comprising SEQ ID NO:208;
b′13) a LCDR1 encoding sequence comprising SEQ ID NO:228, a LCDR2 encoding sequence comprising SEQ ID NO:206 and a LCDR3 encoding sequence comprising SEQ ID NO:216;
c′) a light chain encoding a variable region selected from the group consisting of SEQ ID NO:143, SEQ ID NO:151, SEQ ID NO:159, SEQ ID NO:163, SEQ ID NO:169, SEQ ID NO:174, SEQ ID NO:178, SEQ ID NO:184, SEQ ID NO:190, SEQ ID NO:194, SEQ ID NO:200, SEQ ID NO:210, SEQ ID NO:218, SEQ ID NO:224 and SEQ ID NO:230; and
d′) a fragment encoding at least the variable light chain sequence of the full-length light chain sequence selected from the group consisting of: SEQ ID NO:21, SEQ ID NO:117, SEQ ID NO:119, SEQ ID NO:121, SEQ ID NO:123, SEQ ID NO:125, SEQ ID NO:127, SEQ ID NO:129, and SEQ ID NO:131.

9. The nucleic acid molecule of claim 8, wherein the nucleotide sequence encoding the anti-SARS-CoV-2 antibody or fragment thereof comprises at least one selected from:
a) a nucleotide sequence encoding a heavy chain amino acid sequence selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, and SEQ ID NO:87; and
b) a nucleotide sequence encoding a light chain amino acid sequence selected from the group consisting of: SEQ ID NO:21, SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, SEQ ID NO:111, SEQ ID NO:145, SEQ ID NO:153, SEQ ID NO:159, SEQ ID NO:165, SEQ ID NO:171, SEQ ID NO:176, SEQ ID NO:180, SEQ ID NO:186, SEQ ID NO:192, SEQ ID NO:196, and SEQ ID NO:202.

10. The combination of nucleic acid molecules of claim 8, comprising:
a) a first nucleic acid molecule comprising a nucleotide sequence encoding a heavy chain amino acid sequence of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, and SEQ ID NO:87; and
b) a second nucleic acid molecule comprising a nucleotide sequence encoding a light chain amino acid sequence selected from the group consisting of: SEQ ID NO:21, SEQ ID NO:35, SEQ ID NO:37, SEQ ID NO:39, SEQ ID NO:41, SEQ ID NO:43, SEQ ID NO:89, SEQ ID NO:91, SEQ ID NO:93, SEQ ID NO:95, SEQ ID NO:97, SEQ ID NO:99, SEQ ID NO:101, SEQ ID NO:103; SEQ ID NO:105, SEQ ID NO:107, SEQ ID NO:109, SEQ ID NO:111, SEQ ID NO:145, SEQ ID NO:153, SEQ ID NO:159, SEQ ID NO:165, SEQ ID NO:171, SEQ ID NO:176, SEQ ID NO:180, SEQ ID NO:186, SEQ ID NO:192, SEQ ID NO:196, and SEQ ID NO:202.

11. The nucleic acid molecule of claim 8, wherein the nucleotide sequence encoding the anti-SARS-CoV-2 antibody or fragment thereof comprises at least one selected from:
a) a nucleotide sequence encoding a heavy chain amino acid of: SEQ ID NO:235; and
b) a nucleotide sequence encoding a light chain amino acid sequence selected from the group consisting of: SEQ ID NO:212, SEQ ID NO:220, SEQ ID NO:226, and SEQ ID NO:232.

12. The combination of nucleic acid molecules of claim 8, comprising:
a) a first nucleic acid molecule comprising a nucleotide sequence encoding SEQ ID NO:234; and
b) a first nucleic acid molecule comprising a nucleotide sequence encoding a light chain amino acid sequence selected from the group consisting of: SEQ ID NO:212, SEQ ID NO:220, SEQ ID NO:226, and SEQ ID NO:232.

13. The nucleic acid molecule of any one of claims 8-12, wherein the nucleotide sequence encodes a leader sequence.

14. The nucleic acid molecule of any one of claims 8-12, wherein the nucleic acid molecule comprises an expression vector.

15. A composition comprising at least one anti-SARS-CoV-2 antibody or fragment thereof of any one of claims 1-7.

16. A composition comprising at least one nucleic acid molecule of any one of claims 8-12.

17. A composition comprising a combination of nucleic acid molecules of any one of claims 10-14.

18. The composition of any one of claim 16 or 17, further comprising a pharmaceutically acceptable excipient.

19. A method of preventing or treating a disease in a subject, the method comprising administering to the subject the antibody or antibody fragment of any one of claims 1-7, the nucleic acid molecule of any one of claims 8-14, or a composition of any one of claims 15-16.

20. The method of claim 19, wherein the disease is COVID-19.

21. A glycan-modified binding molecule, or fragment thereof.

22. The glycan-modified binding molecule of claim 21, or heavy chain fragment thereof, comprising at least one glycan-modification in the CH region corresponding to the CH region modifications as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88.

23. The glycan-modified binding molecule of claim 21, or light chain fragment thereof, comprising at least one glycan-modification in the CL region corresponding to the CL region modifications as set forth in SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

24. The glycan-modified binding molecule of claim 21, or heavy chain fragment thereof, comprising a heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88.

25. The glycan-modified binding molecule of claim 21, or light chain fragment thereof, comprising a light chain as set forth in SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112.

26. A composition comprising at least one glycan-modified binding molecule, or fragment thereof of any one of claims 21 to 25.

27. The composition of claim 26, wherein the glycan-modified binding molecule is incorporated into a nanoparticle.

28. The composition of claim 26, further comprising a pharmaceutically acceptable excipient.

29. The composition of claim 26, further comprising an adjuvant.

30. A nucleic acid molecule, or combination of nucleic acid molecules, encoding a glycan-modified binding molecule of claim 21, or fragment thereof.

31. The nucleic acid molecule of claim 30, wherein the nucleic acid molecule comprises a sequence encoding a heavy chain selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87.

32. The nucleic acid molecule of claim 30, wherein the nucleic acid molecule comprises a sequence encoding a light chain selected from the group consisting of: SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

33. The combination of nucleic acid molecules of claim 30, comprising a first nucleic acid molecule comprising a sequence encoding a heavy chain selected from the group consisting of: SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:23, SEQ ID NO:25, SEQ ID NO:27, SEQ ID NO:29, SEQ ID NO:31, SEQ ID NO:47, SEQ ID NO:57, SEQ ID NO:59, SEQ ID NO:61, SEQ ID NO:63, SEQ ID NO:65, SEQ ID NO:67, SEQ ID NO:69, SEQ ID NO:71, SEQ ID NO:73, SEQ ID NO:75, SEQ ID NO:77, SEQ ID NO:79, SEQ ID NO:81, SEQ ID NO:83, SEQ ID NO:85, or SEQ ID NO:87, and a second nucleic acid molecule comprising a sequence encoding a light chain selected from the group consisting of: SEQ ID NO:35, SEQ ID NO:37, SEQ ID NO:39, SEQ ID NO:41, SEQ ID NO:43, SEQ ID NO:51, SEQ ID NO:53, SEQ ID NO:89, SEQ ID NO:91, SEQ ID NO:93, SEQ ID NO:95, SEQ ID NO:97, SEQ ID NO:99, SEQ ID NO:101, SEQ ID NO:103, SEQ ID NO:105, SEQ ID NO:107, SEQ ID NO:109, or SEQ ID NO:111.

34. A method of treating a disease or disorder in a subject in need thereof, the method comprising administering a glycan-modified binding molecule of any one of claims 21 to 25, a composition of any one of claims 26 to 29, or a nucleic acid molecule or combination thereof of any one of claims 30-33 to the subject.

35. A method of protecting a subject in need thereof from COVID-19, the method comprising administering a glycan-modified binding molecule, or heavy chain or light chain thereof, of any one of claims 21 to 25 or a composition of any one of claims 26 to 29, or a nucleic acid molecule or combination thereof of any one of claims 30-33 to the subject.

36. The method of claim 35, wherein the method comprises administering at least one molecule selected from:
a) a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88;
b) a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112; or
c) a combination of a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:102, SEQ ID NO:104, SEQ ID NO:106, SEQ ID NO:108, SEQ ID NO:110, or SEQ ID NO:112.

37. A method of treating a subject in need thereof against SARS-CoV-2, the method comprising administering a glycan-modified binding molecule, or heavy chain or light chain thereof, of any one of claims 21 to 25 or a composition of any one of claims 26 to 29, or a nucleic acid molecule or combination thereof of any one of claims 30-33 to the subject.

38. The method of claim 37, wherein the method comprises administering at least one molecule selected from:
a) a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88;
b) a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:92, SEQ ID NO:94, SEQ ID NO:96, SEQ ID NO:98, SEQ ID NO:100, SEQ ID NO:102, SEQ ID NO:104, SEQ ID NO:106, SEQ ID NO:108, SEQ ID NO:110, or SEQ ID NO:112; or
c) a combination of a glycan-modified heavy chain as set forth in SEQ ID NO:4, SEQ ID NO:10, SEQ ID NO:12, SEQ ID NO:14, SEQ ID NO:16, SEQ ID NO:18, SEQ ID NO:24, SEQ ID NO:26, SEQ ID NO:28, SEQ ID NO:30, SEQ ID NO:32, SEQ ID NO:48, SEQ ID NO:58, SEQ ID NO:60, SEQ ID NO:62, SEQ ID NO:64, SEQ ID NO:66, SEQ ID NO:68, SEQ ID NO:70, SEQ ID NO:72, SEQ ID NO:74, SEQ ID NO:76, SEQ ID NO:78, SEQ ID NO:80, SEQ ID NO:82, SEQ ID NO:84, SEQ ID NO:86, or SEQ ID NO:88, and a glycan-modified light chain as set forth in SEQ ID NO:22, SEQ ID NO:36, SEQ ID NO:38, SEQ ID NO:40, SEQ ID NO:42, SEQ ID NO:44, SEQ ID NO:52, SEQ ID NO:53, SEQ ID NO:90, SEQ ID NO:102, SEQ ID NO:104, SEQ ID NO:106, SEQ ID NO:108, SEQ ID NO:110, or SEQ ID NO:112.

39. A method of protecting a subject in need thereof from a disease or disorder associated with HIV infection, the method comprising administering a glycan-modified binding molecule, or heavy chain or light chain thereof, of any one of claims 21 to 25, a composition of any one of claims 26 to 29, or a nucleic acid molecule or combination thereof of any one of claims 30-33, to the subject.

40. The method of claim 39, wherein the method comprises administering at least one molecule selected from:
a) a glycan-modified heavy chain as set forth in SEQ ID NO:48;
b) a glycan-modified light chain as set forth in SEQ ID NO:52 or SEQ ID NO:54; or
c) a combination of a glycan-modified heavy chain as set forth in SEQ ID NO:48, and a glycan-modified light chain as set forth in SEQ ID NO:52 or SEQ ID NO:54.

41. A method of treating a subject in need thereof against a disease or disorder associated with HIV infection, the method comprising administering a glycan-modified binding molecule, or heavy chain or light chain thereof, of any one of claims 21 to 25, a composition of any one of claims 26 to 29, or a nucleic acid molecule or combination thereof of any one of claims 30-33 to the subject.

42. The method of claim 41, wherein the method comprises administering at least one molecule selected from:
a) a glycan-modified heavy chain as set forth in SEQ ID NO:48;
b) a glycan-modified light chain as set forth in SEQ ID NO:52 or SEQ ID NO:54; or
c) a combination of a glycan-modified heavy chain as set forth in SEQ ID NO:48, and a glycan-modified light chain as set forth in SEQ ID NO:52 or SEQ ID NO:54.
